(19) United States
(12) Patent Application Publication
(10) Pub. No.: US 2022/0202818 A1
(43) Pub. Date: Jun. 30, 2022

(54) COMBINATION WITH CHECKPOINT INHIBITORS TO TREAT CANCER

(71) Applicant: The Regents of the University of Michigan, Ann Arbor, MI (US)

(72) Inventors: Christopher E. Whitehead, Ypsilanti, MI (US); Judith S. Leopold, Ann Arbor, MI (US); Elizabeth Ziemke, Ann Arbor, MI (US)

(21) Appl. No.: 17/604,226

(22) PCT Filed: Apr. 18, 2020
(86) PCT No.: PCT/US2020/028882

§ 371 Date: Oct. 15, 2021

Related U.S. Application Data
(60) Provisional application No. 62/835,903, filed on Apr. 18, 2019.

(51) Int. Cl. A61K 31/517; A61K 39/395; A61P 35/00

(52) U.S. Cl. CPC A61K 31/517; A61P 35/00; A61K 39/3955

(57) ABSTRACT

A combination comprising a compound of Formula (I) and/or Formula (Ia), or a pharmaceutically acceptable salt thereof, and at least one immune checkpoint modulator. Methods for the prevention and treatment of a cancer comprises administering to a subject in need thereof, a therapeutically effective amount of a combination, the combination comprising: a compound of Formula (I) and/or Formula (Ia), or a pharmaceutically acceptable salt thereof, and at least one immune checkpoint modulator.

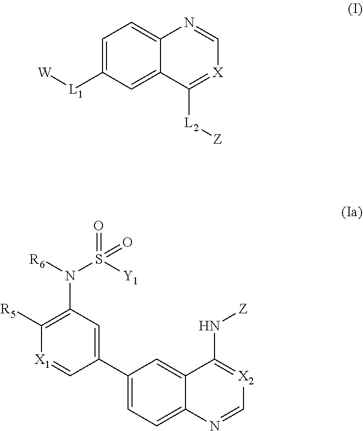

### CROSS REFERENCE TO RELATED APPLICATIONS

This application claims benefit of priority to U.S. Provisional Application No. 62/835,903, filed Apr. 18, 2019, the contents of which are incorporated herein by reference in their entirety.

### TECHNICAL FIELD

The present disclosure is in the field of oncology treatments, including for example, a combination of one, a compound that is a dual inhibitor of EGFR proteins and PI3K proteins, and two, an immune checkpoint inhibitor.

### BACKGROUND

In humans with advanced cancer, anti-tumor immunity is often ineffective due to the tightly regulated interplay of pro- and anti-inflammatory, immune-stimulatory and immunosuppressive signals. For example, loss of the anti-inflammatory signals leads to chronic inflammation and prolonged proliferative signaling. Interestingly, cytokines that both promote and suppress proliferation of the tumor cells are produced at the tumor site. It is the imbalance between the effects of these various processes that results in tumor promotion.

To date, a major barrier to attempts to develop effective immunotherapy for cancer has been an inability to break immunosuppression at the cancer site and restore normal networks of immune reactivity. The physiological approach of immunotherapy is to normalize the immune reactivity so that, for example, the endogenous tumor antigens would be recognized and effective cytolytic responses would be developed against tumor cells. Although it was once unclear if tumor immunosurveillance existed, it is now believed that the immune system constantly monitors and eliminates newly transformed cells. Accordingly, cancer cells may alter their phenotype in response to immune pressure in order to escape attack (immunoediting) and upregulate expression of inhibitory signals. Through immunoediting and other subversive processes, primary tumor and metastasis maintain their own survival.

One of the major mechanisms of anti-tumor immunity subversion is known as ‘T-cell exhaustion’, which results from chronic exposure to antigens and is characterized by the up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA, CD272), T cell Immunoglobulin and Mucin domain-3 (Tim-3), Lymphocyte Activation Gene-3 (Lag-3, OD223), and others are often referred to as checkpoint regulators. They act as molecular “tollbooths,” which allow extracellular information to dictate whether cell cycle progression and other intracellular signaling processes should proceed.

In addition to specific antigen recognition through the TCR, T-cell activation is regulated through a balance of positive and negative signals provided by co-stimulatory receptors. These surface proteins are typically members of either the TNF receptor or B7 superfamilies. Agonistic antibodies directed against activating co-stimulatory molecules and blocking antibodies against negative co-stimulatory molecules may enhance T-cell stimulation to promote tumor destruction.

Tumor cells evade host immune recognition by immune checkpoints utilizing the programmed death-1 (PD-1)/programmed death-ligand 1 (PD-L1) pathway to silence the immune system. PD-L1 is highly expressed on tumor-infiltrating lymphocytes as well as on the surface of many human solid tumors. The interaction of PD-1 and PD-L1 leads to reduction of PTEN activity and SHP2-mediated activation of the PI3K/AKT/mTOR pathway. mTOR inhibitors have been reported to increase antitumor activity in response to PD-1 blockade in a variety of solid tumors, including non-small cell lung cancer, gastric cancer, colorectal cancer, renal cancer, urinary bladder cancer, prostate cancer, breast cancer, head and neck squamous cell carcinoma and hepatocellular tumors.

Accordingly, the present disclosure provides a combination therapy for treating cancer comprising a compound of Formula I and/or Formula Ia and blockade of checkpoint inhibitors with the potential to elicit potent and durable immune responses.

### SUMMARY

The present disclosure provides an effective method for treating and/or preventing cancer and/or the establishment of metastases by administering a therapeutically effective combination comprising a compound of Formula I and/or Formula Ia or a pharmaceutically acceptable salt thereof and a checkpoint inhibitor.

In a first aspect of the disclosure, there is a compound of Formula I and/or Formula Ia or a pharmaceutically acceptable salt thereof, for use in the prevention, treatment, reduction, inhibition or control of a neoplastic disease and/or metastases in a patient intended to undergo checkpoint inhibition therapy simultaneously, separately or sequentially with administration of the compound of Formula I and/or Formula Ia or a pharmaceutically acceptable salt thereof.

In a second aspect of the disclosure, there is a method of preventing, treating, reducing, inhibiting or controlling a neoplasia, tumor or cancer and/or the establishment of metastases in a subject, wherein said method comprises simultaneously, separately or sequentially administering to the subject, (i) one or more checkpoint inhibitors, and (ii) a compound of Formula I and/or Formula Ia or a pharmaceutically acceptable salt thereof, wherein said method results in enhanced therapeutic efficacy relative to administration of the checkpoint inhibitor or a compound of Formula I and/or Formula Ia or a pharmaceutically acceptable salt thereof alone.

In various embodiments, the present disclosure provides a combination comprising a compound of Formula I and/or Formula Ia or a pharmaceutically acceptable salt thereof, or a pharmaceutically acceptable salt thereof, and a checkpoint modulator, for example, an immune checkpoint inhibitor. For example, a compound of Formula I includes a compound represented by the Formula I.

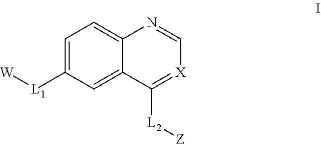

wherein X is N or C—R₄;

L₁ and L₂ are each independently a bond or a C₁-C₆ branched or straight alkylene group, wherein up to three carbon units of said alkylene group are optionally and independently replaced with a bivalent moiety selected from the group consisting of —CO—, —CS—, —CONR—, —CONRNR—, —CO₂—, —OCO—, —NRCO₂—, —O—, —CR═CR—, —C≡C—, —NRCONR—, —OCONR—, —NRNR—, —NRCO—, —S—, —S(O)—, —S(O)₂—, —NR—, —S(O)₂NR—, —NRS(O)₂—, and —NRS(O)₂NR—;

W is selected from the group consisting of halo, 5-10 membered heteroaryl, 5-10 membered heterocyclyl, C₃-C₁₀ carbocyclyl, naphthyl, and phenyl, wherein W is optionally substituted with up to three R₁ substituents;

Z is selected from the group consisting of 5-10 membered heteroaryl, 5-10 membered heterocyclyl, C₃-C₁₀ carbocyclyl, aryl, benzyl, and phenyl, wherein Z is optionally substituted with up to three R₃ substituents;

R₁ is selected from the group consisting of halo, CN, C₁-C₆ alkyl, phenyl, 5-6 membered heteroaryl, 5-6 membered heterocyclyl, C₃-C₆ carbocyclyl, —OR, —CONR₂, —CONRNR₂, —CO₂R, —S(O)₂R, —NR₂, —NRS(O)₂R, —S(O)₂NR₂, and —NRCONR₂, wherein R₁ is optionally substituted with up to two R₂ substituents.

R₂ is selected from the group consisting of halo, C₁-C₆ alkyl, C₁-C₆ alkoxy, phenyl, 5-6 membered heteroaryl, 5-6 membered heterocyclyl, C₃-C₆ carbocyclyl, —OH, oxo, —NR₂, wherein each R₂ is optionally and independently substituted with 5-6 membered heterocyclyl;

R₃ is selected from the group consisting of R, halo, —OR, —O(CH₂)ₙR, and —(CH₂)ₙOR;

R₄ is selected from the group consisting of H, halo, C₁-C₄ alkyl, CN, OH, and —COOH;

R is selected from the group consisting of H, C₁-C₄ alkyl, C₂-C₄ alkenyl, C₂-C₄ alkynyl, phenyl, 5-6 membered heteroaryl, 5-6 membered heterocyclyl, C₃-C₆ carbocyclyl, alkylsulfonyl, and —CONH(C₁-C₄ alkyl); n is 1, 2, or 3; and

provided that the compound of Formula I is not

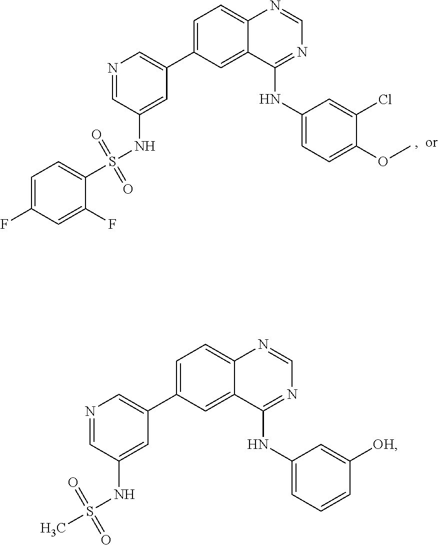

or a pharmaceutically acceptable salt thereof.

In a related aspect, the present disclosure provides a combination of a compound of Formula I and a checkpoint modulator, wherein the compound of Formula I is a compound of Formula Ia or a pharmaceutically acceptable salt thereof. In various embodiments of this aspect, the combination includes: (a). a compound of Formula Ia, or a pharmaceutically acceptable salt thereof,

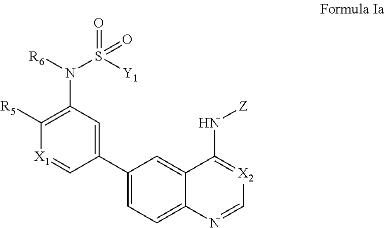

wherein

X₁ is N or CH;

X₂ is N or C—CN;

Z is selected from the group consisting of 5-6 membered heteroaryl and phenyl, wherein Z is optionally substituted with up to three R₃ substituents;

R₅ is H, OH, CN, NH₂, NO₂, O(C₁-C₄ alkyl), C₁-C₄ alkyl, C₂-C₄ alkenyl, C₂-C₄ alkynyl, 5-6 membered heteroaryl, 5-6 membered heterocyclyl, and C₃-C₆ carbocyclyl;

R₆ is H, C₁-C₄ alkyl, or —S(O)₂(C₁-C₄ alkyl);

Y₁ is selected from the group consisting of H, OH, O(C₁-C₄ alkyl), C₁-C₄ alkyl, C₂-C₄ alkyl(R₇), C₂-C₄ alkenyl, C₂-C₄ alkynyl, 5-6 membered heteroaryl, 5-6 membered heterocyclyl, and C₃-C₆ carbocyclyl, wherein Y₁ is optionally substituted with up to two instances of 5-6 membered heterocyclyl, 5-6 membered carbocyclyl, O(C₁-C₄ alkyl), C₁-C₄ alkyl, OH, CN, halo, NO₂, and NH₂; and

R₇ is selected from NH₂, N(H)(C₁-C₄ alkyl), N(C₁-C₄ alkyl)₂, 3-7 membered heterocyclyl; provided that the compound of Formula Ia is not

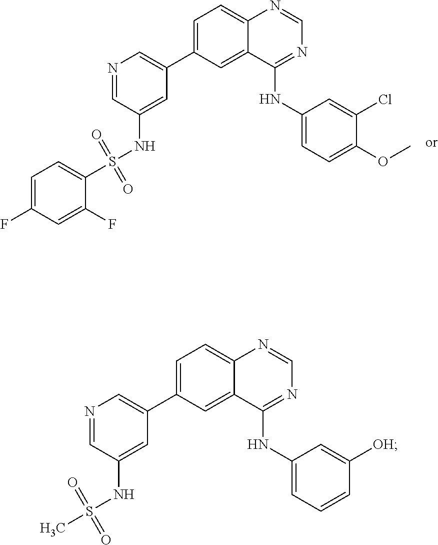

and,

(b). an immune checkpoint modulator, wherein the immune checkpoint inhibitor is an antibody or an antigen binding fragment thereof, that binds to at least one of the following targets: PD-1, PD-L1, PD-L2, CEACAM (e.g., CEACAM-1, -3 and/or -5), CTLA-4, TIM-3, LAG-3, VISTA, BTLA, TIGIT, LAIR1, CD160, 2B4, TGF beta, OX40, 41BB, LIGHT, CD40, GITR, TGF-beta, TIM-3, SIRP-alpha, VSIG8, BTLA, SIGLEC7, SIGLEC9, ICOS, B7H3, B7H4, FAS, or BTNL2, preferably, wherein the checkpoint modulator is an immune checkpoint inhibitor that binds to PD-1, PD-L1, PD-L2, CTLA-4, or combinations thereof, and inhibits the activity of these checkpoint molecules.

In related embodiments, the compound of Formula I and/or Ia or a pharmaceutically acceptable salt thereof, is administered in combination with a checkpoint modulator, for example a checkpoint modulator that modulates the activity of: PD-1, PD-L1, PD-L2, CEACAM (e.g., CEACAM-1, -3 and/or -5), CTLA-4, TIM-3, LAG-3, VISTA, BTLA, TIGIT, LAIR1, CD160, 2B4, TGF beta, OX40, 41BB, LIGHT, CD40, GITR, TGF-beta, TIM-3, SIRP-alpha, VSIG8, BTLA, SIGLEC7, SIGLEC9, ICOS, B7H3, B7H4, FAS, or BTNL2.

In a related embodiment, the checkpoint modulator is an immune checkpoint inhibitor of: PD-1, PD-L1, PD-L2, CEACAM (e.g., CEACAM-1, -3 and/or -5), CTLA-4, TIM-3, LAG-3, VISTA, BTLA, TIGIT, LAIR1, CD160, 2B4, TGF beta, or a combination thereof.

In a related embodiment, the checkpoint modulator is an immune checkpoint inhibitor that binds to PD-1, PD-L1, PD-L2, CTLA-4, or combinations thereof.

In a third aspect of the disclosure, there is a method of preventing, treating, reducing, inhibiting or controlling a neoplasia, tumor or cancer and/or the establishment of metastases in a subject, wherein said method comprises simultaneously, separately or sequentially administering to the subject, (i) a sub-therapeutic amount and/or duration of one or more checkpoint inhibitors, and (ii) a compound of Formula I and/or Formula Ia or a pharmaceutically acceptable salt thereof, wherein said method results in enhanced therapeutic efficacy relative to administration of the checkpoint inhibitor or a compound of Formula I and/or Formula Ia or a pharmaceutically acceptable salt thereof alone.

The present disclosure therefore provides a combination therapy of checkpoint inhibitor therapy together with a specific type of immunotherapy comprising administration of a compound of Formula I and/or Formula Ia or a pharmaceutically acceptable salt thereof. The inventors have found that the combination of both therapies is synergistic beyond simple additive effects of each therapy used individually. These and other aspects of the present disclosure may be more fully understood by reference to the following detailed description, non-limiting examples of specific embodiments, and the appended figures.

### BRIEF DESCRIPTION OF THE DRAWINGS

The disclosure is described with reference to the following drawings, in which:

FIG. 1A-FIG. 1E depict analyses of an in vivo study in mice bearing KPC pancreatic tumors. FIG. 1A shows Kaplan-Meier survival analysis for days of treatment in mice treated with a vehicle control; MOL-211 (50 mg/kg); PD-1 antibody (10 mg/kg); and combination of MOL-211 and PD-1 antibody. FIG. 1B shows body weight change at days post-tumor implantation under the same criteria. FIG. 1C shows the ratio of tumor volume change (treated/control) from first day of treatment to last day of treatment (ΔT/ΔC ratio) for MOL-211 (50 mg/kg); PD-1 antibody (10 mg/kg); and the combination thereof. Objective responses are defined as either partial responder or complete responder. FIG. 1D shows tumor volume changes at days post tumor implantation in mice treated with a vehicle control; MOL-211 (50 mg/kg); PD-1 antibody (10 mg/kg); and combination of MOL-211 and PD-1 antibody. FIG. 1E shows change in tumor volume from baseline at the start of treatment for the indicated treatment groups.

FIG. 2 shows tumor volume over 15 days in KPC-2 NCR Nude vs FBV/N mice.

FIG. 3 shows tumor volume over 15 days in SCC7 NCR Nude vs. C3H mice.

FIG. 4A-FIG. 4D show analyses of an in vivo study in C3H mice bearing SCC7 head and neck tumors. FIG. 4A depicts Kaplan-Meier survival analysis for day of treatment in mice treated with a vehicle control; MOL-211 (50 mg/kg); PD-1 antibody (10 mg/kg); or combination of MOL-211 and PD-1 antibody. The calculated increase in lifespan (“ILS”) is also shown for MOL-211 (206%), PD-1 antibody (106%) and the combination treatment (322%). FIG. 4B shows tumor volume changes at days post tumor implantation for the indicated treatment groups.

FIG. 4C shows change in tumor volume from baseline at the start of treatment for the indicated treatment groups. FIG. 4D shows body weight change at days post-tumor implantation for the indicated treatment groups.

FIG. 5A-FIG. 5D show analyses of an in vivo study in BALB/c mice bearing CT-26 (murine colorectal carcinoma) tumors. FIG. 5A depicts Kaplan-Meier survival analysis for day of treatment in mice treated with a vehicle control; MOL-211 (50 mg/kg); PD-1 antibody (10 mg/kg); or combination of MOL-211 and PD-1 antibody. The calculated increase in lifespan (“ILS”) is also shown for MOL-211 (22%), PD-1 antibody (0%) and the combination treatment (0%). FIG. 5B shows tumor volume changes at days post tumor implantation for the indicated treatment groups. FIG. 5C shows change in tumor volume from baseline at the start of treatment for the indicated treatment groups. FIG. 5D shows body weight change at days post-tumor implantation for the indicated treatment groups.

FIG. 6A-FIG. 6C show analyses of an in vivo study in female BALB/c mice bearing EMT-6 (murine mammary carcinoma) tumors. FIG. 6A shows change in tumor volume from baseline at the start of treatment for the indicated treatment groups. FIG. 6B shows tumor volume changes at days post tumor implantation for the indicated treatment groups. FIG. 6C shows body weight change at days post-tumor implantation for the indicated treatment groups.

FIG. 7A-FIG. 7C shows analysis of PD-L1 expression in tumor cells. FIG. 7A shows flow cytometry plots of tumor cells collected from FVB/N mice bearing KPC-2 tumors and treated with either DMSO or daily MOL-211 (50 mg/kg) and 5 treatments of PD-1 antibody (10 mg/kg once every three days). FIG. 7B shows quantification of live PD-L1 positive cells from the flow cytometry plots in FIG. 7A. FIG. 7C depicts a western blot of KPC-2 cells treated with MOL-211 in vitro for 24 or 48 hours.

### DETAILED DESCRIPTION

The present disclosure provides a method for preventing, treating, reducing, inhibiting or controlling a neoplasia, tumor or cancer and/or the establishment of metastases in a subject involving administering a compound of Formula I and/or Formula Ia or a pharmaceutically acceptable salt thereof and a checkpoint inhibitor. It is based upon the discovery that administration of a compound of Formula I and/or Formula Ia or a pharmaceutically acceptable salt thereof in combination with a checkpoint inhibitor results in more than additive effects, i.e. synergistic anti-tumor activity and/or antitumor activity that is more potent than the administration of a compound of Formula I and/or Formula Ia or a pharmaceutically acceptable salt thereof or a checkpoint inhibitor alone.

For purposes of this disclosure, the chemical elements are identified in accordance with the Periodic Table of the Elements, CAS version, Handbook of Chemistry and Physics, 98th Ed. Additionally, general principles of organic chemistry are described in “Organic Chemistry”, Second Ed., Thomas Sorrell, University Science Books, Sausolito: 2006, and “March's Advanced Organic Chemistry”, 7th Ed., Ed.: Smith, M. B. and March, J., John Wiley & Sons, New York: 2015, the entire contents of which are hereby incorporated by reference.

### Definitions

As used herein, the term “acceptor human framework” refers to a framework comprising the amino acid sequence of a light chain variable domain (V_{L}) framework or a heavy chain variable domain (V_{H}) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the V_{L} acceptor human framework is identical in sequence to the V_{L} human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the equilibrium dissociation constant (K_{D}), a ratio of k_{off}/kₒₙ, between the antibody and its antigen. K_{D} and affinity are inversely related. The K_{D} value relates to the concentration of antibody (the amount of antibody needed for a particular experiment) and so the lower the K_{D} value (lower concentration) and thus the higher the affinity of the antibody. Affinity can be measured by common methods known in the art, including those described herein. Specific, illustrative, and exemplary embodiments for measuring binding affinity can be measured by radioimmunoassays (RIA), Surface Plasmon Resonance (SPR) on a BIAcore® instrument (GE Healthcare Europe GmbH, Glattbrugg, Switzerland) by capturing the antibody on a protein-A coupled CM5 research grade sensor chip (GE Healthcare Europe GmbH, Glattbrugg, Switzerland; BR-1000-14) with a human soluble checkpoint polypeptide used as analyte. Other methods can include radioimmunoassays, and the Kinetic Exclusion Assay. The Kinetic Exclusion Assay is a general purpose immunoassay platform that is capable of measuring equilibrium dissociation constants, and association and dissociation rate constants for antigen/anti-body interactions.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

As used herein, “about” means within acceptable error range for the particular value as determined by one of ordinary skill in the art, which will depend in part on how the value is measured or determined, i.e., the limitations of the measurement system. For example, “about” can mean within 1 or more than 1 standard deviation per the practice in the art. Alternatively, “about” can mea range of up to 20%. When particular values are provided in the application and claims, unless otherwise stated, the meaning of “about” should be assumed to be within acceptable error range for that particular value.

As used herein, the term “additive” or “additive effect” when used in connection with a description of the efficacy of a combination of agents, means any measured effect of the combination which is similar to the effect predicted from a sum of the effects of the individual agents.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fd fragments, dAb fragments, Fab′-SH, F(ab′)₂; diabodies; triabodies; linear antibodies; single-chain antibody molecules (e.g., scFv); and multispecific antibodies formed from antibody fragments, minimal recognition units consisting of the amino acid residues that mimic the hypervariable region of an antibody (e.g., an isolated complementarity determining region (CDR) such as a CDR3 peptide), or a constrained FR3 CDR3 FR4 peptide.

The terms “antigen-binding portion” of an antibody, or “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-display anti-body libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}-V_{L} or V_{L}-V_{L} dimers. Alternatively, the antigen-binding fragment of an antibody may contain a monomeric V_{H} or V_{L} domain.

In certain embodiments, antibody or antigen-binding fragments of the disclosure may be conjugated to a therapeutic moiety (“immunoconjugate”), such as a cytotoxin, a chemo-therapeutic drug, an immunosuppressant or a radioisotope.

An “antibody that competes for binding with” a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The terms “antagonistic antibody” or “antagonist antibody” are used herein equivalently and include an antibody that is capable of inhibiting and/or neutralizing the biological signaling activity of an immune checkpoint.

The terms “agonistic antibody” or agonist antibody” are used herein equivalently and include an antibody that is capable of activating and/or enhancing the biological signaling activity of an immune checkpoint.

A number of CDR definitions are in use and are encompassed herein. The Kabat definition is based on sequence variability and is the most commonly used (Kabat E A et al., supra). Chothia refers instead to the location of the structural loops (Chothia, C. & Lesk, A. M. (1987) J. Mol. Biol. 196: 901-917). The AbM definition is a compromise between the Kabat and the Chothia definitions and is used by Oxford Molecular's AbM antibody modeling software (Martin A C R et al., (1989) Proc. Natl. Acad. Sci. USA, 86: 9268-72; Martin A C R et al., (1991) Methods Enzymol. 203: 121-153; Pedersen J T et al., (1992) Immunomethods, 1: 126-136; Rees A R et. al., (1996) In Sternberg M. J. E. (ed.), Protein Structure Prediction. Oxford University Press, Oxford, 141-172).

The hypervariable region generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., Sequences Of Proteins Of Immunological Interest, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the disclosure are described below.

Throughout the present specification, the Kabat numbering system is generally used when referring to a residue in the variable domain (approximately, residues 1-107 of the light chain variable region and residues 1-113 of the heavy chain variable region) (e.g., Kabat et al., supra (1991)), with the EU number system used for the Fc region. Unless otherwise indicated, hypervariable residues and other residues in the variable domain (e.g., FR residues) are numbered herein according to Kabat et al. Sequences of Proteins of Immunological Interest, 1991.

The CDRs contribute to the formation of the antigen-binding, or more specifically, epitope binding site of antibodies.

In addition to antibodies, other biological molecules may act as checkpoint inhibitors, including peptides having binding affinity to the appropriate target.

The term “antigen-binding portion” of antibody (or simply “antibody portion”), as used herein, refers to one or more fragments of antibody that retain the ability to specifically bind to a receptor and its ligand (e.g., PD-1). including: (i) a Fab fragment, (ii) a F(ab′) 2 fragment; (iii) a Fd fragment consisting of the VH and CHI domains; (iv) a Fv fragment, (v) a dAb fragment (Ward et al, Nature, 341:544-546 (1989)), which consists of a VH domain; and (vi) an isolated complementarity determining region (CDR). Single chain antibodies are also intended to be encompassed within the term “antigen-binding portion” of antibody. These antibody fragments are obtained using conventional techniques known to those with skill in the art, and the fragments are screened for utility in the same manner as are intact antibodies.

The term “biomarker” as used herein refers to an indicator, e.g., a predictive, diagnostic, and/or prognostic indicator, which can be detected in a sample. The biomarker may serve as an indicator of a particular subtype of a disease or disorder (e.g., cancer) characterized by certain, molecular, pathological, histological, and/or clinical features. In some embodiments, the biomarker is a gene. In some embodiments, the biomarker is a variation (e.g., mutation and/or polymorphism) of a gene. In some embodiments, the biomarker is a translocation. Biomarkers include, but are not limited to, polynucleotides (e.g., DNA, and/or RNA), polypeptides, polypeptide and polynucleotide modifications (e.g., posttranslational modifications), carbohydrates, and/or glycolipid-based molecular markers.

As used herein, the term “carrier,” or “pharmaceutically acceptable carrier” includes any and all solvents, dispersion media, coatings, surfactants, antioxidants, preservatives {e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329).

A “checkpoint inhibitor” is an agent which acts on immune checkpoint molecules or checkpoint proteins, e.g., on surface proteins which are members of either the TNF receptor or B7 superfamilies, including agents which bind to negative co-stimulatory molecules selected from, e.g., CTLA-4 (or its ligands, e.g., CD80 and/or CD86); PD-1 (or its ligands, e.g., PD-L1 and/or PD-L2); TIM-3 (or its ligands, e.g., Galectin-9, Phospatidyl serine (PtdSer), HMGB1, and/or CEACAM1); BTLA (or its ligands, e.g., PTPN6/SHP-1, PTPN11/SHP-2, TNFRSF14/HVEM, and/or B7H4); VISTA (or its ligands, e.g., VSIG-3); and/or LAG-3 (or its ligands, e.g., MHC class II). As used herein, a checkpoint inhibitor can be an antibody, an antigen binding portion, an antibody fragment, e.g., a monoclonal antibody, an Fv fragment, an scFv fragment, a di-scFv fragment, an F(ab′)₂ fragment, and Fab fragment, an HCAb, a diabody, a bi-specific antibody, one or more V_{H}H or V_{L} fragments, or one or more CDRs (light or heavy); however, the term “checkpoint inhibitor” can also include any method in which checkpoint proteins are inhibited, or intrinsic checkpoint inhibitors are promoted, e.g., methods that affect checkpoint proteins at the transcriptional and/or translational level.

A “checkpoint molecule,” or “immune checkpoint,” or “checkpoint protein” refers to molecules involved in immunoregulation, e.g., immunosurveillance and/or elimination of foreign cells like cancer; accordingly, immune checkpoints are molecules on certain immune cells—or that interact with certain immune cells or their upstream or downstream binding partners—that need to be activated (or inactivated) to initialize and/or maintain an immune response. Cancer cells affect the activation and/or deactivation of immune checkpoints to avoid immunosurveillance and/or removal; thus, checkpoint inhibitors are agents that target these immune checkpoints.

The term “chimeric” antibody refers to an antibody in which a portion of the heavy and/or light chain is derived from a particular source or species, while the remainder of the heavy and/or light chain is derived from a different source or species.

The term “combination” as used throughout the specification, is meant to encompass the administration of the checkpoint inhibitor simultaneously, separately or sequentially with administration of a compound of Formula I or Formula Ia. Accordingly, the checkpoint inhibitor and the compound of Formula I or Formula Ia may be present in the same or separate pharmaceutical formulations, and administered at the same time or at different times.

A compound of Formula I, or Formula Ia as defined according to the present disclosure, is a component which may stimulate innate and type-1 immunity, including Th1 and macrophage activation and cytotoxic cell activity, as well as independently down-regulating inappropriate anti-Th2 responses via immunoregulatory mechanisms.

As used herein, the terms “concurrent administration” or “concurrently” or “simultaneous” mean that administration occurs on the same day. The terms “sequential administration” or “sequentially” or “separate” mean that administration occurs on different days.

The terms “cytotoxic T lymphocyte-associated antigen-4,” “CTLA-4,” “CTLA4,” and “CTLA-4 antigen” (see, e.g., Murata, Am. J. Pathol. (1999) 155:453-460) are used interchangeably, and include variants, isoforms, species homologs of human CTLA-4, and analogs having at least one common epitope with CTLA-4 (see, e.g., Balzano (1992) Int. J. Cancer Suppl. 7:28-32). The complete CTLA-4 nucleic acid sequence can be found under GenBank Accession No. L15006.

The term “diagnosis” is used herein to refer to the identification or classification of a molecular or pathological state, disease or condition (e.g., an inflammatory disease, for example, inflammatory bowel disease). For example, “diagnosis” may refer to identification of a particular type of autoimmune disease, for example, rheumatoid arthritis. “Diagnosis” may also refer to the classification of a particular subtype of disease, e.g., by histopathological criteria, or by molecular features (e.g., a subtype characterized by expression of one or a combination of biomarkers (e.g., particular genes or proteins encoded by said genes)).

As used herein, an “effective amount” is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, N.Y., 537 (1970). As used herein, “patient” refers to a mammal, including a human.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al.

The CDRs contribute to the formation of the antigen-binding, or more specifically, epitope binding site of antibodies. The term “epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 4 or 5-12 amino acids in a unique spatial conformation. Antibodies that recognize the same epitope can be verified in a simple immunoassay showing the ability of one antibody to block the binding of another antibody to a target antigen, for example “binning”, has identified the amino acid residues that bind to the antibodies of the disclosure.

The term “paratope” is derived from the above definition of “epitope” by reversing the perspective. Thus, the term “paratope” refers to the area or region on the antibody which specifically binds an antigen, i.e., the amino acid residues on the antibody which make contact with the antigen (e.g., an immune checkpoint).

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human consensus framework” is a framework which represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5^{th} Edition, NIH Publication 91-3242, Bethesda Md. (1991), vols. 1-3, (entirely incorporated by reference herein). In one embodiment, for the V_{L}, the subgroup is subgroup kappa I as in Kabat et al., supra. In one embodiment, for the V_{H}, the subgroup is subgroup III as in Kabat et al. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present disclosure are the heavy chain domains, including, the constant heavy (C_{H}) domains and the hinge domains. In the context of IgG antibodies, the IgG isotypes each have three C_{H} regions. Accordingly, “C_{H}” domains in the context of IgG are as follows: “C_{H}1” refers to positions 118-220 according to the EU index as in Kabat. “C_{H}2” refers to positions 237-340 according to the EU index as in Kabat, and “C_{H}3” refers to positions 341-447 according to the EU index as in Kabat.

The term “human” antibody refers to an antibody which possesses an amino acid sequence which corresponds to that of an antibody produced by a human and/or has been made using any of the techniques for making human antibodies as disclosed herein. This definition of a human antibody specifically excludes a humanized antibody comprising non-human antigen binding residues.

The term “humanized antibody” is intended to refer to antibodies in which CDR sequences derived from the germline of another mammalian species, such as a mouse, have been grafted onto human framework sequences. Additional framework region modifications may be made within the human framework sequences.

“Immune checkpoint” refers to molecules involved in immunoregulation, e.g., like vs. unlike cell detection (e.g., “foreign” cell detection); accordingly, immune checkpoints are molecules on certain immune cells—or that interact with certain immune cells or their upstream or downstream binding partners—that need to be activated (or inactivated) to start an immune response. Cancer cells affect the activation and/or deactivation of immune checkpoints to avoid immunosurveillance and/or removal; thus, checkpoint inhibitors are agents that target these immune checkpoints.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells.

The terms “inhibit” or “inhibition of” means to reduce by a measurable amount, or to prevent entirely. The term inhibition as used herein can refer to an inhibition or reduction of at least about 10%, at least about 15%, at least about 20%, at least about 25%, at least about 30%, at least about 35%, at least about 40%, at least about 50%, at least about 60%, at least about 70%, at least about 80%, at least about 90%, at least about 95%, or at least about 99%.

The terms “monoclonal antibody” or “monoclonal antibody composition” as used herein refer to a preparation of antibody molecules of single molecular composition. A monoclonal antibody composition displays a single binding specificity and affinity for a particular epitope.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (V_{H}), also called a variable heavy domain or a heavy chain variable domain, followed by three constant domains (C_{H}1, C_{H}2, and C_{H}3). Similarly, from N- to C-terminus, each light chain has a variable region (V_{L}), also called a variable light domain or a light chain variable domain, followed by a constant light (C_{L}) domain. The light chain of an antibody may be assigned to one of two types, called kappa (κ) and lambda (λ), based on the amino acid sequence of its constant domain.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “neutralizing antibody” includes an antibody that is capable of inhibiting and/or neutralizing the biological activity of an immune checkpoint molecule, for example by blocking binding or substantially reducing binding of a ligand to its receptor, thus inhibiting or reducing the signaling pathway triggered by and/or inhibiting or reducing a checkpoint-mediated cell response.

The use of the alternative (e.g., “or”) should be understood to mean either one, both, or any combination thereof of the alternatives. As used herein, the indefinite articles “a” or “an,” should be understood to refer to “one or more” of any recited or enumerated component.

As used herein the term “parent antibody”, “parent protein”, “precursor polypeptide”, or “precursor protein” as used herein is meant an unmodified antibody or polypeptide that is subsequently modified to generate a variant. Parent polypeptide may refer to the polypeptide itself, compositions that comprise the parent polypeptide, or the amino acid sequence that encodes it. Accordingly, by “parent Fc polypeptide” as used herein is meant an Fc polypeptide that is modified to generate a variant, and by “parent antibody” as used herein is meant an antibody that is modified to generate a variant antibody.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, “pharmaceutically acceptable carrier” includes any and all solvents, dispersion media, coatings, surfactants, antioxidants, preservatives {e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329).

As used herein, “pharmaceutically acceptable carrier” or “pharmaceutical acceptable excipient” includes any material which, when combined with an active ingredient, allows the ingredient to retain biological activity and is non-reactive with the subject's immune system, and can include any and all solvents, diluents, carriers, dispersion media, coatings, antibacterial and antifungal agents, isotonic and absorption delaying agents, and the like that are physiologically compatible, non-toxic, and does not interfere with the mechanism of action of the checkpoint inhibitor antibodies or antigen-binding fragments thereof, the compound of Formula I and/or Formula Ia or a pharmaceutically acceptable salt thereof; and/or both in combination. Preferably, the pharmaceutical acceptable excipient is suitable for intravenous, intramuscular, subcutaneous, parenteral, spinal or epidermal administration (e.g., by injection or infusion). Depending on the route of administration, the combination, i.e., a compound of Formula I and/or Formula Ia or a pharmaceutically acceptable salt thereof and one or more checkpoint inhibitor antibodies and/or antigen-binding fragment thereof, or immunoconjugate, may be coated in a material to protect the compound from the action of acids and other natural conditions that may inactivate the compound. Pharmaceutically acceptable excipients include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

The terms “Programmed Death 1,” “Programmed Cell Death 1,” “Protein PD-1,” “PD-1,” and “PD1,” are used interchangeably, and include variants, isoforms, species homologs of human PD-1, and analogs having at least one common epitope with PD-1. The complete PD-1 sequence can be found under GenBank Accession No. U64863.

The term “recombinant” as used herein to describe a nucleic acid molecule, means a polynucleotide of genomic, mRNA, cDNA, viral, semisynthetic, and/or synthetic origin, which, by virtue of its origin or manipulation, is not associated with all or a portion of the polynucleotide with which it is associated in nature, thus it non-natural. The term recombinant as used with respect to a protein or polypeptide, means a polypeptide produced by expression of a recombinant polynucleotide. The term recombinant as used with respect to a host cell means a host cell into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

“Separate” administration, as defined herein, includes the administration of the a compound of Formula I and/or Formula Ia or a pharmaceutically acceptable salt thereof and agent or procedure comprising checkpoint inhibitor therapy, more than about 12 hours, or about 8 hours, or about 6 hours or about 4 hours or about 2 hours apart.

“Sequential” administration, as defined herein, includes the administration of the compound of Formula I and/or Formula Ia or a pharmaceutically acceptable salt thereof and chemotherapeutic agent each in multiple aliquots and/or doses and/or on separate occasions. The compound of Formula I and/or Formula Ia or a pharmaceutically acceptable salt thereof may be administered to the patient after before and/or after administration of the checkpoint inhibitor. Alternatively, the compound of Formula I and/or Formula Ia or a pharmaceutically acceptable salt thereof is continued to be applied to the patient after treatment with a checkpoint inhibitor.

“Simultaneous” administration, as defined herein, includes the administration of the a compound of Formula I and/or Formula Ia or a pharmaceutically acceptable salt thereof and agent or procedure comprising checkpoint inhibitor therapy within about 2 hours or about 1 hour or less of each other, even more preferably at the same time.

The term “specifically binds,” or the like, means that an antibody or antigen-binding fragment thereof forms a complex with an antigen that is relatively stable under physiological conditions. Specific binding can be characterized by an equilibrium dissociation constant (K_{D}) of about 3000 nM or less (i.e., a smaller K_{D} denotes a tighter binding), about 2000 nM or less, about 1000 nM or less; about 500 nM or less; about 300 nM or less; about 200 nM or less; about 100 nM or less; about 50 nM or less; about 1 nM or less; or about 0.5 nM.

Specific binding for a particular antigen or an epitope can be exhibited, for example, by an antibody having a K_{D} for an antigen or epitope of at least about 1×10⁻⁴ M, at least about 1×10⁻⁵ M, at least about 1×10⁻⁶ M, at least about 1×10⁻⁷ M, at least about 1×10⁻⁸ M, at least about 1×10⁻⁹ M, alternatively at least about 1×10⁻¹⁰ M, at least about 1×10⁻¹¹ M, at least about 1×10⁻¹² M, or greater, where K_{D} refers to a equilibrium dissociation constant of a particular antibody-antigen interaction. Typically, an antibody that specifically binds an antigen will have a K_{D} that is 20-, 50-, 100-, 500-, 1000-, 5,000-, 10,000- or more times greater for a control molecule relative to the antigen or epitope. Also, specific binding for a particular antigen or an epitope can be exhibited, for example, by an antibody having a Kₐ for an antigen or epitope of at least 20-, 50-, 100-, 500-, 1000-, 5,000-, 10,000- or more times greater for the epitope relative to a control, where Kₐ refers to an association rate of a particular antibody-antigen interaction.

The phrase “stable or chemically feasible,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The methods of treatment of the disclosure comprise administering a safe and effective amount of a compound described herein or a pharmaceutically-acceptable salt thereof to a patient in need thereof.

As used herein, “sub-therapeutic dose” means a dose of a therapeutic compound (e.g., antibody) or duration of therapy which is lower than the usual or typical dose of the therapeutic compound or therapy of shorter duration, when administered alone for the treatment of cancer. For example, a sub-therapeutic dose of CTLA-4 antibody is a single dose of the antibody at less than about 3 mg/kg, i.e., the known dose of anti-CTLA-4 antibody.

As used herein, the term “subject” is intended to include human and non-human animals. Preferred subjects include human patients in need of enhancement of an immune response that may be beneficial in the patient's treatment and/or prevention of cancer and/or cancer metastasis. The methods are particularly suitable for treating human patients having a disorder that can be treated by augmenting the T-cell mediated immune response. In a particular embodiment, the methods are particularly suitable for treatment of cancer cells in vivo.

“Such as” has the same meaning as “such as but not limited to.” Similarly, “include” has the same meaning as “include but not limited to,” while “including” has the same meaning as “including but not limited to.”

As used herein, the term “synergy” or “synergistic effect” when used in connection with a description of the efficacy of a combination of agents, means any measured effect of the combination which is greater than the effect predicted from a sum of the effects of the individual agents.

The term “therapeutically effective amount” is defined as amount of a checkpoint inhibitor, in combination with a compound of Formula I and/or Formula Ia or a pharmaceutically acceptable salt thereof, that preferably results in a decrease in severity of disease symptoms, an increase in frequency and duration of disease symptom-free periods, or a prevention of impairment or disability due to the disease affliction. The terms “effective amount” or “pharmaceutically effective amount” refer to a sufficient amount of agent to provide the desired biological or therapeutic result. That result can be reduction, amelioration, palliation, lessening, delaying, and/or alleviation of one or more of the signs, symptoms, or causes of a disease, or any other desired alteration of a biological system. In reference to cancer, an effective amount may comprise amount sufficient to cause a tumor to shrink and/or to decrease the growth rate of the tumor (such as to suppress tumor growth) or to prevent or delay other unwanted cell proliferation. In some embodiments, an effective amount is amount sufficient to delay development, or prolong survival or induce stabilization of the cancer or tumor.

A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

In some embodiments, a therapeutically effective amount is amount sufficient to prevent or delay recurrence. A therapeutically effective amount can be administered in one or more administrations. The therapeutically effective amount of the drug or combination may result in one or more of the following: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

The term “treatment” or “therapy” refers to administering active agent with the purpose to cure, heal, alleviate, relieve, alter, remedy, ameliorate, improve, or affect a condition (e.g., a disease), the symptoms of the condition, or to prevent or delay the onset of the symptoms, complications, biochemical indicia of a disease, or otherwise arrest or inhibit further development of the disease, condition, or disorder in a statistically significant manner.

As used herein, “treat” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative or differentiative disorder. Typically, the growth is uncontrolled. The term “malignancy” refers to invasion of nearby tissue. The term “metastasis” refers to spread or dissemination of a tumor, cancer or neoplasia to other sites, locations or regions within the subject, in which the sites, locations or regions are distinct from the primary tumor or cancer.

The term “variable region” or “variable domain” (used synonymously herein) refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

By “wild type” or “WT” or “native” herein is meant an amino acid sequence or a nucleotide sequence that is found in nature, including allelic variations.

The present disclosure provides a method for preventing, treating, reducing, inhibiting or controlling a neoplasia, a tumor or a cancer in a subject in need thereof, involving administering a therapeutically effective amount of a combination comprising a compound of Formula I and/or Formula Ia, and a checkpoint inhibitor. In one non-limiting embodiment, the method comprises administering a therapeutically effective amount of a combination comprising a compound of Formula I, and/or Formula Ia in combination with an anti-PD1 or an anti-PD-L1 antibody (a checkpoint inhibitor). In various embodiments, the combination provides a cooperative effect, an additive effect, or a synergistic effect in reducing the number of cancer cells when treated with the combination as compared to each treatment alone. In some embodiments, administration of a therapeutically effective amount of a combination comprising a compound of Formula I and/or Formula Ia and a checkpoint inhibitor, results in synergistic anti-tumor activity and/or antitumor activity that is more potent than the additive effect of administration of a compound of Formula I and/or a compound of Formula Ia or anti-PD-1 or anti-PD-L1 antibody alone.

Anti-EGFR and Anti-PI3K Compounds of Formula I

The compounds of Formula I described in the present disclosure are small-molecules having a quinazoline structure or a quinoline structure which function as dual inhibitors of EGFR proteins and PI3K proteins, including PI3K-related kinase mTOR, and their use as therapeutics for the treatment of cancer and other diseases when combined with an immune checkpoint inhibitor as described herein.

The quinazoline compounds and quinoline compounds of the present disclosure embodied in Formula I were accordingly synthesized to target the “active cores” for PI3K and the “active cores” for EGFR, thereby rendering such compounds as having “dual potency” against PI3K and EGFR.

PI3K is negatively regulated by phosphatase and tensin homolog (PTEN) (see, e.g., Hamada K, et al., 2005 Genes Dev 19 (17): 2054-65). Numerous studies have shown a link between PIK3CA mutation/PTEN loss and EGFR targeted resistance leading to poor overall survival (see, e.g., Atreya C E, Sangale Z, Xu N, et al. Cancer Med. 2013; 2: 496-506; Sawai H, et al., BMC Gastroenterol. 2008; 8: 56; Bethune G, et al., J Thorac Dis. 2010; 2: 48-51; Spano J P, et al., Ann Oncol. 2005; 16: 189-194; Heimberger A B, et al., J Transl Med. 2005; 3: 38). The quinazoline compounds and quinoline compounds synthesized during the course of developing embodiments for the present disclosure were designed based on a central hypothesis that dual targeting of EGFR and PIK3CA would be efficacious in patients with colorectal cancer that are EGFR positive and are either PIK3CA mutated or null PTEN expressers (see, e.g., Psyrri A, et al., Am Soc Clin Oncol Educ Book. 2013: 246-255; Lui V W, et al., Cancer Discov. 2013; 3: 761-769; Jin G, et al., Lung Cancer. 2010; 69: 279-283; Buck E, et al., Mol Cancer Ther. 2006; 5: 2676-2684; Fan Q W, et al., Cancer Res. 2007; 67: 7960-7965; Gadgeel S M, et al., Clin Lung Cancer. 2013; 14: 322-332.

The mTOR pathway controls cell growth in response to energy, nutrients, growth factors and other environmental cues, and it figures prominently in cancer. Central to the pathway is the mammalian target of rapamycin (mTOR) protein that belongs to the phosphoinositide 3-kinase (PI3K)-related protein kinase (PIKK) family. mTOR assembles into two complexes with distinct inputs and downstream effects. mTOR Complex 1 (mTORC1) is defined by its RAPTOR subunit, which is replaced by RICTOR in mTORC2. Both complexes also contain the requisite mLST8 subunit, but they differ in a number of other subunits that interact with RAPTOR or RICTOR.

As such, the present disclosure relates to a class of small-molecules having a quinazoline structure or quinoline structure which function as dual inhibitors of EGFR protein and PI3K protein, and their use as therapeutics when combined with an immune checkpoint inhibitor as described herein, for the prevention and treatment of conditions characterized by aberrant EGFR and PI3K expression (e.g., cancer). Indeed, through targeting both EGFR and PI3K, the compounds of the present disclosure are useful in treating subjects with EGFR positive colorectal cancer that harbor an activating mutation in PI3Kα or are PTEN null.

Accordingly, the present disclosure contemplates that exposure of animals (e.g., humans) suffering from a condition characterized by aberrant EGFR protein activity (e.g., ERBB1) and PI3K protein activity (e.g., PI3Kα) (e.g., cancer (e.g., and/or cancer related disorders)) to therapeutically effective amounts of a combination comprising a compound of Formula I and/or Formula Ia or a pharmaceutically acceptable salt thereof having a quinazoline structure (e.g., small molecules having a quinazoline structure) or a quinoline structures (e.g., small molecules having a quinoline structure) that inhibit the activity of both EGFR and PI3K together with an immune checkpoint inhibitor as defined herein, will inhibit the growth of cells characterized by aberrant EGFR and PI3K protein expression (e.g., cancer cells having aberrant EGFR and PI3K protein expression) and/or render such cells as a population more susceptible to the cell death-inducing activity. The present disclosure contemplates that inhibitors of both EGFR and PI3K satisfy an unmet need for the treatment of multiple conditions characterized with aberrant EGFR and PI3K activity (e.g., cancer), when administered as a combination therapy to induce cell growth inhibition, apoptosis and/or cell cycle arrest in such cells (e.g., cancer cells), compared to the corresponding proportion of cells in an animal treated only with the compound of Formula I and/or Formula Ia or a pharmaceutically acceptable salt thereof or the immune checkpoint inhibitor therapy alone.

In certain embodiments of the disclosure wherein the condition being treated is cancer characterized with aberrant EGFR protein activity (e.g., ERBB1) and PI3K protein activity (e.g., PI3Kα), combination treatment of animals with a therapeutically effective amount of a compound of the present disclosure and a course of an immune checkpoint inhibitor as described herein, produces a greater tumor response and clinical benefit in such animals compared to those treated with the compound or immune checkpoint inhibitor alone, i.e. a cooperative, or additive or synergistic effect is produced.

As noted, the Applicants have found that certain quinazoline compounds and quinoline compounds function as inhibitors of both EGFR and PI3K, and serve as therapeutics for the treatment of cancer and other diseases. Thus, the present disclosure relates to quinazoline compounds and quinoline compounds useful for inhibiting EGFR and PI3K activity (e.g., thereby facilitating cell apoptosis), and increasing the sensitivity of cells to inducers of apoptosis and/or cell cycle arrest. Certain quinazoline compounds and quinoline compounds of the present disclosure may exist as stereoisomers including optical isomers. The disclosure includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

A Compound of Formula I

For purposes of this invention, the chemical elements are identified in accordance with the Periodic Table of the Elements, CAS version, Handbook of Chemistry and Physics, 98th Ed. Additionally, general principles of organic chemistry are described in “Organic Chemistry”, Second Ed., Thomas Sorrell, University Science Books, Sausolito: 2006, and “March's Advanced Organic Chemistry”, 7th Ed., Ed.: Smith, M. B. and March, J., John Wiley & Sons, New York: 2015, the entire contents of which are hereby incorporated by reference.

As described herein, compounds of the invention may optionally be substituted with one or more substituents, such as are illustrated generally above, or as exemplified by particular classes, subclasses, and species of the invention.

As used herein, an “alkyl” group refers to a saturated aliphatic hydrocarbon group containing 1-12 (e.g., 1-8, 1-6, or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl.

As used herein, an “alkenyl” group refers to an aliphatic carbon group that contains 2-8 (e.g., 2-12, 2-6, or 2-4) carbon atoms and at least one double bond. Like an alkyl group, an alkenyl group can be straight or branched. Examples of an alkenyl group include, but are not limited to allyl, isoprenyl, 2-butenyl, and 2-hexenyl.

As used herein, an “alkynyl” group refers to an aliphatic carbon group that contains 2-8 (e.g., 2-12, 2-6, or 2-4) carbon atoms and has at least one triple bond. An alkynyl group can be straight or branched. Examples of an alkynyl group include, but are not limited to, propargyl and butynyl.

As used herein, an “alkylene” group refers to a bivalent alkyl group that connects to two attachment points simultaneously, wherein the alkylene unit can be bivalent on the same carbon or two different carbons of the alkyl moiety. Examples of alkylene groups are, without limitation, methylene, ethylene, propylene, and butylene, as well as branched structures, such as —CH₂(CH₂)— (1,1-ethylene) and —CH₂CH₂(CH₂)— (1,2-propylene).

As used herein an “aryl” group refers to a mono-, bi-, or tri-cyclic ring system wherein all rings in the system are aromatic and contain no heteroatoms in the ring. Examples of aryl groups include, but are not limited to phenyl, naphthyl, anthracenyl, and tetracenyl.

As used herein, a “carbocycle” or “carbocyclyl” group refers to a mono-, bi-, or tricyclic (fused or bridged) hydrocarbon ring system that contains no heteroatoms in the ring structures, wherein at least one of the rings in the system is non-aromatic, and can be completely saturated or partially unsaturated. The terms “carbocycle” or “carbocyclyl” encompass a “cycloalkyl” group and a “cycloalkenyl” group, each of which is set forth below.

As used herein, a “cycloalkyl” group refers to a saturated carbocyclic mono-, bi-, or tricyclic (fused or bridged) ring system of 3-20 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, or ((aminocarbonyl)cycloalkyl)cycloalkyl.

A “cycloalkenyl” group, as used herein, refers to a non-aromatic carbocyclic mono-, bi, or tricyclic (fused or bridged) ring system of 3-20 (e.g., 4-8) carbon atoms, wherein at least one ring in the system has one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, or bicyclo[3.3.1]nonenyl.

As used herein, the terms “heterocycle” and “heterocyclyl” are used interchangeably and refer to a mono-, bi-, or tricyclic (fused or bridged) non-aromatic hydrocarbon ring system that contains at least one heteroatom in the ring structure and can be completely saturated or partially unsaturated. The terms “heterocycle” and “heterocyclyl” encompass a “heterocycloalkyl” group and a “heterocycloalkenyl” group, each of which is set forth below.

As used herein, a “heterocycloalkyl” group refers to a 3-20 membered mono-, di-, or tricylic (fused or bridged) (e.g., 5- to 10-membered) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0.3.7]nonyl.

A “heterocycloalkenyl” group, as used herein, refers to a 3-20 membered mono-, di-, or tricylic (fused or bridged) (e.g., 5- to 10-membered) non-aromatic ring structure, in which one or more of the ring atoms is a heteroatom (e.g., N, O, S, or combinations thereof), and wherein at least one of the ring structures has one or more double bonds.

A “heteroaryl” group, as used herein, refers to a monocyclic, bicyclic, or tricyclic ring system having 4 to 15 ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, or 1,8-naphthyridyl.

Without limitation, monocyclic heteroaryls include furyl, thiophenyl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, or 1,3,5-triazyl.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizinyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicaliphatic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxabicyclo[2.2.2]octyl, 1-azabicyclo[2.2.2]octyl, 3-azabicyclo[3.2.1]octyl, and 2,6-dioxa-tricyclo[3.3.1.0.3.7]nonyl.

As used herein, an “alkoxy” group refers to an alkyl-O— group where “alkyl” has been defined previously.

As used herein, a “carbonyl” refers to —C(O)—.

As used herein, an “oxo” refers to ═O.

As used herein a “carboxy” refers to —C(O)O—.

As used herein an “ester” refers to —C(O)O—W, in which W is, for example, alkyl, carbocyclyl, or heterocyclyl.

The phrase “optionally substituted” is used interchangeably with the phrase “substituted or unsubstituted.” As described herein, compounds of the invention can optionally be substituted with one or more substituents, as exemplified by particular classes, subclasses, and species of the invention.

In general, the term “substituted,” whether preceded by the term “optionally” or not, refers to the replacement of hydrogen radicals in a given structure with the radical of a specified substituent. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

The phrase “stable or chemically feasible,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The methods of treatment of the invention comprise administering a safe and effective amount of a combination comprising a compound described herein or a pharmaceutically-acceptable salt thereof and a checkpoint inhibitor to a patient in need thereof.

As used herein, “treat” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, an “effective amount” is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, N.Y., 537 (1970). As used herein, “patient” refers to a mammal, including a human.

Additionally, unless otherwise stated, structures depicted herein are also meant to include compounds that differ only in the presence of one or more isotopically enriched atoms. Examples of isotopes that can be incorporated into compounds of the invention and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷0, ¹⁸0, ³¹P, ³²P ³⁵S, ¹⁸F, ³⁶Cl, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labelled compounds of the present invention, for example those into which radioactive isotopes, such as ³H and ¹⁴C, are incorporated, are useful in drug and/or substrate tissue distribution assays. Tritiated hydrogen (H) and carbon-14 (¹⁴C) isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium (²H) can afford certain therapeutic advantages resulting from greater metabolic stability, for example increased in vivo half-life or reduced dosage requirements and may be preferred in some circumstances. Isotopically labeled compounds of the invention can generally be prepared by carrying out the procedures disclosed in the schemes and/or in the examples below, and substituting a readily available isotopically labeled reagent for a non-isotopically labeled reagent.

Unless otherwise stated, structures depicted herein are also meant to include all isomeric (e.g., enantiomeric, diastereomeric, and geometric (or conformational)) forms of the structure. “Isomer” refers to compounds that have the same composition and molecular weight but differ in physical and/or chemical properties; for example (Z) and (E) double bond isomers, and (Z) and (E) conformational isomers. The structural difference may be in constitution (geometric isomers) or in the ability to rotate the plane of polarized light (stereoisomers); for example, the R and S configurations for each asymmetric center. The compounds of the invention may contain one or more asymmetric centers, also referred to as chiral centers, and may, therefore, exist as individual enantiomers, diastereomers, or other stereoisomeric forms, or as mixtures thereof. All such isomeric forms are included within the present invention, including mixtures thereof. Chiral centers may also be present in a substituent such as an alkyl group.

Where the stereochemistry of a chiral center present in a compound of the invention, or in any chemical structure illustrated herein, is not specified the structure is intended to encompass any stereoisomer and all mixtures thereof. Thus, compounds of the invention containing one or more chiral centers may be used as racemic mixtures, enantiomerically enriched mixtures, or as enantiomerically pure individual stereoisomers.

Individual stereoisomers of a compound of the invention which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

Unless otherwise stated, all tautomeric forms of the compounds of the invention are within the scope of the invention.

Any numerical range disclosed herein encompasses the and lower limits and each intervening value, unless otherwise specified. Other than in the working examples, or where otherwise indicated, numerical values (such as numbers expressing quantities of ingredients, reaction conditions) as used in the specification and claims are modified by the term “about”. Accordingly, unless indicated to the contrary, such numbers are approximations that may vary depending upon the desired properties sought to be obtained. At the very least, and not as an attempt to limit the application of the doctrine of equivalents to the scope of the claims, each numerical parameter should be construed in light of the number of significant digits and ordinary rounding techniques.

While the numerical parameters setting forth the scope of the disclosed subject matter are approximations, the numerical values set forth in the working examples are reported as precisely as possible. Any numerical value, however, inherently contains certain errors necessarily resulting from the standard deviation found in its respective testing measurements.

Unless defined otherwise, the meanings of technical and scientific terms as used herein are those commonly understood by one of ordinary skill in the art to which the disclosed subject matter belongs.

As described herein, compounds of the disclosure may optionally be substituted with one or more substituents, such as are illustrated generally above, or as exemplified by particular classes, subclasses, and species of the disclosure.

Additionally, unless otherwise stated, structures depicted herein are also meant to include compounds that differ only in the presence of one or more isotopically enriched atoms. Examples of isotopes that can be incorporated into compounds of the disclosure and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷0, ¹⁸0, ³¹P, ³²P, ³⁵S, ¹⁸F, ³⁶Cl, ¹²³I and ¹²⁵I.

Compounds of the present disclosure and pharmaceutically acceptable salts of said compounds that contain the aforementioned isotopes and/or other isotopes of other atoms are within the scope of the present disclosure. Isotopically-labelled compounds of the present disclosure, for example those into which radioactive isotopes, such as ³H and ¹⁴C, are incorporated, are useful in drug and/or substrate tissue distribution assays. Tritiated hydrogen (³H) and carbon-14 (¹⁴C) isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Further, substitution with heavier isotopes such as deuterium (²H) can afford certain therapeutic advantages resulting from greater metabolic stability, for example increased in vivo half-life or reduced dosage requirements and may be preferred in some circumstances. Isotopically labeled compounds of the disclosure can generally be prepared by carrying out the procedures disclosed in the schemes and/or in the examples below, and substituting a readily available isotopically labeled reagent for a non-isotopically labeled reagent.

Unless otherwise stated, structures depicted herein are also meant to include all isomeric (e.g., enantiomeric, diastereomeric, and geometric (or conformational)) forms of the structure. “Isomer” refers to compounds that have the same composition and molecular weight but differ in physical and/or chemical properties; for example (Z) and (E) double bond isomers, and (Z) and (E) conformational isomers. The structural difference may be in constitution (geometric isomers) or in the ability to rotate the plane of polarized light (stereoisomers); for example, the R and S configurations for each asymmetric center. The compounds of the disclosure may contain one or more asymmetric centers, also referred to as chiral centers, and may, therefore, exist as individual enantiomers, diastereomers, or other stereoisomeric forms, or as mixtures thereof. All such isomeric forms are included within the present disclosure, including mixtures thereof. Chiral centers may also be present in a substituent such as an alkyl group.

Where the stereochemistry of a chiral center present in a compound of the disclosure, or in any chemical structure illustrated herein, is not specified the structure is intended to encompass any stereoisomer and all mixtures thereof. Thus, compounds of the disclosure containing one or more chiral centers may be used as racemic mixtures, enantiomerically enriched mixtures, or as enantiomerically pure individual stereoisomers.

Individual stereoisomers of a compound of the disclosure which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

Unless otherwise stated, all tautomeric forms of the compounds of the disclosure are within the scope of the disclosure.

In various embodiments, the present disclosure provides a combination comprising a compound of Formula I, or a pharmaceutically acceptable salt thereof, and a checkpoint modulator, for example, an immune checkpoint inhibitor. For example, a compound of Formula I includes a compound represented by the Formula I.

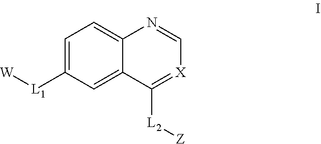

wherein X is N or C—R₄;

L₁ and L₂ are each independently a bond or a C₁-C₆ branched or straight alkylene group, wherein up to three carbon units of said alkylene group are optionally and independently replaced with a bivalent moiety selected from the group consisting of —CO—, —CS—, —CONR—, —CONRNR—, —CO₂—, —OCO—, —NRCO₂—, —O—, —CR═CR—, —C≡C—, —NRCONR—, —OCONR—, —NRNR—, —NRCO—, —S—, —S(O)—, —S(O)₂—, —NR—, —S(O)₂NR—, —NRS(O)₂—, and —NRS(O)₂NR—;

W is selected from the group consisting of halo, 5-10 membered heteroaryl, 5-10 membered heterocyclyl, C₃-C₁₀ carbocyclyl, naphthyl, and phenyl, wherein W is optionally substituted with up to three R₁ substituents;

Z is selected from the group consisting of 5-10 membered heteroaryl, 5-10 membered heterocyclyl, C₃-C₁₀ carbocyclyl, aryl, benzyl, and phenyl, wherein Z is optionally substituted with up to three R₃ substituents;

R₁ is selected from the group consisting of halo, CN, C₁-C₆ alkyl, phenyl, 5-6 membered heteroaryl, 5-6 membered heterocyclyl, C₃-C₆ carbocyclyl, —OR, —CONR₂, —CONRNR₂, —CO₂R, —S(O)₂R, —NR₂, —NRS(O)₂R, —S(O)₂NR₂, and —NRCONR₂, wherein R₁ is optionally substituted with up to two R₂ substituents.

R₂ is selected from the group consisting of halo, C₁-C₆ alkyl, C₁-C₆ alkoxy, phenyl, 5-6 membered heteroaryl, 5-6 membered heterocyclyl, C₃-C₆ carbocyclyl, —OH, oxo, —NR₂, wherein each R₂ is optionally and independently substituted with 5-6 membered heterocyclyl;

R₃ is selected from the group consisting of R, halo, —OR, —O(CH₂)ₙR, and —(CH₂)ₙOR;

R₄ is selected from the group consisting of H, halo, C₁-C₄ alkyl, CN, OH, and —COOH;

R is selected from the group consisting of H, C₁-C₄ alkyl, C₂-C₄ alkenyl, C₂-C₄ alkynyl, phenyl, 5-6 membered heteroaryl, 5-6 membered heterocyclyl, C₃-C₆ carbocyclyl, alkylsulfonyl, and —CONH(C₁-C₄ alkyl); n is 1, 2, or 3; and

provided that the compound of Formula I is not

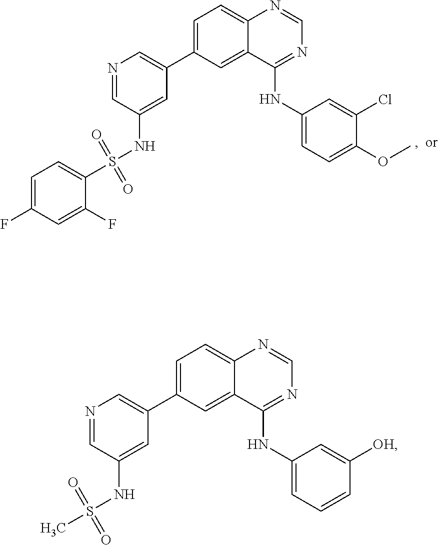

or a pharmaceutically acceptable salt thereof.

In one embodiment, R₄ is CN.

In another embodiment, W is selected from the group consisting of halo, 5-10 membered heteroaryl, and phenyl, wherein W is optionally substituted with up to three R₁ substituents.

In a further embodiment, W is halo, 5-10 membered heteroaryl, or phenyl, wherein W is optionally substituted with one or two R₁ substituents selected from the group consisting of halo, OH, CN, C₁-C₆ alkyl, —OC₁-C₆ alkyl, —NHS(O)₂(C₁-C₆ alkyl), —NHS(O)₂(C₂-C₆ alkenyl), —NHS(O)₂(C₃-C₆ carbocyclyl), —NHS(O)₂ (5-6 membered heterocyclyl), —N(S(O)₂(C₁-C₆ alkyl))₂, —NRS(O)₂-phenyl, —NH₂, —NHC(O)NH(C₁-C₆ alkyl), 5-6 membered heteroaryl, —CO₂(C₁-C₆ alkyl), —COOH, 5-6 membered heterocyclyl, 5-6 membered heteroaryl, and —CONHNHCONH(C₁-C₄ alkyl), wherein R₁ is optionally substituted with up to two R₂ substituents.

In a further embodiment, W is halo, pyridyl, pyrimidinyl, pyrrolo[2,3-b]pyridyl, pyrazolyl, pyrazolo[3,4-b]pyridyl, or phenyl, wherein W is optionally substituted with one or two R₁ substituents selected from the group consisting of halo, OH, CN, C₁-C₆ alkyl, —OC₁-C₆ alkyl, —NHS(O)₂(C₁-C₆ alkyl), —NHS(O)₂(C₂-C₆ alkenyl), —NHS(O)₂(C₃-C₆ carbocyclyl), —NHS(O)₂ (5-6 membered heterocyclyl), —N(S(O)₂(C₁-C₆ alkyl))₂, —NRS(O)₂-phenyl, —NH₂, —NHC(O)NH(C₁-C₆ alkyl), 5-6 membered heteroaryl, —CO₂(C₁-C₆ alkyl), —COOH, 5-6 membered heterocyclyl, 5-6 membered heteroaryl, and —CONHNHCONH(C₁-C₄ alkyl), wherein R₁ is optionally substituted with up to two R₂ substituents.

In still a further embodiment, W is halo, pyridyl, pyrimidinyl, pyrrolo[2,3-b]pyridyl, pyrazolyl, pyrazolo[3,4-b]pyridyl, or phenyl, wherein W is optionally substituted with one or two R₁ substituents selected from the group consisting of halo, OH, CN, hydroxyl(C₁-C₆ alkyl), —OC₁-C₆ alkyl, —NHS(O)₂(C₁-C₆ alkyl), —NHS(O)₂(C₁-C₆ alkyl)-(5-6 membered heterocyclyl), —NHS(O)₂(C₂-C₆ alkenyl), —NHS(O)₂(C₃-C₆ carbocyclyl), —NHS(O)₂ (5-6 membered heterocyclyl), —NHS(O)₂ (5-6 membered heterocyclyl)-(C₁-C₆ alkyl), —N(S(O)₂(C₁-C₆ alkyl))₂, —NRS(O)₂-phenyl-halo, —NH₂, —NHC(O)NH(C₁-C₆ alkyl), 5-6 membered heteroaryl, 5-6 membered heteroaryl-NH(C₁-C₆ alkyl)-(5-6 membered heterocyclyl), —CO₂(C₁-C₆ alkyl), —COOH, 5-6 membered heterocyclyl, 5-6 membered heterocyclyl-oxo, —CONHNHCONH(C₁-C₄ alkyl)-(5-6 membered heterocyclyl), and —CONHNHCONH(C₁-C₄ alkyl).

In still a further embodiment, W is halo, pyridyl, pyrimidinyl, pyrrolo[2,3-b]pyridyl, pyrazolyl, pyrazolo[3,4-b]pyridyl, or phenyl, wherein W is optionally substituted with one or two R₁ substituents selected from the group consisting of halo, OH, —NH₂, —COOH, CN, hydroxymethyl, methoxy, methylsulfonylamino, N-morpholinoethylsulfonylamino, ethenylsulfonylamino, cyclopropylsulfonylamino, N-methyl-N′-morpholinosulfonylamino, bis(methylsulfonyl)amino, fluorophenylsulfonylamino, methylaminocarbonylamino, tetrazolyl, N-morpholinoethylamino-oxadiazolyl, methoxycarbonyl, oxadiazole-2-oneyl, and N-morpholinoethylaminocarbonylhydrazylcarbonyl.

In still a further embodiment, W is selected from Br,

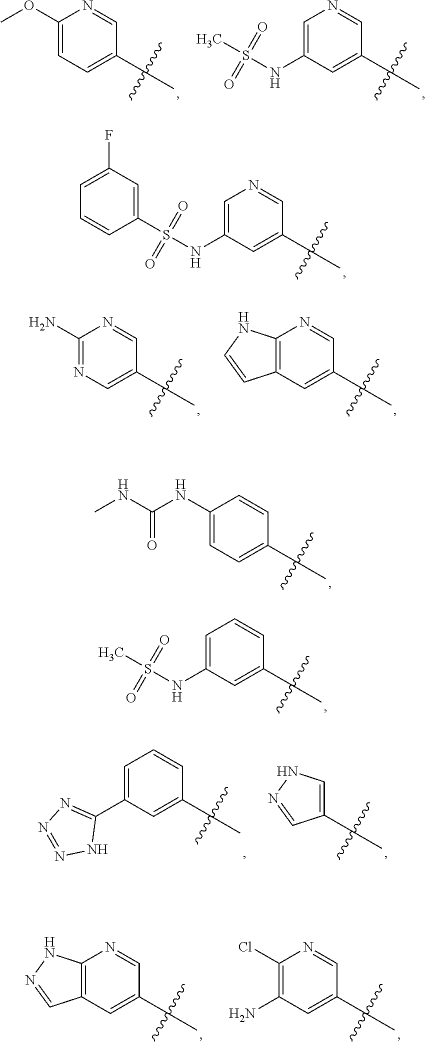

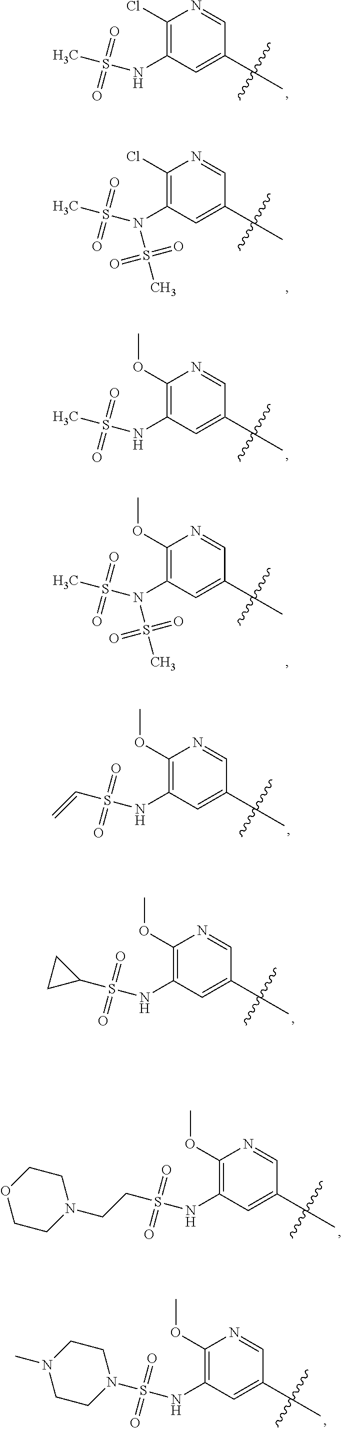

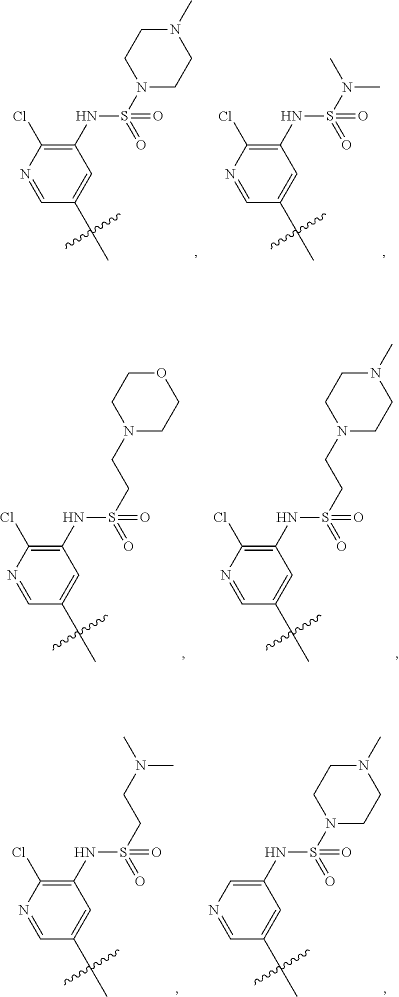 [SMILES: *c1cnc(Cl)c(NS(=O)(=O)N2CCN(C)CC2)c1 ; *c1cnc(Cl)c(NS(=O)(=O)N(C)C)c1 ; *c1cnc(Cl)c(NS(=O)(=O)CCN2CCOCC2)c1 ; *c1cnc(Cl)c(NS(=O)(=O)CCN2CCN(C)CC2)c1 ; *c1cnc(Cl)c(NS(=O)(=O)CCN(C)C)c1 ; *c1cncc(NS(=O)(=O)N2CCN(C)CC2)c1]

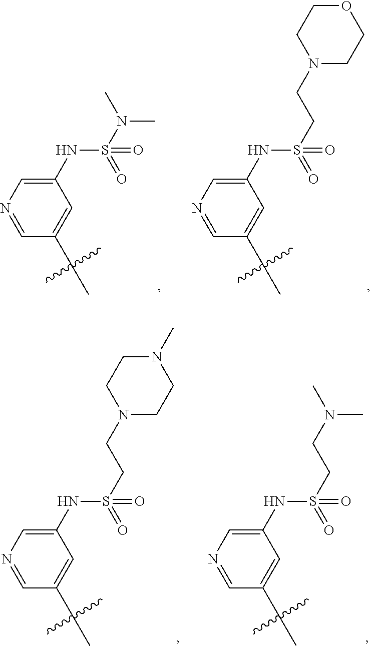 [SMILES: *c1cncc(NS(=O)(=O)N(C)C)c1 ; *c1cncc(NS(=O)(=O)CCN2CCOCC2)c1 ; *c1cncc(NS(=O)(=O)CCN2CCN(C)CC2)c1 ; *c1cncc(NS(=O)(=O)CCN(C)C)c1]

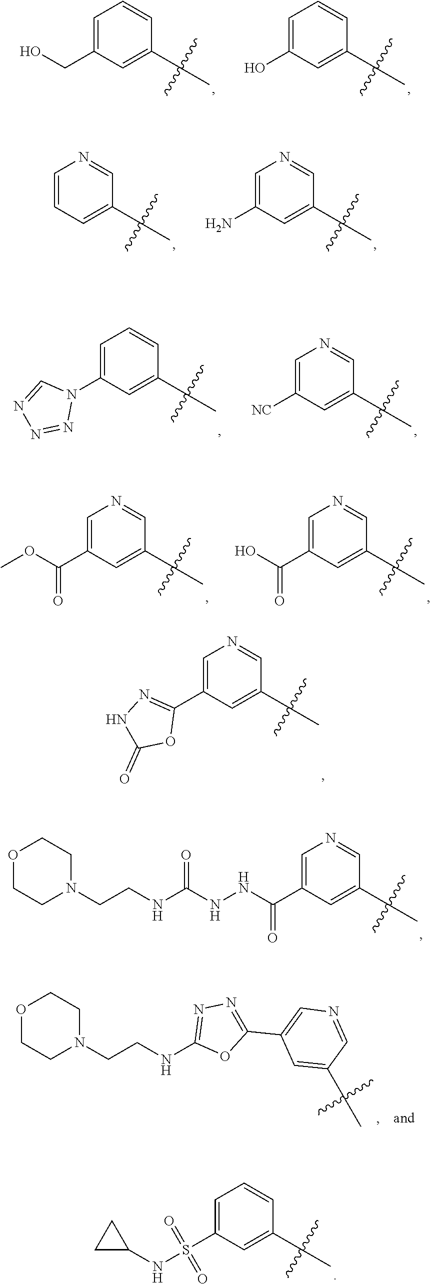

In one embodiment, Z is selected from the group consisting of 5-6 membered heteroaryl, aryl, benzyl, and phenyl, wherein Z is optionally substituted with up to three R₃ substituents.

In another embodiment, Z is selected from the group consisting of 5-6 membered heteroaryl, aryl, benzyl, and phenyl, wherein Z is optionally substituted with up to three substituents selected from halo, —O(C₁-C₄ alkyl), —O(5-6 membered heteroaryl), C₁-C₄ alkyl, C₂-C₄ alkynyl, —OCH₂ (5-6 membered heteroaryl), and —CH₂O (5-6 membered heteroaryl).

In a further embodiment, Z is selected from the group consisting of pyridyl and phenyl, wherein Z is optionally substituted with up to three substituents selected from halo, —O(C₁-C₄ alkyl), —O(5-6 membered heteroaryl), C₂-C₄ alkynyl, and —OCH₂ (5-6 membered heteroaryl).

In still a further embodiment, Z is selected from the group consisting of fluorochlorophenyl, methoxychlorophenyl, ethynylphenyl, chloropyridyl, chlorophenyl, bromophenyl, pyridyloxyphenyl, phenyl, and pyridylmethyloxyphenyl.

In yet a further embodiment, Z is selected from the group consisting of

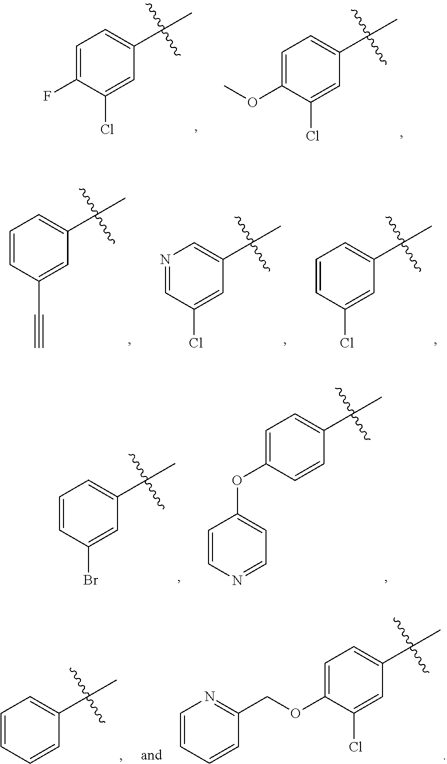

In one embodiment, L₁ is selected from the group consisting of a bond or a C₁-C₆ branched or straight alkylene group, wherein up to three carbon units of said alkylene group are optionally and independently replaced with a bivalent moiety selected from the group consisting of —CO—, —CONH—, —CO₂—, —O—, —C≡C—, —NHCO—, —S(O)₂—, —NH—, —S(O)₂NH—, and —NHS(O)₂—.

In a further embodiment, L₁ is selected from the group consisting of a bond and —C≡C—

In still a further embodiment, L₁ is a bond.

In one embodiment, L₂ is selected from the group consisting of a bond or a C₁-C₆ branched or straight alkylene group, wherein up to three carbon units of said alkylene group are optionally and independently replaced with a bivalent moiety selected from the group consisting of —CONR—, —CO₂—, —O—, —NRCO—, —NR—, —S(O)₂NR—, and —NRS(O)₂—.

In a further embodiment, L₂ is selected from the group consisting of —NH— and —NHCH₂—.

In still a further embodiment, L₂ is —NH—.

In one embodiment, X is N.

In some embodiments, a compound of Formula I for use in the combination and methods described herein for the prevention of cancer, and metastasis and/or for the treatment of cancer and/or metastasis, is a compound of Formula Ia, or a pharmaceutically acceptable salt thereof:

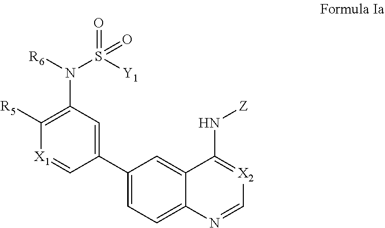

wherein

### X₁ is N or CH;

### X₂ is N or C—CN;

Z is selected from the group consisting of 5-6 membered heteroaryl and phenyl, wherein Z is optionally substituted with up to three R₃ substituents;
R₅ is H, OH, CN, NH₂, NO₂, O(C₁-C₄ alkyl), C₁-C₄ alkyl, C₂-C₄ alkenyl, C₂-C₄ alkynyl, 5-6 membered heteroaryl, 5-6 membered heterocyclyl, and C₃-C₆ carbocyclyl;
R₆ is H, C₁-C₄ alkyl, or —S(O)₂(C₁-C₄ alkyl); and
Y₁ is selected from the group consisting of H, OH, O(C₁-C₄ alkyl), C₁-C₄ alkyl, C₂-C₄ alkyl(R₇), C₂-C₄ alkenyl, C₂-C₄ alkynyl, 5-6 membered heteroaryl, 5-6 membered heterocyclyl, and C₃-C₆ carbocyclyl, wherein Y₁ is optionally substituted with up to two instances of 5-6 membered heterocyclyl, 5-6 membered carbocyclyl, O(C₁-C₄ alkyl), C₁-C₄ alkyl, OH, CN, halo, NO₂, and NH₂; and
R₇ is selected from NH₂, N(H)(C₁-C₄ alkyl), N(C₁-C₄ alkyl)₂, 3-7 membered heterocyclyl; provided that the compound of Formula Ia is not

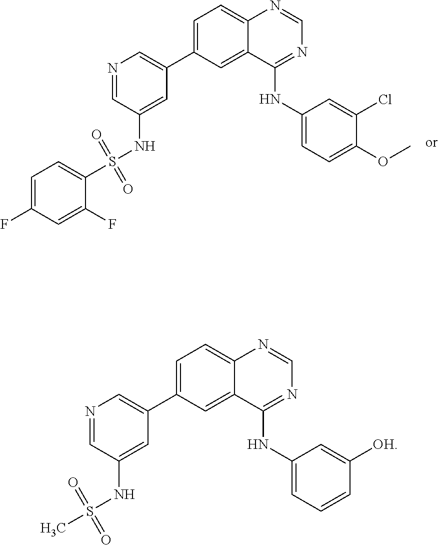

In some embodiments, the compound of Formula I, or Formula Ia, is selected from the following compounds or their pharmaceutically acceptable salts thereof:

| IUPAC Name and Chemical Structure | Compound ID |
|---|---|
| 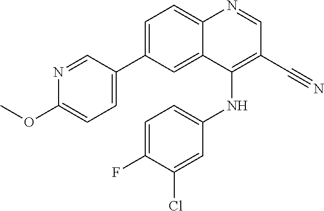 [SMILES: COc1ccc(-c2ccc3ncc(C#N)c(Nc4ccc(F)c(Cl)c4)c3c2)cn1] 4-((3-chloro-4-fluorophenyl)amino)-6-(6-methoxypyridin-3-yl)quinoline-3- carbonitrile | MOL-150 |
| 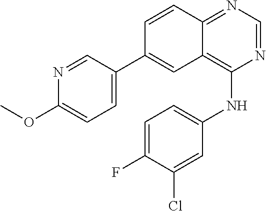 [SMILES: COc1ccc(-c2ccc3ncnc(Nc4ccc(F)c(Cl)c4)c3c2)cn1] N-(3-chloro-4-fluorophenyl)-6-(6-methoxypyridin-3-yl)quinazolin-4-amine | MOL-151 |
| 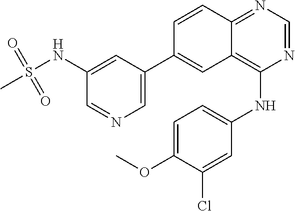 [SMILES: COc1ccc(Nc2ncnc3ccc(-c4cncc(NS(C)(=O)=O)c4)cc23)cc1Cl] N-(5-(4-((3-chloro-4-methoxyphenyl)amino)quinazolin-6-yl)pyridin-3- yl)methanesulfonamide | MOL-153 |
| 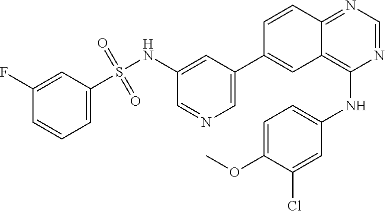 [SMILES: COc1ccc(Nc2ncnc3ccc(-c4cncc(NS(=O)(=O)c5cccc(F)c5)c4)cc23)cc1Cl] N-(5-(4-((3-chloro-4-methoxyphenyl)amino)quinazolin-6-yl)pyridin-3-yl)-3- fluorobenzenesulfonamide | MOL-154 |
| 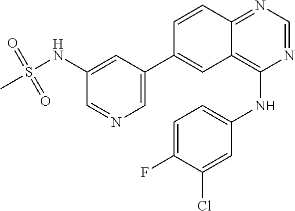 [SMILES: CS(=O)(=O)Nc1cncc(-c2ccc3ncnc(Nc4ccc(F)c(Cl)c4)c3c2)c1] N-(5-(4-((3-chloro-4-fluorophenyl)amino)quinazolin-6-yl)pyridin-3- yl)methanesulfonamide | MOL-160 |
| 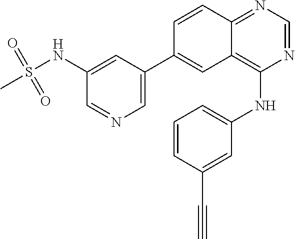 [SMILES: C#Cc1cccc(Nc2ncnc3ccc(-c4cncc(NS(C)(=O)=O)c4)cc23)c1] N-(5-(4-((3-ethynylphenyl)amino)quinazolin-6-yl)pyridin-3- yl)methanesulfonamide | MOL-161 |
| 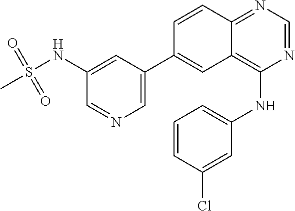 [SMILES: CS(=O)(=O)Nc1cncc(-c2ccc3ncnc(Nc4cccc(Cl)c4)c3c2)c1] N-(5-(4-((3-chlorophenyl)amino)quinazolin-6-yl)pyridin-3- yl)methanesulfonamide | MOL-162 |
| 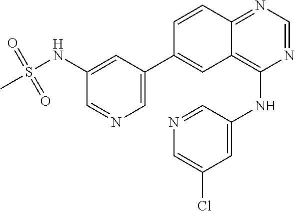 [SMILES: CS(=O)(=O)Nc1cncc(-c2ccc3ncnc(Nc4cncc(Cl)c4)c3c2)c1] N-(5-(4-((5-chloropyridin-3-yl)amino)quinazolin-6-yl)pyridin-3- yl)methanesulfonamide | MOL-163 |
| 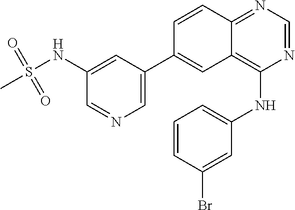 [SMILES: CS(=O)(=O)Nc1cncc(-c2ccc3ncnc(Nc4cccc(Br)c4)c3c2)c1] N-(5-(4-((3-bromophenyl)amino)quinazolin-6-yl)pyridin-3- yl)methanesulfonamide | MOL-165 |
| 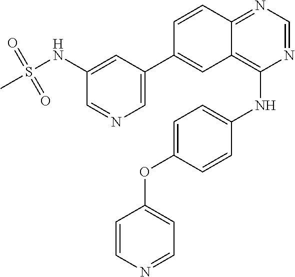 [SMILES: CS(=O)(=O)Nc1cncc(-c2ccc3ncnc(Nc4ccc(Oc5ccncc5)cc4)c3c2)c1] N-(5-(4-((4-(pyridin-4-yloxy)phenyl)amino)quinazolin-6-yl)pyridin-3- yl)methanesulfonamide | MOL-166 |
| 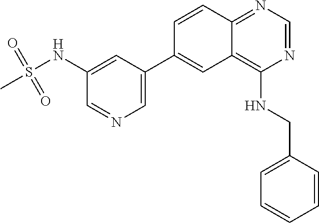 [SMILES: CS(=O)(=O)Nc1cncc(-c2ccc3ncnc(NCc4ccccc4)c3c2)c1] N-(5-(4-(benzylamino)quinazolin-6-yl)pyridin-3-yl)methanesulfonamide | MOL-167 |
| 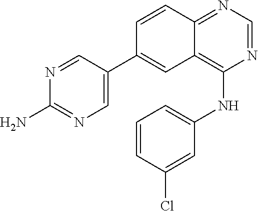 [SMILES: Nc1ncc(-c2ccc3ncnc(Nc4cccc(Cl)c4)c3c2)cn1] 6-(2-aminopyrimidin-5-yl)-N-(3-chlorophenyl)quinazolin-4-amine | MOL-171 |
| 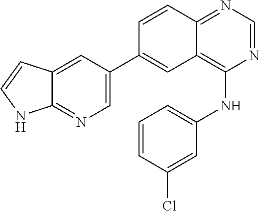 [SMILES: Clc1cccc(Nc2ncnc3ccc(-c4cnc5[nH]ccc5c4)cc23)c1] N-(3-chlorophenyl)-6-(1H-pyrrolo[2,3-b]pyridin-5-yl)quinazolin-4-amine | MOL-172 |
| 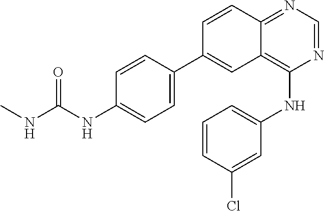 [SMILES: CNC(=O)Nc1ccc(-c2ccc3ncnc(Nc4cccc(Cl)c4)c3c2)cc1] 1-(4-(4-((3-chlorophenyl)amino)quinazolin-6-yl)phenyl)-3-methylurea | MOL-173 |
| 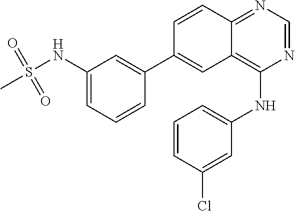 [SMILES: CS(=O)(=O)Nc1cccc(-c2ccc3ncnc(Nc4cccc(Cl)c4)c3c2)c1] N-(3-(4-((3-chlorophenyl)amino)quinazolin-6-yl)phenyl)methanesulfonamide | MOL-174 |
| 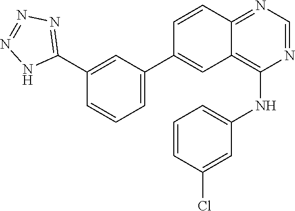 [SMILES: Clc1cccc(Nc2ncnc3ccc(-c4cccc(-c5nnn[nH]5)c4)cc23)c1] 6-(3-(1H-tetrazol-5-yl)phenyl)-N-(3-chlorophenyl)quinazolin-4-amine | MOL-175 |
| 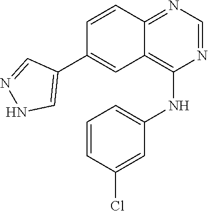 [SMILES: Clc1cccc(Nc2ncnc3ccc(-c4cn[nH]c4)cc23)c1] N-(3-chlorophenyl)-6-(1H-pyrazol-4-yl)quinazolin-4-amine | MOL-176 |
| 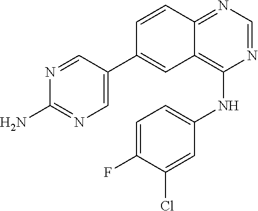 [SMILES: Nc1ncc(-c2ccc3ncnc(Nc4ccc(F)c(Cl)c4)c3c2)cn1] 6-(2-aminopyrimidin-5-yl)-N-(3-chloro-4-fluorophenyl)quinazolin-4-amine | MOL-181 |
| 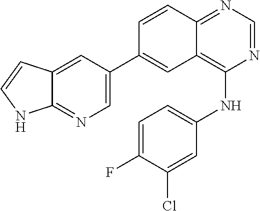 [SMILES: Fc1ccc(Nc2ncnc3ccc(-c4cnc5[nH]ccc5c4)cc23)cc1Cl] N-(3-chloro-4-fluorophenyl)-6-(1H-pyrrolo[2,3-b]pyridin-5-yl)quinazolin-4- amine | MOL-182 |
| 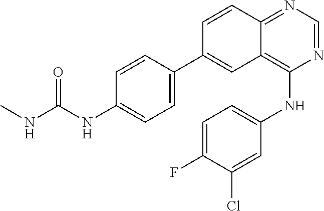 [SMILES: CNC(=O)Nc1ccc(-c2ccc3ncnc(Nc4ccc(F)c(Cl)c4)c3c2)cc1] 1-(4-(4-((3-chloro-4-fluorophenyl)amino)quinazolin-6-yl)phenyl)-3- methylurea | MOL-183 |
| 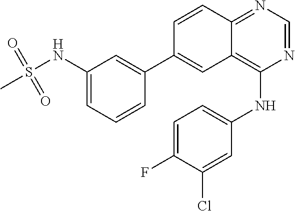 [SMILES: CS(=O)(=O)Nc1cccc(-c2ccc3ncnc(Nc4ccc(F)c(Cl)c4)c3c2)c1] N-(3-(4-((3-chloro-4-fluorophenyl)amino)quinazolin-6- yl)phenyl)methanesulfonamide | MOL-184 |
| 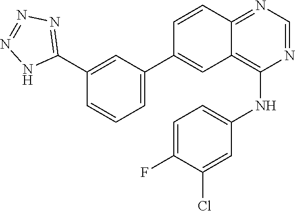 [SMILES: Fc1ccc(Nc2ncnc3ccc(-c4cccc(-c5nnn[nH]5)c4)cc23)cc1Cl] 6-(3-(1H-tetrazol-5-yl)phenyl)-N-(3-chloro-4-fluorophenyl)quinazolin-4- amine | MOL-185 |
| 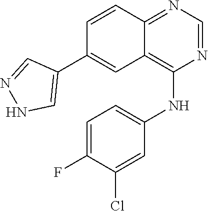 [SMILES: Fc1ccc(Nc2ncnc3ccc(-c4cn[nH]c4)cc23)cc1Cl] N-(3-chloro-4-fluorophenyl)-6-(1H-pyrazol-4-yl)quinazolin-4-amine | MOL-186 |
| 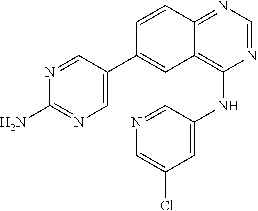 [SMILES: Nc1ncc(-c2ccc3ncnc(Nc4cncc(Cl)c4)c3c2)cn1] 6-(2-aminopyrimidin-5-yl)-N-(5-chloropyridin-3-yl)quinazolin-4-amine | MOL-191 |
| 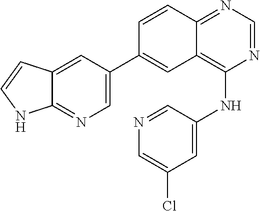 [SMILES: Clc1cncc(Nc2ncnc3ccc(-c4cnc5[nH]ccc5c4)cc23)c1] N-(5-chloropyridin-3-yl)-6-(1H-pyrrolo[2,3-b]Apyridin-5-yl)quinazolin-4- amine | MOL-192 |
| 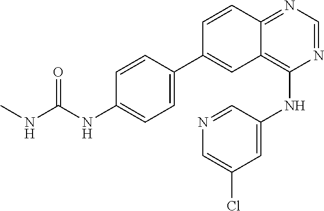 [SMILES: CNC(=O)Nc1ccc(-c2ccc3ncnc(Nc4cncc(Cl)c4)c3c2)cc1] 1-(4-(4-((5-chloropyridin-3-yl)amino)quinazolin-6-yl)phenyl)-3-methylurea | MOL-193 |
| 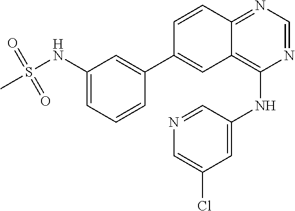 [SMILES: CS(=O)(=O)Nc1cccc(-c2ccc3ncnc(Nc4cncc(Cl)c4)c3c2)c1] N-(3-(4-((5-chloropyridin-3-yl)amino)quinazolin-6- yl)phenyl)methanesulfonamide | MOL-194 |
| 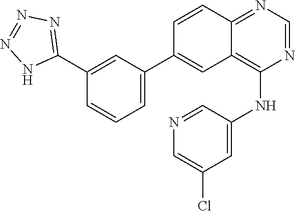 [SMILES: Clc1cncc(Nc2ncnc3ccc(-c4cccc(-c5nnn[nH]5)c4)cc23)c1] 6-(3-(1H-tetrazol-5-yl)phenyl)-N-(5-chloropyridin-3-yl)quinazolin-4-amine | MOL-195 |
| 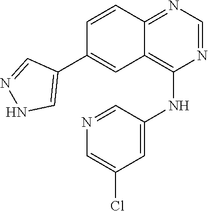 [SMILES: Clc1cncc(Nc2ncnc3ccc(-c4cn[nH]c4)cc23)c1] N-(5-chloropyridin-3-yl)-6-(1H-pyrazol-4-yl)quinazolin-4-amine | MOL-196 |
| 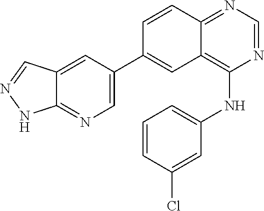 [SMILES: Clc1cccc(Nc2ncnc3ccc(-c4cnc5[nH]ncc5c4)cc23)c1] N-(3-chlorophenyl)-6-(1H-pyrazolo[3,4-b]pyridin-5-yl)quinazolin-4-amine | MOL-177 |
| 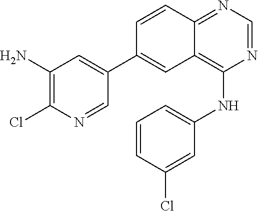 [SMILES: Nc1cc(-c2ccc3ncnc(Nc4cccc(Cl)c4)c3c2)cnc1Cl] 6-(5-amino-6-chloropyridin-3-yl)-N-(3-chlorophenyl)quinazolin-4-amine | MOL-200 |
| 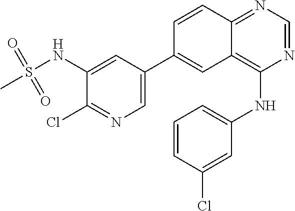 [SMILES: CS(=O)(=O)Nc1cc(-c2ccc3ncnc(Nc4cccc(Cl)c4)c3c2)cnc1Cl] N-(2-chloro-5-(4-((3-chlorophenyl)amino)quinazolin-6-yl)pyridin-3- yl)methanesulfonamide | MOL-201 |
| 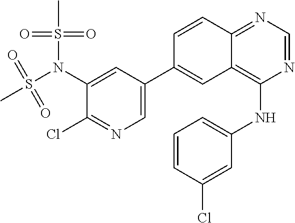 [SMILES: CS(=O)(=O)N(c1cc(-c2ccc3ncnc(Nc4cccc(Cl)c4)c3c2)cnc1Cl)S(C)(=O)=O] N-(2-chloro-5-(4-((3-chlorophenyl)amino)quinazolin-6-yl)pyridin-3-yl)-N- (methylsulfonyl)methanesulfonamide | MOL-201B |
| 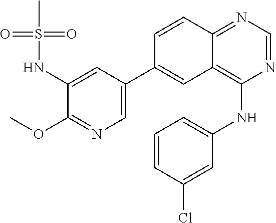 [SMILES: COc1ncc(-c2ccc3ncnc(Nc4cccc(Cl)c4)c3c2)cc1NS(C)(=O)=O] N-(5-(4-((3-chlorophenyl)amino)quinazolin-6-yl)-2-methoxypyridin-3- yl)methanesulfonamide | MOL-202 |
| 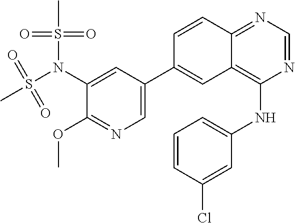 [SMILES: COc1ncc(-c2ccc3ncnc(Nc4cccc(Cl)c4)c3c2)cc1N(S(C)(=O)=O)S(C)(=O)=O] N-(5-(4-((3-chlorophenyl)amino)quinazolin-6-yl)-2-methoxypyridin-3-yl)-N- (methylsulfonyl)methanesulfonamide | MOL-202B |
| 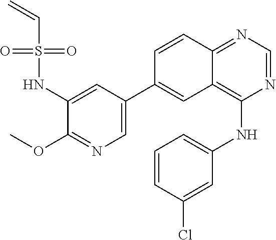 [SMILES: C=CS(=O)(=O)Nc1cc(-c2ccc3ncnc(Nc4cccc(Cl)c4)c3c2)cnc1OC] N-(5-(4-((3-chlorophenyl)amino)quinazolin-6-yl)-2-methoxypyridin-3- yl)ethenesulfonamide | MOL-203 |
| 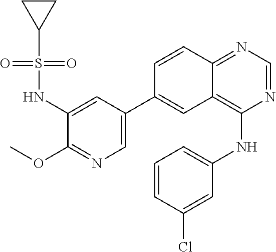 [SMILES: COc1ncc(-c2ccc3ncnc(Nc4cccc(Cl)c4)c3c2)cc1NS(=O)(=O)C1CC1] N-(5-(4-((3-chlorophenyl)amino)quinazolin-6-yl)-2-methoxypyridin-3- yl)cyclopropanesulfonamide | MOL-204 |
| 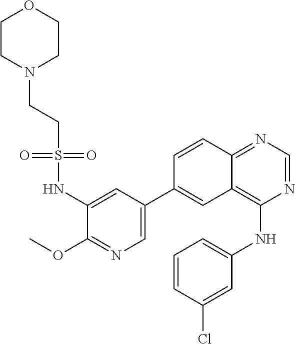 [SMILES: COc1ncc(-c2ccc3ncnc(Nc4cccc(Cl)c4)c3c2)cc1NS(=O)(=O)CCN1CCOCC1] N-(5-(4-((3-chlorophenyl)amino)quinazolin-6-yl)-2-methoxypyridin-3-yl)-2- morpholinoethane-1-sulfonamide | MOL-205 |
| 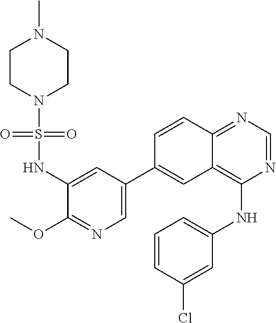 [SMILES: COc1ncc(-c2ccc3ncnc(Nc4cccc(Cl)c4)c3c2)cc1NS(=O)(=O)N1CCN(C)CC1] N-(5-(4-((3-chlorophenyl)amino)quinazolin-6-yl)-2-methoxypyridin-3-yl)-4- methylpiperazine-1-sulfonamide | MOL-207 |
| 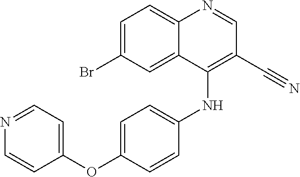 [SMILES: N#Cc1cnc2ccc(Br)cc2c1Nc1ccc(Oc2ccncc2)cc1] 6-bromo-4-((4-(pyridin-4-yloxy)phenyl)amino)quinoline-3-carbonitrile | MOL-400 |
| 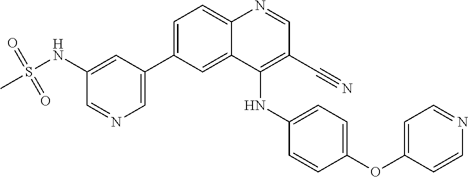 [SMILES: CS(=O)(=O)Nc1cncc(-c2ccc3ncc(C#N)c(Nc4ccc(Oc5ccncc5)cc4)c3c2)c1] N-(5-(3-cyano-4-((4-(pyridin-4-yloxy)phenyl)amino)quinolin-6-yl)pyridin-3- yl)methanesulfonamide | MOL-401 |
| 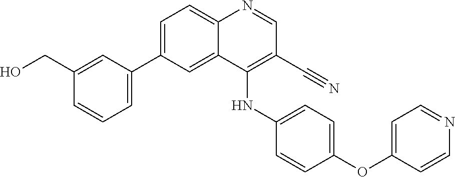 [SMILES: N#Cc1cnc2ccc(-c3cccc(CO)c3)cc2c1Nc1ccc(Oc2ccncc2)cc1] 6-(3-(hydroxymethyl)phenyl)-4-((4-(pyridin-4- yloxy)phenyl)amino)quinoline-3-carbonitrile | MOL-402 |
| 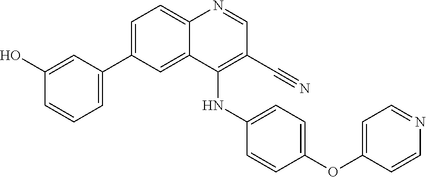 [SMILES: N#Cc1cnc2ccc(-c3cccc(O)c3)cc2c1Nc1ccc(Oc2ccncc2)cc1] 6-(3-hydroxyphenyl)-4-((4-(pyridin-4-yloxy)phenyl)amino)quinoline-3- carbonitrile | MOL-403 |
| 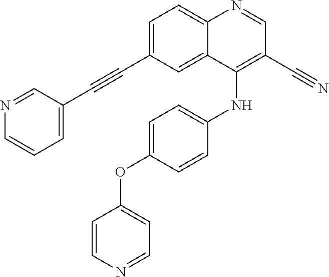 [SMILES: N#Cc1cnc2ccc(C#Cc3cccnc3)cc2c1Nc1ccc(Oc2ccncc2)cc1] 6-(pyridin-3-ylethynyl)-4-((4-(pyridin-4-yloxy)phenyl)amino)quinoline-3- carbonitrile | MOL-404 |
| 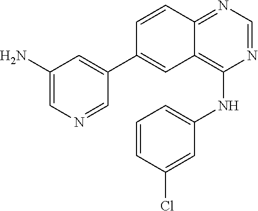 [SMILES: Nc1cncc(-c2ccc3ncnc(Nc4cccc(Cl)c4)c3c2)c1] 6-(5-aminopyridin-3-yl)-N-(3-chlorophenyl)quinazolin-4-amine | MOL-310 |
| 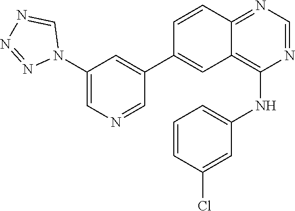 [SMILES: Clc1cccc(Nc2ncnc3ccc(-c4cncc(-n5cnnn5)c4)cc23)c1] 6-(5-(1H-tetrazol-1-yl)pyridin-3-yl)-N-(3-chlorophenyl)quinazolin-4-amine | MOL-311 |
| 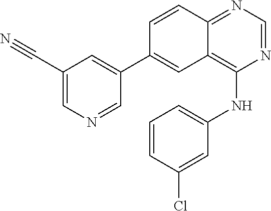 [SMILES: N#Cc1cncc(-c2ccc3ncnc(Nc4cccc(Cl)c4)c3c2)c1] 5-(4-((3-chlorophenyl)amino)quinazolin-6-yl)nicotinonitrile | MOL-312 |
| 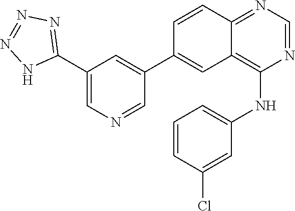 [SMILES: Clc1cccc(Nc2ncnc3ccc(-c4cncc(-c5nnn[nH]5)c4)cc23)c1] 6-(5-(1H-tetrazol-5-yl)pyridin-3-yl)-N-(3-chlorophenyl)quinazolin-4-amine | MOL-313 |
| 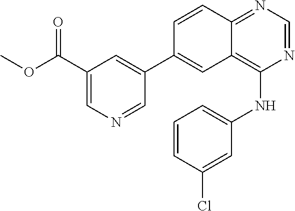 [SMILES: COC(=O)c1cncc(-c2ccc3ncnc(Nc4cccc(Cl)c4)c3c2)c1] methyl 5-(4-((3-chlorophenyl)amino)quinazolin-6-yl)nicotinate | MOL-318 |
| 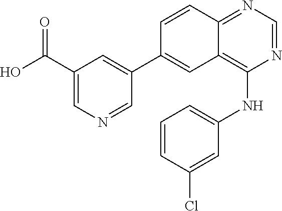 [SMILES: O=C(O)c1cncc(-c2ccc3ncnc(Nc4cccc(Cl)c4)c3c2)c1] 5-(4-((3-chlorophenyl)amino)quinazolin-6-yl)nicotinic acid | MOL-314 |
| 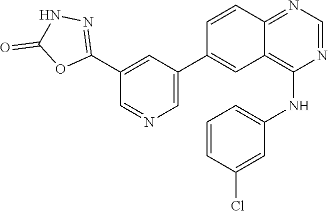 [SMILES: O=c1[nH]nc(-c2cncc(-c3ccc4ncnc(Nc5cccc(Cl)c5)c4c3)c2)o1] 5-(5-(4-((3-chlorophenyl)amino)quinazolin-6-yl)pyridin-3-yl)-1,3,4- oxadiazol-2(3H)-one | MOL-315 |
| 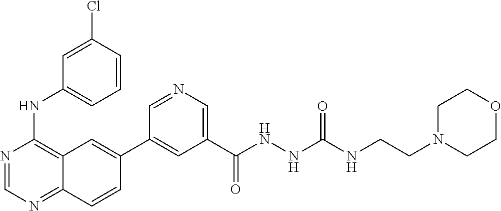 [SMILES: O=C(NCCN1CCOCC1)NNC(=O)c1cncc(-c2ccc3ncnc(Nc4cccc(Cl)c4)c3c2)c1] 2-(5-(4-((3-chlorophenyl)amino)quinazolin-6-yl)nicotinoyl)-N-(2- morpholinoethyl)hydrazine-1-carboxamide | MOL-316 |
| 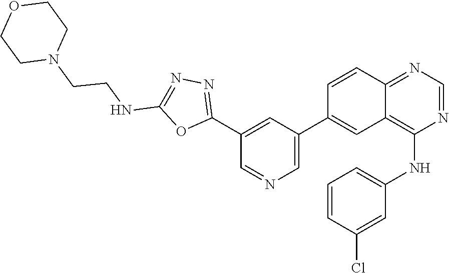 [SMILES: Clc1cccc(Nc2ncnc3ccc(-c4cncc(-c5nnc(NCCN6CCOCC6)o5)c4)cc23)c1] 5-(5-(4-((3-chlorophenyl)amino)quinazolin-6-yl)pyridin-3-yl)-N-(2- morpholinoethyl)-1,3,4-oxadiazol-2-amine | MOL-317 |
| 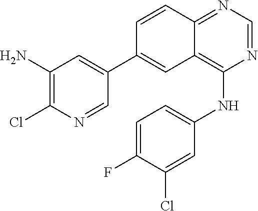 [SMILES: Nc1cc(-c2ccc3ncnc(Nc4ccc(F)c(Cl)c4)c3c2)cnc1Cl] 6-(5-amino-6-chloropyridin-3-yl)-N-(3-chloro-4-fluorophenyl)quinazolin-4- amine | MOL-210 |
| 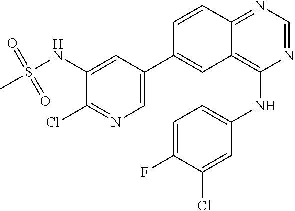 [SMILES: CS(=O)(=O)Nc1cc(-c2ccc3ncnc(Nc4ccc(F)c(Cl)c4)c3c2)cnc1Cl] N-(2-chloro-5-(4-((3-chloro-4-fluorophenyl)amino)quinazolin-6-yl)pyridin-3- yl)methanesulfonamide | MOL-211 |
| 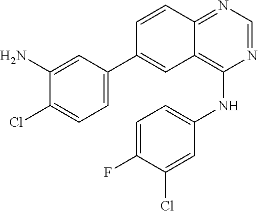 [SMILES: Nc1cc(-c2ccc3ncnc(Nc4ccc(F)c(Cl)c4)c3c2)ccc1Cl] 6-(3-amino-4-chlorophenyl)-N-(3-chloro-4-fluorophenyl)quinazolin-4-amine | MOL-212 |
| 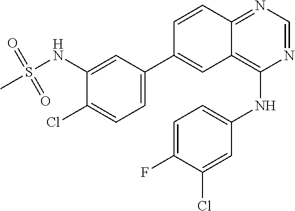 [SMILES: CS(=O)(=O)Nc1cc(-c2ccc3ncnc(Nc4ccc(F)c(Cl)c4)c3c2)ccc1Cl] N-(2-chloro-5-(4-((3-chloro-4-fluorophenyl)amino)quinazolin-6- yl)phenyl)methanesulfonamide | MOL-213 |
| 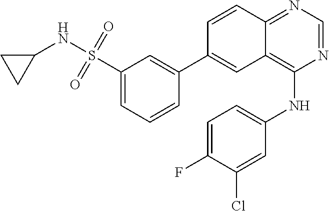 [SMILES: O=S(=O)(NC1CC1)c1cccc(-c2ccc3ncnc(Nc4ccc(F)c(Cl)c4)c3c2)c1] 3-(4-((3-chloro-4-fluorophenyl)amino)quinazolin-6-yl)-N- cyclopropylbenzenesulfonamide | MOL-214 |
| 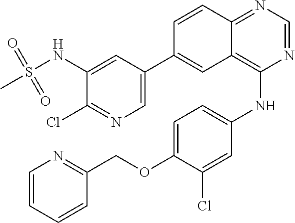 [SMILES: CS(=O)(=O)Nc1cc(-c2ccc3ncnc(Nc4ccc(OCc5ccccn5)c(Cl)c4)c3c2)cnc1Cl] N-(2-chloro-5-(4-((3-chloro-4-(pyridin-2- ylmethoxy)phenyl)amino)quinazolin-6-yl)pyridin-3-yl)methanesulfonamide | MOL-215 |
| 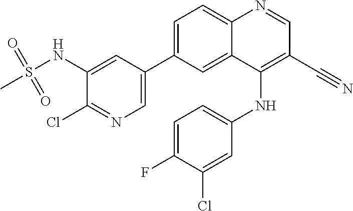 [SMILES: CS(=O)(=O)Nc1cc(-c2ccc3ncc(C#N)c(Nc4ccc(F)c(Cl)c4)c3c2)cnc1Cl] N-(2-chloro-5-(4-((3-chloro-4-fluorophenyl)amino)-3-cyanoquinolin-6- yl)pyridin-3-yl)methanesulfonamide | MOL-216 |
| 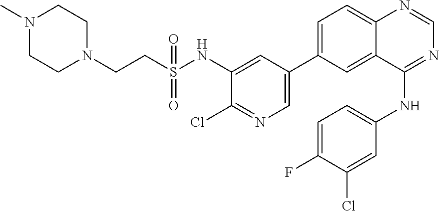 [SMILES: CN1CCN(CCS(=O)(=O)Nc2cc(-c3ccc4ncnc(Nc5ccc(F)c(Cl)c5)c4c3)cnc2Cl)CC1] N-(2-chloro-5-(4-((3-chloro-4-fluorophenyl)amino)quinazolin-6-yl)pyridin-3-yl)-2-(4- methylpiperazin-1-yl)ethane-1-sulfonamide | MOL-220 |
| 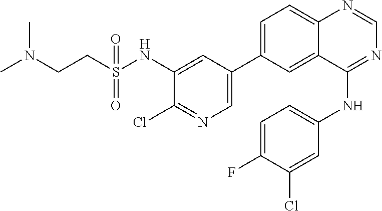 [SMILES: CN(C)CCS(=O)(=O)Nc1cc(-c2ccc3ncnc(Nc4ccc(F)c(Cl)c4)c3c2)cnc1Cl] N-(2-chloro-5-(4-((3-chloro-4-fluorophenyl)amino)quinazolin-6-yl)pyridin-3-yl)-2- (dimethylamino)ethane-1-sulfonamide | MOL-221 |
| 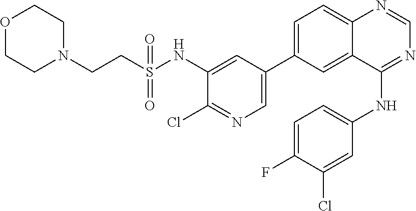 [SMILES: O=S(=O)(CCN1CCOCC1)Nc1cc(-c2ccc3ncnc(Nc4ccc(F)c(Cl)c4)c3c2)cnc1Cl] N-(2-chloro-5-(4-((3-chloro-4-fluorophenyl)amino)quinazolin-6-yl)pyridin-3-yl)-2- morpholinoethane-1-sulfonamide | MOL-222 |
| 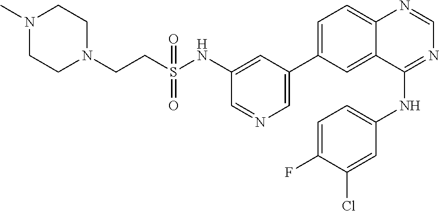 [SMILES: CN1CCN(CCS(=O)(=O)Nc2cncc(-c3ccc4ncnc(Nc5ccc(F)c(Cl)c5)c4c3)c2)CC1] N-(5-(4-((3-chloro-4-fluorophenyl)amino)quinazolin-6-yl)pyridin-3-yl)-2-(4- methylpiperazin-1-yl)ethane-1-sulfonamide | MOL-230 |
| 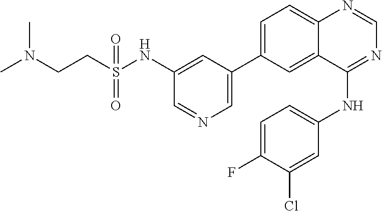 [SMILES: CN(C)CCS(=O)(=O)Nc1cncc(-c2ccc3ncnc(Nc4ccc(F)c(Cl)c4)c3c2)c1] N-(5-(4-((3-chloro-4-fluorophenyl)amino)quinazolin-6-yl)pyridin-3-yl)-2- (dimethylamino)ethane-1-sulfonamide | MOL-231 |
| 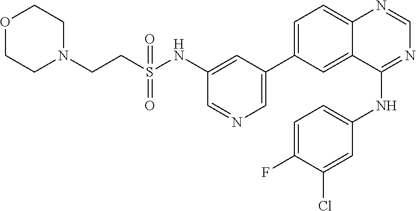 [SMILES: O=S(=O)(CCN1CCOCC1)Nc1cncc(-c2ccc3ncnc(Nc4ccc(F)c(Cl)c4)c3c2)c1] N-(5-(4-((3-chloro-4-fluorophenyl)amino)quinazolin-6-yl)pyridin-3-yl)-2- morpholinoethane-1-sulfonamide | MOL-232 |

In various embodiments, a compound of Formula I may include compound MOL-201, MOL-202, MOL-205, MOL-211, MOL-215, MOL-221, MOL-222, MOL-160, MOL-161, MOL-162, or a pharmaceutically acceptable salt of any of the foregoing.

In one embodiment, a compound of Formula I is:

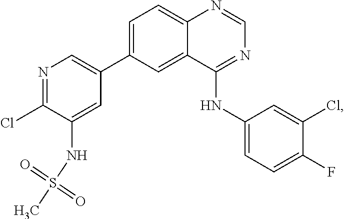 [SMILES: CS(=O)(=O)Nc1cc(-c2ccc3ncnc(Nc4ccc(F)c(Cl)c4)c3c2)cnc1Cl]

or a pharmaceutically acceptable salt thereof.

### Checkpoint Inhibitors

Immune checkpoints refer to inhibitory pathways in the immune system that are responsible for maintaining self-tolerance and modulating the degree of immune system response to minimize peripheral tissue damage. However, tumor cells can also activate immune system checkpoints to decrease the effectiveness of immune response (‘block’ the immune response) against tumor tissues. In contrast to the majority of anti-cancer agents, checkpoint inhibitors do not target tumor cells directly, but rather target lymphocyte receptors or their ligands in order to enhance the endogenous antitumor activity of the immune system (Pardoll, 2012, Nature Reviews Cancer 12:252-264). Therapy with antagonistic checkpoint blocking antibodies against immune system checkpoints such as CTLA4, PD1 and PD-L1 are one of the most promising new avenues of immunotherapy for cancer and other diseases. Additional checkpoint targets, such as TIM-3, LAG-3, various B-7 ligands, CHK 1 and CHK2 kinases, BTLA, A2aR, and others, are also under investigation. Currently, a number of checkpoint inhibitors have received approval from the U.S. Food and Drug Administration for cancer treatment, including ipilimumab (Yervoy®), a CTLA-4 inhibitor, and pembrolizumab (Keytruda®) nivolumab (Opdivo®) both PD-1 inhibitors and avelumab (Bavencio®) and durvalumab (Infinzi®). In addition, several checkpoint inhibitor agents are in clinical trials.

Programmed Cell Death Protein 1, (PD-1 or CD279), a 55-kD type 1 transmembrane protein, is a member of the CD28 family of T cell co-stimulatory receptors that include immunoglobulin superfamily member CD28, CTLA-4, inducible co-stimulator (ICOS), and BTLA. PD-1 is highly expressed on activated T cells and B cells. PD-1 expression can also be detected on memory T-cell subsets with variable levels of expression. Two ligands specific for PD-1 have been identified: programmed death-ligand 1 (PD-L1, also known as B7-H1 or CD274) and PD-L2 (also known as B7-DC or CD273). PD-L1 and PD-L2 have been shown to down-regulate T cell activation upon binding to PD-1 in both mouse and human systems (Okazaki et al., Int Immunol., 2007; 19: 813-824). The interaction of PD-1 with its ligands, PD-L1 and PD-L2, which are expressed on antigen-presenting cells (APCs) and dendritic cells (DCs), transmits negative regulatory stimuli to down-modulate the activated T cell immune response. Blockade of PD-1 suppresses this negative signal and amplifies T cell responses.

Numerous studies indicate that the cancer microenvironment manipulates the PD-L1-/PD-1 signaling pathway and that induction of PD-L1 expression is associated with inhibition of immune responses against cancer, thus permitting cancer progression and metastasis. The PD-L1/PD-1 signaling pathway is a primary mechanism of cancer immune evasion for several reasons. First, and most importantly, this pathway is involved in negative regulation of immune responses of activated T effector cells, found in the periphery. Second, PD-L1 is up-regulated in cancer microenvironments, while PD-1 is also up-regulated on activated tumor infiltrating T cells, thus possibly potentiating a vicious cycle of inhibition. Third, this pathway is intricately involved in both innate and adaptive immune regulation through bi-directional signaling. These factors make the PD-1/PD-L1 complex a central point through which cancer can manipulate immune responses and promote its own progression.

The first immune-checkpoint inhibitor to be tested in a clinical trial was ipilimumab (Yervoy, Bristol-Myers Squibb), an CTLA-4 mAb. CTLA-4 belongs to the immunoglobulin superfamily of receptors, which also includes PD-1, BTLA, TIM-3, and V-domain immunoglobulin suppressor of T cell activation (VISTA). Anti-CTLA-4 mAb is a powerful checkpoint inhibitor which removes “the brake on the immune system,” i.e., from both naive and antigen-experienced cells. Therapy enhances the antitumor function of CD8+ T cells, increases the ratio of CD8+ T cells to Foxp3+ T regulatory cells, and inhibits the suppressive function of T regulatory cells. The major drawback to anti-CTLA-4 mAb therapy is the generation of autoimmune toxicities due to on-target effects of an over-exuberant immune system which has lost the ability to turn itself down. It has been reported that up to 25% of patients treated with ipilimumab developed serious grade 3-4 adverse events/autoimmune-type side effects including dermatitis, enterocolitis, hepatitis, endocrinopathies (including hypophysitis, thyroiditis, and adrenalitis), arthritis, uveitis, nephritis, and aseptic meningitis. In contrast to the anti-CTLA-4 experience, anti-PD-1 therapy appears to be better-tolerated and induces a relatively lower rate of autoimmune-type side effects.

TIM-3 has been identified as another important inhibitory receptor expressed by exhausted CD8+ T cells. In mouse models of cancer, it has been shown that the most dysfunctional tumor-infiltrating CD8+ T cells actually co-express PD-1 and TIM-3.

LAG-3 is another recently identified inhibitory receptor that acts to limit effector T-cell function and augment the suppressive activity of T regulatory cells. It has recently been revealed that PD-1 and LAG-3 are extensively co-expressed by tumor-infiltrating T cells in mice, and that combined blockade of PD-1 and LAG-3 provokes potent synergistic antitumor immune responses in mouse models of cancer.

PD-1 pathway blockade can be combined with vaccines or other antibodies for improved therapeutic efficacy (Hirano, F. et al, Cancer Res., 65(3): 1089-1096 (2005); Li, B. et al, Clin. Cancer Res., 15: 1507-1509 (2009); and Curran, M. A. et al, Proc. Natl. Acad. Set, 107(9):4275-4280 (2010)).

Currently, antagonist mAbs against both PD-1 and ligand PD-L1 are in various stages of development for the treatment of cancer, and recent human trials have shown promising results in cancer patients with advanced, treatment-refractory disease.

The first of the agents blocking the B7-H1/PD-1 pathway to enter phase I clinical trials was Nivolumab (MDX-1106/BMS-936558/ONO-4538), a fully human IgG4 anti-PD-1 mAb developed by Bristol-Myers Squibb. Another PD-1 mAb undergoing clinical evaluation is CT-011, a humanized IgGI mAb specific for PD-1 developed by CureTech Ltd. Other agents include Lambrolizumab (MK-3475—Merck), a humanized monoclonal IgG4 PD-1 antibody; BMS-936559, a fully human IgG4 PD-L1 antibody and Roche's MPDL3280A, a human monoclonal antibody that targets the PD-L1 pathway.

Human cancers harbor numerous genetic and epigenetic alterations, generating neoantigens potentially recognizable by the immune system (Sjoblom et al. (2006) Science 314:268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have begun to provide new immunotherapeutic approaches for treating cancer, including the development of antibody (Ab), ipilimumab (YERVOY®), that binds to and inhibits CTLA-4 for the treatment of patients with advanced melanoma (Hodi et al. (2010) N Engl J Med 363:711-23) and the development of antibodies such as nivolumab and pembrolizumab (formerly lambrolizumab; USAN Council Statement (2013) Pembrolizumab: Statement on a nonproprietary name adopted by the USAN Council (ZZ-165), Nov. 27, 2013) that bind specifically to the Programmed Death-1 (PD-1) receptor and block the inhibitory PD-1/PD-1 ligand pathway (Topalian et al. (2012a) N Engl J Med 366:2443-54; Topalian et al. (2012b) Curr Opin Immunol 24:207-12; Topalian et al. (2014) J Clin Oncol 32(10):1020-30; Hamid et al. (2013) N Engl J Med 369:134-144; Hamid and Carvajal (2013) Expert Opin Biol Ther 13(6):847-61; McDermott and Atkins (2013) Cancer Med 2(5):662-73).

PD-1 is a key immune checkpoint receptor expressed by activated T and B cells and mediates immunosuppression. Nivolumab (formerly designated 5C₄, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al. (2014) In vitro characterization of the anti-PD-1 antibody nivolumab, BMS-936558, and in vivo toxicology in non-human primates. Nivolumab has been approved for the treatment of patients with unresectable or metastatic melanoma and disease progression following ipilimumab and, if BRAF V600 mutation positive, a BRAF inhibitor and for the treatment of squamous non-small cell lung cancer.

Recent data suggest a secondary mechanism of anti-CTLA-4 antibodies, which could occur within the tumor itself. CTLA-4 has been found to be expressed in tumors at higher levels on regulatory T-cells (also referred to herein as “Treg cells”) as compared with intra-tumoral effector T-cells (also referred to herein as “Teff cells”), resulting in the hypothesis of anti-CTLA-4 preferentially impacting the Treg cell. “Therapeutic use of anti-CTLA-4 antibodies”, Christian U. Blank and Alexander Enk, International Immunology, Vol. 27, No. 1, pp. 3-10. A recent study of a PD-1 and CTLA-4 combination show that the combination blockade of the CTLA-4 and PD-1 pathways also cooperates to increase the ratio of Teff cells to both regulatory T-cells and MDSCs, thereby reducing suppression and promoting inflammation in the tumor microenvironment. “Combination of CTLA-4 and PD-1 blockade expands infiltrating T-cells and reduces regulatory T and myeloid cells within B16 melanoma tumors”, Curran et al., PNAS|Mar. 2, 2010; vol. 107 (no. 9); pp. 4275-4280, the disclosure of which is incorporated herein by reference in its entirety. The combination of a checkpoint inhibitor and another therapeutic agent(s) may enhance or prolong anti-tumor response of the checkpoint inhibitor and/or effects of the therapeutic agent. In this regard, WO 2015/069770 discloses a combination treatment based on activating the adaptive immune response, in particular the combination of CTLA-4 and PD-1 inhibitors, for the treatment of cancer. The disclosure of WO 2015/069770 is incorporated by reference in its entirety in the disclosure of this application.

One mechanism by which the checkpoint blockade anti-CTLA-4 antibodies mediate anti-tumor effect is by decreasing regulatory T-cells. Due to the distinct mechanism of action of anti-CTLA-4 antibodies, they can successfully combine with the anti-PD1 checkpoint blockade antibodies which work to release the suppressive signaling conferred to effector T-cells. Dual blockade with these antibodies combine to improve anti-tumor response both preclinically (Proc Natl Acad Sci USA 2010, 107, 4275-4280) and in the clinic (N Engl J Med 2013, 369, 122-133; N Engl J Med 2015, 372, 2006-2017).

CTLA-4 attenuates the early activation of naïve and memory T cells through interactions with its ligands B7-1 (CD80) and B7-2 (CD86). PD-1 is an receptor expressed on the surface of activated mature T cells, activated NK cells, B cells, monocytes and multiple normal tissues and plays a crucial role in the maintenance of peripheral tolerance [20-21]. In contrast to CTLA-4, PD-1 acts via interactions with its ligands PD-L1 (also known as B7-H1 or CD274) and is involved mainly in T cell activity modulation in peripheral tissues as well as providing a major immune resistance mechanism within the tumor microenvironment.

Expression Inhibitors

A checkpoint inhibitor can be any molecule, agent, treatment and/or method of inhibiting an immune checkpoint, and/or promoting an inhibitor of an immune checkpoint protein, e.g., by promoting an intrinsic immune checkpoint inhibitor; inhibiting a transcription factor involved in the expression of an immune checkpoint; and/or by acting in concert with some additional extrinsic factor. For example, a checkpoint inhibitor could include a treatment that inhibits transcription factors involved the expression of immune checkpoint genes, or promotes the expression of transcription factors for tumor-suppressor genes, e.g., BACH2 (Luan et al., (2016). Transcription Factors and Checkpoint Inhibitor Expression with Age: Markers of Immunosenescence. Blood, 128(22), 5983). Moreover, a checkpoint inhibitor can inhibit the transcription of immune checkpoint genes; the modification and/or processing of immune checkpoint mRNA; the translation of immune checkpoint proteins; and/or molecules involved in immunity or the immune checkpoint pathway, e.g., PD-1 transcription factors such as HIF-1, STAT3, NF-κB, and AP-1, or the activation of common oncogenic pathways such as JAK/STAT, RAS/ERK, or PI3K/AKT/mTOR (Zerdes et al., Genetic, transcriptional and post-translational regulation of the programmed death protein ligand 1 in cancer: biology and clinical correlations, Oncogene, volume 37, p. 4639-4661 (2018), the disclosure of which is incorporated herein by reference in its entirety).

Checkpoint inhibitors can include treatments, molecules, agents, and/or methods that regulate immune checkpoints at the transcriptional level, e.g., using the RNA-interference pathway co-suppression, and/or post-transcriptional gene silencing (PTGS) (e.g., microRNAs, miRNA; silencing-RNA, small-interfering-RNA, or short-interfering-RNA (siRNA). Transcriptional regulation of checkpoint molecules has been shown to involve mir-16, which has been shown to target the 3′UTR of the checkpoint mRNAs CD80, CD274 (PD-L1) and CD40 (Leibowitz et al., Post-transcriptional regulation of immune checkpoint genes by mir-16 in melanoma, Annals of Oncology (2017) 28; v428-v448). Mir-33a has also been shown to be involved in regulating the expression of PD-1 in cases of lung adenocarcinoma (Boldini et al., Role of microRNA-33a in regulating the expression of PD-1 in lung adenocarcinoma, Cancer Cell Int. 2017; 17: 105, the disclosure of which is incorporated herein by reference in its entirety).

T-cell-specific aptamer-siRNA chimeras have been suggested as a highly specific method of inhibiting molecules in the immune checkpoint pathway (Hossain et al., The aptamer-siRNA conjugates: reprogramming T cells for cancer therapy, Ther. Deliv. 2015 January; 6(1): 1-4, the disclosure of which is incorporated herein by reference in its entirety).

Alternatively, members of the immune checkpoint pathway can be inhibited using treatments that affect associated pathways, e.g., metabolism. For example, oversupplying the glycolytic intermediate pyruvate in mitochondria from CAD macrophages promoted expression of PD-L1 via induction of the bone morphogenetic protein 4/phosphorylated SMAD1/5/IFN regulatory factor 1 (BMP4/p-SMAD1/5/IRF1) signaling pathway. Accordingly, implementing treatments that modulate the metabolic pathway can result in subsequent modulation of the immunoinhibitory PD-1/PD-L1 checkpoint pathway (Watanabe et al., Pyruvate controls the checkpoint inhibitor PD-L1 and suppresses T cell immunity, J Clin Invest. 2017 Jun. 30; 127(7): 2725-2738).

Checkpoint immunity can be regulated via oncolytic viruses that selectively replicate within tumor cells and induce acute immune responses in the tumor-micro-environment, i.e., by acting as genetic vectors that carry specific agents (e.g., antibodies, miRNA, siRNA, etc.) to cancer cells and effecting their oncolysis and secretion of cytokines and chemokines to synergize with immune checkpoint inhibition (Shi et al., Cancer Immunotherapy: A Focus on the Regulation of Immune Checkpoints, Int J Mol Sci. 2018 May; 19(5): 1389). Currently, there are clinical trials underway that utilize the following viruses as checkpoint inhibitors: poliovirus, measles virus, adenoviruses, poxviruses, herpes simplex virus (HSV), coxsackieviruses, reovirus, Newcastle disease virus (NDV), T-VEC (a herpes virus encoded with GM-CSF (granulocyte-macrophage colony stimulating factor)), and H101 (Shi et al., supra).

Checkpoint inhibitors can operate at the translational level of checkpoint immunity. The translation of mRNA into protein represents a key event in the regulation of gene expression, thus inhibition of immune checkpoint translation is a method in which the immune checkpoint pathway can be inhibited.

Inhibition of the immune checkpoint pathway can occur at any stage of the immune checkpoint translational process. For example, drugs, molecules, agents, treatments, and/or methods can inhibit the initiation process (whereby the 40S ribosomal subunit is recruited to the 5′ end of the mRNA and scans the 5′UTR of the mRNA toward its 3′ end. Inhibition can occur by targeting the anticodon of the initiator methionyl-transfer RNA (tRNA) (Met-tRNAi), its base-pairing with the start codon, or the recruitment of the 60S subunit to begin elongation and sequential addition of amino acids in the translation of immune-checkpoint-specific genes. Alternatively, a checkpoint inhibitor can inhibit checkpoints at the translational level by preventing the formation of the ternary complex (TC), i.e., eukaryotic initiation factor (eIF)2 (or one or more of its α, β, and γ subunits); GTP; and Met-tRNAi.

Checkpoint inhibition can occur via destabilization of eIF2α by precluding its phosphorylation via protein kinase R (PKR), PERK, GCN2, or HRI, or by precluding TCs from associating with the 40S ribosome and/or other initiation factors, thus preventing the preinitiation complex (PIC) from forming; inhibiting the eIF4F complex and/or its cap-binding protein eIF4E, the scaffolding protein eIF4G, or eIF4A helicase. Methods discussing the translational control of cancer are discussed in Truitt et al., New frontiers in translational control of the cancer genome, Nat Rev Cancer. 2016 Apr. 26; 16(5): 288-304, the disclosure of which is incorporated herein by reference in its entirety.

Receptor and/or Ligand Inhibitors

Checkpoint inhibitors can also include treatments, molecules, agents, and/or methods that regulate immune checkpoints at the cellular and/or protein level, e.g., by inhibiting an immune checkpoint receptor. Inhibition of checkpoints can occur via the use of antibodies, antibody fragments, antigen-binding fragments, small-molecules, and/or other drugs, agents, treatments, and/or methods. Alternatively, checkpoint inhibitors can include treatments, molecules, agents, and/or methods that regulate checkpoint protein receptors, ligands, or the cells carrying said receptors and/or ligands themselves. Accordingly, a checkpoint inhibitor can inhibit, e.g., a ligand such as PD-L1, a receptor such as PD-1, a tumor cell displaying/expressing a checkpoint protein ligand, and/or a T cell displaying/expressing a checkpoint protein receptor.

Immune Checkpoints or Checkpoint Proteins

CTLA-4 (also known as Cytotoxic T-lymphocyte-associated protein 4, CTLA4, CTLA-4, CD152, cluster of differentiation 152; ALPS5, CD, CELIAC3, GRD4, GSE, and IDDM12). CTLA-4 is a ˜24.6-kDa single-pass type I membrane protein that plays an inhibitory role in T-cell function. CTLA-4 was originally identified by differential screening of a murine cytolytic T cell cDNA library, See Brunet et al., A new member of the immunoglobulin superfamily—CTLA-4, Nature. 1987 Jul. 16-22; 328(6127):267-70. CTLA- has been shown to interact with the b7 family ligands CD80 (also known as Cluster of differentiation 80, and B7-1); and CD86 (also known as Cluster of Differentiation 86 or B7-2). See Linsley et al., CTLA-4 is a second receptor for the B cell activation antigen B7, J Exp Med. 1991 Sep. 1; 174(3):561-9. Sequence comparison between the human CTLA-4 DNA encoding region, and that of CD28, reveals significant homology between both sequences, with the greatest similarity between juxtamembrane and cytoplasmic regions; accordingly, CTLA-4 is implicated in abrogating/reducing T-cell activity, and opposes the activity of CD28. CTLA-4 deficient mice have been shown to exhibit massive lymphoproliferation. Chambers et al., Lymphoproliferation in CTLA-4-deficient mice is mediated by costimulation-dependent activation of CD4+ T cells, Immunity. 1997 December; 7(6):885-95. It has been reported that CTLA-4 blockade augments T-cell responses both in vitro and in vivo, enhances an induced autoimmune disease, and exacerbates antitumor immunity. (See Luhder, J. Exp. Med. 1998; 187:427-432; Walunas et al., Immunity. 1994; 1:405-413; Kearney, J. Immunol. 1995; 155:1032-1036); Leach, Science 1996; 271:1734-1736). CTLA-4 has also been reported as having alternative and/or additional impact on the initial character of the T-cell immune response (Chambers, Curr. Opin. Immunol. 1997; 9:396-404; Bluestone, J. Immunol. 1997; 158:1989-1993; Thompson, Immunity 1997; 7:445-450).

PD-1 (also known as Programmed Death 1, CD279, PDCD1) is a cell surface receptor with a critical role in regulating the balance between stimulatory and inhibitory signals in the immune system and maintaining peripheral tolerance (Ishida, Y et al. 1992 EMBO J. 11 3887; Kier, Mary E et al. 2008 Annu Rev Immunol 26 677-704; Okazaki, Taku et al. 2007 International Immunology 19 813-824). PD-1 is an inhibitory member of the immunoglobulin super-family with homology to CD28. The structure of PD-1 is a monomeric type 1 transmembrane protein, consisting of one immunoglobulin variable-like extracellular domain and a cytoplasmic domain containing an immunoreceptor tyrosine-based inhibitory motif (ITIM) and an immunoreceptor tyrosine-based switch motif (ITSM). Expression of PD-1 is inducible on T cells, B cells, natural killer (NK) cells and monocytes, for example upon lymphocyte activation via T cell receptor (TCR) or B cell receptor (BCR) signaling (Kier, Mary E et al. 2008 Annu Rev Immunol 26 677-704; Agata, Y et al 1996 Int Immunol 8 765-72). PD-1 is a receptor for the ligands CD80, CD86, PD-L1 (B7-H1, CD274) and PD-L2 (B7-DC, CD273), which are cell surface expressed members of the B7 family (Freeman, Gordon et al. 2000 J Exp Med 192 1027; Latchman, Y et al. 2001 Nat Immunol 2 261). Upon ligand engagement, PD-1 recruits phosphatases such as SHP-1 and SHP-2 to its intracellular tyrosine motifs which subsequently dephosphorylate effector molecules activated by TCR or BCR signaling (Chemnitz, J et al. 2004 J Immunol 173 945-954; Riley, James L 2009 Immunological Reviews 229 114-125) In this way, PD-1 transduces inhibitory signals into T and B cells only when it is engaged simultaneously with the TCR or BCR.

PD-1 has been demonstrated to down-regulate effector T cell responses via both cell-intrinsic and cell-extrinsic functional mechanisms. Inhibitory signaling through PD-1 induces a state of unresponsiveness in T cells, resulting in the cells being unable to clonally expand or produce optimal levels of effector cytokines. PD-1 may also induce apoptosis in T cells via its ability to inhibit survival signals from co-stimulation, which leads to reduced expression of key anti-apoptotic molecules such as Bcl-XL (Kier, Mary E et al. 2008 Annu Rev Immunol 26 677-704). In addition to these direct effects, recent publications have implicated PD-1 as being involved in the suppression of effector cells by promoting the induction and maintenance of regulatory T cells (TREG). For example, PD-L1 expressed on dendritic cells was shown to act in synergy with TGF-β to promote the induction of CD4+ FoxP3+ TREG with enhanced suppressor function (Francisco, Loise M et al. 2009 J Exp Med 206 3015-3029).

TIM-3 (also known as T-cell immunoglobulin and mucin-domain containing-3, TIM-3, Hepatitis A virus cellular receptor 2, HAVCR2, HAVcr-2, KIM-3, TIMD-3, TIMD3, Tim-3, and CD366) is a ˜33.4-kDa single-pass type I membrane protein involved in immune responses (Sanchez-Fueyo et al., Tim-3 inhibits T helper type 1-mediated auto- and alloimmune responses and promotes immunological tolerance, Nat. Immunol. 4:1093-1101 (2003)).

TIM-3 is selectively expressed on Th1-cells, and phagocytic cells (e.g., macrophages and dendritic cells). The use of siRNA or a blocking antibody to reduce the expression of human resulted in increased secretion of interferon γ (IFN-γ) from CD4 positive T-cells, implicating the inhibitory role of TIM-3 in human T cells. Analysis of clinical samples from autoimmune disease patients showed no expression of TIM-3 in CD4 positive cells. In particular, expression level of TIM-3 is lower and secretion of IFN-γ is higher in T cell clones derived from the cerebrospinal fluid of patients with multiple sclerosis than those in clones derived from normal healthy persons (Koguchi K et al., J Exp Med. 203:1413-8. (2006)).

TIM-3 is the receptor for the ligands Galectin-9, which is a member of galectin family, molecules ubiquitously expressed on a variety of cell types and which binds β-galactoside; Phospatidyl serine (PtdSer) (DeKryff et al., T cell/transmembrane, Ig, and mucin-3 allelic variants differentially recognize phosphatidylserine and mediate phagocytosis of apoptotic cells, J Immunol. 2010 Feb. 15; 184(4):1918-30); High Mobility Group Protein 1 (also known as HMGB1, HMG1, HMG3, SBP-1, HMG-1, and high mobility group box 1) Chiba et al., Tumor-infiltrating DCs suppress nucleic acid-mediated innate immune responses through interactions between the receptor TIM-3 and the alarmin HMGB1, Nat Immunol. 2012 September; 13(9):832-42); and Carcinoembryonic Antigen Related Cell Adhesion Molecule 1 (also known as CEACAM1, BGP, BGP1, BGPI, carcinoembryonic antigen related cell adhesion molecule 1) (Huang et al., CEACAM1 regulates TIM-3-mediated tolerance and exhaustion, Nature. 2015 Jan. 15; 517(7534):386-90).

BTLA (also known as B- and T-lymphocyte attenuator, BTLA1, CD272, and B and T lymphocyte associated) is a ˜27.3-kDa single-pass type I membrane protein involved in lymphocyte inhibition during immune response. BTLA is constitutively expressed in both B and T cells. BTLA interacts with HVEM (herpes virus-entry mediator), a member of the tumor-necrosis factor receptor (TNFR) family (Gonzalez et al., Proc. Natl. Acad. Sci. USA, 2005, 102: 1116-21). The interaction of BTLA, which belongs to the CD28 family of the immunoglobulin superfamily, and HVEM, a costimulatory tumor-necrosis factor (TNF) receptor (TNFR), is unique in that it defines a cross talk between these two families of receptors. BTLA contains a membrane proximal immunoreceptor tyrosine-based inhibitory motif (ITIM) and membrane distal immunoreceptor tyrosine-based switch motif (ITSM). Disruption of either the ITIM or ITSM abrogated the ability of BTLA to recruit either SHP1 or SHP2, suggesting that BTLA recruits SHP1 and SHP2 in a manner distinct from PD-1 and both tyrosine motifs are required to block T cell activation. The BTLA cytoplasmic tail also contains a third conserved tyrosine-containing motif within the cytoplasmic domain, similar in sequence to a Grb-2 recruitment site (YXN). Also, a phosphorylated peptide containing this BTLA N-terminal tyrosine motif can interact with GRB2 and the p85 subunit of PI3K in vitro, although the functional effects of this interaction remain unexplored in vivo (Gavrieli et al., Bioochem. Biophysi Res Commun, 2003, 312, 1236-43). BTLA is the receptor for the ligands PTPN6/SHP-1; PTPN1I1/SHP-2; TNFRSF14/HVEM; and B7H4.

VISTA (also known as V-domain Ig suppressor of T cell activation VSIR, B7-H5, B7H5, GI24, PP2135, SISP1, DD1alpha, VISTA, C10orf54, chromosome 10 open reading frame 54, PD-1H, and V-set immunoregulatory receptor) is a ˜33.9-kDa single-pass type I membrane protein involved in T-cell inhibitory response, embryonic stem cells differentiation via BMP4 signaling inhibition, and MMPP14-mediated MMP2 activation (Yoon et al., Control of signaling-mediated clearance of apoptotic cells by the tumor suppressor p53, Science. 2015 Jul. 31; 349(6247): 1261669). VISTA interacts with the ligand VSIG-3 (Wang et al., VSIG-3 as a ligand of VISTA inhibits human T-cell function, Immunology. 2019 January; 156(1):74-85)

LAG-3 (also known as Lymphocyte-activation gene 3, LAG3, CD223, and lymphocyte activating 3) is a ˜57.4-kDa single-pass type I membrane protein involved in lymphocyte activation that also binds to HLA class-II antigens. LAG-3 is a member of the immunoglobulin supergene family, and is expressed on activated T cells (Huard et al., 1994, Immunogenetics 39:213), NK cells (Triebel et al., 1990, J. Exp. Med. 171:1393-1405), regulatory T cells (Huang et al., 2004, Immunity 21:503-513; Camisaschi et al., 2010, J Immunol. 184:6545-6551; Gagliani et al., 2013, Nat Med 19:739-746), and plasmacytoid dendritic cells (DCs) (Workman et al., 2009, J Immunol 182:1885-1891). LAG-3 is a membrane protein encoded by a gene located on chromosome 12, and is structurally and genetically related to CD4. Similar to CD4, LAG-3 can interact with MHC class II molecules on the cell surface (Baixeras et al., 1992, J. Exp. Med. 176:327-337; Huard et al., 1996, Eur. J. Immunol. 26:1180-1186). It has been suggested that the direct binding of LAG-3 to MHC class II plays a role in down-regulating antigen-dependent stimulation of CD4+ T lymphocytes (Huard et al., 1994, Eur. J. Immunol. 24:3216-3221) and LAG-3 blockade has also been shown to reinvigorate CD8+ lymphocytes in both tumor or self-antigen (Gross et al., 2007, J Clin Invest. 117:3383-3392) and viral models (Blackburn et al., 2009, Nat. Immunol. 10:29-37). Further, the intra-cytoplasmic region of LAG-3 can interact with LAP (LAG-3-associated protein), which is a signal transduction molecule involved in the downregulation of the CD3/TCR activation pathway (Iouzalen et al., 2001, Eur. J. Immunol. 31:2885-2891). Moreover, CD4+CD25+ regulatory T cells (Treg) have been shown to express LAG-3 upon activation, which contributes to the suppressor activity of Treg cells (Huang, C. et al., 2004, Immunity 21:503-513). LAG-3 can also negatively regulate T cell homeostasis by Treg cells in both T cell-dependent and independent mechanisms (Workman, C. J. and Vignali, D. A., 2005, J. Immunol. 174:688-695).

LAG-3 has been shown to interact with MHC class II molecules (Huard et al., CD4/major histocompatibility complex class II interaction analyzed with CD4- and lymphocyte activation gene-3 (LAG-3)-Ig fusion proteins, Eur J Immunol. 1995 September; 25(9):2718-21).

Additionally, several kinases are known to be checkpoint inhibitors. For example, CHEK-1, CHEK-2, and A2aR.

CHEK-1 (also known as CHK 1 kinase, CHK1, and checkpoint kinase 1) is a ˜54.4-kDa serine/threonine-protein kinase that is involved with checkpoint-mediated cell cycle arrest, and the activation of DNA repair in response to the DNA damage and/or unreplicated DNA.

CHEK-2 (also known as CHK2 kinase, CDS1, CHK2, HuCdsl, LFS2, PP1425, RAD53, hCdsl, and checkpoint kinase 2) is a ˜60.9-kDA serine/threonine-protein kinase involved in checkpoint-mediated cell cycle arrest, DNA-repair activation, and double-strand break-mediated apoptosis.

A2aR (also known as adenosine A2A receptor, ADORA2A, adenosine A2a receptor, A2aR, ADORA2, and RDC8) is a ˜44.7-kDa multi-pass membrane receptor for adenosine and other ligands.

Other checkpoint proteins include agonists of stimulatory checkpoint pathways e.g., OX40, OX40L, ICOS, B7RP1, GITR, GITRL, 4-1BB, 4-IBBL CD40, CD40L, CD70, CD27; and antagonists or inhibitory checkpoint proteins, e.g., B7-H3, MHC I, MHC II, TCR, KIR; and proteins that play both an agonistic and antagonistic role, e.g., CD155/CD112, and CD226/TIGT (Marin-Acevedo et al., Next generation of immune checkpoint therapy in cancer: new developments and challenges, J Hematol Oncol. 2018; 11: 39).

In an embodiment of the disclosure, the checkpoint inhibitor therapy, in combination therapy with a compound of Formula I, and/or Formula Ia, or a pharmaceutically acceptable salt thereof is used to reduce or inhibit metastasis of a primary tumor or cancer to other sites, or the formation or establishment of metastatic tumors or cancers at other sites distal from the primary tumor or cancer thereby inhibiting or reducing tumor or cancer relapse or tumor or cancer progression.

In a further embodiment of the disclosure, there is provided a combination therapy for treating cancer, comprising a compound of Formula I and/or Formula Ia, or a pharmaceutically acceptable salt thereof, and blockade of checkpoint inhibitors with the potential to elicit potent and durable immune responses with enhanced therapeutic benefit and more manageable toxicity.

In a further embodiment of the disclosure, there is provided a combination therapy for treating cancer, comprising (1) a compound of Formula I, and/or Formula Ia, or a pharmaceutically acceptable salt thereof, which is a pan-PI3K/mTOR inhibitor, and potently inhibits three distinct kinase activities, i.e., PI3Kγ, PI3Kδ, and mTOR, that are all implicated in immune suppression, and EGFR; and (2) a checkpoint inhibitor of checkpoint proteins (e.g., PD-1 and/or PD-L1) as described herein.

In an embodiment of the disclosure is provided a method for treating cancer and/or preventing the establishment of metastases by employing a checkpoint inhibitor which act synergistically with a compound of Formula I and/or Formula Ia, or a pharmaceutically acceptable salt thereof.

In further embodiments, methods of the disclosure include, one or more of the following: 1) reducing or inhibiting growth, proliferation, mobility or invasiveness of tumor or cancer cells that potentially or do develop metastases, 2) reducing or inhibiting formation or establishment of metastases arising from a primary tumor or cancer to one or more other sites, locations or regions distinct from the primary tumor or cancer; 3) reducing or inhibiting growth or proliferation of a metastasis at one or more other sites, locations or regions distinct from the primary tumor or cancer after a metastasis has formed or has been established, 4) reducing or inhibiting formation or establishment of additional metastasis after the metastasis has been formed or established, 5) prolonged overall survival, 6) prolonged progression free survival, or 7) disease stabilization.

In an embodiment of the disclosure, administration of the checkpoint inhibitor therapy, in combination therapy with a compound of Formula I, and/or Formula Ia, or a pharmaceutically acceptable salt thereof, provides a detectable or measurable improvement in a condition of a given subject, such as alleviating or ameliorating one or more adverse (physical) symptoms or consequences associated with the presence of a cell proliferative or cellular hyperproliferative disorder, neoplasia, tumor or cancer, or metastasis, i.e., a therapeutic benefit or a beneficial effect.

A therapeutic benefit or beneficial effect is any objective or subjective, transient, temporary, or long-term improvement in the condition or pathology, or a reduction in onset, severity, duration or frequency of adverse symptom associated with or caused by cell proliferation or a cellular hyperproliferative disorder such as a neoplasia, tumor or cancer, or metastasis. It may lead to improved survival. A satisfactory clinical endpoint of a treatment method in accordance with the disclosure is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumor or cancer, or metastasis. A therapeutic benefit or improvement therefore may be, but is not limited to destruction of target proliferating cells (e.g., neoplasia, tumor or cancer, or metastasis) or ablation of one or more, most or all pathologies, adverse symptoms or complications associated with or caused by cell proliferation or the cellular hyperproliferative disorder such as a neoplasia, tumor or cancer, or metastasis. However, a therapeutic benefit or improvement need not be a cure or complete destruction of all target proliferating cells (e.g., neoplasia, tumor or cancer, or metastasis) or ablation of all pathologies, adverse symptoms or complications associated with or caused by cell proliferation or the cellular hyperproliferative disorder such as a neoplasia, tumor or cancer, or metastasis. For example, partial destruction of a tumor or cancer cell mass, or a stabilization of the tumor or cancer mass, size or cell numbers by inhibiting progression or worsening of the tumor or cancer, can reduce mortality and prolong lifespan even if only for a few days, weeks or months, even though a portion or the bulk of the tumor or cancer mass, size or cells remain.

Specific non-limiting examples of therapeutic benefit include a reduction in neoplasia, tumor or cancer, or metastasis volume (size or cell mass) or numbers of cells, inhibiting or preventing an increase in neoplasia, tumor or cancer volume (e.g., stabilizing), slowing or inhibiting neoplasia, tumor or cancer progression, worsening or metastasis, or inhibiting neoplasia, tumor or cancer proliferation, growth or metastasis.

In an embodiment of the disclosure, administration of the combination (e.g., a checkpoint inhibitor and a compound of Formula I and/or Formula Ia or a pharmaceutically acceptable salt thereof), provides a detectable or measurable improvement or overall response according to the immune-related response criteria (irRC) (as derived from time-point response assessments and based on tumor burden), including one of more of the following: (i) immune-related complete response (irCR): complete disappearance of all lesions, whether measurable or not, and no new lesions (confirmation by a repeat, consecutive assessment no less than 4 weeks from the date first documented), (ii) immune-related partial response (irPR): decrease in tumor burden ≥50% relative to baseline (confirmed by a consecutive assessment at least 4 weeks after first documentation).

In some embodiments, the disclosed method may not take effect immediately. For example, treatment may be followed by an increase in the neoplasia, tumor or cancer cell numbers or mass, but over time eventual stabilization or reduction in tumor cell mass, size or numbers of cells in a given subject may subsequently occur.

Additional adverse symptoms and complications associated with neoplasia, tumor, cancer and metastasis that can be inhibited, reduced, decreased, delayed or prevented include, for example, nausea, lack of appetite, lethargy, pain and discomfort. Thus, a partial or complete decrease or reduction in the severity, duration or frequency of adverse symptom or complication associated with or caused by a cellular hyperproliferative disorder, an improvement in the subjects quality of life and/or well-being, such as increased energy, appetite, psychological well-being, are all particular non-limiting examples of therapeutic benefit.

A therapeutic benefit or improvement therefore can also include a subjective improvement in the quality of life of a treated subject. In additional embodiment, a method prolongs or extends lifespan (survival) of the subject. In a further embodiment, a method improves the quality of life of the subject.

In one embodiment, administration of the combination (i.e., one or more checkpoint inhibitors or fragments thereof, in combination with a compound of Formula I and/or Formula Ia or a pharmaceutically acceptable salt thereof) results in a clinically relevant improvement in one or more markers of disease status and progression selected from one or more of the following: (i): overall survival, (ii): progression-free survival, (iii): overall response rate, (iv): reduction in metastatic disease, (v): circulating levels of tumor antigens such as carbohydrate antigen 19.9 (CA19.9) and carcinembryonic antigen (CEA) or others depending on tumor, (vii) nutritional status (weight, appetite, serum albumin), (viii): pain control or analgesic use, (ix): CRP/albumin ratio.

In some embodiments, the present disclosure provides a combination of a compound of Formula I, and/or a compound of Formula Ia, or a pharmaceutically acceptable salt of these compounds, in combination with a checkpoint inhibitor selected from a CTLA-4 inhibitor, for example, Tremelimumab, Abatacept, AK104, and or Ipilimumab.

In some embodiments, the present disclosure provides a combination of a compound of Formula I, and/or a compound of Formula Ia, or a pharmaceutically acceptable salt of these compounds, in combination with a checkpoint inhibitor selected from a PD-1 inhibitor, for example, REGN2810 (cemiplimab), Nivolumab, Pembrolizumab, Sintilimab (IBI308), Tislelizumab (BGB-A317), SHR-1210 (Camrelizumab), Spartalizumab (PDR001), JS001, TSR-042, JNJ-63723283, BCD-100, and/or TORIPALIMAB.

In some embodiments, the present disclosure provides a combination of a compound of Formula I, and/or a compound of Formula Ia, or a pharmaceutically acceptable salt of these compounds, in combination with a checkpoint inhibitor selected from a PD-L1 inhibitor, for example, Avelumab, atezolizumab, TQB2450, KN035, CS1001, and/or Durvalumab (MEDI4736).

In some embodiments, the present disclosure provide a combination comprising

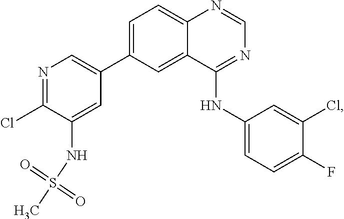 [SMILES: CS(=O)(=O)Nc1cc(-c2ccc3ncnc(Nc4ccc(F)c(Cl)c4)c3c2)cnc1Cl]

or a pharmaceutically acceptable salt thereof and at least one checkpoint inhibitor selected from the group: Tremelimumab, Abatacept, AK104, REGN2810 (Cemiplimab), Nivolumab, Pembrolizumab, Sintilimab (IBI308), Tislelizumab (BGB-A317), SHR-1210 (Camrelizumab), Spartalizumab (PDR001), JS001, TSR-042, JNJ-63723283, BCD-100, TORIPALIMAB, Avelumab, Atezolizumab, TQB2450, KN035, CS1001, and/or Durvalumab (MEDI4736).

Methods of Making Antibodies and Antigen-Binding Fragments Thereof

The disclosure also provides methods of generating, selecting, and making checkpoint inhibitor antibodies. The antibodies of this disclosure can be made by procedures known in the art. In some embodiments, antibodies of the present disclosure can be made using hybridoma technology. It is contemplated that any mammalian subject including humans or antibody producing cells therefrom can be manipulated to serve as the basis for production of mammalian, including human, hybridoma cell lines. The route and schedule of immunization of the host animal are generally in keeping with established and conventional techniques for antibody stimulation and production, as further described herein. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C., 1975, Nature 256:495-497. Available myeloma lines, can be used in the hybridization procedure. Generally, the hybridoma technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the checkpoint inhibitor monoclonal antibodies of the subject disclosure. The hybridomas or other immortalized B-cells are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies specific for a checkpoint molecule, or a portion thereof.

Once the desired hybridomas producing high-affinity antibodies are identified, the cells can be grown in vitro or in vivo using known procedures. The monoclonal antibodies from the selected hybridoma cells can be isolated from the culture media or body fluids, by conventional immunoglobulin purification procedures as known in the art. Immunization of a host animal with a checkpoint molecule polypeptide, (for example, human PD-1, mouse or other species) or a PD-1 fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfo-succinimide ester (conjugation through cysteine residues), N-hydroxysuccinimide (through lysine residues), glutaraldehyde, succinic anhydride, SOC12, or R¹N═C═NR, where R¹ and R are different alkyl groups, can yield a population of antibodies (e.g., monoclonal antibodies, or polyclonal antibodies).

In some embodiments, antibodies may be made recombinantly and expressed using any method known in the art. In some embodiments, antibodies may be prepared and selected by phage display technology. See, for example, Winter et al., Annu. Rev. Immunol. 12:433-455, 1994, and methods exemplified in various U.S. Pat. Nos. 5,565,332; 5,580,717; 5,733,743; and 6,265, 150; the disclosures of which are hereby incorporated by reference. In some embodiments, phage display technology (McCafferty et al., Nature 348:552-553, 1990) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats. Several sources of V-gene segments can be used for phage display.

A repertoire of V genes from human donors can be constructed and antibodies to a diverse array of antigens can be isolated essentially following the techniques described by Mark et al., 1991, J. Mol. Biol. 222:581-597, or Griffith et al., 1993, EMBO J. 12:725-734, both of which are incorporated herein by reference in their entireties. Somatic hypermutation can be used to produce B cells displaying high-affinity surface immunoglobulin. These B-cells are preferentially replicated and differentiated during subsequent antigen challenge. This natural process can be mimicked by employing the technique known as “chain shuffling.” (Marks et al., 1992, Bio/Technol. 10:779-783). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody libraries has been described by Waterhouse et al., Nucl. Acids Res. 21:2265-2266, 1993. Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213). Unlike traditional humanization of rodent antibodies by CDR grafting, this technique provides completely human antibodies, which have no framework or CDR residues of rodent origin.

In various exemplary methods, a checkpoint inhibitor antibody (monoclonal or poly-clonal) directed to a checkpoint molecule of interest (e.g., PD-1) may be sequenced and the polynucleotide sequence may then be cloned into a vector for expression or propagation. The sequence encoding the antibody or antigen-binding fragment thereof of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

In some embodiments, methods for producing the recombinant antibodies can include the steps of culturing a host cell containing isolated nucleic acid(s) encoding the antibodies of the present disclosure. Methods for culturing a host cell containing isolated nucleic acid(s) encoding the antibodies of the present disclosure can be done in a variety of ways, depending on the nature of the antibody. In some embodiments, in the case where the antibodies of the disclosure are full length traditional antibodies, for example, a heavy chain variable region and a light chain variable region under conditions such that an antibody is produced and can be isolated.

In general, nucleic acids are provided that encode the antibodies or antigen-binding fragments thereof of the present disclosure. Such polynucleotides encode for both the variable and constant regions of each of the heavy and light chains, although other combinations are also contemplated by the present disclosure. The present disclosure also contemplates oligonucleotide fragments derived from the disclosed polynucleotides and nucleic acid sequences complementary to these polynucleotides.

The polynucleotides can be in the form of RNA, DNA, cDNA, genomic DNA, nucleic acid analogs, and synthetic DNA. The DNA may be double-stranded or single-stranded, and if single stranded, may be the coding (sense) strand or non-coding (anti-sense) strand. The coding sequence that encodes the polypeptide may be identical to the coding sequence or may be a different coding sequence, which sequence, as a result of the redundancy or degeneracy of the genetic code, encodes the same polypeptides.

In some embodiments, nucleic acid(s) encoding the antibodies of the present disclosure are incorporated into expression vectors, which can be extrachromosomal or designed to integrate into the genome of the host cell into which it is introduced. Expression vectors can contain any number of appropriate regulatory sequences (including, but not limited to, transcriptional and translational control sequences, promoters, ribosomal binding sites, enhancers, origins of replication, etc.) or other components (selection genes, etc.), all of which are operably linked as is well known in the art. In some cases two nucleic acids are used and each put into a different expression vector (e.g. heavy chain in a first expression vector, light chain in a second expression vector), or alternatively they can be put in the same expression vector. It will be appreciated by those skilled in the art that the design of the expression vector(s), including the selection of regulatory sequences may depend on such factors as the choice of the host cell, the level of expression of protein desired, etc.

In general, the nucleic acids and/or expression can be introduced into a suitable host cell to create a recombinant host cell using any method appropriate to the host cell selected (e.g., transformation, transfection, electroporation, infection), such that the nucleic acid molecule(s) are operably linked to one or more expression control elements (e.g., in a vector, in a construct created by processes in the cell, integrated into the host cell genome). The resulting recombinant host cell can be maintained under conditions suitable for expression (e.g. in the presence of an inducer, in a suitable non-human animal, in suitable culture media supplemented with appropriate salts, growth factors, antibiotics, nutritional supplements, etc.), whereby the encoded polypeptide(s) are produced. In some cases, the heavy chains are produced in one cell and the light chain in another.

Mammalian cell lines available as hosts for expression are known in the art and include many immortalized cell lines available from the American Type Culture Collection (ATCC), Manassas, Va. USA. including but not limited to Chinese hamster ovary (CHO) cells, HEK 293 cells, NSO cells, HeLa cells, baby hamster kidney (BHK) cells, monkey kidney cells (COS), human hepatocellular carcinoma cells (e.g., Hep G2), and a number of other cell lines. Non-mammalian cells including but not limited to bacterial, yeast, insect, and plants can also be used to express recombinant antibodies. In some embodiments, the antibodies can be produced in transgenic animals such as cows or chickens.

Exemplary and illustrative recombinant methods for antibody molecular biology, expression, purification, and screening are described, for example, in Antibody Engineering, edited by Kontermann & Dubel, Springer, Heidelberg, 2001 and 2010 Hayhurst & Georgiou, 2001, Curr. Opin. Chem. Biol. 5:683-689; Maynard & Georgiou, 2000, Annu. Rev. Biomed. Eng. 2:339-76; and Morrison, S. (1985) Science 229:1202, the disclosures of which are incorporated herein by reference in their entireties.

In various embodiments, the polynucleotide sequence encoding the selected variable heavy and light chains may be used for genetic manipulation to humanize the antibody or to improve the affinity, or other characteristics of the antibody. Antibodies may also be customized for use, for example, in dogs, cats, primate, equines and bovines.

In some embodiments, fully human antibodies may be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Abgenix, Inc. (Fremont, Calif.) and HuMAb-Mouse® and TC Mouse™ from Medarex, Inc. (Princeton, N.J.).

Checkpoint inhibitor antibodies of the present disclosure can be made recombinantly by first isolating the antibodies and antibody producing cells from host animals, obtaining the gene sequence, and using the gene sequence to express the antibody recombinantly in host cells (e.g., CHO cells). Another method which may be employed is to express the antibody sequence in plants (e.g., tobacco) or in yeast cells (e.g. Pichia pastoris or Saccharomyces cerevisiae. Methods for expressing antibodies recombinantly in plants or yeast have been disclosed. See, for example, Peeters, et al. Vaccine 19:2756, 2001; Lonberg, N. and D. Huszar Int. Rev. Immunol 13:65, 1995; and Horwitz, A. H. et al., Proc. Natl. Acad. Sci. 85:8678-8682; the disclosures of which are hereby incorporated by reference in their entireties. Methods for making derivatives of antibodies, e.g., domain, single chain, etc. are known in the art.

Immunoassays and flow cytometry sorting techniques such as fluorescence activated cell sorting (FACS) can also be employed to isolate antibodies that are specific for checkpoint molecules.

In some embodiments, a polynucleotide comprises a sequence encoding the heavy chain and/or the light chain variable regions of the checkpoint inhibitor antibody or antigen-binding fragment thereof of the present disclosure. The sequence encoding the antibody or antigen-binding fragment thereof of interest may be maintained in a vector in a host cell and the host cell can then be expanded and frozen for future use. Vectors (including expression vectors) and host cells are further described herein.

The disclosure includes affinity matured checkpoint inhibitor antibodies. For example, affinity matured antibodies can be produced by procedures known in the art (Marks et al., 1992, Bio/Technology, 10:779-783; Barbas et al., 1994, Proc Nat. Acad. Sci. USA 91:3809-3813. One way of characterizing a CDR of an antibody and/or altering (such as improving) the binding affinity of a polypeptide, such as an antibody, termed “library scanning mutagenesis”. An exemplary method for providing affinity matures antibodies and antigen-binding fragments can include replacing one or more amino acid positions in the CDR with two or more (such as 3, 4, 5, 6, 7, 8, 9, 10, 11, 12, 13, 14, 15, 16, 17, 18, 19, or 20) amino acids using art recognized methods. a library of clones are generated, each with a complexity of two or more members (if two or more amino acids are substituted at every position). Generally, the library also includes a clone comprising the native (unsubstituted) amino acid. A small number of clones, e.g., about 20-80 clones (depending on the complexity of the library), from each library are screened for binding affinity to the target polypeptide (or other binding target), and candidates with increased, the same, decreased, or no binding are identified. Methods for determining binding affinity are well-known in the art. Binding affinity may be determined using, for example, Biacore™ surface plasmon resonance analysis, which detects differences in binding affinity of about 2-fold or greater, Kinexa® Biosensor, scintillation proximity assays, ELISA, ORIGEN® immunoassay, fluorescence quenching, fluorescence transfer, and/or yeast display. Binding affinity may also be screened using a suitable bioassay. Biacore™ is particularly useful when the starting antibody already binds with a relatively high affinity, for example a K_{D} of about 10 nM or lower. The library of clones can then be recombinantly introduced into a selection construct using any method known in the art for selection, including phage display, yeast display, and ribosome display.

The antibodies may also be modified, e.g., in the variable domains of the heavy and/or light chains, e.g., to alter a binding property of the antibody. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. For example, a mutation may be made in one or more of the CDR regions to increase or decrease the K_{D} of the antibody directed to a checkpoint molecule, to increase or decrease kₒₙ or to alter the binding specificity of the antibody. Techniques in site-directed mutagenesis are well-known in the art. See, e.g., Sambrook et al. and Ausubel et al.

Pharmaceutical Compositions and Formulations

In another aspect, the present disclosure provides a composition, e.g., a pharmaceutical composition, comprising an effective amount of one or more checkpoint inhibitor antibody or an antigen-binding fragment thereof, of the present disclosure, and a pharmaceutically acceptable excipient. In other exemplary embodiments, the pharmaceutical compositions may be administered as part of a combination therapy, e.g., a compound of Formula I, and/or Formula Ia, and one or more checkpoint inhibitor antibodies (e.g., PD-1). In some embodiments, the combination of one or more compounds of Formula I and/or Ia, a checkpoint inhibitor, may further include a third active agent or medical procedure used in the field to treat a cancer. The combination therapy can include one or more compounds of Formula I and/or Ia, a checkpoint inhibitor antibody, or antigen binding fragment thereof, of the present disclosure combined with at least one other therapy wherein the therapy may be surgery, immunotherapy, chemotherapy, radiation treatment, or drug therapy. For example, a pharmaceutical composition of the disclosure can comprise a combination of one or more checkpoint inhibitor antibodies that bind to different epitopes on the target checkpoint molecule, or that have complementary activities. In another example, the combination therapy can include a compound of Formula I and/or Formula Ia; one or more checkpoint inhibitor antibodies, or their antigen binding fragments thereof, and each or both combined with at least one other active agent used to treat cancer, including, but not limited to, chemotherapeutic antineoplastics, apoptosis-modulating agents, antimicrobials, antivirals, antifungals, and anti-inflammatory agents, and/or a therapeutic technique (e.g., surgical intervention, and/or radiotherapies) as described herein.

In some embodiments, the chemotherapeutic agent can include a MEK inhibitor, which can be used in the same formulation as the compound of Formula I and/or Formula Ia, or in separate formulations, to be combined with one or more checkpoint inhibitors, for example, Tremelimumab, Abatacept, AK104, REGN2810 (Cemiplimab), Nivolumab, Pembrolizumab, Sintilimab (IBI308), Tislelizumab (BGB-A317), SHR-1210 (Camrelizumab), Spartalizumab (PDR001), JS001, TSR-042, JNJ-63723283, BCD-100, TORIPALIMAB, Avelumab, Atezolizumab, TQB2450, KN035, CS1001, and/or Durvalumab (MEDI4736).

In some aspects, a compound of Formula I and/or Formula Ia, or a pharmaceutically acceptable salt thereof is soluble in formulation buffer (e.g. aqueous formulation buffer) at a concentration of at least 10 mM. In some embodiments, a compound of Formula I and/or Formula Ia is soluble in formulation buffer at a concentration of at least 100 mM. In some aspects, a compound of Formula I and/or Formula Ia, or a pharmaceutically acceptable salt thereof is soluble in formulation buffer (e.g. aqueous formulation buffer) at a concentration of at least 100 μg/ml, at least 1 mg/ml, at least 50 mg/ml, at least about 100 mg/ml, at least about 200 mg/ml, or at least about 300 mg/ml.

A compound of Formula I and/or Formula Ia or its prodrug, or a pharmaceutically acceptable salt thereof, can be formulated as pharmaceutical compositions comprising a therapeutically or prophylactically effective amount of a compound of Formula I and/or Formula Ia or its prodrug, or a pharmaceutically acceptable salt thereof and one or more pharmaceutically compatible (acceptable) ingredients. In some aspects, pharmaceutical compositions of a compound of Formula I and/or Formula Ia and pharmaceutical excipients are provided in which an effective amount of a compound of Formula I and/or Formula Ia is in admixture with the excipients, suitable for administration to a mammal In preferred aspects, a compound of Formula I and/or Formula Ia is formulated for administration to a human. According, the present disclosure provides a pharmaceutical composition comprising a compound of Formula I and/or Formula Ia, or a pharmaceutically acceptable salt thereof is formulated for administration to a human subject in need thereof. The formulated composition comprising a compound of Formula I and/or Formula Ia, or a pharmaceutically acceptable salt thereof will generally comprise one or more pharmaceutically compatible (acceptable) ingredients.

Exemplary pharmaceutical or non-pharmaceutical compositions typically include one or more carriers (e.g., sterile liquids, such as water and oils, including those of petroleum, animal, vegetable or synthetic origin, such as peanut oil, soybean oil, mineral oil, sesame oil and the like). Water is a more typical carrier when the pharmaceutical composition is administered intravenously. Saline solutions and aqueous dextrose and glycerol solutions can also be employed as liquid carriers, particularly for injectable solutions. Suitable excipients include, for example, amino acids, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin. Such compositions will typically contain a therapeutically effective amount of a compound of Formula I, and/or Formula Ia, or a pharmaceutically acceptable salt thereof is typically in purified form, together with a suitable amount of carrier so as to provide the form for proper administration to the subject. The formulations correspond to the mode of administration.

The pharmaceutically acceptable carrier or vehicle can be particulate, so that the compositions are, for example, in tablet or powder form. The carrier(s) can be liquid, with the compositions being, for example, an oral syrup, flavored water, or injectable liquid.

When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

When the composition is in the form of a capsule, e.g., a gelatin capsule, it can contain, in addition to materials of the above type, a liquid carrier such as polyethylene glycol, cyclodextrin or fatty oil.

The composition can be in the form of a liquid, e.g., an elixir, syrup, solution, emulsion or suspension. The liquid can be useful for oral administration or for delivery by injection. When intended for oral administration, a composition can comprise one or more of a sweetening agent, preservatives, dye/colorant, and flavor enhancer. In some aspects, the composition is formulated into a powder and the end user mixes the power in aqueous solution for oral administration. In a composition for administration by injection (as described above), one or more of a surfactant, preservative, wetting agent, dispersing agent, suspending agent, buffer, stabilizer and isotonic agent can also be included.

The composition and preparation of capsules are well known in the art. For example, capsules may be prepared from gelatin (e.g., Type A, Type B), carrageenan (e.g., kappa, iota, lambda) and/or modified cellulose (e.g., hydroxypropyl methyl cellulose, methyl cellulose, hydroxypropyl methyl cellulose acetate succinate, hydroxypropyl methyl cellulose phthalate, cellulose acetate phthalate), and optionally one or more excipients such as oils (e.g., fish oil, olive oil, corn oil, soybean oil, coconut oil, tri-, di- and monoglycerides), plasticizers (e.g., glycerol, glycerin, sorbitol, polyethylene glycol, citric acid, citric acid esters such as triethylcitrate, polyalcohols), co-solvents (e.g., triacetin, propylene carbonate, ethyl lactate, propylene glycol, oleic acid, dimethylisosorbide, stearyl alcohol, cetyl alcohol, cetostearyl alcohol, glyceryl behenate, glyceryl palmitostearate), surfactants, buffering agents, lubricating agents, humectants, preservatives, colorants and flavorants. Capsules may be hard or soft. Examples of hard capsules include ConiSnap®, DRcaps®, OceanCaps®, Pearlcaps®, Plantcaps®, DUOCAP®, Vcaps®. and Vcaps®. Plus capsules available from Capsugel®. Hard capsules may be prepared, for example, by forming two telescoping capsule halves, filling one of the halves with a fill comprising a compound of Formula I and/or Formula Ia, or a pharmaceutically acceptable salt thereof, and sealing the capsule halves together. The fill may be in any suitable form, such as dry powder, granulation, suspension or liquid. Examples of soft capsules include soft gelatin (also called softgel or soft elastic) capsules, such as SGcaps®. Soft capsules may be prepared, for example, by rotary die, plate, reciprocating die or Accogel® machine method. In embodiments, the capsule may be a liquid-filled hard capsule or a soft-gelatin capsule.

Tablets can be made by compression or molding, optionally with one or more accessory ingredients. Compressed tablets can be prepared by compressing in a suitable machine a compound of formula (I) or pharmaceutically acceptable salt thereof in a free-flowing form such as a powder or granules, optionally mixed with a binder, lubricant, inert diluent, preservative, surface-active or dispersing agent. Molded tablets can be made by molding in a suitable machine a mixture of the powdered compound moistened with an inert liquid diluent. The tablets can be optionally coated or scored and can be formulated so as to provide sustained, extended, delayed or controlled release. Methods of formulating such sustained, extended, delayed or controlled release compositions are known in the art and disclosed in issued U.S. patents, including but not limited to U.S. Pat. Nos. 4,369,174, 4,842,866, and the references cited therein. Coatings, for example enteric coatings, can be used for delivery of compounds to the intestine (see, e.g., U.S. Pat. Nos. 6,638,534, 5,217,720, 6,569,457, and the references cited therein). In addition to tablets, other dosage forms, such as capsules, granulations and gel-caps, can be formulated to provide sustained, extended, delayed or controlled release.

In one embodiment, the pharmaceutical composition is formulated for parenteral administration. Examples of a pharmaceutical composition suitable for parenteral administration include aqueous sterile injection solutions and non-aqueous sterile injection solutions, each containing, for example, anti-oxidants, buffers, bacteriostatic agents and/or solutes that render the formulation isotonic with the blood of the intended recipient; and aqueous sterile suspensions and non-aqueous sterile suspensions, each containing, for example, suspending agents and/or thickening agents. The formulations can be presented in unit-dose or multi-dose containers, for example, sealed ampules or vials, and can be stored in a freeze dried (lyophilized) condition requiting only the addition of a sterile liquid carrier, such as water, immediately prior to use. In one embodiment, the pharmaceutical composition is formulated for intravenous administration.

In some embodiments, the pharmaceutical composition further includes a pharmaceutically acceptable excipient. A pharmaceutically acceptable excipient may be any substance, not itself a therapeutic agent, used as a carrier, diluent, adjuvant, binder, and/or vehicle for delivery of a therapeutic agent to a patient, or added to a pharmaceutical composition to improve its handling or storage properties or to permit or facilitate formation of a compound or pharmaceutical composition into a unit dosage form for administration. Pharmaceutically acceptable excipients are known in the pharmaceutical arts and are disclosed, for example, in Remington: The Science and Practice of Pharmacy, 21.sup.st Ed. (Lippincott Williams & Wilkins, Baltimore, Md., 2005). As will be known to those in the art, pharmaceutically acceptable excipients can provide a variety of functions and can be described as wetting agents, buffering agents, suspending agents, lubricating agents, emulsifiers, disintegrants, absorbents, preservatives, surfactants, colorants, flavorants, and sweeteners. Examples of pharmaceutically acceptable excipients include without limitation: (1) sugars, such as lactose, glucose and sucrose; (2) starches, such as corn starch and potato starch; (3) cellulose and its derivatives, such as sodium carboxymethyl cellulose, ethyl cellulose, cellulose acetate, hydroxypropyl methylcellulose, and hydroxypropylcellulose; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of a compound of Formula I, and/or Formula Ia, or a pharmaceutically acceptable salt thereof the manner of administration, the composition employed, and the severity of the disease or condition being treated.

In addition to administering the compound as a raw chemical, the compounds of the disclosure may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The pharmaceutical compositions of the disclosure may be administered to any patient which may experience the beneficial effects of the compounds of the disclosure. Foremost among such patients are mammals, e.g., humans, although the disclosure is not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present disclosure are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this disclosure are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C12). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present disclosure. Various modifications and alterations of the compositions and methods described above can readily be achieved using expertise available in the art and are within the scope of the disclosure.

In some embodiments, a carrier, i.e., a diluent, adjuvant or excipient, can be used to prepare a formulation for administration, which includes a compound of Formula I, and/or Formula Ia, or a checkpoint inhibitor, and/or both. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to animal, a compound of Formula I and/or Formula Ia, or a pharmaceutically acceptable salt thereof or compositions and pharmaceutically acceptable carriers are sterile. Water is a preferred carrier when a compound of Formula I and/or Formula Ia, or a pharmaceutically acceptable salt thereof are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Pharmaceutical compositions containing a compound of Formula I and/or Formula Ia, or a pharmaceutically acceptable salt thereof, according to the present disclosure will comprise an effective amount of a compound of Formula I, and/or Formula Ia, or a pharmaceutically acceptable salt thereof, a checkpoint inhibitor or fragment thereof, and/or both, typically dispersed in a pharmaceutically acceptable carrier. The phrases “pharmaceutically or pharmacologically acceptable” refers to molecular entities and compositions that do not produce adverse, allergic or other untoward reaction when administered to animal, such as, for example, a human, as appropriate. The preparation of an pharmaceutical composition that contains the combination or its constituent parts will be known to those of skill in the art in light of the present disclosure, as exemplified by Remington's Pharmaceutical Sciences, 18th Ed. Mack Printing Company, 1990, Moreover, for animal (e.g., human) administration, it will be understood that preparations should meet sterility, pyrogenicity, general safety and purity standards. A specific example of a pharmacologically acceptable carrier as described herein is borate buffer or sterile saline solution (0.9% NaCl).

Formulations of the antibodies used in accordance with the present disclosure can be prepared for storage by mixing an antibody having the desired degree of purity with optional pharmaceutically acceptable carriers, excipients or stabilizers as amply described and illustrated in Remington's Pharmaceutical Sciences 16^{th} edition, Osol, A. Ed. [1980], in the form of lyophilized formulations or aqueous solutions and/or suspensions. Acceptable carriers, excipients, buffers or stabilizers are nontoxic to recipients at the dosages and concentrations employed, and include suitable aqueous and/or non-aqueous excipients that may be employed in the pharmaceutical compositions of the disclosure, for example, water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants, buffers such as phosphate, citrate, and other organic acids. Antioxidants may be included, for example, (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like; preservatives (such as octade-cyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues). Other exemplary pharmaceutically acceptable excipients may include polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In one illustrative embodiment, the pharmaceutical compositions can optionally contain pharmaceutically acceptable auxiliary substances as required to approximate physiological conditions such as pH adjusting and buffering agents and toxicity adjusting agents, for example, sodium acetate, sodium chloride, potassium chloride, calcium chloride and sodium lactate. In some embodiments, the checkpoint inhibitor antibodies or antigen-binding fragments thereof of the present disclosure are formulated for and can be lyophilized for storage and reconstituted in a suitable excipient prior to use according to art-known lyophilization and reconstitution techniques. In one exemplary pharmaceutical composition containing one or more checkpoint inhibitor antibodies or antigen-binding fragment thereof, the composition is formulated as a sterile, preservative-free solution of one or more checkpoint inhibitor antibodies or antigen-binding fragment thereof for intravenous or subcutaneous administration. The formulation can be supplied as either a single-use, prefilled pen, as a single-use, for example containing about 1 mL prefilled glass syringe, or as a single-use institutional use vial. Preferably, the pharmaceutical composition containing the checkpoint inhibitor antibody or antigen-binding fragment thereof is clear and colorless, with a pH of about 6.9-5.0, preferably a pH of 6.5-5.0, and even more preferably a pH ranging from about 6.0 to about 5.0. In various embodiments, the formulations comprising the pharmaceutical compositions can contain from about 500 mg to about 10 mg, or from about 400 mg to about 20 mg, or from about 300 mg to about 30 mg or from about 200 mg to about 50 mg of the checkpoint inhibitor antibody or antigen-binding fragment thereof per mL of solution when reconstituted and administered to the subject. Exemplary injection or infusion excipients can include mannitol, citric acid monohydrate, dibasic sodium phosphate dihydrate, monobasic sodium phosphate dihydrate, polysorbate 80, sodium chloride, sodium citrate and water for parenteral administration, for example, intravenously, intramuscularly, intraperitoneally, or subcutaneous administration.

In another exemplary embodiment, one or more checkpoint inhibitor antibodies, or antigen-binding fragment thereof is formulated for intravenous or subcutaneous administration as a sterile aqueous solution containing 1-75 mg/mL, or more preferably, about 5-60 mg/mL, or yet more preferably, about 10-50 mg/mL, or even more preferably, about 10-40 mg/mL of antibody, with sodium acetate, polysorbate 80, and sodium chloride at a pH ranging from about 5 to 6. Preferably, the intravenous or subcutaneous formulation is a sterile aqueous solution containing 5, 10, 15, 20, 25, 30, 35, 40, 45, or 50 mg/mL of checkpoint inhibitor antibody or an antigen-binding fragment thereof, with 20 mM sodium acetate, 0.2 mg/mL polysorbate 80, and 140 mM sodium chloride at pH 5.5. Further, a solution comprising a checkpoint inhibitor antibody or an antigen-binding fragment thereof, can comprise, among many other compounds, histidine, mannitol, sucrose, trehalose, glycine, poly(ethylene)glycol, EDTA, methionine, and any combination thereof, and many other compounds known in the relevant art.

In one embodiment, a pharmaceutical composition of the present disclosure comprises the following components: 5-50 mg checkpoint inhibitor antibody or antigen-binding fragment of the present disclosure, 10 mM histidine, 5% sucrose, and 0.01% polysorbate 80 at pH 5.8. This composition may be provided as a lyophilized powder. When the powder is reconstituted at full volume, the composition retains the same formulation. Alternatively, the powder may be reconstituted at half volume, in which case the composition comprises 10-100 mg checkpoint inhibitor antibody or antigen-binding fragment thereof of the present disclosure, 20 mM histidine, 10% sucrose, and 0.02% polysorbate 80 at pH 5.8.

In one embodiment, part of the dose is administered by an intravenous bolus and the rest by infusion of the antibody formulation. For example, from about 0.001 to about 200 mg/kg, for example, from about 0.001 mg/kg to about 100 mg/kg, or from about 0.001 mg/kg to about 50 mg/kg, or from about 0.001 mg/kg to about 10 mg/kg intravenous injection of the checkpoint inhibitor antibody, or antigen-binding fragment thereof, may be given as a bolus, and the rest of the antibody dose may be administered by intravenous injection. A predetermined dose of the checkpoint inhibitor antibody, or antigen-binding fragment thereof, may be administered, for example, over a period of an hour to two hours to five hours.

In a further embodiment, part of the dose is administered by a subcutaneous injection and/or infusion in the form of a bolus and the rest by infusion of the antibody formulation. In some exemplary doses, the antibody formulation can be administered subcutaneously in a dose ranging from about 0.001 to about 200 mg/kg, for example, from about 0.001 mg/kg to about 100 mg/kg, or from about 0.001 mg/kg to about 50 mg/kg, or from about 0.001 mg/kg to about 10 mg/kg intravenous injection of the checkpoint inhibitor antibody, or antigen-binding fragment thereof. In some embodiments the dose may be given as a bolus, and the rest of the antibody dose may be administered by subcutaneous or intravenous injection. A predetermined dose of the checkpoint inhibitor antibody, or antigen-binding fragment thereof, may be administered, for example, over a period of an hour to two hours to five hours.

The formulation herein may also contain more than one active compound as necessary for the particular indication being treated, preferably those with complementary activities that do not adversely affect each other. For example, it may be desirable to provide antibodies with other specificities. Alternatively, or in addition, the composition may comprise an anti-inflammatory agent, a chemotherapeutic agent, a cytotoxic agent, a cytokine, a growth inhibitory agent and/or a small molecule antagonist. Such molecules are suitably present in combination in amounts that are effective for the purpose intended.

The formulations to be used for in vivo administration should be sterile, or nearly so. This is readily accomplished by filtration through sterile filtration membranes.

In various embodiments, illustrative formulations of the pharmaceutical compositions described herein can be prepared using methods widely known in the field of pharmaceutical formulations. In general, such preparatory methods can include the step of bringing the active ingredient into association with a carrier or one or more other accessory ingredients, and then, if desirable, packaging the product into a desired single- or multi-dose unit.

In some embodiments, the pharmaceutical composition can be also delivered in a vesicle, in particular, a liposome containing one or more liposomal surface moieties for example, polyethylene glycol, antibodies and antibody fragments thereof, which are selectively transported into specific cells or organs, thus enhance targeted drug delivery.

The optimum concentration of the active ingredient(s) in the chosen medium can be determined empirically, according to procedures well known to the skilled artisan, and will depend on the ultimate pharmaceutical formulation desired and the use to be employed.

The present disclosure also provides a pharmaceutical pack or kit comprising one or more containers filled with one or more of the ingredients of the pharmaceutical compositions of the disclosure, which at minimum will include a compound of Formula I and/or Formula Ia, or a pharmaceutically acceptable salt thereof, and one or more checkpoint inhibitor antibodies or antigen-binding fragment thereof as described herein. In other embodiments, the kit may contain one or more further containers providing a pharmaceutically acceptable excipient, for example a diluent. In one embodiment a kit may comprise at least one container, wherein the container can include a compound of Formula I, and/or Formula Ia, or a pharmaceutically acceptable salt thereof, a checkpoint inhibitor antibody or an antigen-binding fragment thereof of the present disclosure. The kit may also include a set of instructions for preparing and administering the final pharmaceutical composition to the subject in need thereof, for the treatment of a checkpoint molecule-mediated disease or disorder.

Methods of Treatment

In some embodiments of the present disclosure, the combination, i.e., the compound of Formula I and/or Formula Ia, or a pharmaceutically acceptable salt thereof, and one or more checkpoint inhibitors or antigen-binding fragments thereof, can be employed under a variety of conditions and therapeutic uses to treat a variety of immunological conditions, including cancer.

The dose to be administered to a subject in need thereof may vary depending upon a variety of factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, size, condition, general health, the prior medical history of the patient being treated, target disease, the purpose of the treatment, conditions, the immunogenicity of the entity, and the accessibility of the target cells in the biological matrix and the like. When the combination of the present disclosure is used for treating various conditions and diseases directly or indirectly associated with immune checkpoints, in an adult subject, it is advantageous to intravenously or subcutaneously administer the antibody of the present disclosure.

In various embodiments, the appropriate dose of the active agents described herein is made by the clinician, e.g., using parameters or factors known or suspected in the art to affect treatment or predicted to affect treatment. In some embodiments, sound medical practice will dictate that the initial dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced, tissue damage, or estimated activity or stage in a cancer disease course. In some embodiments, the actual dosage levels of the active ingredients in the pharmaceutical compositions of the present disclosure may be varied so as to obtain an amount of the active ingredient which is effective to achieve the desired therapeutic response for a particular subject, composition, and mode of administration, without being toxic to the patient. Compositions comprising the combination of the disclosure can be administered to the subject, for example, a human subject by one or more administration modalities, for example, continuous infusion, or by doses at intervals of, e.g., one day, one week, or 1-7 times per week. Doses may be provided parenterally, for example, intravenously, or subcutaneously.

By way of illustration only, and taking into consideration various factors for determining appropriate doses and dosing frequencies, an exemplary dose of the combination (i.e., a combination comprising a compound of Formula I and/or Formula Ia, or a pharmaceutically acceptable salt thereof, and one or more checkpoint inhibitor antibodies or antigen-binding fragments thereof) to be administered to a patient in need thereof can include a single dose of each active agent (i.e. a compound of Formula I and/or Formula Ia, or a pharmaceutically acceptable salt thereof, and one or more checkpoint inhibitor antibodies or antigen-binding fragments thereof) about 0.01 to about 100 mg/kg body weight, more preferably about 0.02 to about 7, about 0.03 to about 5, or about 0.05 to about 3 mg/kg body weight dosed, once or more times per day, and/or one or more times per week, for example, for one to four weeks, or one to eight weeks, or one to twelve weeks, or one to fourteen weeks. In some embodiments, an exemplary dosing regimen can include administration of a maximal dose or dosing frequency that avoids significant undesirable side effects. In some embodiments, a total weekly dose of each active agent of the combination, independently may be at least 0.05 μg/kg body weight, at least 0.2 μg/kg, at least 0.5 μg/kg, at least 1 μg/kg, at least 10 μg/kg, at least 100 μg/kg, at least 0.2 mg/kg, at least 0.5 mg/kg, at least 1.0 mg/kg, at least 2.0 mg/kg, at least 10 mg/kg, at least 15 mg/kg, at least 20 mg/kg, at least 25 mg/kg, or at least 50 mg/kg, or at least or at least 100 mg/kg. In another example, an illustrative dose of each active agent of the combination of the disclosure, to be administered to a patient in need thereof may be about 0.001 mg/kg to about 200 mg/kg of the patient's body weight. The dosage to a subject in need thereof, may be between 0.001 mg/kg and 200 mg/kg, 0.001 mg/kg and 100 mg/kg, 0.001 mg/kg and 50 mg/kg, 0.001 mg/kg and 25 mg/kg, 0.001 mg/kg and 10 mg/kg, 0.001 mg/kg and 5 mg/kg, 0.001 mg/kg and 1 mg/kg, 0.001 mg/kg and 0.5 mg/kg and any dosage amount there between. As non-limiting examples, treatment according to the present disclosure may be provided as a daily dosage of an each active agent of the combination, in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

Depending on the severity of the condition, and the various factors discussed herein, the dose, frequency and the duration of the treatment can be adjusted accordingly, in view of proper medical standards known to those of skill in the art. In certain exemplary embodiments, each active agent of the combination of the disclosure can be administered as an initial dose of at least about 0.1 mg to about 800 mg, about 1 to about 500 mg, about 5 to about 300 mg, or about 10 to about 200 mg, to about 100 mg, or to about 50 mg. The first dose of one or both active agents of the combination may be an initial loading dose, to be followed subsequently by a plurality of maintenance doses. In certain exemplary embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks, or doses of the combination of the disclosure may be repeated and the administrations may be separated by at least 1 day, 2 days, 3 days, 5 days, 10 days, 15 days, 30 days, 45 days, 2 months, 75 days, 3 months, or at least 6 months.

The route of administration of the compositions containing each active agent of the combination of active agents, i.e. a combination comprising a compound of Formula I and/or Formula Ia, or a pharmaceutically acceptable salt thereof, and one or more checkpoint inhibitor antibodies or antigen-binding fragments thereof of the present disclosure may be independently administered, or administered as a combination in a single formulation by, e.g., topical or cutaneous application, injection or infusion by intravenous, intraperitoneal, subcutaneous, intracerebral, intramuscular, intraocular, intraarterial, intradermal, intracerebrospinal, intralesional, subcuticular, intraarticular, subcapsular, subarachnoid, intraspinal, epidural and intrasternal injection and infusion, or by sustained release systems or an implant. The injectable preparations may include dosage forms for intravenous, subcutaneous, intracutaneous and intramuscular injections, drip infusions, etc. Numerous reusable pen and autoinjector delivery devices have applications in the subcutaneous delivery of a pharmaceutical composition of the present disclosure. Examples include, but certainly are not limited to AUTOPEN™ (Owen Mumford, Inc., Woodstock, UK), DIS-ETRONIC™ pen (Disetronic Medical Systems, Burghdorf, Switzerland), HUMALOG MIX 75/25™ pen, HUMA-LOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nor-disk, Copenhagen, Denmark), BD™ pen (Becton Dickinson, Franklin Lakes, N.J.), OPTIPEN™, OPTIPEN PRO™ OPTIPEN STARLET™, and OPTICLIK™ (Sanofi-Aventis, Frankfurt, Germany), as exemplary pen based delivery methods contemplated herein in the administration of the present combination. Illustrative examples of pen based devices having applications in subcutaneous delivery of a pharmaceutical composition of the present disclosure include, the SOLOSTAR™ pen (Sanofi-Aventis), the FLEXPEN™ (Novo Nordisk), and the KWIKPEN™ (Eli Lilly).

Generally, the oral dosage of a compound of Formula I and/or Formula Ia, or a pharmaceutically acceptable salt thereof, administered to an animal, for example a human subject, is about 0.01 mg/kg to about 100 mg/kg of the animal's body weight, more typically about 5 mg/kg, 10 mg/kg, 25 mg/kg, 50 mg/kg, 100 mg/kg, 150 mg/kg, 200 mg/kg, 250 mg/kg, or 300 mg/kg to about 500 mg/kg of the animal's body weight. In some aspects, the dosage of a compound of Formula I and/or Formula Ia, or a pharmaceutically acceptable salt thereof administered to animal is about 1 mg, about 5 mg, or about 10 mg to about 350 mg per day, or from about 1 mg, about 5 mg, about 10 mg, about 15 mg or about 20 mg to about 100 mg per day.

A compound of Formula I and/or Formula Ia, or a pharmaceutically acceptable salt thereof, or a pharmaceutical composition thereof and a checkpoint inhibitor antibody or a functional fragment thereof, each independently or in combination can be administered on a daily, weekly, biweekly or monthly schedule, according to the desired effect. In some aspects, a compound of Formula I or a pharmaceutical composition thereof can be administered from about 1 to 5, about 1 to about 10, about 1 to about 15, or more cycles, wherein each cycle is a month in duration. The doses within each cycle can be given on daily (including once daily, twice daily, or more than twice daily), every other day, twice weekly, weekly, bi-weekly, once every three weeks or monthly basis. A cycle may optionally include a resting period. Alternatively, a resting period can be included between cycles. In some aspects, administration will be for the duration of the disease.

As described herein, the amount of a compound of Formula I and/or Formula Ia, or a pharmaceutically acceptable salt thereof, and the amount of a checkpoint inhibitor antibody or a functional fragment thereof, each independently or in combination that is effective in the methods described herein will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

In some embodiments of the present disclosure, a compound of Formula I and/or Formula Ia or a pharmaceutically acceptable salt thereof of the disclosure and one or more checkpoint inhibitor antibodies or antigen binding fragment thereof are administered to an animal under one or more of the following conditions: at different periodicities, at different durations, at different concentrations, by different administration routes, etc. In some embodiments, the compound is administered prior to the checkpoint inhibitor antibody or antigen binding fragment thereof, e.g., 0.5, 1, 2, 3, 4, 5, 10, 12, or 18 hours, 1, 2, 3, 4, 5, or 6 days, or 1, 2, 3, or 4 weeks prior to the administration of the checkpoint inhibitor antibody or antigen binding fragment thereof. In some embodiments, the compound of Formula I and/or Formula Ia or a pharmaceutically acceptable salt thereof is administered after the one or more checkpoint inhibitor antibodies or antigen binding fragment thereof, e.g., 0.5, 1, 2, 3, 4, 5, 10, 12, or 18 hours, 1, 2, 3, 4, 5, or 6 days, or 1, 2, 3, or 4 weeks after the administration of the one or more checkpoint inhibitor antibodies or antigen binding fragment thereof. In some embodiments, the compound of Formula I and/or Formula Ia or a pharmaceutically acceptable salt thereof and the checkpoint inhibitor antibody or antigen binding fragment thereof are administered concurrently but on different schedules, e.g., the compound of Formula I and/or Formula Ia or a pharmaceutically acceptable salt thereof is administered daily while the checkpoint inhibitor antibody or antigen binding fragment thereof is administered once a week, once every two weeks, once every three weeks, or once every four weeks. In other embodiments, the compound of Formula I and/or Formula Ia or a pharmaceutically acceptable salt thereof is administered once a week while the checkpoint inhibitor antibody or antigen binding fragment thereof is administered daily, once a week, once every two weeks, once every three weeks, or once every four weeks.

Compositions within the scope of this disclosure include all compositions wherein the compound of Formula I and/or Formula Ia or a pharmaceutically acceptable salt thereof of the present disclosure are contained in an amount which is effective to achieve its intended purpose. While individual needs vary, determination of optimal ranges of effective amounts of each component is within the skill of the art. Typically, the compound of Formula I and/or Formula Ia or a pharmaceutically acceptable salt thereof may be administered to mammals, e.g. humans, orally at a dose of 0.0025 to 100 mg/kg, or an equivalent amount of the pharmaceutically acceptable salt thereof, per day of the body weight of the mammal being treated for the cancer being treated. In one embodiment, about 0.01 to about 25 mg/kg is orally administered to treat, ameliorate, or prevent such disorders. For intravenous injection or infusion injection, the dose of the checkpoint inhibitor antibody or antigen binding fragment thereof would be about 0.1 to about 1000 mg/kg, or from about 0.1 mg/kg to about 500 mg/kg patient weight.

The unit oral dose of the compound of Formula I and/or Formula Ia or a pharmaceutically acceptable salt thereof may comprise from about 0.01 mg to about 1000 mg, for example, about 0.1 to about 100 mg of the compound. The unit dose may be administered one or more times daily as one or more tablets or capsules each containing from about 0.1 to about 10 mg, conveniently about 0.25 to 50 mg of the compound or its solvates.

In a topical formulation, the compound of Formula I and/or Formula Ia or a pharmaceutically acceptable salt thereof may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

The compound of Formula I and/or Formula Ia or a pharmaceutically acceptable salt thereof according to the disclosure is administered in combination with a checkpoint inhibitor for example, a checkpoint inhibitor antibody or functional fragment thereof, for example, any one or more antibodies selected from: Tremelimumab, Abatacept, AK104, REGN2810 (Cemiplimab), Nivolumab, Pembrolizumab, Sintilimab (IBI308), Tislelizumab (BGB-A317), SHR-1210 (Camrelizumab), Spartalizumab (PDR001), JS001, TSR-042, JNJ-63723283, BCD-100, TORIPALIMAB, Avelumab, Atezolizumab, TQB2450, KN035, CS1001, and/or Durvalumab (MEDI4736).

In some embodiments, the checkpoint inhibition therapy comprises administration of a blocking agent, selected from a cell, protein, peptide, antibody or antigen binding fragment thereof, directed against CTLA-4, PD-1, PD-L1, TIM-3, BTLA, VISTA, LAG-3 and combinations thereof.

In another embodiment, the checkpoint inhibition therapy comprises administration of a sub-therapeutic amount and/or duration of a blocking agent, selected from a cell, protein, peptide, antibody or antigen binding fragment thereof, directed against CTLA-4, PD-1, PD-L1, TIM-3, BTLA, VISTA, LAG-3 and combinations thereof.

In a further embodiment, the checkpoint inhibition therapy comprises administration of a blocking agent, selected from a cell, protein, peptide, antibody or antigen binding fragment thereof, directed against PD-1 and/or, PD-L1, simultaneously, separately or sequentially with administration of a blocking antibody or antigen binding fragment thereof, directed against CTLA-4.

The term “combination” as used throughout the specification, is meant to encompass the administration of the checkpoint inhibitor simultaneously, separately or sequentially with administration of a compound of Formula I and/or Formula Ia or a pharmaceutically acceptable salt thereof and/or its derivative compounds. Accordingly, one or more checkpoint inhibitors and the compound of Formula I and/or Formula Ia or a pharmaceutically acceptable salt thereofmay be present in the same or separate pharmaceutical formulations, and administered at the same time or at different times.

Thus, a compound of Formula I and/or Formula Ia or a pharmaceutically acceptable salt thereof and one or more checkpoint inhibitors may be provided as separate medicaments for administration at the same time or at different times.

In some embodiments, compound of Formula I and/or Formula Ia or a pharmaceutically acceptable salt thereof and checkpoint inhibitor are provided as separate medicaments for administration at different times. When administered separately and at different times, either the compound of Formula I and/or Formula Ia or a pharmaceutically acceptable salt thereof or checkpoint inhibitor may be administered first; however, in some situations, it may be more suitable to administer checkpoint inhibitor followed by the compound of Formula I and/or Formula Ia or a pharmaceutically acceptable salt thereof, and vice versa. In addition, both can be administered on the same day or at different days, and they can be administered using the same schedule or at different schedules during the treatment cycle.

In some embodiments, the mode of administration of the combination, i.e., a compound of Formula I and/or Formula Ia or a pharmaceutically acceptable salt thereof, a checkpoint inhibitor or fragment thereof, both, or a pharmaceutical composition thereof, is left to the discretion of the practitioner, and will depend in-part upon the site of the medical condition, and the type of medical condition/ailment. In one embodiment, the combination or its constituent parts, or compositions containing a compound of Formula I and/or Formula Ia or a pharmaceutically acceptable salt thereof and/or one or more checkpoint inhibitors, are administered parenterally. In another embodiment, the combination or compositions comprising a compound of Formula I and/or Formula Ia, or a pharmaceutically acceptable salt thereof, and/or one or more checkpoint inhibitors are administered orally.

In another embodiment, a compound of Formula I and/or Formula Ia or a pharmaceutically acceptable salt thereof can be delivered in a vesicle, in particular a liposome (see Langer, Science 249:1527-1533 (1990); Treat et al., in LIPOSOMES IN THE THERAPY OF INFECTIOUS DISEASE AND CANCER, Lopez-Berestein and Fidler (eds.), Liss, N.Y., pp. 353-365 (1989); Lopez-Berestein, ibid., pp. 317-327; see generally ibid.).

In yet another embodiment, a compound of Formula I and/or Formula Ia, or a pharmaceutically acceptable salt thereof, or compositions containing the combination, can be delivered in a controlled release system. In one embodiment, a pump can be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see MEDICAL APPLICATIONS OF CONTROLLED RELEASE, Langer and Wise (eds.), CRC Pres., Boca Raton, Fla. (1974); CONTROLLED DRUG BIOAVAILABILITY, DRUG PRODUCT DESIGN AND PERFORMANCE, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). Other controlled-release systems discussed in the review by Langer (Science 249:1527-1533 (1990)) can be used.

Generally, the optimal amount of a compound of Formula I and/or Formula Ia, or a pharmaceutically acceptable salt thereof and the checkpoint inhibitor that is effective in the treatment of cancer can be determined by standard clinical techniques. In addition, in vitro assays may optionally be employed to help identify optimal dosage ranges. The precise dose to be employed in the formulation will also depend on the route of administration, and the stage of malignancy, and should be decided according to the judgment of the practitioner and each patient's circumstances. Effective doses may be extrapolated from dose-response curves derived from in vitro or animal model test systems.

In a further aspect, the checkpoint inhibitor and a compound of Formula I and/or Formula Ia or a pharmaceutically acceptable salt thereof are administered simultaneously or sequentially, in either order. In a specific aspect, the checkpoint inhibitor is a PD-1 inhibitor or CTLA-4 inhibitor. In another specific aspect, the checkpoint inhibitor is a PD-1 inhibitor.

In some embodiments, a compound of Formula I and/or Formula Ia or a pharmaceutically acceptable salt thereof and the checkpoint inhibitor will be administered to a subject at the Maximal Tolerable Dose (MTD) or the Optimal Biological Dose (OBD). It is within the art to determine MTD or OBD. In some aspects, a compound of Formula I and/or Formula Ia, or a pharmaceutically acceptable salt thereof, will be provided at its MTD or OBD and the checkpoint inhibitor will be dosed at 50%-100%, preferably at 50% to 90% of the MTD or OBD. Alternatively, the checkpoint inhibitor will be dosed at its MTD or OBD and a compound of Formula I and/or Formula Ia, or a pharmaceutically acceptable salt thereof, will be dosed at 50%-100%, preferably at 50% to 90% of the MTD or OBD. In some aspects, both a compound of Formula I and/or Formula Ia, or a pharmaceutically acceptable salt thereof, and the checkpoint inhibitor will be dosed at 60% to 90% of the MTD or OBD.

As used in this disclosure, the combination regimen can be given simultaneously or can be given in a staggered regimen, with the checkpoint inhibitor being given at a different time during the course of therapy than a compound of Formula I and/or Formula Ia or a pharmaceutically acceptable salt thereof. This time differential may range from several minutes, hours, days, weeks, or longer between administration of the two agents. Therefore, the term combination does not necessarily mean administered at the same time or as a unitary dose, but that each of the components are administered during a desired treatment period. The agents may also be administered by different routes.

Also provided herein is the prodrug of a compound of Formula I and/or Formula Ia or a pharmaceutically acceptable salt thereof or a pharmaceutically acceptable salt of a compound of Formula I or its prodrug. Accordingly, in any of the various embodiments provided herein, the prodrug of a compound of Formula I and/or Formula Ia or a pharmaceutically acceptable salt thereof or its prodrug can be used.

In aspect of the disclosure, the effective amount of the compound of Formula I and/or Formula Ia or a pharmaceutically acceptable salt thereof may be administered as a single dose. Alternatively, the effective amount of the a compound of Formula I and/or Formula Ia or a pharmaceutically acceptable salt thereof may be administered in multiple (repeat) doses, for example two or more, three or more, four or more, five or more, ten or more, or twenty or more repeat doses. The a compound of Formula I and/or Formula Ia or a pharmaceutically acceptable salt thereof may be administered between about 4 weeks and about 1 day prior to checkpoint inhibition therapy, such as between about 4 weeks and 1 week, or about between 3 weeks and 1 week, or about between 3 weeks and 2 weeks. Administration may be presented in single or multiple doses.

In one embodiment of the disclosure, there is a compound of Formula I and/or Formula Ia or a pharmaceutically acceptable salt thereof for use in the treatment of neoplastic disease, that is used in combination with one or more checkpoint inhibitors or fragments thereof, wherein said compound of Formula I and/or Formula Ia or a pharmaceutically acceptable salt thereof is administered to the subject before, concurrently with and/or after the checkpoint inhibitor is administered.

In another embodiment of the disclosure, there is a method of treating, reducing, inhibiting or controlling a neoplasia, tumor or cancer in a subject, wherein said method comprises simultaneously, separately or sequentially administering to the subject, (i) one or more checkpoint inhibitors (e.g., PD-1); and (ii) a compound of Formula I and/or Formula Ia or a pharmaceutically acceptable salt thereof, wherein said method results in a synergistic effect that manifests itself as enhanced therapeutic efficacy relative to administration of either the checkpoint inhibitor or a compound of Formula I and/or Formula Ia or a pharmaceutically acceptable salt thereof alone.

In another embodiment of the disclosure, there is a method of treating, reducing, inhibiting or controlling a neoplasia, tumor or cancer in a subject, wherein said method comprises simultaneously, separately or sequentially administering to the subject, (i) one or more checkpoint inhibitors, and (ii) a compound of Formula I and/or Formula Ia, or a pharmaceutically acceptable salt thereof, wherein said checkpoint inhibition therapy comprises administration of a blocking agent, selected from a cell, protein, peptide, antibody or antigen binding fragment thereof, directed against CTLA-4, PD-1, PD-L1, TIM-3, BTLA, VISTA, LAG-3 and combinations thereof.

In some embodiments the disclosure is a method of treating, reducing, inhibiting or controlling a neoplasia, tumor or cancer in a subject, wherein said method comprises simultaneously, separately or sequentially administering to the subject, (i) one or more checkpoint inhibitors, and (ii) a compound of Formula I and/or Formula Ia or a pharmaceutically acceptable salt thereof, wherein said checkpoint inhibition therapy comprises administration of a blocking agent, selected from a cell, protein, peptide, antibody or antigen binding fragment thereof, directed against CTLA-4, PD-1 and/or PD-L1.

In yet other embodiments, the disclosure is a method of treating, reducing, inhibiting or controlling a neoplasia, tumor or cancer in a subject, wherein said method comprises simultaneously, separately or sequentially administering to the subject, (i) one or more checkpoint inhibitors, and (ii) a compound of Formula I and/or Formula Ia or a pharmaceutically acceptable salt thereof, wherein said checkpoint inhibition therapy comprises administration of a blocking agent, selected from a cell, protein, peptide, antibody or antigen binding fragment thereof, directed against CTLA-4, PD-1, PD-L1, TIM-3, BTLA, VISTA, LAG-3, and combinations thereof, and wherein said checkpoint inhibition therapy comprises administration of a sub-therapeutic amount and/or duration of said blocking antibody or antigen binding fragment thereof.

In some embodiments, patients can be selected based on the expression and/or overexpression of a checkpoint protein ligand in a suspected tumor cell population as determined by immunofluorescence or immunohistochemistry.

In yet other embodiments, the disclosure is a method of treating, reducing, inhibiting or controlling a neoplasia, tumor or cancer in a subject, wherein said method comprises simultaneously, separately or sequentially administering to the subject, (i) one or more checkpoint inhibitors, and (ii) a compound of Formula I and/or Formula Ia or a pharmaceutically acceptable salt thereof, wherein said checkpoint inhibition therapy comprises administration of a blocking agent, selected from a cell, protein, peptide, antibody or antigen binding fragment thereof, directed against CTLA-4, PD-1, PD-L1, TIM-3, BTLA, VISTA, LAG-3, and combinations thereof, and wherein said method of treating comprises administration of a sub-therapeutic amount and/or duration of compound of Formula I and/or Formula Ia or a pharmaceutically acceptable salt thereof.

In yet other embodiments, the disclosure is a method of treating, reducing, inhibiting or controlling a neoplasia, tumor or cancer in a subject, wherein said method comprises simultaneously, separately or sequentially administering to the subject, (i) two or more checkpoint inhibitors, and (ii) a compound of Formula I and/or Formula Ia or a pharmaceutically acceptable salt thereof, wherein said checkpoint inhibition therapy comprises administration of a blocking agent, selected from a cell, protein, peptide, antibody or antigen binding fragment thereof, directed against CTLA-4, PD-1, PD-L1, TIM-3, BTLA, VISTA, LAG-3 and combinations thereof, wherein said checkpoint inhibition therapy optionally comprises administration of a sub-therapeutic amount and/or duration of said blocking agent, selected from a cell, protein, peptide, antibody or antigen binding fragment thereof.

In yet other embodiments, the method of treating, reducing, inhibiting or controlling a neoplasia, tumor or cancer in a subject, comprises simultaneously, separately or sequentially administering to the subject, (1) a blocking agent, selected from a cell, protein, peptide, antibody or antigen binding fragment thereof, directed against PD-1 or PD-L1; and (2) a compound of Formula I and/or Formula Ia or a pharmaceutically acceptable salt thereof.

In some embodiments, the method of treating, reducing, inhibiting or controlling a neoplasia, tumor or cancer in a subject, comprises simultaneously, separately or sequentially administering to the subject, (1) a blocking agent, selected from a cell, protein, peptide, antibody or antigen binding fragment thereof, directed against PD-1; and (2) a compound of Formula I and/or Formula Ia or a pharmaceutically acceptable salt thereof.

In some embodiments, a method of treating, reducing, inhibiting or controlling a neoplasia, tumor or cancer in a subject, comprises simultaneously, separately or sequentially administering to the subject, (1) a blocking agent, selected from a cell, protein, peptide, antibody or antigen binding fragment thereof, directed against PD-1, e.g., Pembrolizumab (commercially available as Keytruda® from Merck®), Nivolumab (commercially available as Opdivo® from Bristol-Myers Squibb®), or Cemiplimab (commercially available as Libtayo® from Regeneron Pharmaceuticals, Inc® and Sanofi-Aventis®); and (2) a compound of Formula I and/or Formula Ia or a pharmaceutically acceptable salt thereof.

In some embodiments, a method of treating, reducing, inhibiting or controlling a neoplasia, tumor or cancer in a subject, comprises simultaneously, separately or sequentially administering to the subject, (1) a blocking agent, selected from a cell, protein, peptide, antibody or antigen binding fragment thereof, comprising Tremelimumab, Abatacept, AK104, REGN2810 (Cemiplimab), Nivolumab, Pembrolizumab, Sintilimab (IBI308), Tislelizumab (BGB-A317), SHR-1210 (Camrelizumab), Spartalizumab (PDR001), JS001, TSR-042, JNJ-63723283, BCD-100, TORIPALIMAB, Avelumab, Atezolizumab, TQB2450, KN035, CS1001, and/or Durvalumab (MEDI4736); and (2) a compound of Formula I and/or Formula Ia or a pharmaceutically acceptable salt thereof.

In some embodiments, the method of treating, reducing, inhibiting or controlling a neoplasia, tumor or cancer in a subject, comprises simultaneously, separately or sequentially administering to the subject, (1) a blocking agent, selected from a cell, protein, peptide, antibody or antigen binding fragment thereof, directed against PD-1, e.g., Pembrolizumab (commercially available as Keytruda®), and (2) a compound of Formula I and/or Formula Ia, or a pharmaceutically acceptable salt thereof.

In certain embodiments, the method of treating, reducing, inhibiting or controlling a neoplasia, tumor or cancer in a subject, comprises simultaneously, separately or sequentially administering to the subject, (1) an antibody or a functional fragment thereof, is selected from the group: Tremelimumab, Abatacept, AK104, REGN2810 (Cemiplimab), Nivolumab, Pembrolizumab, Sintilimab (IBI308), Tislelizumab (BGB-A317), SHR-1210 (Camrelizumab), Spartalizumab (PDR001), JS001, TSR-042, JNJ-63723283, BCD-100, TORIPALIMAB, Avelumab, Atezolizumab, TQB2450, KN035, CS1001, Durvalumab (MEDI4736), or combinations thereof, and (2) a compound of Formula I and/or Formula Ia or a pharmaceutically acceptable salt thereof, wherein the amount of the antibody or functional fragment thereof comprises a dose ranging from about 0.001 mg/kg to about 1000 mg/kg.

In one embodiment, the method of treating, reducing, inhibiting or controlling a neoplasia, tumor or cancer in a subject, comprises simultaneously, separately or sequentially administering to the subject, (1) Pembrolizumab (commercially available as Keytruda®), and (2) a compound of Formula I and/or Formula Ia or a pharmaceutically acceptable salt thereof, wherein the amount of Pembrolizumab comprises a dose of 200 mg administered as an intravenous infusion over 30 minutes every 3 weeks, or up to 24 months in subjects without disease progression.

In another embodiment, the method of treating, reducing, inhibiting or controlling a neoplasia, tumor or cancer in a subject, comprises simultaneously, separately or sequentially administering to a subject who is a child, (1) Pembrolizumab (commercially available as Keytruda®), and (2) a compound of Formula I and/or Formula Ia or a pharmaceutically acceptable salt thereof, wherein the amount of Pembrolizumab comprises a dose of 2 mg/kg (up to a maximum of 200 mg), administered as an intravenous infusion over 30 minutes every 3 weeks until disease progression or unacceptable toxicity, or up to 24 months in patients without disease progression.

Pembrolizumab is commercially available as Keytruda®, which can be prepared, stored, and administered as follows: first, reconstitute Keytruda® for Injection (Lyophilized Powder) by adding 2.3 mL of Sterile Water for Injection, USP by injecting the water along the walls of the vial and not directly on the lyophilized powder (resulting concentration 25 mg/mL). Slowly swirl the vial. Allow up to 5 minutes for the bubbles to clear. Do not shake the vial. To prepare, visually inspect the solution for particulate matter and discoloration prior to administration. The solution is clear to slightly opalescent, colorless to slightly yellow. Discard the vial if visible particles are observed. Dilute Keytruda® injection (solution) or reconstituted lyophilized powder prior to intravenous administration. Next, withdraw the required volume from the vial(s) of Keytruda® and transfer into an intravenous (IV) bag containing 0.9% Sodium Chloride Injection, USP or 5% Dextrose Injection, USP. Mix diluted solution by gentle inversion. The final concentration of the diluted solution should be between 1 mg/mL to 10 mg/mL (discard any unused portion left in the vial). When storing, it should be noted that the product does not contain a preservative, thus, store the reconstituted and diluted solution from the Keytruda® 50 mg vial either: (1) at room temperature for no more than 6 hours from the time of reconstitution (this includes room temperature storage of reconstituted vials, storage of the infusion solution in the IV bag, and the duration of infusion); or (2) under refrigeration at 2° C. to 8° C. (36° F. to 46° F.) for no more than 24 hours from the time of reconstitution. If refrigerated, allow the diluted solution to come to room temperature prior to administration. Store the diluted solution from the Keytruda® 100 mg/4 mL vial either: (1) at room temperature for no more than 6 hours from the time of dilution (this includes room temperature storage of the infusion solution in the IV bag, and the duration of infusion); or (2) under refrigeration at 2° C. to 8° C. (36° F. to 46° F.) for no more than 24 hours from the time of dilution. If refrigerated, allow the diluted solution to come to room temperature prior to administration. Do not freeze. To administer, push the infusion solution intravenously over 30 minutes through an intravenous line containing a sterile, non-pyrogenic, low-protein binding 0.2 micron to 5 micron in-line or add-on filter. Do not co-administer other drugs through the same infusion line.

In some embodiments, the method of treating, reducing, inhibiting or controlling a neoplasia, tumor or cancer in a subject, comprises simultaneously, separately or sequentially administering to the subject, (1) Nivolumab (commercially available as Opdivo®); and (2) a compound of Formula I and/or Formula Ia or a pharmaceutically acceptable salt thereof, wherein the amount of Nivolumab comprises a dose ranging from about 0.001 mg/kg to about 100 mg/kg.

In some embodiments, the method of treating, reducing, inhibiting or controlling a neoplasia, tumor or cancer in a subject, comprises simultaneously, separately or sequentially administering to the subject, (1) Nivolumab (commercially available as Opdivo®); and (2) a compound of Formula I and/or Formula Ia or a pharmaceutically acceptable salt thereof, wherein the amount of Nivolumab comprises a dose of 240 mg every 2 weeks.

In some embodiments, the method of treating, reducing, inhibiting or controlling a neoplasia, tumor or cancer in a subject, comprises simultaneously, separately or sequentially administering to the subject, (1) Nivolumab (commercially available as Opdivo®); and (2) a compound of Formula I and/or Formula Ia or a pharmaceutically acceptable salt thereof, wherein the amount of Nivolumab comprises a dose of 480 mg every 4 weeks.

Nivolumab can be used in concert with other anti-cancer modalities (e.g., the monoclonal antibody Ipilimumab). In such cases, the recommended dose of Nivolumab is 1 mg/kg administered as an intravenous infusion over 30 minutes, followed by ipilimumab 3 mg/kg administered as an intravenous infusion over 90 minutes on the same day, every 3 weeks for a maximum of 4 doses or until unacceptable toxicity, whichever occurs earlier. After completing 4 doses of the combination of Nivolumab and Ipilimumab, it is recommended to administer Nivolumab as a single agent, either: (1) 240 mg every 2 weeks; or (2) 480 mg every 4 weeks, as an intravenous infusion over 30 minutes until disease progression or unacceptable toxicity.

In some embodiments, the method of treating, reducing, inhibiting or controlling a neoplasia, tumor or cancer in a subject, comprises simultaneously, separately or sequentially administering to the subject, (1) Nivolumab (commercially available as Opdivo®); and (2) a compound of Formula I and/or Formula Ia or a pharmaceutically acceptable salt thereof, wherein the amount of Nivolumab comprises a dosing schedule of 1 mg/kg of Nivolumab administered as an intravenous infusion over 30 minutes, every 3 weeks for a maximum of 4 doses or until unacceptable toxicity, whichever occurs earlier, followed by administration of Nivolumab as a single agent, either: (1) 240 mg every 2 weeks; or (2) 480 mg every 4 weeks, as an intravenous infusion over 30 minutes until disease progression or unacceptable toxicity.

Nivolumab is commercially available as Opdivo®; briefly, its preparation, storage, and administration are as follows: to prepare, withdraw the required volume of Nivolumab and transfer into an intravenous container. Next, dilute Nivolumab with either 0.9% Sodium Chloride Injection, USP or 5% Dextrose Injection, USP to prepare an infusion with a final concentration ranging from 1 mg/mL to 10 mg/mL. The total volume of infusion must not exceed 160 mL. When the subject is an adult or pediatric patient with body weights less than 40 kg, the total volume of infusion must not exceed 4 mL/kg of body weight. Mix diluted solution by gentle inversion, but do not shake. Storage of an infusion of Nivolumab should not exceed 8 hours at room temperature, from the time of preparation—this includes room temperature storage of the infusion in the IV container and time for administration of the infusion or under refrigeration at 2° C. to 8° C. (36° F. to 46° F.) for no more than 24 hours from the time of infusion preparation. Nivolumab should be administered over 30 minutes through an intravenous line containing a sterile, non-pyrogenic, low protein binding in-line filter (pore size of 0.2 micrometer to 1.2 micrometer).

In certain embodiments, the method of treating, reducing, inhibiting or controlling a neoplasia, tumor or cancer in a subject, comprises simultaneously, separately or sequentially administering to the subject, (1) Cemiplimab (commercially available as Libtayo®); and (2) a compound of Formula I and/or Formula Ia or a pharmaceutically acceptable salt thereof, wherein the amount of Cemiplimab comprises a dose ranging from about 0.001 mg/kg to about 100 mg/kg.

In one embodiments, the method of treating, reducing, inhibiting or controlling a neoplasia, tumor or cancer in a subject, comprises simultaneously, separately or sequentially administering to the subject, (1) Cemiplimab (commercially available as Libtayo®); and (2) a compound of Formula I and/or Formula Ia or a pharmaceutically acceptable salt thereof, wherein the amount of Cemiplimab comprises a dose of 350 mg IV over 30 minutes every 3 weeks until disease progression or unacceptable toxicity.

Cemiplimab is commercially available as Libtayo®, which can be prepared, stored, and administered as follows: first, visually inspect for particulate matter and discoloration prior to administration. Libtayo® is a clear to slightly opalescent, colorless to pale yellow solution that may contain trace amounts of translucent to white particles. Discard the vial if the solution is cloudy, discolored or contains extraneous particulate matter other than trace amounts of translucent to white particles. Next, avoiding shaking, withdraw 7 mL from a vial and dilute with 0.9% Sodium Chloride Injection, USP or 5% Dextrose Injection, USP to a final concentration between 1 mg/mL to 20 mg/mL. Mix diluted solution by gentle inversion (do not shake, and discard any unused medicinal product or waste material). After preparation, store at room temperature up to 25° C. (77° F.) for no more than 8 hours from the time of preparation to the end of the infusion or at 2° C. to 8° C. (36° F. to 46° F.) for no more than 24 hours from the time of preparation to the end of infusion. Allow the diluted solution to come to room temperature prior to administration (do not freeze). Administer by intravenous infusion over 30 minutes through an intravenous line containing a sterile, in-line or add-on 0.2-micron to 5-micron filter.

In some embodiments, the method of treating, reducing, inhibiting or controlling a neoplasia, tumor or cancer in a subject, comprises simultaneously, separately or sequentially administering to the subject, (1) a blocking agent, selected from a cell, protein, peptide, antibody or antigen binding fragment thereof, directed against PD-L1, e.g., Atezolizumab (commercially available as Tecentriq® from Genentech®), Avelumab (commercially available as Bavencio® from EMD Serono® and Pfizer®), or Durvalumab (commercially available as Imfinzi® from AstraZeneca®); and (2) a compound of Formula I and/or Formula Ia or a pharmaceutically acceptable salt thereof.

In certain embodiments, the method of treating, reducing, inhibiting or controlling a neoplasia, tumor or cancer in a subject, comprises simultaneously, separately or sequentially administering to the subject, (1) Atezolizumab (commercially available as Tecentriq®); and (2) a compound of Formula I and/or Formula Ia or a pharmaceutically acceptable salt thereof, wherein the amount of Atezolizumab comprises a dose ranging from about 0.001 mg/kg to about 100 mg/kg

In one embodiment, the method of treating, reducing, inhibiting or controlling a neoplasia, tumor or cancer in a subject, comprises simultaneously, separately or sequentially administering to the subject, (1) Atezolizumab (commercially available as Tecentriq®); and (2) a compound of Formula I and/or Formula Ia or a pharmaceutically acceptable salt thereof, wherein the amount of Atezolizumab comprises a dose of 1200 mg as an intravenous infusion over 60 minutes every 3 weeks until disease progression or unacceptable toxicity.

Atezolizumab is commercially available as Tecentriq® from Genentech®. The methods of preparing, storing, and administering Tecentriq® are as follows: to prepare, first visually inspect drug product for particulate matter and discoloration prior to administration, whenever solution and container permit. Discard the vial if the solution is cloudy, discolored, or visible particles are observed. Do not shake the vial. Next, prepare the solution for infusion as follows: withdraw 20 mL of Tecentriq® from the vial; dilute into a 250 mL polyvinyl chloride (PVC), polyethylene (PE), or polyolefin (PO) infusion bag containing 0.9% Sodium Chloride Injection, USP; dilute with 0.9% Sodium Chloride Injection only; mix diluted solution by gentle inversion. Do not shake. Discard used or empty vials of Tecentriq®. Tecentriq® does not contain a preservative so should be administered immediately. If a diluted Tecentriq® infusion solution is not used immediately, store solution either: (1) at room temperature for no more than 6 hours from the time of preparation (this includes room temperature storage of the infusion in the infusion bag and time for administration of the infusion); or (2) under refrigeration at 2° C. to 8° C. (36° F. to 46° F.) for no more than 24 hours from time of preparation. Administer the initial infusion over 60 minutes through an intravenous line with or without a sterile, non-pyrogenic, low-protein binding in-line filter (pore size of 0.2-0.22 micron). If the first infusion is tolerated, all subsequent infusions may be delivered over 30 minutes. Note: do not coadminister other drugs through the same intravenous line, nor administer as an intravenous push or bolus.

In some embodiments, the method of treating, reducing, inhibiting or controlling a neoplasia, tumor or cancer in a subject, comprises simultaneously, separately or sequentially administering to the subject, (1) Avelumab (commercially available as Bavencio®); and (2) a compound of Formula I and/or Formula Ia or a pharmaceutically acceptable salt thereof, wherein the amount of Avelumab comprises a dose of 10 mg/kg administered as an intravenous infusion over 60 minutes every 2 weeks until disease progression or unacceptable toxicity.

It is recommended that subjects receiving Avelumab be premedicated with antihistamine and acetaminophen prior to the first 4 infusions of Avelumab. Avelumab is commercially available as Bavencio®. Briefly, the preparation, storage, and administration of Bavencio® is as follows: first, visually inspect vial for particulate matter and discoloration. Bavencio® is a clear, colorless to slightly yellow solution. Discard vial if the solution is cloudy, discolored, or contains particulate matter. Next, withdraw the required volume of Bavencio® from the vial(s) and inject it into a 250 mL infusion bag containing either 0.9% Sodium Chloride Injection or 0.45% Sodium Chloride Injection. Gently invert the bag to mix the diluted solution and avoid foaming or excessive shearing. Inspect the solution to ensure it is clear, colorless, and free of visible particles. Discard any partially used or empty vials. Storage conditions are as follows: first, ensure Bavencio® is protected from light. The solution can be stored at (1) room temperature up to 77° F. (25° C.) for no more than 4 hours from the time of dilution; or (2) under refrigeration at 36° F. to 46° F. (2° C. to 8° C.) for no more than 24 hours from the time of dilution. If refrigerated, allow the diluted solution to come to room temperature prior to administration. Do not freeze or shake diluted solution. To administer, push the diluted solution over 60 minutes through an intravenous line containing a sterile, non-pyrogenic, low protein binding in-line filter (pore size of 0.2 micron).

In certain embodiments, the method of treating, reducing, inhibiting or controlling a neoplasia, tumor or cancer in a subject, comprises simultaneously, separately or sequentially administering to the subject, (1) Durvalumab (commercially available as Imfinzi®); and (2) a compound of Formula I and/or Formula Ia or a pharmaceutically acceptable salt thereof, wherein the amount of Durvalumab comprises a dose ranging from about 0.001 mg/kg to about 100 mg/kg.

In one embodiment, the method of treating, reducing, inhibiting or controlling a neoplasia, tumor or cancer in a subject, comprises simultaneously, separately or sequentially administering to the subject, (1) Durvalumab (commercially available as Imfinzi®); and (2) a compound of Formula I and/or Formula Ia or a pharmaceutically acceptable salt thereof, wherein the amount of Durvalumab comprises a dose of 10 mg/kg administered as an intravenous infusion over 60 minutes every 2 weeks, until disease progression or unacceptable toxicity.

In some embodiments, the method of treating, reducing, inhibiting or controlling a neoplasia, tumor or cancer in a subject, comprises simultaneously, separately or sequentially administering to the subject, (1) Durvalumab (commercially available as Imfinzi®); and (2) a compound of Formula I and/or Formula Ia or a pharmaceutically acceptable salt thereof, wherein the amount of Durvalumab comprises a dose of 10 mg/kg administered as an intravenous infusion over 60 minutes every 2 weeks until disease progression, unacceptable toxicity, or a maximum of 12 months.

Durvalumab is commercially available as Imfinzi®; briefly, the preparation, storage, and administration of Imfinzi® is as follows: to prepare, first visually inspect the drug product for particulate matter and discoloration prior to administration, whenever solution and container permit. Discard the vial if the solution is cloudy, discolored, or visible particles are observed (do not shake the vial). Next, withdraw the required volume from the vial(s) of Imfinzi® and transfer into an intravenous bag containing 0.9% Sodium Chloride Injection, USP or 5% Dextrose Injection, USP. Mix diluted solution by gentle inversion. Do not shake the solution. The final concentration of the diluted solution should be between 1 mg/mL and 15 mg/mL (discard partially used or empty vials of Imfinzi®). To store, it should be noted that Imfinzi® does not contain a preservative, thus should be administered immediately once prepared. If infusion solution is not administered immediately and needs to be stored, the total time from vial puncture to the start of the administration should not exceed: (1) 24 hours in a refrigerator at 2° C. to 8° C. (36° F. to 46° F.); or (2) 4 hours at room temperature up to 25° C. (77° F.). Note: do not freeze and do not shake. Administration of Imfinzi® is as follows: administer infusion solution intravenously over 60 minutes through an intravenous line containing a sterile, low-protein binding 0.2 or 0.22 micron in-line filter. Do not co-administer other drugs through the same infusion line.

In one embodiment of the present disclosure, the combination may be in the form of a medicament administered to the patient in a dosage form.

A container according to the disclosure in certain instances, may be a vial, ampoule, a syringe, capsule, tablet or a tube. In some cases, the combination and/or one or more of its constituent parts (e.g., a checkpoint inhibitor) may be lyophilized and formulated for resuspension prior to administration. However, in other cases, the combination may be suspended in a volume of a pharmaceutically acceptable liquid. In some embodiments there is provided a container comprising a single unit dose of the combination suspended in pharmaceutically acceptable carrier wherein the unit dose has about 0.001 mg/kg to about 100 mg/kg of the patient weight of a compound of Formula I and/or Ia, or a pharmaceutically acceptable salt thereof.

Embodiments discussed in the context of a method of treating and/or composition of the disclosure may be employed with respect to any other method or composition described herein. Thus, an embodiment pertaining to one method or composition may be applied to other methods and compositions of the disclosure as well.

In some embodiments, the combination is administered to specific sites on or in a subject. For example, the combination or composition thereof, according to the disclosure, such as those comprising a compound of Formula I and/or Formula Ia or a pharmaceutically acceptable salt thereof and one or more checkpoint inhibitors or fragments thereof, may be administered adjacent to tumors or adjacent to lymph nodes, such as those that drain tissue surrounding a tumor. Thus, in certain instances, site-specific administration of the combination may be near the posterior cervical, tonsillar, axillary, inguinal, anterior or cervical, sub-mandibular, sub mental or superclavicular lymph nodes.

The combination, e.g., a compound of Formula I and/or Formula Ia or a pharmaceutically acceptable salt thereof and one or more checkpoint inhibitors, may be administered for the length of time the cancer or tumor(s) is present in a patient or until such time the cancer has regressed or stabilized. The combination may also be continued to be administered to the patients once the cancer or tumor has regressed or stabilized.

In another embodiment, the combination (e.g., a compound of Formula I and/or Formula Ia or a pharmaceutically acceptable salt thereof, one or more checkpoint inhibitors, and/or both), is administered via a parenteral route selected from subcutaneous, intradermal, subdermal, intraperitoneal, intravenous and intravesicular injection. Intradermal injection enables delivery of an entire proportion of the combination or a composition thereof to a layer of the dermis that is accessible to immune surveillance and thus capable of electing anti-cancer immune response and promoting immune cell proliferation at local lymph nodes.

In some embodiments of the disclosure, the combination is administered by direct intradermal injection, it is also contemplated that other methods of administration may be used in some case. Thus in certain instances the combination of the present disclosure can be administered by injection, infusion, continuous infusion, intravenously, intradermally, intraarterially, intraperitoneally, intralesionally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, intracranially, intraarticularly, intraprostaticaly, intrapleurally, intratracheally, intranasally, topically, locally, inhalation (e.g. aerosol inhalation), via a catheter, via a lavage, or by other method or any combination of the forgoing as would be known to one of ordinary skill in the art (see, for example, Remington's Pharmaceutical Sciences, 18th Ed. Mack Printing Company, 1990).

Diseases and Disorders

The present disclosure may be used to treat a neoplastic disease, such as solid or non-solid cancers. As used herein, “treatment” encompasses the prevention, reduction, control and/or inhibition of a neoplastic disease. Such diseases include a sarcoma, carcinoma, adenocarcinoma, melanoma, myeloma, blastoma, glioma, lymphoma or leukemia. Exemplary cancers include, for example, carcinoma, sarcoma, adenocarcinoma, melanoma, neural (blastoma, glioma), mesothelioma and reticuloendothelial, lymphatic or hematopoietic neoplastic disorders (e.g., myeloma, lymphoma or leukemia). In particular aspects, a neoplasm, tumor or cancer includes a lung adenocarcinoma, lung carcinoma, diffuse or interstitial gastric carcinoma, colon adenocarcinoma, prostate adenocarcinoma, esophagus carcinoma, breast carcinoma, pancreas adenocarcinoma, ovarian adenocarcinoma, adenocarcinoma of the adrenal gland, adenocarcinoma of the endometrium or uterine adenocarcinoma and carcinomas of the head and neck.

Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. Cancers that may be treated according to the disclosure include but are not limited to cells or neoplasms of the bladder, blood, bone, bone marrow, brain, breast, colon, esophagus, gastrointestinal system, gum, head, kidney, liver, lung, nasopharynx, neck, ovary, prostate, skin, stomach, testis, tongue, or uterus. In addition, the cancer may specifically be of the following histological type, though it is not limited to the following: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma, gastrinoma, malignant; cholangiocarcinoma, hepatocellular carcinoma; combined hepatocellular carcinoma and cholangiocarcinoma, trabecular adenocarcinoma, adenoid cystic carcinoma; adenocarcinoma in adenomatous polyp; adenocarcinoma, familial polyposis coli, solid carcinoma; carcinoid tumor, malignant; bronchiolo-alveolar adenocarcinoma, papillary adenocarcinoma, chromophobe carcinoma; acidophil carcinoma; oxyphilic adenocarcinoma, basophil carcinoma; clear cell adenocarcinoma, granular cell carcinoma; follicular adenocarcinoma, papillary and follicular adenocarcinoma, nonencapsulating sclerosing carcinoma; adrenal cortical carcinoma; endometroid carcinoma; skin appendage carcinoma; apocrine adenocarcinoma, sebaceous adenocarcinoma, ceruminous adenocarcinoma, mucoepidermoid carcinoma; cystadenocarcinoma, papillary cystadenocarcinoma, papillary serous cystadenocarcinoma, mucinous cystadenocarcinoma, mucinous adenocarcinoma, signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma with squamous metaplasia, thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma, glomangiosarcoma, malignant melanoma; amelanotic melanoma; superficial spreading melanoma; malignant melanoma in giant pigmented nevus; epithelioid cell melanoma; blue nevus, malignant; sarcoma; fibrosarcoma, fibrous histiocytoma, malignant; myxosarcoma, liposarcoma, leiomyosarcoma, rhabdomyosarcoma, embryonal rhabdomyosarcoma, alveolar rhabdomyosarcoma, stromal sarcoma; mixed tumor; Mullerian mixed tumor; nephroblastoma, hepatoblastoma, carcinosarcoma, mesenchymoma, malignant; Brenner tumor, malignant; phyllodes tumor, malignant; synovial sarcoma; mesothelioma, malignant; dysgerminoma, embryonal carcinoma; teratoma, malignant; struma ovarii, malignant; choriocarcinoma, mesonephroma, malignant; hemangiosarcoma, hemangioendothelioma, malignant; Kaposi's sarcoma; hemangiopericytoma, malignant; lymphangiosarcoma, osteosarcoma, juxtacortical osteosarcoma, chondrosarcoma, chondroblastoma, malignant; mesenchymal chondrosarcoma, giant cell tumor of bone; Ewing's sarcoma; odontogenic tumor, malignant; ameloblastic odontosarcoma, ameloblastoma, malignant; ameloblastic fibrosarcoma, pinealoma, malignant; chordoma, glioma, malignant; ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma; ganglioneuroblastoma, neuroblastoma, retinoblastoma, olfactory neurogenic tumor; meningioma, malignant; neurofibrosarcoma, neurilemmoma, malignant; granular cell tumor, malignant; malignant lymphoma; Hodgkin's disease; Hodgkin's; paragranuloma, malignant lymphoma, small lymphocytic, malignant lymphoma, large cell, diffuse; malignant lymphoma, follicular; mycosis fungoides, other specified non-Hodgkin's lymphomas; malignant histiocytosis, multiple myeloma, mast cell sarcoma; immunoproliferative small intestinal disease; leukemia; lymphoid leukemia; plasma cell leukemia; erythroleukemia, lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Preferably, the neoplastic disease may be tumors associated with a cancer selected from prostate cancer, liver cancer, renal cancer, lung cancer, breast cancer, colorectal cancer, pancreatic cancer, brain cancer, hepatocellular cancer, lymphoma, leukemia, gastric cancer, cervical cancer, ovarian cancer, thyroid cancer, melanoma, carcinomas of the head and neck, head and neck cancer, skin cancer and soft tissue sarcoma and/or other forms of carcinoma. The tumor may be metastatic or a malignant tumor.

In some embodiments, the neoplastic disease to be treated is pancreatic cancer, breast cancer, lung cancer, prostate cancer and skin cancer. Most preferably, the neoplastic disease to be treated is pancreatic cancer, colorectal cancer and/or carcinomas of the head and neck.

The efficacy of the method described herein can be determined by evaluating biomarkers in the immune checkpoint pathway (see Gibney et al., Predictive biomarkers for checkpoint inhibitor-based immunotherapy, Lancet Oncol. 2016 December; 17 (12): e542-e551).

### EXAMPLES

### Example 1

This example shows the University of Michigan Quinazoline Library 3-Experimentals (Synthesis of MOL-160-163, and MOL-165).

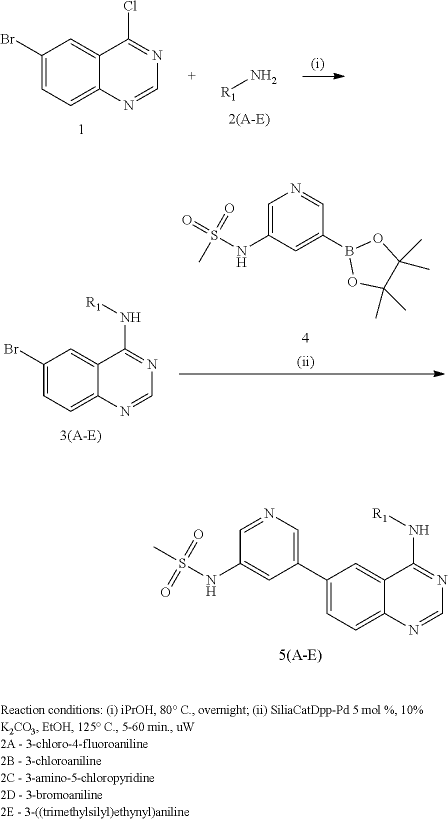

### N-(5-(4-((3-chloro-4-fluorophenyl)amino)quinazolin-6-yl)pyridin-3-yl)methanesulfonamide, MOL-160

To a solution consisting of 6-bromo-4-chloroquinazoline (0.3 g, 1.30 mmol) in 2-propanol (30 mL) was added 3-chloro-4-fluoroaniline (0.189 g, 1.30 mmol). The reaction mixture was heated (80° C.) and stirred overnight under a flow of N2. The reaction mixture was cooled to room temperature and then the reaction mixture was filtered over a fritted funnel. The filtered solid was rinsed with excess 2-propanol and dried under high vacuum to afford 6-bromo-N-(3-chloro-4-fluorophenyl)quinazolin-4-amine (3A) as an off-white solid (350 mg, 85% yield). MS: (ESI⁺ m/z 353.9, ESI⁻ m/z 351.9) A solution consisting of 6-bromo-N-(3-chloro-4-fluorophenyl)quinazolin-4-amine (0.185 g, 0.526 mmol) in anhydrous ethanol (3 mL) was placed in a 5 mL microwave reaction vial containing a stir bar. Next, 5-(methylsulfonamido)pyridine-3-yl boronic acid pinacol ester (4, 0.164 g, 0.553 mmol) was added followed by SiliCat DPP-Pd (5 mol %, 0.26 mmol/g loading, 0.101 g) and 10% aqueous potassium carbonate solution (2 equivalents, 0.76 mL, 1.05 mmol). The reaction mixture was placed under N2 atmosphere, capped, and then heated at 125° C. for one hour in a Biotage Emrys Optimizer microwave. The reaction mixture was allowed to cool to room temperature and then filtered over a fritted funnel to collect SiliCat DPP-Pd. The filtered solid was rinsed with excess ethanol and the filtrate was concentrated under reduced pressure to afford the crude product. Purification of the crude product by Biotage Isolera flash chromatography using a gradient of 4-100% ethyl acetate in heptane, followed by 0-10% methanol in dichloromethane afforded N-(5-(4-((3-chloro-4-fluorophenyl)amino)quinazolin-6-yl)pyridin-3-yl)methanesulfonamide (5A, MOL-160, 96 mg, 41% yield, 96% purity) as a white solid; ¹H NMR (400 MHz, DMSO-d₆) δ 10.17 (br. s, 1H), 10.03 (s, 1H), 8.83-8.87 (m, 2H), 8.66 (s, 1H), 8.49 (d, J=2.38 Hz, 1H), 8.13-8.20 (m, 2H), 7.90-7.98 (m, 2H), 7.83 (ddd, J=2.65, 4.25, 9.01 Hz, 1H), 7.47 (t, J=9.15 Hz, 1H), 3.14 (s, 3H); MS: (ESI⁺ m/z 444.1, ESI⁻ m/z 442.0); TLC: (90:10:0.5, DCM:MeOH:NH₄OH) R_{f}=0.32.

### N-(5-(4-((3-chlorophenyl)amino)quinazolin-6-yl)pyridin-3-yl)methanesulfonamide, MOL-162

To a solution consisting of 6-bromo-4-chloroquinazoline (0.448 g, 1.84 mmol) in 2-propanol (10 mL) was added 3-chloroaniline (0.246 g, 1.93 mmol). The reaction mixture was heated (80° C.) and stirred overnight under a flow of N2. The reaction mixture was cooled to room temperature and then the reaction mixture was filtered over a fritted funnel. The filtered solid was rinsed with excess 2-propanol and dried under high vacuum to afford 6-bromo-N-(3-chlorophenyl)quinazolin-4-amine (3B) as an off-white solid (490 mg, 79% yield, 98% purity). MS (ESI⁺ m/z 335.9, ESI⁻ m/z 333.9.) A solution consisting of 6-bromo-N-(3-chlorophenyl)quinazolin-4-amine (0.200 g, 0.597 mmol) in anhydrous ethanol (3 mL) was placed in a 5 mL microwave reaction vial containing a stir bar. Next, 5-(methylsulfonamido)pyridine-3-yl boronic acid pinacol ester (4, 0.187 g, 0.627 mmol) was added followed by SiliCat DPP-Pd (5 mol %, 0.26 mmol/g loading, 0.115 g) and 10% aqueous potassium carbonate solution (2 equivalents, 0.87 mL, 1.20 mmol). The reaction mixture was placed under N2 atmosphere, capped, and then heated at 100° C. for 30 minutes in a Biotage Emrys Optimizer microwave. The reaction mixture was allowed to cool to room temperature and then filtered over a fritted funnel to collect SiliCat DPP-Pd. The filtered solid was rinsed with excess ethanol and the filtrate was concentrated under reduced pressure to afford the crude product. Purification of the crude product by Biotage Isolera flash chromatography using a gradient of 4-100% ethyl acetate in heptane, followed by 0-10% methanol in dichloromethane afforded N-(5-(4-((3-chlorophenyl)amino)quinazolin-6-yl)pyridin-3-yl)methanesulfonamide (5B, MOL-162, 78 mg, 31% yield, 97% purity) as a white solid; ¹H NMR (400 MHz, DMSO-d₆) δ 10.20 (br. s., 1H), 10.04 (s, 1H), 8.89 (dd, J=1.74, 13.45 Hz, 1H), 8.70 (s, 1H), 8.50 (d, J=2.38 Hz, 1H), 8.19 (dd, J=1.65, 8.60 Hz, 1H), 8.11 (t, J=2.01 Hz, 1H), 7.91-8.04 (m, 1H), 7.67-7.91 (m, 1H), 7.45 (t, J=8.14 Hz, 1H), 7.22 (m, 1H), 3.16 (s, 3H); MS: (ESI⁺ m/z 426.1, ESI⁻ m/z 424.0); TLC: (90:10:0.5, DCM:MeOH:NH₄OH) R_{f}=0.49.

### N-(5-(4-((5-chloropyridin-3-yl)amino)quinazolin-6-yl)pyridin-3-yl)methanesulfonamide, MOL-163

To a solution consisting of 6-bromo-4-chloroquinazoline (0.448 g, 1.84 mmol) in 2-propanol (10 mL) was added 3-amino-5-chloropyridine (0.248 g, 1.93 mmol). The reaction mixture was heated (80° C.) and stirred overnight under a flow of N2. The reaction mixture was cooled to room temperature and then the reaction mixture was filtered over a fritted funnel. The filtered solid was rinsed with excess 2-propanol and dried under high vacuum to afford 6-bromo-N-(5-chloropyridin-3-yl)quinazolin-4-amine (3C) as an off-white solid (575 mg, 93% yield, 93% purity). MS (ESI⁺ m/z 336.9, ESI⁻ m/z 334.9). A solution consisting of 6-bromo-N-(5-chloropyridin-3-yl)quinazolin-4-amine (0.136 g, 0.405 mmol) in anhydrous ethanol (3 mL) was placed in a 5 mL microwave reaction vial containing a stir bar. Next, 5-(methylsulfonamido)pyridine-3-yl boronic acid pinacol ester (4, 0.127 g, 0.425 mmol) was added followed by SiliCat DPP-Pd (5 mol %, 0.26 mmol/g loading, 0.082 g) and 10% aqueous potassium carbonate solution (2 equivalents, 0.59 mL, 0.81 mmol). The reaction mixture was placed under N₂ atmosphere, capped, and then heated at 100° C. for 30 minutes in a Biotage Emrys Optimizer microwave. The reaction mixture was allowed to cool to room temperature and then filtered over a fritted funnel to collect SiliCat DPP-Pd. The filtered solid was rinsed with excess ethanol and the filtrate was concentrated under reduced pressure to afford the crude product. Purification of the crude product by Biotage Isolera flash chromatography using a gradient of 4-100% ethyl acetate in heptane, followed by 0-10% methanol in dichloromethane afforded N-(5-(4-((5-chloropyridin-3-yl)amino)quinazolin-6-yl)pyridin-3-yl)methanesulfonamide (5C, MOL-163, 70 mg, 40% yield, 98% purity) as a white solid; ¹H NMR (400 MHz, DMSO-d₆) δ 10.21 (br. s., 2H), 8.94-9.03 (m, 1H), 8.86-8.88 (d, J=4.65 Hz, 2H), 8.73 (s, 1H), 8.59 (s, 1H), 8.50 (d, J=2.01 Hz, 1H), 8.32-8.44 (m, 1H), 8.20 (d, J=8.97 Hz, 1H), 7.90-8.04 (m, 2H), 3.15 (s, 3H); MS: (ESI⁺ m/z 427.0, ESI⁻ m/z 425.0); TLC: (90:10:0.5, DCM:MeOH:NH₄OH) R_{f}=0.47.

### N-(5-(4-((5-bromopyridin-3-yl)amino)quinazolin-6-yl)pyridin-3-yl)methanesulfonamide, MOL-165

To a solution consisting of 6-bromo-4-chloroquinazoline (0.448 g, 1.84 mmol) in 2-propanol (10 mL) was added 3-bromoaniline (0.332 g, 1.93 mmol). The reaction mixture was heated (80° C.) and stirred overnight under a flow of N₂. The reaction mixture was cooled to room temperature and then the reaction mixture was filtered over a fritted funnel. The filtered solid was rinsed with excess 2-propanol and dried under high vacuum to afford 6-bromo-N-(5-bromopyridin-3-yl)quinazolin-4-amine (3D) as an off-white solid (605 mg, 87% yield, 98% purity). MS (ESI⁺ m/z 379.9, ESI⁻ m/z 377.8). A solution consisting of 6-bromo-N-(5-bromopyridin-3-yl)quinazolin-4-amine (0.150 g, 0.395 mmol) in anhydrous ethanol (4 mL) was placed in a 5 mL microwave reaction vial containing a stir bar. Next, 5-(methylsulfonamido)pyridine-3-yl boronic acid pinacol ester (4, 0.120 g, 0.400 mmol) was added followed by SiliCat DPP-Pd (5 mol %, 0.26 mmol/g loading, 0.080 g) and 10% aqueous potassium carbonate solution (2 equivalents, 0.60 mL, 0.79 mmol). The reaction mixture was placed under N₂ atmosphere, capped, and then heated at 100° C. for 30 minutes in a Biotage Emrys Optimizer microwave. The reaction mixture was allowed to cool to room temperature and then filtered over a fritted funnel to collect SiliCat DPP-Pd. The filtered solid was rinsed with excess ethanol and the filtrate was concentrated under reduced pressure to afford the crude product. Purification of the crude product by Biotage Isolera flash chromatography using a gradient of 4-100% ethyl acetate in heptane, followed by 0-10% methanol in dichloromethane afforded N-(5-(4-((5-bromopyridin-3-yl)amino)quinazolin-6-yl)pyridin-3-yl)methanesulfonamide (5D, MOL-165, 39 mg, 21% yield, 85% purity) as a white solid; This product is 85:15 mixture of 5D:5D-B N-(5′-((6-bromoquinazolin-4-yl)amino)-[3,3′-bipyridin]-5-yl)methanesulfonamide which occurs as by product from the Suzuki coupling reaction. ¹H NMR (400 MHz, DMSO-d₆) δ 10.17 (br. s., 1H), 10.00 (s, 1H), 8.84-8.94 (m, 2H), 8.70 (s, 1H), 8.51 (d, J=2.38 Hz, 1H), 8.15-8.25 (m, 2H), 7.89-8.03 (m, 2H), 7.33-7.41 (m, 2H), 3.16 (s, 3H); MS: (ESI⁺ m/z 470, 472); TLC: (90:10:0.5, DCM:MeOH:NH₄OH) R_{f}=0.62.

### N-(5-(4-((3-ethynylphenyl)amino)quinazolin-6-yl)pyridin-3-yl)methanesulfonamide, MOL-161

To a solution consisting of 6-bromo-4-chloroquinazoline (1.2 g, 4.9 mmol) and 3-((trimethylsilyl)ethynyl)aniline (1.1 g, 5.9 mmol, prepared as describe by Ute F. Rohrig, J M C, 2012, 55(11), 5270-5290) in 30 mL of 1,4-dioxane was heated at 90° C. for 3 hour. The reaction mixture was cooled to room temperature, diluted with diethyl ether and filtered through fritted glass. The solid was triturated under 20 mL of isopropyl alcohol, filtered and dried to give 6-bromo-N-(3-((trimethylsilyl)ethynyl)phenyl)quinazolin-4-amine (3E) as a solid (940 mg, 48%); ¹H NMR (400 MHz, DMSO-d₆) δ 11.8 (br s, 1H), 9.29 (d, J=1.7 Hz, 1H), 9.00 (s, 1H), 8.26 (dd, J=1.7, 8.8 Hz, 1H), 7.95 (d, J=8.9 Hz, 1H), 7.89 (s, 1H), 7.81 (d, J=8.1 Hz, 1H), 7.51 (t, J=7.9 Hz, 1H), 7.41 (d, J=7.7 Hz, 1H), 0.25 (s, 9H). A solution consisting of 6-bromo-N-(3-((trimethylsilyl)ethynyl)phenyl)quinazolin-4-amine (0.250 g, 0.631 mmol) in anhydrous ethanol (4 mL) was placed in a 5 mL microwave reaction vial containing a stir bar. Next, 5-(methylsulfonamido)pyridine-3-yl boronic acid pinacol ester (4, 0.200 g, 0.662 mmol) was added followed by SiliCat DPP-Pd (5 mol %, 0.26 mmol/g loading, 0.126 g) and 10% aqueous potassium carbonate solution (2 equivalents, 0.91 mL, 1.26 mmol). The reaction mixture was placed under N₂ atmosphere, capped, and then heated at 100° C. for 5 minutes in a Biotage Emrys Optimizer microwave. The reaction mixture was allowed to cool to room temperature and then filtered over a fritted funnel to collect SiliCat DPP-Pd. The filtered solid was rinsed with excess ethanol and the filtrate was concentrated under reduced pressure to afford the crude product. Purification of the crude product by Biotage Isolera flash chromatography using a gradient of 5-65% ethyl acetate in heptane, followed by 0-10% methanol in dichloromethane afforded a mixture of 5E with TMS-protected 5E. This mixture was dissolved in methanol and then treated with excess 10% potassium carbonate (1 mL). The solution was heated to 35° C. and the TMS removal was followed by TLC (90:10:0.5, DCM:MeOH:NH₄OH). After the reaction was complete, the solution was acidified (1N HCl) to pH ˜5 and then extracted three times with DCM:MeOH (90:10 mixture, 75 mL). The organic layer was concentrated under reduced pressure to afford the crude product. Purification of the deprotected crude product by Biotage Isolera flash chromatography using a gradient of 1-13% methanol in dichloromethane afforded N-(5-(4-((3-ethynylphenyl)amino)quinazolin-6-yl)pyridin-3-yl)methanesulfonamide (5E, MOL-161, 68 mg, 26% yield, 97.5% purity) as a yellow solid. ¹H NMR (400 MHz, DMSO-d₆) δ 10.16 (br. s., 1H), 9.97 (s, 1H), 8.75-8.94 (m, 2H), 8.66 (s, 1H), 8.48 (d, J=2.38 Hz, 1H), 8.16 (dd, J=1.65, 8.60 Hz, 1H), 8.04 (s, 1H), 7.85-7.98 (m, 4H), 7.42 (t, J=7.87 Hz, 1H), 7.42 (d, J=7.69 Hz, 1H), 4.21 (s, 1H), 3.13 (s, 3H); MS: (ESI⁺ m/z 416.1, ESI⁻ m/z 414.0); TLC: (90:10:0.5, DCM:MeOH:NH₄OH) R_{f}=0.6.

### Example 2

This example shows University of Michigan Quinazoline Experimentals (Synthesis of MOL-166-167, and MOL-153).

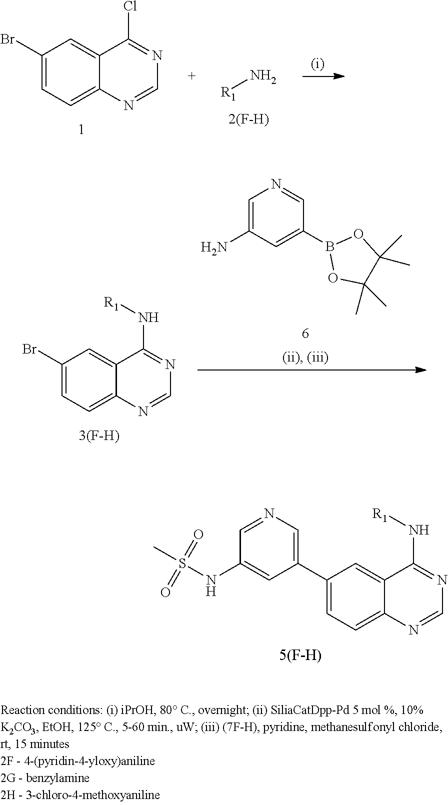

### N-(5-(4-((4-(pyridin-4-yloxy)phenyl)amino)quinazolin-6-yl)pyridin-3-yl)methanesulfonamide, MOL-166

To a solution consisting of 6-bromo-4-chloroquinazoline (0.448 g, 1.84 mmol) in 2-propanol (10 mL) was added 4-(pyridine-4-yloxy)aniline (0.360 g, 1.93 mmol). The reaction mixture was heated (80° C.) and stirred overnight under a flow of N₂. The reaction mixture was cooled to room temperature and then the reaction mixture was filtered over a fritted funnel. The filtered solid was rinsed with excess 2-propanol and dried under high vacuum to afford 6-bromo-N-(4-(pyridin-4-yloxy)phenyl)quinazolin-4-amine (3F) as an off-white solid (313 mg, 43% yield, 97% purity). MS (ESI⁺ m/z 394.0, ESI⁻ m/z 392.0). Next a solution consisting of 6-bromo-N-(4-(pyridin-4-yloxy)phenyl)quinazolin-4-amine (0.306 g, 0.77 mmol) in anhydrous ethanol (10 mL) was placed in a 20 mL microwave reaction vial containing a stir bar. Next, 3-aminopyridine-5-boronic acid pinacol ester (6, 0.176 g, 0.80 mmol) was added followed by SiliCat DPP-Pd (5 mol %, 0.26 mmol/g loading, 0.150 g) and 10% aqueous potassium carbonate solution (2 equivalents, 1.15 mL, 1.6 mmol). The reaction mixture was placed under N₂ atmosphere, capped, and then heated at 125° C. for one hour in a Biotage Emrys Optimizer microwave. The reaction mixture was allowed to cool to room temperature and then filtered over a fritted funnel to collect SiliCat DPP-Pd. The filtered solid was rinsed with excess ethanol and the filtrate was concentrated under reduced pressure to afford the crude product. Purification of the crude product by Biotage Isolera flash chromatography using a gradient of 4-100% ethyl acetate in heptane, followed by 0-10% methanol in dichloromethane afforded 7F 6-(5-aminopyridin-3-yl)-N-(4-(pyridin-4-yloxy)phenyl)quinazolin-4-amine (50 mg, 15% yield, 92% purity) as an off-white solid. MS (ESI⁺ m/z 407.1, ESI⁻ m/z 405.1). To a room temperature solution of 6-(5-aminopyridin-3-yl)-N-(4-(pyridin-4-yloxy)phenyl)quinazolin-4-amine (50 mg, 0.12 mmol) in pyridine (3 mL) was added methanesulfonyl chloride (56 mg, 0.5 mmol). The reaction mixture turned dark red which persisted and was stirred for 15 minutes. The reaction mixture was poured into a saturated solution of sodium bicarbonate and the organic material was extracted with ethyl acetate. The organic phase was washed with water and brine, dried over magnesium sulfate, filtered and concentrated under vacuum. The crude solid was dissolved in methanol and “dry loaded” on to a silica column eluted with a gradient of 1/9 to 3/7 methanol/ethyl acetate to give N-(5-(4-((4-(pyridin-4-yloxy)phenyl)amino)quinazolin-6-yl)pyridin-3-yl)methanesulfonamide (5F, MOL-166, 20 mg, 33% yield, 96% purity) as a solid. ¹H NMR (400 MHz, DMSO-d₆) δ 10.07 (s, 1H), 8.91 (s, 1H), 8.79 (d, J=1.9 Hz, 1H), 8.62 (s, 1H), 8.4-8.5 (m, 3H), 8.15 (dd, J=1.7, 8.6 Hz, 1H), 7.85-8.0 (m, 4H), 7.24 (d, J=8.9 Hz, 2H), 6.94 (d, J=4.7 Hz, 2H), 3.08 (s, 3H); MS. (ESI⁺ m/z 485.1, ESI⁻ m/z 483.0).

### 5G, N-(5-(4-(benzylamino)quinazolin-6-yl)pyridin-3-yl)methanesulfonamide, MOL-167

A mixture of 6-bromo-4-chloroquinazoline (1.2 g, 4.9 mmol) and benzylamine (633 mg, 5.9 mmol) in 30 mL of 1,4-dioxane was heated at 45° C. for 2 hours then at 90° C. for 1 hour. An additional amount of benzylamine (500 mg, 4.7 mmol) was added and the reaction mixture was heated at 90° C. for an additional 2 hours. The reaction mixture was cooled to room temperature, diluted with diethyl ether and filtered through fritted glass. The filtrate was concentrated under vacuum and the crude solid was triturated under isopropyl alcohol, filtered and dried to give N-benzyl-6-bromoquinazolin-4-amine (3G) as a solid (950 mg, 62% yield). ¹H NMR (400 MHz, DMSO-d₆) δ 8.91 (t, J=5.9 Hz, 1H), 8.60 (d, J=2.2 Hz, 1H), 8.46 (s, 1H), 7.88 (dd, J=2.2, 8.9 Hz, 1H), 7.62 (d, J=8.9 Hz, 1H), 7.25-7.40 (m, 4H), 7.23 (t, J=9 Hz, 1H), 4.75 (d, J=5.8 Hz, 2H). Next a solution consisting of N-benzyl-6-bromoquinazolin-4-amine (0.314 g, 1.0 mmol) in anhydrous ethanol (10 mL) was placed in a 20 mL microwave reaction vial containing a stir bar. Next, 3-aminopyridine-5-boronic acid pinacol ester (6, 0.231 g, 1.05 mmol) was added followed by SiliCat DPP-Pd (5 mol %, 0.26 mmol/g loading, 0.200 g) and 10% aqueous potassium carbonate solution (2 equivalents, 1.5 mL, 1.26 mmol). The reaction mixture was placed under N₂ atmosphere, capped, and then heated at 100° C. for 5 minutes in a Biotage Emrys Optimizer microwave. The reaction mixture was allowed to cool to room temperature and then filtered over a fritted funnel to collect SiliCat DPP-Pd. The filtered solid was rinsed with excess ethanol and the filtrate was concentrated under reduced pressure to afford the crude product. Purification of the crude product by Biotage Isolera flash chromatography using a gradient of 4-100% ethyl acetate in heptane, followed by 0-10% methanol in dichloromethane afforded 6-(5-aminopyridin-3-yl)-N-benzylquinazolin-4-amine (7G) as a white solid (59 mg, 18% yield, 85% purity); MS: (ESI⁺ m/z 328.1, ESI⁻ m/z 326.1). To a room temperature solution of 6-(5-aminopyridin-3-yl)-N-benzylquinazolin-4-amine (59 mg, 0.18 mmol) in pyridine (4 mL) was added methanesulfonyl chloride (83 mg, 0.72 mmol). The reaction mixture turned dark red which persisted and was stirred for 1 hour. The reaction mixture was poured into a saturated solution of sodium bicarbonate and the organic material was extracted with ethyl acetate. The organic phase was washed with water and brine, dried over magnesium sulfate, filtered and concentrated under vacuum. The crude solid was dissolved in methanol and “dry loaded” on to a silica column eluted with a gradient of 1/9 to 3/7 methanol/ethyl acetate resulting in a partially purified pale yellow solid. This crude solid was triturated under a solution of 2-propanol/dichloromethane/ethyl acetate, filtered, and dried to give N-(5-(4-(benzylamino)quinazolin-6-yl)pyridin-3-yl)methanesulfonamide (5G, MOL-167, 6 mg, 8% yield, 96% purity) as a white powder; MS: (ESI⁺ m/z 406.1, ESI⁻ m/z 404.1).

### 5H, N-(5-(4-((3-chloro-4-methoxyphenyl)amino)quinazolin-6-yl)pyridin-3-yl)methanesulfonamide, MOL-153

A mixture of 6-bromo-4-chloroquinazoline (1.65 g, 6.5 mmol) and 3-chloro-4-methoxyaniline (1.2 g, 7.8 mmol) in 40 mL of 1,4-dioxane was heated at 90° C. for 3 hour. The reaction mixture was cooled to room temperature, diluted with diethyl ether and filtered through fritted glass. The solid was washed with diethyl ether and dried to give 6-bromo-N-(3-chloro-4-methoxyphenyl)quinazolin-4-amine (3H) as a yellow-gold solid (2.1 g, 89%); ¹H NMR (400 MHz, DMSO-d₆) δ 11.5 (br s, 1H), 9.15 (s, 1H), 8.92 (s, 1H), 8.21 (d, J=9 Hz, 1H), 7.8-8.0 (m, 2H), 7.66 (dd, J=8.9, 2.3 Hz, 1H), 7.25 (d, J=8.9 Hz, 1H), 3.95 (s, 3H); MS: (ESI⁺ m/z 364.0, 366.0 (Br isotope), ESI⁻ m/z 362.0, 364.0 (Br isotope)). A solution of 6-bromo-N-(3-chloro-4-methoxyphenyl)quinazolin-4-amine (1.85 g, 5.08 mmol) and 3-aminopyridine-5-boronic acid pinacol ester (6, 932 mg, 4.23 mmol) in 1,4-dioxane (90 mL) and water (7.6 mL) was degassed. To the solution was added cesium carbonate (6.9 g, 21.1 mmol) and [1,1′-bis(diphenylphosphino)ferrocene]dichloropalladium(II) (366 mg). The reaction mixture was heated at 90° C.-95° C. under N₂ for 4 hours. The reaction mixture was diluted with ethyl acetate, dichloromethane and methanol, washed with saturated sodium bicarbonate, water and brine, dried over magnesium sulfate, filtered and concentrated under vacuum. The crude material was purified by silica gel column chromatography eluting with a gradient of 2/98 to 25/75 methanol/ethyl acetate to afford 6-(5-aminopyridin-3-yl)-N-(3-chloro-4-methoxyphenyl)quinazolin-4-amine (7H) as an off white solid (524 mg, 33% yield). To a room temperature solution of 6-(5-aminopyridin-3-yl)-N-(3-chloro-4-methoxyphenyl)quinazolin-4-amine (250 mg, 0.66 mmol) in pyridine (15 mL) was added methanesulfonyl chloride (303 mg, 2.65 mmol). The reaction mixture turned dark red which persisted and was stirred for 1 hour. The reaction mixture was poured into a saturated solution of sodium bicarbonate and the organic material was extracted with ethyl acetate. The organic phase was washed with water and brine, dried over magnesium sulfate, filtered and concentrated under vacuum. The crude yellow solid was dissolved in methanol. Ethyl acetate and diethyl ether were added until cloudiness was observed. The mixture was stirred for 1 hour and the resulting solid was filtered, washed with ethyl acetate and dried to give N-(5-(4-((3-chloro-4-methoxyphenyl)amino)quinazolin-6-yl)pyridin-3-yl)methanesulfonamide (5H, MOL-153, 120 mg, 40% yield, 94% purity) as a bright yellow powder; ¹H NMR (400 MHz, DMSO-d₆) δ 11.6 (br s, 1H), 10.3 (br s, 1H), 9.17 (s, 1H), 8.95 (s, 1H), 8.88 (br s, 1H), 8.52 (br s, 1H), 8.40 (dd, J=1.3, 8.6 Hz, 1H), 8.0-8.1 (m, 1H), 7.89 (d, J=2.6 Hz, 1H), 7.66 (dd, J=2.6, 8.9 Hz, 1H), 7.28 (d, J=9.0 Hz, 1H), 3.90 (s, 3H), 3.16 (s, 3H); MS: (ESI⁺ m/z 456).

### Example 3

This example shows University of Michigan Quinazoline Experimentals (Synthesis of MOL-154).

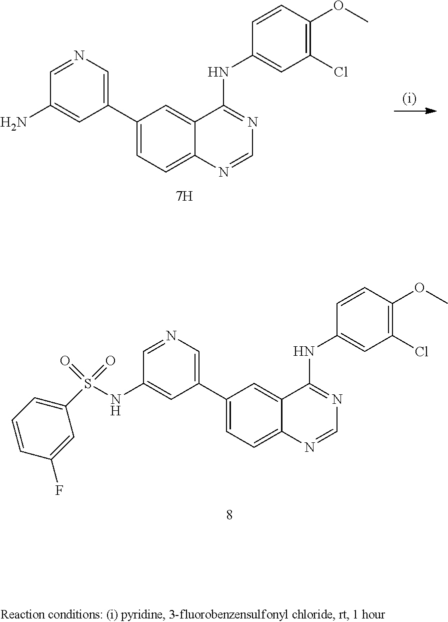 [SMILES: COc1ccc(Nc2ncnc3ccc(-c4cncc(N)c4)cc23)cc1Cl ; COc1ccc(Nc2ncnc3ccc(-c4cncc(NS(=O)(=O)c5cccc(F)c5)c4)cc23)cc1Cl]

### 8, (N-(5-(4-((3-chloro-4-methoxyphenyl)amino)quinazolin-6-yl)pyridin-3-yl)-3-fluorobenzenesulfonamide, MOL-154)

To a room temperature solution of 6-(5-aminopyridin-3-yl)-N-(3-chloro-4-methoxyphenyl)quinazolin-4-amine (7H, 250 mg, 0.66 mmol) in pyridine (15 mL) was added 3-fluorobenzenesulfonyl chloride (516 mg, 2.65 mmol). The reaction mixture turned dark red which persisted and was stirred for 1 hour. The reaction mixture was poured into a saturated solution of sodium bicarbonate and the organic material was extracted with ethyl acetate. The organic phase was washed with water and brine, dried over magnesium sulfate, filtered and concentrated under vacuum. Roto-evaporation with heptane provided a crude yellow solid which was triturated under a mixture of methanol and ethyl acetate and diethyl ether for 1 hour and the resulting solid was filtered and dried to give N-(5-(4-((3-chloro-4-methoxyphenyl)amino)quinazolin-6-yl)pyridin-3-yl)-3-fluorobenzenesulfonamide (8, MOL-154, 120 mg, 34% yield, 100% purity) as a yellow powder; ¹H NMR (400 MHz, DMSO-d₆) δ 10.8 (br s, 1H), 10.0 (br s, 1H), 8.82 (s, 2H), 8.62 (s, 1H), 8.29 (d, J=2.2 Hz, 1H), 8.07 (d, J=7.5 Hz, 1H), 7.96 (d, J=2.3 Hz, 1H), 7.93 (d, J=1.9 Hz, 1H), 7.87 (d, J=8.9 Hz, 1H), 7.72 (dd, J=2.4, 8.9 Hz, 1H), 7.6-7.7 (m, 2H), 7.45-7.55 (m, 1H), 7.28 (d, J=9.0 Hz, 1H), 3.88 (s, 3H); MS. (ESI⁺ m/z 536).

### Example 4

This example shows University of Michigan Quinazoline Library 4-Experimentals (Synthesis of MOL-171-177, MOL-181-186, and MOL-191-196)

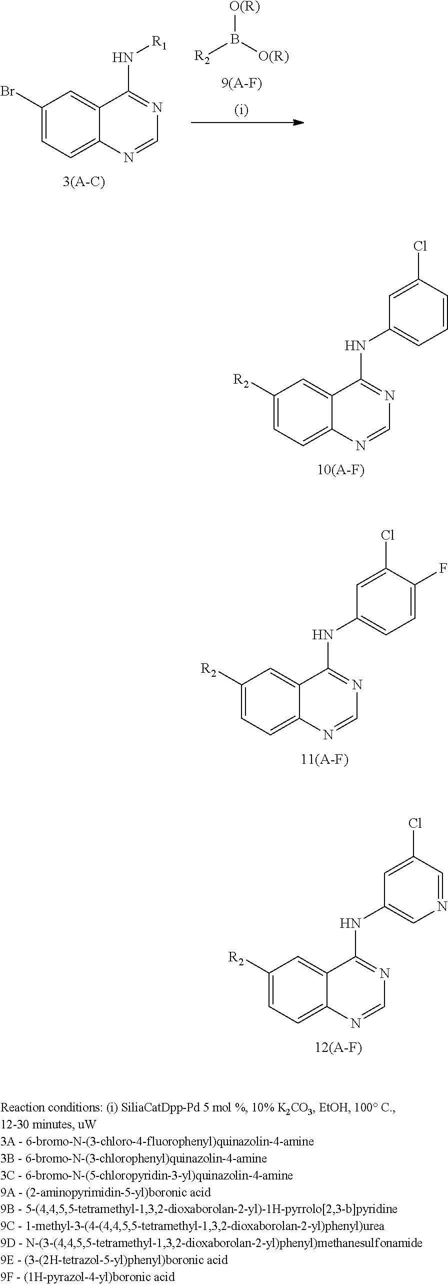

### 10A, 6-(2-aminopyrimidin-5-yl)-N-(3-chlorophenyl)quinazolin-4-amine, MOL-171

To a solution consisting of 6-bromo-N-(3-chlorophenyl)quinazolin-4-amine (3B, 0.115 g, 0.343 mmol) in anhydrous ethanol (4 mL) was placed in a 5 mL microwave reaction vial containing a stir bar. Next, (2-aminopyrimidin-5-yl)boronic acid (9A, 0.50 g, 0.361 mmol) was added followed by SiliCat DPP-Pd (5 mol %, 0.26 mmol/g loading, 0.068 g) and 10% aqueous potassium carbonate solution (2 equivalents, 0.50 mL, 0.68 mmol). The reaction mixture was placed under N₂ atmosphere, capped, and then heated at 100° C. for 15 minutes in a Biotage Emrys Optimizer microwave. The reaction mixture was allowed to cool to room temperature and then filtered over a fritted funnel to collect SiliCat DPP-Pd. The filtered solid was rinsed with excess ethanol and the filtrate was concentrated under reduced pressure to afford the crude product. Purification of the crude product by Biotage Isolera flash chromatography using a gradient of 4-100% ethyl acetate in heptane, followed by 0-10% methanol in dichloromethane afforded 6-(2-aminopyrimidin-5-yl)-N-(3-chlorophenyl)quinazolin-4-amine (10A, MOL-171, 26.4 mg, 22% yield, 95% purity) as a white solid; ¹H NMR (400 MHz, DMSO-d₆) δ 9.88 (s, 1H), 8.83 (s, 1H), 8.76 (m, 1H), 8.65 (s, 1H), 8.20 (dd, J=1.65, 8.60 Hz, 1H), 8.10 (t, J=1.92 Hz, 1H), 7.73-7.99 (m, 2H), 7.45 (t, J=8.14 Hz, 1H), 7.07-7.31 (m, 1H), 6.95 (s, 2H); MS: (ESI⁺ m/z 348.8, ESI⁻ m/z 346.8); TLC: (90:10:0.5, DCM:MeOH:NH₄OH) R_{f}=0.52.

Each of the following (10B-10F) was prepared in the manner described for 10A unless otherwise noted:

### 10B, N-(3-chlorophenyl)-6-(1H-pyrrolo[2,3-b]pyridin-5-yl)quinazolin-4-amine, MOL-172

5-(4,4,5,5-tetramethyl-1,3,2-dioxaborolan-2-yl)-1H-pyrrolo[2,3-b]pyridine 9B was used instead of 9A to afford the title compound as an off-white solid (0.022 g, 20% yield, 97% purity); ¹H NMR (400 MHz, DMSO-d₆) δ 11.81 (br. s., 1H), 10.06 (br. s., 1H), 8.86 (s, 1H), 8.75 (d, J=1.83 Hz, 1H), 8.56 (br. s., 1H), 8.42 (d, J=2.01 Hz, 1H), 8.22 (d, J=8.05 Hz, 1H), 8.05 (br. s., 1H), 7.68-7.93 (m, 2H), 7.56 (d, J=3.29 Hz, 1H), 7.40 (t, J=8.14 Hz, 1H), 7.12 (d, J=7.14 Hz, 1H), 6.56 (d, J=5.51 Hz, 1H); MS: (ESI⁺ m/z 371.8, ESI⁻ m/z 369.8); TLC: (90:10:0.5, DCM:MeOH:NH₄OH) R_{f}=0.54.

### 10C, 1-(4-(4-((3-chlorophenyl)amino)quinazolin-6-yl)phenyl)-3-methylurea, MOL-173

1-methyl-3-(4-(4,4,5,5-tetramethyl-1,3,2-dioxaborolan-2-yl)phenyl)urea 9C was used instead of 9A to afford the title compound as an off-white solid (0.037 g, 28% yield, 96% purity); ¹H NMR (400 MHz, DMSO-d₆) δ 9.96 (s, 1H), 8.77 (d, J=1.83 Hz, 1H), 8.71 (s, 1H), 8.64 (s, 1H), 8.14-8.41 (m, 1H), 8.01-8.14 (m, 1H), 7.69-7.96 (m, 2H), 7.59 (d, J=8.60 Hz, 1H), 7.45 (t, J=8.14 Hz, 1H), 7.06-7.31 (m, 1H), 6.07 (d, J=4.57 Hz, 1H), 3.33 (s, 3H); MS: (ESI⁺ m/z 403.8, ESI⁻ m/z 401.8); TLC: (90:10:0.5, DCM:MeOH:NH₄OH) R_{f}=0.54.

### 10D, N-(3-(4-((3-chlorophenyl)amino)quinazolin-6-yl)phenyl)methanesulfonamide, MOL-174

N-(3-(4,4,5,5-tetramethyl-1,3,2-dioxaborolan-2-yl)phenyl)methanesulfonamideurea 9D was used instead of 9A to afford the title compound as an off-white solid (0.049 g, 39% yield, 96% purity); ¹H NMR (400 MHz, DMSO-d₆) δ 10.04 (s, 1H), 9.91 (s, 1H), 8.81 (d, J=1.83 Hz, 1H), 8.68 (s, 1H), 8.02-8.22 (m, 2H), 7.75-8.01 (m, 2H), 7.59-7.67 (m, 1H), 7.50-7.59 (m, 1H), 7.45 (t, J=8.14 Hz, 1H), 7.31 (d, J=8.60 Hz, 1H), 7.20 (dd, J=1.74, 7.78 Hz, 1H) 3.07 (s, 3H); MS: (ESI⁺ m/z 425.8, ESI⁻ m/z 423.7); TLC: (90:10:0.5, DCM:MeOH:NH₄OH) R_{f}=0.62.

### 10E, 6-(3-(1H-tetrazol-5-yl)phenyl)-N-(3-chlorophenyl)quinazolin-4-amine, MOL-175

(3-(2H-tetrazol-5-yl)phenyl)boronic acid 9E was used instead of 9A to afford the title compound as an off-white solid (0.049 g, 21% yield, 98% purity); ¹H NMR (400 MHz, DMSO-d₆) δ 10.10 (br. s., 1H), 8.94 (s, 1H), 8.54 (s, 1H), 8.29 (d, J=8.78 Hz, 1H), 8.03-8.19 (m, 2H), 7.96 (d, J=8.60 Hz, 1H), 7.64-7.92 (m, 2H), 7.46 (t, J=8.05 Hz, 1H), 7.22 (dd, J=1.30, 7.90 Hz, 1H); MS: (ESI⁺ m/z 400.0, ESI⁻ m/z 398.1); TLC: (90:10:0.5, DCM:MeOH:NH₄OH) R_{f}=0.56.

### 10F, N-(3-chlorophenyl)-6-(1H-pyrazol-4-yl)quinazolin-4-amine, MOL-176

(1H-pyrazol-4-yl)boronic acid 9F was used instead of 9A to afford the title compound as a white solid (0.010 g, 9% yield, 98% purity); ¹H NMR (400 MHz, DMSO-d₆) δ 13.11 (br. s., 1H), 9.80 (s, 1H), 8.72 (s, 1H), 8.62 (s, 1H), 8.35 (br. s., 1H) 8.09-8.21 (m, 2H), 7.88 (dd, J=1.80, 8.00 Hz, 1H), 7.80 (d, J=8.78 Hz, 1H), 7.46 (t, J=8.14 Hz, 1H), 7.20 (dd, J=1.80, 8.34 Hz, 1H); MS: (ESI⁺ m/z 322.0, ESI⁻ m/z 320.0); TLC: (90:10:0.5, DCM:MeOH:NH₄OH) R_{f}=0.54.

### N-(3-chlorophenyl)-6-(1H-pyrazolo[3,4-b]pyridin-5-yl)quinazolin-4-amine, MOL-177

To a solution consisting of 6-bromo-N-(3-chlorophenyl)quinazolin-4-amine (0.133 g, 0.36 mmol) and 1H-pyrazolo[3,4-b]pyridine-5-boronic acid pinacol ester (0.133 g, 0.54 mmol) in 1,4-dioxane (2 mL) in a 2 mL microwave reaction vial containing a stir bar was added 2M K₂CO₃ (0.72 mL, 1.44 mmol). The mixture was degassed (vacuum/nitrogen, 3 times) before the addition of SiliCat DPP-Pd (0.10 g, 0.26 mmol/g loading) and then heated three times at 140° C. for 20 minutes in a Biotage Emrys Optimizer microwave. The reaction mixture was allowed to cool to room temperature, the aqueous layer was removed with a disposable pipette, and the remaining organic phase was filtered through a fritted funnel to collect SiliCat DPP-Pd. The filtered solid was rinsed with room temperature methanol and the filtrate was set aside. The filtered solids were then washed well with hot methanol and the filtrate was concentrated under reduced pressure to afford the title compound as a pale yellow solid (43 mg, 32%, 94.9% purity); TLC R_{f} 0.10 (solvent system: 7:3 v/v ethyl acetate-heptane); MS (ES−API+) m/z 373.0 (M+1), 375.0 (Cl isotope), (ES−API−) m/z 371.0 (M−1), 373.0 (Cl isotope); ¹H NMR (400 MHz, DMSO-d₆) □ 9.01 (d, J=1.28 Hz, 1H), 8.86 (s, 1H), 8.62 (s, 1H), 8.53 (s, 1H), 8.18-8.25 (m, 2H), 8.01 (s, 1H), 7.80 (d, J=8.69 Hz, 1H), 7.75 (br d, J=8.23 Hz, 1H), 7.37 (t, J=7.96 Hz, 1H), 7.09 (br d, J=7.87 Hz, 1H).

### 11A, 6-(2-aminopyrimidin-5-yl)-N-(3-chloro-4-fluorophenyl)quinazolin-4-amine, MOL-181

To a solution consisting of 6-bromo-N-(3-chloro-4-fluorophenyl)quinazolin-4-amine (3A, 0.150 g, 0.425 mmol) in anhydrous ethanol (4 mL) was placed in a 5 mL microwave reaction vial containing a stir bar. Next, (2-aminopyrimidin-5-yl)boronic acid (9A, 0.62 g, 0.447 mmol) was added followed by SiliCat DPP-Pd (5 mol %, 0.26 mmol/g loading, 0.085 g) and 10% aqueous potassium carbonate solution (2 equivalents, 0.62 mL, 0.85 mmol). The reaction mixture was placed under N₂ atmosphere, capped, and then heated at 100° C. for 15 minutes in a Biotage Emrys Optimizer microwave. The reaction mixture was allowed to cool to room temperature and then filtered over a fritted funnel to collect SiliCat DPP-Pd. The filtered solid was rinsed with excess ethanol and the filtrate was concentrated under reduced pressure to afford the crude product. Purification of the crude product by Biotage Isolera flash chromatography using a gradient of 4-100% ethyl acetate in heptane, followed by 0-10% methanol in dichloromethane afforded 6-(2-aminopyrimidin-5-yl)-N-(3-chloro-4-fluorophenyl)quinazolin-4-amine (11A, MOL-181, 75 mg, 48% yield, 95% purity) as a white solid; ¹H NMR (400 MHz, DMSO-d₆) δ 9.91 (s, 1H), 8.82 (s, 1H), 8.69-8.78 (m, 1H), 8.63 (s, 1H), 8.04-8.29 (m, 1H), 7.78-7.92 (m, 1H), 7.49 (t, J=9.06 Hz, 1H), 6.96 (s, 2H); MS: (ESI⁺ m/z 367.0, ESI⁻ m/z 365.0); TLC: (90:10:0.5, DCM:MeOH:NH₄OH) R_{f}=0.58.

Each of the following (11B-11F) was prepared in the manner described for 11A unless otherwise noted:

### 11B, N-(3-chloro-4-fluorophenyl)-6-(1H-pyrrolo[2,3-b]pyridin-5-yl)quinazolin-4-amine, MOL-182

5-(4,4,5,5-tetramethyl-1,3,2-dioxaborolan-2-yl)-1H-pyrrolo[2,3-b]pyridine 9B was used instead of 9A to afford the title compound as an off-white solid (0.067 g, 41% yield, 98% purity); ¹H NMR (400 MHz, DMSO-d₆) δ 11.84 (br. s., 1H), 10.01 (s, 1H), 8.83-8.99 (m, 1H), 8.78 (d, J=2.01 Hz, 1H), 8.65 (s, 1H), 8.44 (d, J=2.01 Hz, 1H), 8.17-8.37 (m, 2H), 7.83-7.95 (m, 1H), 7.57 (t, J=2.93 Hz, 1H), 7.49 (t, J=9.15 Hz, 1H), 6.41-6.67 (m, 1H); MS: (ESI⁺ m/z 390.1, ESI⁻ m/z 388.1); TLC: (90:10:0.5, DCM:MeOH:NH₄OH) R_{f}=0.63

### 11C, 1-(4-(4-((3-chloro-4-fluorophenyl)amino)quinazolin-6-yl)phenyl)-3-methylurea, MOL-183

1-methyl-3-(4-(4,4,5,5-tetramethyl-1,3,2-dioxaborolan-2-yl)phenyl)urea 9C was used instead of 9A to afford the title compound as an off-white solid (0.022 g, 13% yield, 100% purity); ¹H NMR (400 MHz, DMSO-d₆) δ 9.98 (s, 1H), 8.75 (d, J=1.40 Hz, 1H), 8.71 (s, 1H), 8.63 (s, 1H), 8.06-8.27 (m, 1H), 7.70-7.91 (m, 2H), 7.59 (d, J=8.60 Hz, 1H), 7.49 (t, J=9.06 Hz, 1H), 6.08 (d, J=4.76 Hz, 1H), 3.33 (s, 3H), 2.67 (d, J=4.57 Hz, 2H); MS: (ESI⁺ m/z 422.1, ESI⁻ m/z 420.1); TLC: (90:10:0.5, DCM:MeOH:NH₄OH) R_{f}=0.58.

### 11D, N-(3-(4-((3-chloro-4-fluorophenyl)amino)quinazolin-6-yl)phenyl)methanesulfonamide, MOL-184

N-(3-(4,4,5,5-tetramethyl-1,3,2-dioxaborolan-2-yl)phenyl)methanesulfonamideurea 9D was used instead of 9A to afford the title compound as an off-white solid (0.056 g, 30% yield, 96% purity); ¹H NMR (400 MHz, DMSO-d₆) δ 10.06 (s, 1H), 9.91 (s, 1H), 8.77 (s, 1H), 8.66 (s, 1H), 8.19 (dd, J=2.47, 6.86 Hz, 1H), 8.11 (dd, J=1.37, 8.69 Hz, 1H), 7.72-7.99 (m, 2H), 7.41-7.65 (m, 3H), 7.30 (d, J=7.87 Hz, 1H), 3.07 (s, 3H); MS: (ESI⁺ m/z 443.1, ESI⁻ m/z 441.1); TLC: (90:10:0.5, DCM:MeOH:NH₄OH) R_{f}=0.66.

### 11E, 6-(3-(1H-tetrazol-5-yl)phenyl)-N-(3-chloro-4-fluorophenyl)quinazolin-4-amine, MOL-185

(3-(2H-tetrazol-5-yl)phenyl)boronic acid 9E was used instead of 9A to afford the title compound as an off-white solid (0.007 g, 4% yield, 83% purity); ¹H NMR (400 MHz, DMSO-d₆) δ 10.24 (br. s., 1H), 9.03 (s, 1H), 8.66 (m, 2H), 8.28 (m, 2H), 8.10 (d, J=7.32 Hz, 1H), 7.81-8.03 (m, 2H), 7.68 (t, J=7.32 Hz, 1H), 7.48 (t, J=9.00 Hz, 1H); MS: (ESI⁺ m/z 418.0, ESI⁻ m/z 416.0); TLC: (90:10:0.5, DCM:MeOH:NH₄OH) R_{f}=0.22.

### 11F, N-(3-chloro-4-fluorophenyl)-6-(1H-pyrazol-4-yl)quinazolin-4-amine, MOL-186

(1H-pyrazol-4-yl)boronic acid 9F was used instead of 9A to afford the title compound as a white solid (0.022 g, 15% yield, 97% purity); ¹H NMR (400 MHz, DMSO-d₆) δ 13.11 (br. s., 1H), 9.80 (s, 1H), 8.69 (s, 1H), 8.59 (s, 1H), 8.35 (br. s., 1H) 8.02-8.28 (m, 2H), 7.80-7.92 (m, 1H), 7.79 (d, J=8.78 Hz, 1H), 7.49 (t, J=9.14 Hz, 1H), 7.20 (dd, J=1.80, 8.34 Hz, 1H); MS: (ESI⁺ m/z 340.0, ESI⁻ m/z 338.0); TLC: (90:10:0.5, DCM:MeOH:NH₄OH) R_{f}=0.54.

### 3-(4-((3-chloro-4-fluorophenyl)amino)quinazolin-6-yl)-N-cyclopropylbenzenesulfonamide, MOL-214

A mixture consisting of 6-bromo-N-(3-chlorophenyl)quinazolin-4-amine—HCl (100 mg 0.26 mmol), (3-(N-cyclopropylsulfamoyl)phenyl)boronic acid (94 mg, 0.39 mmol) and 1.4M K₂CO₃ (1.1 mL) in 3 mL of 1,4-dioxane was degassed (vacuum/nitrogen, 3 times). To the reaction mixture was added SiliCat DPP-Pd (50 mg, 0.26 mmol/g loading). The reaction mixture was sealed and heated at 100° C. for 12 minutes in a Biotage Emrys Optimizer microwave. To the reaction mixture was added additional 2-chloro-5-(4,4,5,5-tetramethyl-1,3,2-dioxaborolan-2-yl)aniline (40 mg, 0.16 mmol) and SiliCat DPP-Pd (30 mg). The reaction mixture was heated again at 120° C. for 15 minutes and cooled. The aqueous phase was removed and the remaining organic phase was filtered through a glass frit. The solids were washed with methanol. The filtrate was concentrated under reduced pressure. The white solid residue was applied to a 40 g silica column using the dry loading method and eluted with a gradient of 4:6 ethyl acetate-heptane to 100% ethyl acetate to give 20 mg (16%, purity 96%) of the title compound as a pale yellow solid; MS (ES−API+) m/z 469.0 (M+1), 471.0 (Cl isotope); ¹H NMR (400 MHz, DMSO-d₆) □ 10.13 (s, 1H), 8.87 (s, 1H), 8.67 (s, 1H), 8.14-8.27 (m, 4H), 8.01 (d, J=2.65 Hz, 1H), 7.95 (d, J=8.69 Hz, 1H), 7.87-7.92 (m, 1H), 7.79-7.87 (m, 2H), 7.49 (t, J=9.06 Hz, 1H), 2.17 (dt,

J=3.34, 6.75 Hz, 1H), 0.37-0.54 (m, 4H).

### 13 N-(3-chloro-4-fluorophenyl)-6-(6-methoxypyridin-3-yl)quinazolin-4-amine, MOL-151

A solution of 6-bromo-N-(3-chloro-4-fluorophenyl)quinazolin-4-amine (3A, 275 mg, 0.78 mmol) and (6-methoxypyridin-3-yl)boronic acid (9G, 119 mg, 0.78 mmol) in 1,4-dioxane (15 mL) and water (1.4 mL) was degassed. To the solution was added cesium carbonate (1.0 g, 3.1 mmol) and [1,1′-bis(diphenylphosphino)ferrocene]dichloropalladium(II) (44 mg). The reaction mixture was heated at 80° C. under N₂ for 2 hours. The reaction mixture was diluted with toluene and the volatiles were removed under vacuum and the crude material was purified by silica gel column chromatography eluting with a gradient of 3/7 to 6/4 ethyl acetate/heptane to give N-(3-chloro-4-fluorophenyl)-6-(6-methoxypyridin-3-yl)quinazolin-4-amine (13, MOL-151, 40 mg, 13%, 95% purity by HPLC) as a yellow solid; ¹H NMR (400 MHz, DMSO-d₆) δ 9.93 (s, 1H), 8.77 (d, J=1.5 Hz, 1H), 8.69 (d, J=2.6 Hz, 1H), 8.63 (s, 1H), 8.1-8.24 (m, 3H), 7.78-7.92 (m, 2H), 7.46 (t, J=9.15 Hz, 1H), 7.00 (d, J=8.8 Hz, 1H), 3.92 (s, 3H); MS: (ESI⁺ m/z 381.1, ESI m/z 379.1).

### 12A, 6-(2-aminopyrimidin-5-yl)-N-(5-chloropyridin-3-yl)quinazolin-4-amine, MOL-191

To a solution consisting of 6-bromo-N-(5-chloropyridin-3-yl)quinazolin-4-amine (3C, 0.150 g, 0.447 mmol) in anhydrous ethanol (4 mL) was placed in a 5 mL microwave reaction vial containing a stir bar. Next, (2-aminopyrimidin-5-yl)boronic acid (9A, 0.65 g, 0.469 mmol) was added followed by SiliCat DPP-Pd (5 mol %, 0.26 mmol/g loading, 0.090 g) and 10% aqueous potassium carbonate solution (2 equivalents, 0.65 mL, 0.89 mmol). The reaction mixture was placed under N₂ atmosphere, capped, and then heated at 100° C. for 15 minutes in a Biotage Emrys Optimizer microwave. The reaction mixture was allowed to cool to room temperature and then filtered over a fritted funnel to collect SiliCat DPP-Pd. The filtered solid was rinsed with excess ethanol and the filtrate was concentrated under reduced pressure to afford the crude product. Purification of the crude product by Biotage Isolera flash chromatography using a gradient of 4-100% ethyl acetate in heptane, followed by 0-10% methanol in dichloromethane afforded 6-(2-aminopyrimidin-5-yl)-N-(5-chloropyridin-3-yl)quinazolin-4-amine (12A, MOL-191, 44 mg, 28% yield, 95% purity) as a white solid; ¹H NMR (400 MHz, DMSO-d₆) δ 10.06 (s, 1H), 9.01 (s, 1H), 8.83 (s, 1H), 8.70 (s, 1H), 8.62 (br. s., 1H), 8.39 (d, J=1.50 Hz, 1H), 8.23 (d, J=8.23 Hz 1H), 7.89 (d, J=8.60 Hz, 1H), 6.97 (s, 2H); MS: (ESI⁺ m/z 350.0, ESI⁻ m/z 348.0); TLC: (90:10:0.5, DCM:MeOH:NH₄OH) R_{f}=0.40.

Each of the following (12B-12F) was prepared in the manner described for 12A unless otherwise noted:

### 12B, N-(5-chloropyridin-3-yl)-6-(1H-pyrrolo[2,3-b]pyridin-5-yl)quinazolin-4-amine, MOL-192

5-(4,4,5,5-tetramethyl-1,3,2-dioxaborolan-2-yl)-1H-pyrrolo[2,3-b]pyridine 9B was used instead of 9A to afford the title compound as an off-white solid (0.052 g, 31% yield, 98% purity); ¹H NMR (400 MHz, DMSO-d₆) δ 11.84 (br. s., 1H), 10.16 (s, 1H), 9.03 (d, J=2.01 Hz, 1H), 8.89 (m, 1H), 8.78 (d, J=2.01 Hz, 1H), 8.71 (s, 1H), 8.63 (t, J=2.01 Hz, 1H), 8.44 (d, J=2.01 Hz, 1H), 8.14-8.41 (m, 2H), 7.93 (d, J=8.60 Hz, 1H), 7.57 (t, J=2.93 Hz, 1H), 6.57 (dd, J=1.83, 3.48 Hz, 1H); MS: (ESI⁺ m/z 373.1, ESI⁻ m/z 371.1); TLC: (90:10:0.5, DCM:MeOH:NH₄OH) R_{f}=0.50.

### 12C, 1-(4-(4-((5-chloropyridin-3-yl)amino)quinazolin-6-yl)phenyl)-3-methylurea, MOL-193

1-methyl-3-(4-(4,4,5,5-tetramethyl-1,3,2-dioxaborolan-2-yl)phenyl)urea 9C was used instead of 9A to afford the title compound as a white solid (0.016 g, 9% yield, 98% purity); ¹H NMR (400 MHz, DMSO-d₆) δ 10.14 (br. s., 1H), 9.02 (br. s., 1H), 8.65-8.88 (m, 2H), 8.62 (br. s., 1H), 8.38 (br. s., 1H), 8.21 (d, J=8.78 Hz, 1H), 7.88 (d, J=8.42 Hz, 1H), 7.79 (d, J=8.42 Hz, 1H), 7.59 (d, J=8.42 Hz, 1H), 6.08 (d, J=4.76 Hz, 1H), 3.33 (s, 3H), 2.67 (d, J=4.21 Hz, 2H); MS: (ESI⁺ m/z 405.1, ESI⁻ m/z 403.1); TLC: (90:10:0.5, DCM:MeOH:NH₄OH) R_{f}=0.49.

### 12D, N-(3-(4-((5-chloropyridin-3-yl)amino)quinazolin-6-yl)phenyl)methanesulfonamide, MOL-194

N-(3-(4,4,5,5-tetramethyl-1,3,2-dioxaborolan-2-yl)phenyl)methanesulfonamideurea 9D was used instead of 9A to afford the title compound as a white solid (0.049 g, 26% yield, 97% purity); ¹H NMR (400 MHz, DMSO-d₆) δ 10.23 (s, 1H), 9.93 (s, 1H), 9.00 (s, 1H), 8.80 (s, 1H), 8.73 (s, 1H), 8.61 (br. s., 1H), 8.39 (d, J=2.01 Hz, 1H), 8.14 (dd, J=1.37, 8.69 Hz, 1H), 7.95 (d, J=8.78 Hz, 1H), 7.43-7.65 (m, 2H), 7.32 (d, J=7.87 Hz, 1H), 3.08 (s, 3H); MS: (ESI⁺ m/z 426.0, ESI⁻ m/z 424.0); TLC: (90:10:0.5, DCM:MeOH:NH₄OH) R_{f}=0.51.

### 12E, 6-(3-(1H-tetrazol-5-yl)phenyl)-N-(5-chloropyridin-3-yl)quinazolin-4-amine, MOL-195

(3-(2H-tetrazol-5-yl)phenyl)boronic acid 9E was used instead of 9A to afford the title compound as an off-white solid (0.030 g, 17% yield, 95% purity); ¹H NMR (400 MHz, DMSO-d₆) δ 10.28 (s, 1H), 9.01 (d, J=1.83 Hz, 1H), 8.93 (s, 1H), 8.74 (s, 1H), 8.61 (t, J=1.83 Hz, 2H), 8.54 (s, 1H), 8.40 (d, J=2.01 Hz, 1H), 8.32 (dd, J=1.46, 8.78 Hz, 1H), 8.10 (dd, J=8.05, 13.91 Hz, 2H), 7.99 (t, J=8.60 Hz, 1H), 7.81 (t, J=7.78 Hz, 1H); MS: (ESI⁺ m/z 401.0, ESI⁻ m/z 399.1); TLC: (90:10:0.5, DCM:MeOH:NH₄OH) R_{f}=0.08.

### 12F, N-(5-chloropyridin-3-yl)-6-(1H-pyrazol-4-yl)quinazolin-4-amine, MOL-196

(1H-pyrazol-4-yl)boronic acid 9F was used instead of 9A to afford the title compound as a white solid (0.010 g, 7% yield, 99% purity); ¹H NMR (400 MHz, DMSO-d₆) δ 13.12 (br. s., 1H), 9.98 (s, 1H), 9.01 (br. s., 1H), 8.60-8.72 (m, 3H), 8.38 (d, J=2.01 Hz, 1H), 8.18 (d, J=8.42 Hz, 2H), 7.83 (d, J=8.23 Hz, 1H), 7.68 (s, 1H); MS: (ESI⁺ m/z 323.0, ESI⁻ m/z 321.0); TLC: (90:10:0.5, DCM:MeOH:NH₄OH) R_{f}=0.37.

### Example 5

This example shows EMD Quinazoline Experimentals (Synthesis of EMD-151)

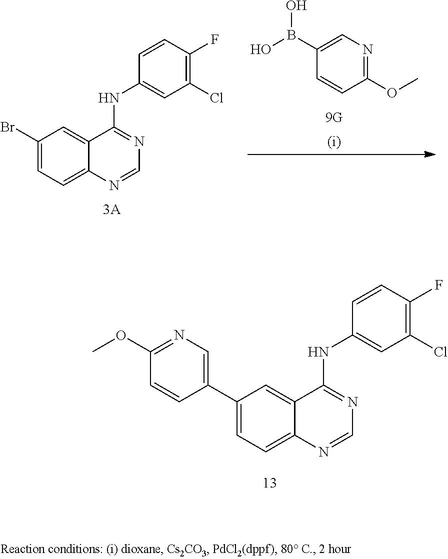 [SMILES: Fc1ccc(Nc2ncnc3ccc(Br)cc23)cc1Cl ; COc1ccc(B(O)O)cn1 ; COc1ccc(-c2ccc3ncnc(Nc4ccc(F)c(Cl)c4)c3c2)cn1]

### 13 N-(3-chloro-4-fluorophenyl)-6-(6-methoxypyridin-3-yl)quinazolin-4-amine, EMD-151

A solution of 6-bromo-N-(3-chloro-4-fluorophenyl)quinazolin-4-amine (3A, 275 mg, 0.78 mmol) and (6-methoxypyridin-3-yl)boronic acid (9G, 119 mg, 0.78 mmol) in 1,4-dioxane (15 mL) and water (1.4 mL) was degassed. To the solution was added cesium carbonate (1.0 g, 3.1 mmol) and [1,1′-bis(diphenylphosphino)ferrocene]dichloropalladium(II) (44 mg). The reaction mixture was heated at 80° C. under N₂ for 2 hours. The reaction mixture was diluted with toluene and the volatiles were removed under vacuum and the crude material was purified by silica gel column chromatography eluting with a gradient of 3/7 to 6/4 ethyl acetate/heptane to give N-(3-chloro-4-fluorophenyl)-6-(6-methoxypyridin-3-yl)quinazolin-4-amine (13, EMD-151, 40 mg, 13%, 95% purity by HPLC) as a yellow solid; ¹H NMR (400 MHz, DMSO-d₆) δ 9.93 (s, 1H), 8.77 (d, J=1.5 Hz, 1H), 8.69 (d, J=2.6 Hz, 1H), 8.63 (s, 1H), 8.1-8.24 (m, 3H), 7.78-7.92 (m, 2H), 7.46 (t, J=9.15 Hz, 1H), 7.00 (d, J=8.8 Hz, 1H), 3.92 (s, 3H); MS: (ESI⁺ m/z 381.1, ESI⁻ m/z 379.1).

### Example 6

This example describes the synthesis of additional quinazoline based compounds of the present invention.

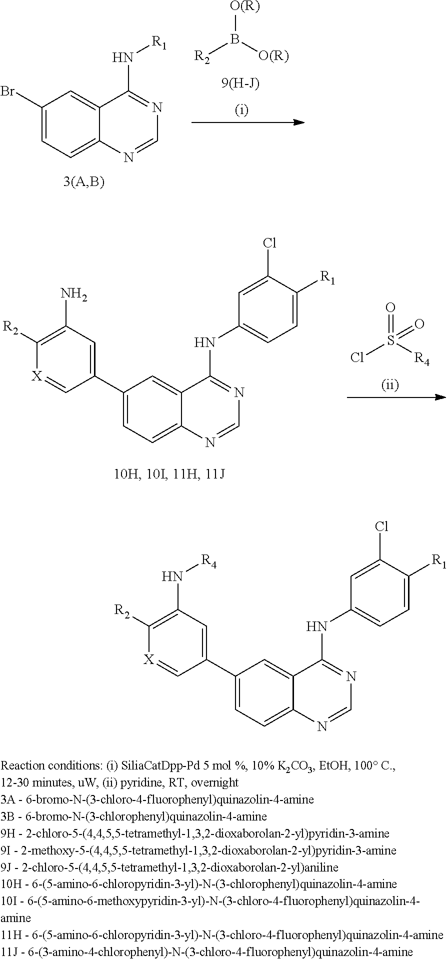

### 6-(5-amino-6-chloropyridin-3-yl)-N-(3-chlorophenyl)quinazolin-4-amine (3B), MOL-200

To a solution consisting of 6-bromo-N-(3-chlorophenyl)quinazolin-4-amine (10.0 g, 26.9 mmol) and 2-chloro-5-(4,4,5,5-tetramethyl-1,3,2-dioxaborolan-2-yl)pyridin-3-amine (9H) (6.8 g, 26.9 mmol) in 1,4-dioxane (250 mL) was added 1.4M K₂CO₃ (58 mL, 81 mmol). The mixture was degassed (vacuum/nitrogen, 3 times) before the addition of SiliCat DPP-Pd (3.5 g, 0.26 mmol/g loading) and then heated at 95° C. overnight with stirring. The reaction mixture was allowed to cool to room temperature and was diluted with ethyl acetate, methanol and dichloromethane. The mixture was washed with water twice, then brine. The organic phase was dried over magnesium sulfate, filtered, and concentrated under reduce pressure. The residue was triturated under a mix of solvents, 50 mL ethyl acetate, 40 mL dichloromethane, 10 mL methanol, 0.25 mL ammonium hydroxide, for 1 hour and filtered. The solid was washed with ethyl acetate and dried in high vacuum to afford the title compound (5.92 g, 57%). The filtrate was applied to a silica column eluted with 2:35:63 methanol-ethyl acetate-dichloromethane to afford another lot of the title compound as a white solid (0.2 g, 100% purity). TLC Rf 0.16 (solvent system: 65:35 v/v ethyl acetate-heptane); MS (ES−API+) m/z 382.1 (M+1), 384.1 (Cl isotope), (ES−API−) m/z 380.0 (M−1), 382.0 (Cl isotope); ¹H NMR (400 MHz, DMSO-d₆) □ 10.00 (s, 1H), 8.82 (d, J=1.74 Hz, 1H),

8.67 (s, 1H), 8.05-8.15 (m, 3H), 7.89 (d, J=8.60 Hz, 1H), 7.82-7.87 (m, 1H), 7.51 (d, J=2.20 Hz, 1H), 7.43 (t, J=8.14 Hz, 1H), 7.15-7.22 (m, 1H), 5.74 (s, 2H).

### N-(2-chloro-5-(4-((3-chlorophenyl)amino)quinazolin-6-yl)pyridin-3-yl)methanesulfonamide, MOL-201

To a mixture consisting of 6-(5-amino-6-chloropyridin-3-yl)-N-(3-chlorophenyl)quinazolin-4-amine (1.99 g, 5.2 mmol) in pyridine (25 mL) was added methanesulfonyl chloride (0.35 g, 3.0 mmol) followed by another addition of methanesulfonyl chloride (0.35 g, 3.0 mmol) after 3 hours and another (0.46 mg, 4.0 mmol) after 30 minutes. The reaction mixture was stirred at room temperature overnight. To the ice cold reaction mixture was added 2N NaOH (5 mL, 10 mmol), allowed to warm to room temperature, followed by another addition (5 mL, 10 mmol) at 0° C. after 3 hours. The mixture was allowed to stir for 1 hour while warming to room temperature and 1N HCl (3 mL, 3 mmol) and brine were added. Organic material was extracted twice with ethyl acetate-methanol (8:2). The combined organic phase was washed with brine, dried over magnesium sulfate, filtered, and concentrated under reduced pressure. The residue was suspended in toluene and concentrated, followed by ethyl acetate and concentrated, to give near white solid. The solid was triturated under 20 mL/30 mL of methanol/ethyl acetate overnight and filtered to afford the title compound as an off-white solid (1.55 g, 65%, 99.6% purity). MS (ES−API+) m/z 460.0 (M+1), 462.0 (Cl isotope), (ES−API−) m/z 457.9 (M−1), 459.9 (Cl isotope); ¹H NMR (400 MHz, DMSO-d₆) □ 10.04 (s, 1H), 9.93 (br s, 1H), 8.88 (s, 1H), 8.67 (s, 1H), 8.60 (s, 1H), 8.14-8.23 (m, 2H), 8.09 (t, J=1.92 Hz, 1H), 7.91 (d, J=8.69 Hz, 1H), 7.83 (dd, J=1.01, 8.33 Hz, 1H), 7.43 (t, J=8.10 Hz, 1H), 7.19 (d, J=8.14 Hz, 1H), 3.07 (s, 3H).

### N-(2-chloro-5-(4-((3-chlorophenyl)amino)quinazolin-6-yl)pyridin-3-yl)-N-(methylsulfonyl)methanesulfonamide, MOL-201B

To a mixture consisting of 6-(5-amino-6-chloropyridin-3-yl)-N-(3-chlorophenyl)quinazolin-4-amine (255 mg, 0.67 mmol) in pyridine (1.2 mL) was added methanesulfonyl chloride (458 mL, 4.0 mmol) in small portions. The reaction mixture was stirred at room temperature for 5 hours then stored at 3° C. overnight. The crystalline material was filtered, washed with 2 mL of methanol and triturated under 5 mL of methanol for 3 hours. The solid was filtered and dried in high vacuum to give the title compound (125 mg, 23%, 88% purity); MS (ES−API+) m/z 538 (M+1), 541 (Cl isotope), (ES−API−) m/z 535.9 (M−1), 537.9 (Cl isotope); ¹H NMR (400 MHz, DMSO-d₆) □ 11.47 (br s, 1H), 9.57 (s, 1H), 9.20 (d, J=2.29 Hz, 1H), 8.97 (d, J=2.29 Hz, 1H), 8.87 (s, 1H), 8.52 (dd, J=1.60, 8.74 Hz, 1H), 8.14 (t, J=1.88 Hz, 1H), 8.02 (d, J=8.78 Hz, 1H), 7.93 (d, J=7.64 Hz, 1H), 7.49 (t, J=8.10 Hz, 1H), 7.30 (d, J=7.57 Hz, 1H), 3.76 (s, 6H).

### 6-(5-amino-6-methoxypyridin-3-yl)-N-(3-chlorophenyl)quinazolin-4-amine (10I), MOL202A

A mixture consisting of 6-bromo-N-(3-chlorophenyl)quinazolin-4-amine—HCl (800 mg 2.15 mmol), 2-methoxy-5-(4,4,5,5-tetramethyl-1,3,2-dioxaborolan-2-yl)pyridin-3-amine (550 mg, 2.20 mmol) and 1.4M K₂CO₃ (6.1 mL) in 10 mL of 1,4-dioxane was degassed (vacuum/nitrogen, 3 times). To the reaction mixture was added SiliCat DPP-Pd (250 mg, 0.26 mmol/g loading). The reaction mixture was sealed and heated at 100° C. for 8 minutes in a Biotage Emrys Optimizer microwave. The reaction mixture was cooled, the aqueous phase removed and the remaining organic phase was filtered through a glass frit. The solids were washed with methanol. This reaction procedure was repeated 9 times. The combined filtrates were concentrated under reduced pressure. The residue was triturated under a mix of ethyl acetate, methanol, dichloromethane, and heptane overnight. The suspension was filtered to give after drying under high vacuum 2.46 g of the title compound as a gray-brown solid. The filtrate was applied to a 120 g silica column and it was eluted with a gradient of 1:1 ethyl acetate-heptane to 100% ethyl acetate to give 1.20 g of the title compound as a dull yellow solid. Total: 3.66 g (45%); MS (ES−API+) m/z 378.1 (M+1), 380.1 (Cl isotope); ¹H NMR (400 MHz, DMSO-d₆) □ 9.97 (s, 1H), 8.74 (d, J=1.55 Hz, 1H), 8.64 (s, 1H), 8.10 (t, J=1.92 Hz, 1H), 8.05 (d, J=8.69 Hz, 1H), 7.81-7.90 (m, 3H), 7.42 (t, J=8.14 Hz, 1H), 7.30 (d, J=2.20 Hz, 1H), 7.17 (d, J=7.67 Hz, 1H), 5.13 (s, 2H), 3.92 (s, 3H).

### N-(5-(4-((3-chlorophenyl)amino)quinazolin-6-yl)-2-methoxypyridin-3-yl)methanesulfonamide, MOL-202

To a mixture consisting of 6-(5-amino-6-methoxypyridin-3-yl)-N-(3-chlorophenyl)quinazolin-4-amine (300 mg, 0.79 mmol) in pyridine (2 mL) was added methanesulfonyl chloride (121 mg, 1.06 mmol). The reaction mixture was stirred at room temperature for 2.75 hours. The reaction mixture was filtered and the solids were washed with ethyl acetate and triturated under 2-propanol for 3 hours. The mixture was filtered and dried under high vacuum to give the title compound (267 mg, 74%) as a pale off-white solid; TLC Rf 0.25 (solvent system: 1:1 v/v ethyl acetate-heptane); MS (ES−API+) m/z 456 (M+1), 458 (Cl isotope), (ES−API−) m/z 453.9 (M−1), 456.0 (Cl isotope); ¹H NMR (400 MHz, DMSO-d₆) □ 12.35 (br s, 1H), 9.62 (br s, 1H), 9.40 (s,

1H), 8.89 (s, 1H), 8.72 (d, J=2.29 Hz, 1H), 8.39 (br d, J=8.87 Hz, 1H), 8.17 (d, J=2.10 Hz, 1H), 8.12 (d, J=8.60 Hz, 1H), 8.03 (s, 1H), 7.86 (br d, J=8.33 Hz, 1H), 7.48 (t, J=8.14 Hz, 1H), 7.34 (br d, J=8.42 Hz, 1H), 3.98 (s, 3H), 3.17 (s, 3H).

### N-(5-(4-((3-chlorophenyl)amino)quinazolin-6-yl)-2-methoxypyridin-3-yl)-N-(methylsulfonyl)methanesulfonamide, MOL-202B

To a mixture consisting of N-(5-(4-((3-chlorophenyl)amino)quinazolin-6-yl)-2-methoxypyridin-3-yl)methanesulfonamide (150 mg, 0.33 mmol) in pyridine (0.5 mL) was added methanesulfonyl chloride (227 mg, 1.98 mmol). The reaction mixture was stirred at room temperature for 2 hours followed by 4 hours at 40° C. The reaction mixture was stored at 0° C. overnight, diluted with 1 mL of dichloromethane and 3 drops of morpholine, (addition of morpholine resulted in a homogeneous solution) and applied directly to a 25 g column of silica gel for purification. The column was eluted with a gradient of 4:6 to 8:2 v/v ethyl acetate-heptane to isolate the title compound (18 mg, 10%) as a pale brown solid; TLC R_{f} 0.36 (solvent system: 1:1 v/v ethyl acetate-heptane); MS (ES−API+) m/z 534 (M+1), 536 (Cl isotope), (ES−API−) m/z 532 (M−1), 534 (Cl isotope).

### N-(5-(4-((3-chlorophenyl)amino)quinazolin-6-yl)-2-methoxypyridin-3-yl)cyclopropanesulfonamide, MOL-204

To a mixture consisting of 6-(5-amino-6-methoxypyridin-3-yl)-N-(3-chlorophenyl)quinazolin-4-amine (200 mg, 0.53 mmol) in pyridine (0.8 mL) was added cyclopropanesulfonyl chloride (278 mg, 1.98 mmol) in two equal portions, 1 hour apart. The reaction mixture was stirred at room temperature for an additional 2.25 hours. To the reaction mixture was added methanol (185 mg, 5.3 mmol) in 1 mL of dichloromethane and 3 drops of morpholine, (addition of morpholine resulted in a homogeneous solution) and the mixture was applied directly to a 40 g column of silica gel for purification. The column was eluted with a gradient of 0:100 to 10:90 v/v methanol-ethyl acetate to isolate the title compound (45 mg, 18%) as a solid; TLC R_{f} 0.25 (solvent system: 1:1 v/v ethyl acetate-heptane); MS (ES−API+) m/z 482 (M+1), 484 (Cl isotope), (ES−API−) m/z 480 (M−1), 482 (Cl isotope); ¹H NMR (400 MHz, DMSO-d₆) □ 9.97 (s, 1H), 9.44 (s, 1H), 8.80 (s, 1H), 8.65 (s, 1H), 8.53 (d, J=2.10 Hz, 1H), 8.17 (dd, J=1.33, 8.74 Hz, 1H), 8.05-8.11 (m, 2H), 7.88 (d, J=8.69 Hz, 1H), 7.80-7.86 (m, 1H), 7.43 (t, J=8.10 Hz, 1H), 7.15-7.21 (m, 1H), 3.99 (s, 3H), 2.69-2.79 (m, 1H), 1.96 (s, 1H), 0.83-0.98 (m, 4H).

### N-(5-(4-((3-chlorophenyl)amino)quinazolin-6-yl)-2-methoxypyridin-3-yl)-2-morpholinoethane-1-sulfonamide, MOL-205

In two separate reaction vessels: To each of the two reaction vessels consisting of a suspension of 6-(5-amino-6-methoxypyridin-3-yl)-N-(3-chlorophenyl)quinazolin-4-amine (300 mg, 0.79 mmol) and N-methylmorpholine (239 mg, 2.37 mmol) in dichloromethane (20 mL) was slowly added 2-chloroethanesulfonyl chloride (258 mg, 1.58 mmol). After 4 hours of stirring at room temperature 2-chloroethanesulfonyl chloride (280 mg, 1.7 mmol) and N-methylmorpholine (276 mg, 2.7 mmol) were added. After about 3 hours, to both reaction mixtures was added morpholine (241 mg, 2.8 mmol) and the reaction was stirred at room temperature overnight. The reaction mixtures were combined and loaded directly onto a 120 gram silica column that had been equilibrated with ethyl acetate-heptane (8:2 v/v) and using enough dichloromethane to help keep the crude material in solution. The silica column was eluted with a gradient of methanol-ethyl acetate (0:100 v/v to 10:90 v/v). The resulting precipitate from the partial concentration of the proper fractions was filtered to give the title compound as a near white solid (125 mg, 28%); TLC R_{f} 0.13 (solvent system: ethyl acetate); MS (ES−API+) m/z 555 (M+1), 557 (Cl isotope), (ES−API−) m/z 553 (M−1), 555 (Cl isotope); ¹H NMR (400 MHz, DMSO-d₆) □ 9.97 (s, 1H), 9.47 (br s, 1H), 8.79 (s, 1H), 8.65 (s, 1H), 8.51 (d, J=2.01 Hz, 1H), 8.15 (dd, J=1.46, 8.69 Hz, 1H), 8.05-8.12 (m, 2H), 7.88 (d, J=8.69 Hz, 1H), 7.80-7.86 (m, 1H), 7.84 (dd, J=1.88, 8.19 Hz, 1H), 7.43 (t, J=8.14 Hz, 1H), 7.18 (dd, J=2.01, 7.96 Hz, 1H), 3.99 (s, 3H), 3.49 (t, J=4.48 Hz, 4H), 3.34-3.42 (m, 2H), 2.76 (br t, J=7.18 Hz, 2H), 2.37 (m, 4H).

### N-(5-(4-((3-chlorophenyl)amino)quinazolin-6-yl)-2-methoxypyridin-3-yl)-4-methylpiperazine-1-sulfonamide, MOL-207

To a mixture consisting of 6-(5-amino-6-methoxypyridin-3-yl)-N-(3-chlorophenyl)quinazolin-4-amine (25 mg, 0.07 mmol) in pyridine (0.5 mL) was added 4-methylpiperazine-1-sulfonyl chloride (40 mg, 0.20 mmol). The reaction mixture was stirred at 40° C. overnight, cooled to room temperature and set idle for 44 days. The mixture was filtered, washed with 2 mL of methanol and dried under high vacuum at room temperature to give the title compound (13 mg, 34%) as a solid; MS (ES−API+) m/z 540.1 (M+1), 542.1 (Cl isotope), (ES−API−) m/z 538.0 (M−1), 540.0 (Cl isotope); ¹H NMR (400 MHz, DMSO-d₆ and D20) □ 8.80 (s, 1H), 8.64 (s, 1H), 8.53 (d, J=1.95 Hz, 1H), 8.12-8.20 (m, 1H), 8.10 (d, J=1.95 Hz, 1H), 8.03 (s, 1H), 7.90 (d, J=8.99 Hz, 1H), 7.80 (br d, J=7.82 Hz, 1H), 7.43 (t, J=8.01 Hz, 1H), 7.19 (br d, J=8.21 Hz, 1H), 3.31-3.50 (m, 4H), 3.00 (br t, J=11.14 Hz, 2H), 2.75 (s, 3H), 2.67 (br t, J=12.51 Hz, 2H).

### 6-(5-amino-6-chloropyridin-3-yl)-N-(3-chloro-4-fluorophenyl)quinazolin-4-amine (11H), MOL-210

A mixture consisting of 6-bromo-N-(3-chlorophenyl)quinazolin-4-amine—HCl (700 mg 1.80 mmol), 2-methoxy-5-(4,4,5,5-tetramethyl-1,3,2-dioxaborolan-2-yl)pyridin-3-amine (467 mg, 1.80 mmol) and 1.4M K₂CO₃ (5.1 mL) in 15 mL of 1,4-dioxane was degassed (vacuum/nitrogen, 3 times). To the reaction mixture was added SiliCat DPP-Pd (300 mg, 0.26 mmol/g loading). The reaction mixture was sealed and heated at 100° C. for 10 minutes in a Biotage Emrys Optimizer microwave. The reaction mixture was cooled, the aqueous phase removed and the remaining organic phase was filtered through a glass frit. The solids were washed with methanol. The filtrate was concentrated under reduced pressure. The residue was dissolved in a mix of ethyl acetate, methanol, dichloromethane, and heptane and was applied to a 120 g silica column and it was eluted with a gradient of 35:65 to 75:25 ethyl acetate-heptane to give 399 mg (55%) of the title compound as a solid; MS (ES−API+) m/z 400.0 (M+1), 402.0 (Cl isotope), (ES−API−) m/z 397.9 (M−1), 400.0 (Cl isotope); ¹H NMR (400 MHz, DMSO-d₆) □ 10.02 (s, 1H), 8.78 (s, 1H), 8.64 (s, 1H), 8.18 (dd, J=2.61, 6.91 Hz, 1H), 8.04-8.12 (m, 2H), 7.88 (d, J=8.69 Hz, 1H),

7.80-7.86 (m, 1H), 7.50 (d, J=2.20 Hz, 1H), 7.41-7.48 (t, 1H), 5.74 (s, 2H).

### N-(2-chloro-5-(4-((3-chloro-4-fluorophenyl)amino)quinazolin-6-yl)pyridin-3-yl)methanesulfonamide, MOL-211

To a stirring room temperature mixture consisting of 6-(5-amino-6-chloropyridin-3-yl)-N-(3-chloro-4-fluorophenyl)quinazolin-4-amine (300 mg, 0.75 mmol) in 3 mL of pyridine was added two portions of methanesulfonyl chloride (92 mg, 0.6 mmol (2×)) 4 hours apart. The reaction mixture was then stirred overnight. To the reaction mixture was added 2N NaOH (1.0 mL, 2 mmol) and it was stirred for 30 minutes. The reaction mixture was diluted with a saturated solution of ammonium chloride and 1 mL of 1N HCl (pH=9). The mixture was extracted with ethyl acetate. The organic phase was washed with a saturated solution of ammonium chloride then brine, dried over magnesium sulfate, filtered, and concentrated. The solid residue was chromatographed on an 80 g column of silica eluted with a gradient of 7:3 ethyl acetate-heptane to 100% ethyl acetate. The solid material obtained from the proper fractions was triturated under ethyl acetate (4 mL) and methanol (2 mL), filtered, and dried in high vacuum to give 167 mg (46%, purity 95%) of the title compound; MS (ES−API+) m/z 478.0 (M+1), 480.0 (Cl isotope), (ES−API−) m/z 476.0 (M−1), 478.0 (Cl isotope); ¹H NMR (400 MHz, DMSO-d₆) □ 10.05 (br s, 1H), 9.96 (br s, 1H), 8.88 (s, 1H), 8.80 (d, J=2.10 Hz, 1H), 8.67 (s, 1H), 8.28 (d, J=2.10 Hz, 1H),

8.22-8.27 (m, 1H), 8.18 (dd, J=2.38, 6.86 Hz, 1H), 7.93 (d, J=8.69 Hz, 1H), 7.77-7.89 (m, 1H), 7.49 (t, J=9.06 Hz, 1H), 3.19 (s, 3H).

### 6-(3-amino-4-chlorophenyl)-N-(3-chloro-4-fluorophenyl)quinazolin-4-amine (11J), MOL-212

A mixture consisting of 6-bromo-N-(3-chlorophenyl)quinazolin-4-amine—HCl (350 mg 0.90 mmol), 2-chloro-5-(4,4,5,5-tetramethyl-1,3,2-dioxaborolan-2-yl)aniline (251 mg, 0.99 mmol) and 1.4M K₂CO₃ (2.8 mL) in 10 mL of 1,4-dioxane was degassed (vacuum/nitrogen, 3 times). To the reaction mixture was added SiliCat DPP-Pd (150 mg, 0.26 mmol/g loading). The reaction mixture was sealed and heated at 100° C. for 12 minutes in a Biotage Emrys Optimizer microwave. To the reaction mixture was added additional 2-chloro-5-(4,4,5,5-tetramethyl-1,3,2-dioxaborolan-2-yl)aniline (40 mg, 0.16 mmol) and SiliCat DPP-Pd (30 mg). The reaction mixture was heated again at 100° C. for 6 minutes and cooled. The aqueous phase was removed and the remaining organic phase was filtered through a glass frit. The solids were washed with methanol. The filtrate was concentrated under reduced pressure. The residue was applied to a 120 g silica column and eluted with a gradient of 35:65 to 75:25 ethyl acetate-heptane to give 126 mg (35%) of the title compound as a colorless solid; MS (ES−API+) m/z 399.0 (M+1), 401.0 (Cl isotope), (ES−API−) m/z 397.0 (M−1), 399.0 (Cl isotope); ¹H NMR (400 MHz, DMSO-d₆) □ 10.04 (s, 1H), 8.73 (d, J=1.74 Hz, 1H), 8.64 (s, 1H), 8.20 (dd, J=2.65, 6.86 Hz, 1H), 8.06 (dd, J=1.83, 8.69 Hz, 1H), 7.83-7.90 (m, 2H), 7.47 (t, J=9.10 Hz, 1H), 7.37 (d, J=8.23 Hz, 1H), 7.23 (d, J=2.20 Hz, 1H), 7.03 (dd, J=2.20, 8.23 Hz, 1H), 5.51 (s, 2H).

### N-(2-chloro-5-(4-((3-chloro-4-fluorophenyl)amino)quinazolin-6-yl)phenyl)methanesulfonamide, MOL-213

To a stirring room temperature mixture consisting of 6-(3-amino-4-chlorophenyl)-N-(3-chloro-4-fluorophenyl)quinazolin-4-amine (126 mg, 0.32 mmol) in 1.5 mL of pyridine was added methanesulfonyl chloride (45 mg, 0.39 mmol). The reaction mixture was then stirred overnight. To the reaction mixture was added 2N NaOH (1.0 mL, 2 mmol) and it was stirred for 10 minutes. The reaction mixture was diluted with a saturated solution of ammonium chloride and 0.5 mL of 1N HCl. The mixture was extracted with ethyl acetate. The organic phase was washed with a saturated solution of ammonium chloride then brine, dried over magnesium sulfate, filtered, and concentrated. The solid residue was triturated under ethyl acetate (4 mL) and methanol (2 mL) for 20 hours, filtered, and dried in high vacuum to give 83 mg (54%, purity 97%) of the title compound; MS (ES−API+) m/z 477.0 (M+1), 479.0 (Cl isotope), (ES−API−) m/z 474.9 (M−1), 477.0 (Cl isotope); ¹H NMR (400 MHz, DMSO-d₆) □ 10.05 (s, 1H), 9.67 (s, 1H), 8.79 (d, J=1.56 Hz, 1H), 8.64 (s, 1H), 8.12-8.19 (m, 2H), 7.84-7.92 (m, 2H), 7.81 (ddd, J=2.74, 4.30, 9.06 Hz, 1H), 7.73-7.78 (m, 1H), 7.68-7.73 (m, 1H), 7.46 (t, J=9.10 Hz, 1H), 3.10 (s, 3H).

### 6-bromo-N-(3-chloro-4-(pyridin-2-ylmethoxy)phenyl)quinazolin-4-amine hydrochloride

A mixture consisting of 6-bromo-4-chloroquinazoline (1.0 g, 4.1 mmol) and 3-chloro-4-(pyridin-2-ylmethoxy)aniline (1.15 g, 4.9 mmol) in 40 mL of 1,4-dioxane was heated at 80° C. overnight. The reaction mixture was cooled to room temperature, diluted with 20 mL of diethyl ether and filtered. The solids were dried in high vacuum to give 1.98 g (100%, purity 90%) of the title compound; MS (ES−API+) m/z 441.0 (M+1) 443.0 (Cl/Br isotope), (ES−API−) m/z 439.0 (M−1) 441.0 (Cl/Br isotope); ¹H NMR (400 MHz, DMSO-d₆) □ 11.49 (br s, 1H), 9.15 (d, J=1.92 Hz, 1H), 8.91 (s, 1H), 8.61 (d, J=5.03 Hz, 1H), 8.20 (dd, J=2.01, 8.87 Hz, 1H), 7.90-7.96 (m, 2H), 7.87 (d, J=8.97 Hz, 1H), 7.59-7.69 (m, 2H), 7.41 (dd, J=4.99, 6.54 Hz, 1H), 7.34 (d, J=9.06 Hz, 1H), 5.34 (s, 2H).

### 6-(5-amino-6-chloropyridin-3-yl)-N-(3-chloro-4-(pyridin-2-ylmethoxy)phenyl)quinazolin-4-amine

A mixture consisting of 6-bromo-N-(3-chloro-4-(pyridin-2-ylmethoxy)phenyl)quinazolin-4-amine—HCl (900 mg, 1.88 mmol), 2-methoxy-5-(4,4,5,5-tetramethyl-1,3,2-dioxaborolan-2-yl)pyridin-3-amine (832 mg, 4.3 mmol) and 2.0M K₂CO₃ (4.4 mL) in 15 mL of 1,4-dioxane was degassed (vacuum/nitrogen, 3 times). To the reaction mixture was added SiliCat DPP-Pd (450 mg, 0.26 mmol/g loading). The reaction mixture was sealed and heated at 100° C. for 20 minutes in a Biotage Emrys Optimizer microwave. The reaction mixture was cooled, the aqueous phase was removed, and the mixture was filtered through a glass frit. The solids were washed with methanol then hot methanol. The filtrate was concentrated under reduced pressure. The residue was diluted with methanol and ethyl acetate, concentrated under reduced pressure to give a solid. The solid was suspended in 20 mL of ethyl acetate. The addition of 2 mL of methanol resulted in a homogeneous solution. The slow addition of 15 mL of heptane resulted in precipitation of a solid and the suspension was stirred for 30 minutes and filtered and dried to give 530 mg of the title compound as a light green/brown solid. The mother liquor was set overnight and produced a precipitate that was filtered to give 300 mg of additional pale green solid. Total: 880 mg (96%, purity 90%); MS (ES−API+) m/z 489.1 (M+1), 491.0 (Cl isotope), (ES−API−) m/z 487.0 (M−1), 489.0 (Cl isotope); ¹H NMR (400 MHz, DMSO-d₆) □ 9.84-10.20 (br s, 1H), 8.79 (br s, 1H), 8.58 (br d, J=4.39 Hz, 1H), 8.50 (br s, 1H), 8.07 (br d, J=1.65 Hz, 1H), 7.95-8.04 (m, 2H), 7.82-7.90 (m, 1H), 7.78 (br d, J=8.88 Hz, 1H), 7.69 (br d, J=7.69 Hz, 1H), 7.53-7.60 (m, 2H), 7.30-7.38 (m, 1H), 7.24 (br d, J=8.78 Hz, 1H), 5.73 (s, 2H), 5.27 (s, 2H).

### N-(2-chloro-5-(4-((3-chloro-4-(pyridin-2-ylmethoxy)phenyl)amino)quinazolin-6-yl)pyridin-3-yl)methanesulfonamide, MOL-215

To a stirring room temperature mixture consisting of 6-(5-amino-6-chloropyridin-3-yl)-N-(3-chloro-4-(pyridin-2-ylmethoxy)phenyl)quinazolin-4-amine (300 mg, 0.61 mmol) in 3.5 mL of pyridine was added two portions of methanesulfonyl chloride (140 mg, 2.45 mmol (2×)) 2 hours apart. The reaction mixture was stirred overnight. To the reaction mixture was added 2N NaOH (1.5 mL, 3 mmol). At 0.5 hour an additional amount of 2N NaOH (0.5 mL, 1 mmol) was added and stirring was continued for another 0.5 hour. To the reaction was added 2N NaOH (2.0 mL, 4 mmol) and after 30 minutes the reaction (hydrolysis) appeared to be complete by TLC. The reaction mixture was diluted with a saturated solution of sodium bicarbonate and ethyl acetate and shaken in a separatory funnel. To the mixture was added water, brine, methanol and isopropanol (25 mL) to brake the emulsion. The mixture was extracted twice with ethyl acetate. The combined organic phase was washed with brine, dried over magnesium sulfate, filtered, and concentrated under reduced pressure. The residue was taken up in toluene and concentrated. The solid was taken up in methanol/ethyl acetate, filtered and the filtrate was applied to a 120 g silica column eluted with 9:1 ethyl acetate-heptane to 100% ethyl acetate to 1:9 methanol-ethyl acetate to give 140 mg (40%, purity 97%) of the title compound as a yellow solid; MS (ES−API+) m/z 567.0 (M+1), 569.1 (Cl isotope), (ES−API−) m/z 565.0 (M−1), 567.0 (Cl isotope); ¹H NMR (400 MHz, DMSO-d₆) □ 9.96 (br s, 2H), 8.88 (s, 1H), 8.81 (d, J=2.10 Hz, 1H), 8.62 (s, 1H), 8.60 (br d, J=4.39 Hz, 1H), 8.29 (d, J=2.01 Hz, 1H), 8.24 (br d, J=8.78 Hz, 1H), 8.02 (d, J=2.47 Hz, 1H), 7.86-7.93 (m, 2H), 7.72 (dd, J=2.38, 8.87 Hz, 1H), 7.59 (d, J=7.87 Hz, 1H), 7.34-7.41 (m, 1H), 7.31 (d, J=9.06 Hz, 1H), 5.31 (s, 2H), 3.20 (s, 3H).

### 6-(5-aminopyridin-3-yl)-N-(3-chlorophenyl)quinazolin-4-amine, MOL-310

A mixture consisting of 6-bromo-N-(3-chlorophenyl)quinazolin-4-amine—HCl (500 mg 1.49 mmol), 5-(4,4,5,5-tetramethyl-1,3,2-dioxaborolan-2-yl)pyridin-3-amine (274 mg, 1.24 mmol) and 2.0M K₂CO₃ (3.1 mL) in 15 mL of 1,4-dioxane was degassed (vacuum/nitrogen, 3 times). To the reaction mixture was added SiliCat DPP-Pd (60 mg, 0.26 mmol/g loading). The reaction mixture was sealed and heated at 95° C. for 1.25 hours. To the reaction was added 5-(4,4,5,5-tetramethyl-1,3,2-dioxaborolan-2-yl)pyridin-3-amine (90 mg, 0.41 mmol) and heated again at 95° C. overnight. The reaction mixture was cooled and filtered through a glass frit. The solids were washed with ethanol. The filtrate was concentrated under reduced pressure. The residue was chromatographed on a 40 g silica column using the dry loading method and eluted with a gradient of 1:99 to 15:85 methanol-ethyl acetate to give 126 mg (24%, purity 97.4%) of the title compound; MS (ES−API+) m/z 348.0 (M+1), 350.0 (Cl isotope), (ES−API−) m/z 346.0 (M−1), 348.0 (Cl isotope); ¹H NMR (400 MHz, DMSO-d₆) □ 9.99 (s, 1H), 8.81 (d, J=1.65 Hz, 1H), 8.66 (s, 1H), 8.24 (d, J=1.92 Hz, 1H), 8.05-8.14 (m, 2H), 7.99 (d, J=2.47 Hz, 1H), 7.81-7.93 (m, 2H), 7.42 (t, J=8.10 Hz, 1H), 7.29 (t, J=2.29 Hz, 1H), 7.18 (d, J=8.18 Hz, 1H), 5.48 (s, 2H).

### 6-(5-(1H-tetrazol-1-yl)pyridin-3-yl)-N-(3-chlorophenyl)quinazolin-4-amine, MOL-311

To a mixture consisting of 6-(5-aminopyridin-3-yl)-N-(3-chlorophenyl)quinazolin-4-amine (100 mg, 0.29 mmol) in 2 mL of acetic acid was added trimethylorthoformate (92 mg, 0.86 mmol) and sodium azide (56 mg, 0.86 mmol). The reaction mixture was heated at 80° C. for 4 hours. The reaction was quenched with a saturated solution of sodium bicarbonate and extracted with ethyl acetate. The organic phase was dried over magnesium sulfate, filtered, and concentrated under reduce pressure to a yellow solid. The solid was triturated under 4:1 dichloromethan-ethyl acetate followed by trituration under dichloromethane-ethyl acetate-tetrahydrofuran and filtered to give 40 mg (34, purity 91%) of the title compound; MS (ES-API+) m/z 401.1 (M+1), 403.0 (Cl isotope), (ES−API−) m/z 399.0 (M−1), 401.0 (Cl isotope); ¹H NMR (400 MHz, DMSO-d₆) □ 10.26 (s, 1H), 10.00 (s, 1H), 9.33 (s, 1H), 9.19 (d, J=2.10 Hz, 1H), 9.01 (s, 1H), 8.76-8.88 (m, 1H), 8.69 (s, 1H), 8.38 (d, J=8.60 Hz, 1H), 8.08 (s, 1H), 7.95 (d, J=8.60 Hz, 1H), 7.84 (br d, J=8.23 Hz, 1H), 7.44 (t, J=8.10 Hz, 1H), 7.20 (d, J=7.96 Hz, 1H).

### 5-(4-((3-chlorophenyl)amino)quinazolin-6-yl)nicotinonitrile, MOL-312

A mixture consisting of 6-bromo-N-(3-chlorophenyl)quinazolin-4-amine—HCl (500 mg 1.49 mmol), 5-(4,4,5,5-tetramethyl-1,3,2-dioxaborolan-2-yl)nicotinonitrile (286 mg, 1.24 mmol) and 2.0M K₂CO₃ (3.1 mL) in 15 mL of 1,4-dioxane was degassed (vacuum/nitrogen, 3 times). To the reaction mixture was added SiliCat DPP-Pd (70 mg, 0.26 mmol/g loading). The reaction mixture was sealed and heated at 95° C. for 4 hours. The reaction mixture was cooled and filtered through a glass frit. The solids were washed with ethanol. The filtrate was concentrated under reduced pressure. Toluene was added to the residue and concentrated under reduced pressure. The residue was chromatographed on a 40 g silica column using the dry loading method and eluted with a gradient of 25:75 to 95:5 ethyl acetate-dichloromethane followed by the addition of 5% methanol up to 9% methanol in the 95:5 ethyl acetate-dichloromethane system to give 147 mg (33%) of the title compound as a pale yellow solid; MS (ES−API+) m/z 358.0 (M+1), 360.0 (Cl isotope), (ES−API−) m/z 356.0 (M−1), 358.0 (Cl isotope); ¹H NMR (400 MHz, DMSO-d₆) □ 9.95 (s, 1H), 9.41 (d, J=2.20 Hz, 1H), 9.08 (d, J=1.83 Hz, 1H), 8.94 (d, J=1.74 Hz, 1H), 8.82 (t, J=2.10 Hz, 1H), 8.69 (s, 1H), 8.34 (dd, J=1.83, 8.69 Hz, 1H), 8.07 (t, J=1.97 Hz, 1H), 7.92 (d, J=8.69 Hz, 1H), 7.84 (dd, J=1.19, 8.23 Hz, 1H), 7.44 (t, J=8.14 Hz, 1H), 7.20 (dd, J=1.33, 7.91 Hz, 1H).

### 6-(5-(1H-tetrazol-5-yl)pyridin-3-yl)-N-(3-chlorophenyl)quinazolin-4-amine, MOL-313

A mixture consisting of 5-(4-((3-chlorophenyl)amino)quinazolin-6-yl)nicotinonitrile (50 mg, 0.14 mmol), sodium azide (18 mg, 0.28 mmol), ammonium chloride (15 mg, 0.28 mmol) and lithium chloride (1.2 mg) was heated at 100° C. overnight. The reaction was cooled, toluene was added and the mixture was concentrated under reduced pressure to less than 1 mL. To the residue was added a mixture of 0.5:5:95 acetic acid-methanol-dichloromethane and the mixture was filtered. The filtrate was applied to a 25 g silica column which was eluted with a gradient of 0.5:10:90 to 0.5:40:60 acetic acid-methanol-dichloromethane to give XX mg (XX %, purity 96%) of the title compound as a solid; MS (ES−API+) m/z 401.0 (M+1), 403.1 (Cl isotope); ¹H NMR (400 MHz, DMSO-d₆) □ 10.25 (br s, 1H), 9.18 (s, 1H), 9.03 (s, 1H), 9.01 (s, 1H), 8.71 (s, 1H), 8.67 (s, 1H), 8.29 (d, J=8.69 Hz, 1H), 8.13 (s, 1H), 7.92 (d, J=8.69 Hz, 1H), 7.88 (br d, J=8.42 Hz, 1H), 7.42 (t, J=8.10 Hz, 1H), 7.18 (d, J=7.96 Hz, 1H).

### Example 7

This example shows the synthesis procedure for additional quinoline based compounds of the present invention.

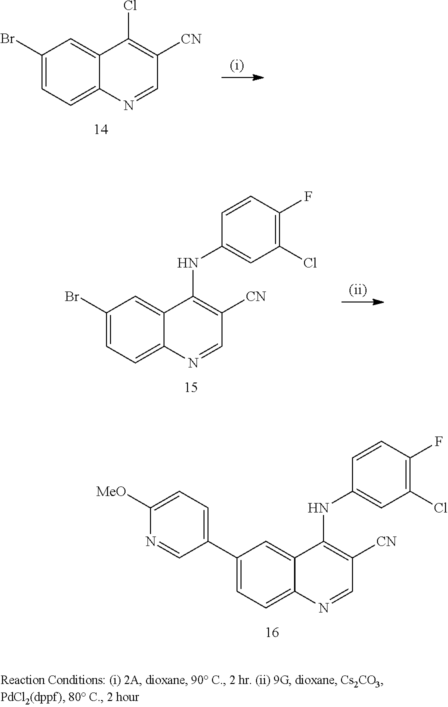 [SMILES: N#Cc1cnc2ccc(Br)cc2c1Cl ; N#Cc1cnc2ccc(Br)cc2c1Nc1ccc(F)c(Cl)c1 ; COc1ccc(-c2ccc3ncc(C#N)c(Nc4ccc(F)c(Cl)c4)c3c2)cn1]

### 4-((3-chloro-4-fluorophenyl)amino)-6-(6-methoxypyridin-3-yl)quinoline-3-carbonitrile, MOL-150

A mixture of 6-bromo-4-chloroquinoline-3-carbonitrile (14, 200 mg, 0.75 mmol) and 3-chloro-4-fluoroaniline (2A, 130 mg, 0.90 mmol) in 4 mL of 1,4-dioxane was heated at 90° C. for 2 hour. The reaction mixture was cooled to room temperature, diluted with diethyl ether, cooled to 0° C. and filtered through fritted glass. The solid was washed with diethyl ether and dried to give 6-bromo-4-((3-chloro-4-fluorophenyl)amino)quinoline-3-carbonitrile (15, 280 mg, 100%) as a dull yellow solid. A solution of 6-bromo-4-((3-chloro-4-fluorophenyl)amino)quinoline-3-carbonitrile (278 mg, 0.77 mmol) and (6-methoxypyridin-3-yl)boronic acid (9G, 118 mg, 0.77 mmol) in 1,4-dioxane (15 mL) and water (1.4 mL) was degassed. To the solution was added cesium carbonate (1.0 g, 3.1 mmol) and [1,1′-bis(diphenylphosphino)ferrocene]dichloropalladium(II) (44 mg). The reaction mixture was heated at 80° C. under N₂ for 2 hours. The reaction mixture was diluted with toluene and the volatiles were removed under vacuum and the crude material was purified by silica gel column chromatography eluting with a gradient of 3/7 to 7/3 ethyl acetate/heptane. The yellow solid was triturated under dichloromethane/diethyl ether, filtered and dried to give 4-((3-chloro-4-fluorophenyl)amino)-6-(6-methoxypyridin-3-yl)quinoline-3-carbonitrile (16, MOL-150, 44 mg, 14%, 100% purity) as a white solid; ¹H NMR (400 MHz, DMSO-d₆) δ 9.95 (s, 1H), 8.75 (d, J=1.9 Hz, 1H), 8.70 (d, J=1.9 Hz, 1H), 8.58 (s, 1H), 8.21 (t, J=6.2 Hz, 2H), 7.99 (d, J=8.4 Hz, 1H), 7.64 (d, J=6.6 Hz, 1H), 7.48 (t, J=8.8 Hz, 1H), 7.3-7.4 (m, 1H), 6.99 (d, J=8.5 Hz, 1H), 3.91 (s, 3H); MS: (ESI⁺ m/z 405.1, ESI⁻ m/z 403.1).

### 6-bromo-4-((4-(pyridin-4-yloxy)phenyl)amino)quinoline-3-carbonitrile hydrochloride, MOL-400

A mixture consisting of 6-bromo-4-chloroquinoline-3-carbonitrile (440 mg, 1.64 mmol) and 4-(pyridin-4-yloxy)aniline (291 mg, 1.56 mmol) in 3 mL of ethoxyethanol was heated at 125° C. for 2 hours in a sealed vessel. The reaction mixture was cooled to room temperature and filtered to give 193 mg of the title compound as a light brown solid. The filtrate was diluted with ethyl acetate and washed with a saturated solution of sodium bicarbonate. The aqueous phase was extracted two time with ethyl acetate. The combined organic phase was washed with brine, dried over magnesium sulfate, filtered, and concentrated under reduced pressure. The residue was chromatographed on 25 g of silica eluted with a gradient of 45:55 ethyl acetate-heptane to 100% ethyl acetate to 2:98 methanol-ethyl acetate to give 160 mg of the title compound as a tan solid. Total: 353 mg (54%,). A sample of the light brown solid was mostly dissolved in 5 mL of 2:8 methanol-dichloromethane and while stirring 15 mL of diethyl ether and 5 mL of heptane were added. The suspension was stirred overnight and filtered. The filtrate was set at room temperature and the crystalline material which formed was filtered to give near white solid (99.9% pure); MS (ES−API+) m/z 417.0 (M+1) 419.0 (Br isotope), (ES−API−) m/z 414.9 (M−1) 417.0 (Br isotope); ¹H NMR (400 MHz, DMSO-d₆) □ 10.03 (br s, 1H), 8.78 (d, J=1.92 Hz, 1H), 8.57 (s, 1H), 8.44-8.51 (m, 2H), 7.97 (dd, J=2.10, 8.87 Hz, 1H), 7.85 (d, J=8.87 Hz, 1H), 7.45 (d, J=8.69 Hz, 2H), 7.21-7.30 (m, 2H), 6.97-7.03 (m, 2H).

### 6-(3-(hydroxymethyl)phenyl)-4-((4-(pyridin-4-yloxy)phenyl)amino)quinoline-3-carbonitrile, MOL-402

A mixture consisting of 6-bromo-4-((4-(pyridin-4-yloxy)phenyl)amino)quinoline-3-carbonitrile hydrochloride (40 mg 0.096 mmol), (3-(hydroxymethyl)phenyl)boronic acid (19 mg, 0.125 mmol) and 2.0M K₂CO₃ (0.24 mL) in 2 mL of 1,4-dioxane and 1 mL of ethanol was degassed (vacuum/nitrogen, 3 times). To the reaction mixture was added SiliCat DPP-Pd (25 mg, 0.26 mmol/g loading). The reaction mixture was sealed and heated at 95° C. for 2 hours. The reaction mixture was cooled and filtered through a glass frit. The solids were washed with ethanol. The filtrate was concentrated under reduced pressure. The residue was triturated under 1.5 mL of methanol and filtered to give 25 mg (58%, purity 98.4%) of the title compound as a solid; MS (ES−API+) m/z 445.2 (M+1), (ES−API−) m/z 443.2 (M−1); ¹H NMR (400 MHz, DMSO-d₆) □ 10.11 (br s, 1H), 8.75-8.88 (m, 1H), 8.54 (s, 1H), 8.44 (d, J=5.37 Hz, 2H), 8.17 (dd, J=1.69, 8.65 Hz, 1H), 7.99 (d, J=8.60 Hz, 1H), 7.83 (s, 1H), 7.76 (br d, J=7.96 Hz, 1H), 7.43-7.54 (m, 3H), 7.38 (d, J=7.50 Hz, 1H), 7.26 (d, J=8.78 Hz, 2H), 6.93-7.02 (m, 2H), 5.28 (t, J=5.67 Hz, 1H), 4.60 (d, J=5.58 Hz, 2H).

### N-(5-(3-cyano-4-((4-(pyridin-4-yloxy)phenyl)amino)quinolin-6-yl)pyridin-3-yl)methanesulfonamide, MOL-401

A mixture consisting of 6-bromo-4-((4-(pyridin-4-yloxy)phenyl)amino)quinoline-3-carbonitrile hydrochloride (80 mg 0.19 mmol), N-(5-(4,4,5,5-tetramethyl-1,3,2-dioxaborolan-2-yl)pyridin-3-yl)methanesulfonamide (74 mg, 0.25 mmol) and 2.0M K₂CO₃ (0.47 mL) in 4 mL of 1,4-dioxane and 2 mL of ethanol was degassed (vacuum/nitrogen, 3 times). To the reaction mixture was added SiliCat DPP-Pd (50 mg, 0.26 mmol/g loading). The reaction mixture was sealed and heated at 95° C. for 2 hours. The reaction mixture was cooled and filtered through a glass frit. The solids were washed with ethanol. The filtrate was concentrated under reduced pressure. The residue was chromatographed on a 12 g silica column eluted with a gradient of 100% ethyl acetate to 25:75 methanol-ethyl acetate to give 65 mg of a yellow solid. The solid was triturated under a mix of methanol-ethyl acetate-dichloromethane and filtered to give 32 mg of the title compound as a yellow solid (33%, purity 91%); MS (ES−API+) m/z 509.1 (M+1), (ES−API−) m/z 507.0 (M−1); ¹H NMR (400 MHz, DMSO-d₆) □ 10.14 (s, 2H), 8.86 (s, 2H), 8.58 (s, 1H), 8.44-8.51 (m, 3H), 8.13-8.22 (m, 1H), 8.13-8.22 (m, 1H), 8.05 (br d, J=8.60 Hz, 1H), 8.00 (t, J=2.10 Hz, 1H), 7.49 (br d, J=8.33 Hz, 2H), 7.27 (d, J=8.69 Hz, 2H), 6.98 (d, J=5.37 Hz, 2H), 3.13 (s, 3H).

### 6-(3-hydroxyphenyl)-4-((4-(pyridin-4-yloxy)phenyl)amino)quinoline-3-carbonitrile, MOL-403

A mixture consisting of 6-bromo-4-((4-(pyridin-4-yloxy)phenyl)amino)quinoline-3-carbonitrile hydrochloride (80 mg 0.19 mmol), (3-hydroxyphenyl)boronic acid (34 mg, 0.25 mmol) and 2.0M K₂CO₃ (0.47 mL) in 4 mL of 1,4-dioxane and 2 mL of ethanol was degassed (vacuum/nitrogen, 3 times). To the reaction mixture was added SiliCat DPP-Pd (50 mg, 0.26 mmol/g loading). The reaction mixture was sealed and heated at 95° C. for 2 hours. The reaction mixture was cooled and filtered through a glass frit. The solids were washed with ethanol. The filtrate was diluted with toluene and concentrated under reduced pressure. The residue was chromatographed on a 12 g silica column eluted with a gradient of 8:2 ethyl acetate-dichloromethane to 100% ethyl acetate then to 1:9 methanol-ethyl acetate to give 15 mg (18%, purity 95.9%) of the title compound; MS (ES−API+) m/z 431.1 (M+1), (ES−API−) m/z 429.1 (M−1); ¹H NMR (400 MHz, DMSO-d₆) □ 9.91-10.48 (br s, 1H), 9.47-9.91 (br s, 1H), 8.75 (s, 1H), 8.38-8.52 (m, 3H), 8.07 (br d, J=7.96 Hz, 1H), 7.92 (br d, J=8.69 Hz, 1H), 7.41 (br d, J=8.05 Hz, 2H), 7.25-7.36 (m, 3H), 7.22 (br d, J=8.60 Hz, 2H), 6.96 (d, J=6.13 Hz, 2H), 6.82 (br d, J=7.23 Hz, 1H).

### 6-bromo-4-((3-chloro-4-fluorophenyl)amino)quinoline-3-carbonitrile hydrochloride

A mixture consisting of 6-bromo-4-chloroquinoline-3-carbonitrile (1.0 g, 3.7 mmol) and 3-chloro-4-fluoroaniline (653 mg, 4.5 mmol) in 40 mL of 1,4-dioxane was heated at 80° C. overnight. The reaction mixture was cooled to room temperature, diluted with 20 mL of diethyl ether and filtered. The solids were dried in high vacuum to give 1.36 g (89%) of the title compound; MS (ES−API+) m/z 376.0 (M+1) 378.0 (Cl/Br isotope), (ES−API−) m/z 373.9 (M−1) 375.9 (Cl/Br isotope); ¹H NMR (400 MHz, DMSO-d₆) □ 9.07 (d, J=1.83 Hz, 1H), 8.99 (s, 1H),

8.16 (dd, J=1.92, 8.97 Hz, 1H), 8.00 (d, J=8.88 Hz, 1H), 7.75 (dd, J=2.52, 6.63 Hz, 1H), 7.50-7.59 (m, 1H), 7.43-7.50 (m, 1H).

### 6-(5-amino-6-chloropyridin-3-yl)-4-((3-chloro-4-fluorophenyl)amino)quinoline-3-carbonitrile

A mixture consisting of 6-bromo-4-((3-chloro-4-fluorophenyl)amino)quinoline-3-carbonitrile—HCl (1.2 g, 2.9 mmol), 2-methoxy-5-(4,4,5,5-tetramethyl-1,3,2-dioxaborolan-2-yl)pyridin-3-amine (1.1 g, 4.3 mmol) and 2.0M K₂CO₃ (5.8 mL) in 15 mL of 1,4-dioxane was degassed (vacuum/nitrogen, 3 times). To the reaction mixture was added SiliCat DPP-Pd (650 mg, 0.26 mmol/g loading). The reaction mixture was sealed and heated at 100° C. for 20 minutes in a Biotage Emrys Optimizer microwave. The reaction mixture was cooled and filtered through a glass frit. The solids were washed with methanol then hot methanol. The filtrate was concentrated under reduced pressure. The residue was diluted with toluene, concentrated under reduced pressure then triturated under ethyl acetate for one hour. The solid was filtered and dried to give 2.98 g of the title compound as a solid; MS (ES−API+) m/z 424.0 (M+1), 426.0 (Cl isotope), (ES−API−) m/z 422.0 (M−1), 423.9 (Cl isotope); ¹H NMR (400 MHz, DMSO-d₆) □ 8.52 (s, 1H), 7.88 (d, J=2.01 Hz, 1H), 7.78 (s, 1H), 7.62-7.68 (m, 1H), 7.38-7.50 (m, 2H), 7.08 (t, J=9.24 Hz, 1H), 6.77 (br d, J=6.68 Hz, 1H), 6.60-6.69 (m, 1H), 5.61 (s, 2H).

### N-(2-chloro-5-(4-((3-chloro-4-fluorophenyl)amino)-3-cyanoquinolin-6-yl)pyridin-3-yl)methanesulfonamide, MOL-216

To a stirring room temperature mixture consisting of 6-(5-amino-6-chloropyridin-3-yl)-4-((3-chloro-4-fluorophenyl)amino)quinoline-3-carbonitrile (1.00 g, 2.35 mmol) in 12 mL of pyridine was added two portions of methanesulfonyl chloride (0.54 g, 9.4 mmol (2×)) 2 hours apart. The reaction mixture was stirred a total of 5 hours. To the reaction mixture was added 2N NaOH (5.0 mL, 10 mmol). At 1.5 hours an additional amount of 2N NaOH (3 mL, 6 mmol) was added and stirring was continued for another 0.5 hours. To the dark orange/red reaction mixture was added dropwise 6N HCl (1 mL, 6 mmol). The red/brown reaction mixture was diluted with a saturated solution of sodium chloride and the mixture was extracted twice with ethyl acetate. The combined organic phase was washed with brine, dried over magnesium sulfate, filtered, and concentrated. The solid residue was triturated under a mixture of methanol and ethyl acetate and was filtered. The mother liquor was applied to a 120 g silica column eluted with a gradient of 65:35 ethyl acetate-heptane to 100% ethyl acetate to 15:85 methanol-ethyl acetate. The clean fractions containing product were combined and pale yellow solid was allowed to precipitate. It was filtered and dried to give 30 mg (2.5%) of the title compound. The filtered solid from above was dissolved in hot methanol-ethyl acetate (9:1, 250 mL). To the solution was added 25 g of silica and this mixture was used to dry load the sample on to a 220 g silica column eluted with a gradient of 65:35 ethyl acetate-heptane to 100% ethyl acetate to 1:9 methanol-ethyl acetate. The fractions containing clean product were concentrated under reduced pressure to give 52 mg (4.2%) of the title compound as an off-white solid. MS (ES−API+) m/z 502.0 (M+1), 504.0 (Cl isotope), (ES−API−) m/z 500.0 (M−1), 501.9 (Cl isotope); ¹H NMR (400 MHz, DMSO-d₆) □ 10.06 (s, 1H), 9.93 (br s, 1H), 8.84 (s, 1H), 8.80 (s, 1H), 8.62 (s, 1H), 8.28 (s, 1H), 8.24 (br d, J=9.15 Hz, 1H), 8.05 (br d, J=8.51 Hz, 1H), 7.67 (br d, J=4.67 Hz, 1H), 7.45-7.54 (m, 1H), 7.42 (br s, 1H), 3.16 (s, 3H).

### Example 8: A Role for MOL-211 in Immunotherapy Based Treatment of Cancer

Independent of genomic subtype, there exists strong rationale for investigating the ability of MOL-211 to improve therapeutic outcome in response to immune checkpoint inhibition. MOL-211, which is a pan-PI3K/mTOR inhibitor, potently inhibits three distinct kinase activities, PI3Kγ, PI3Kδ, and mTOR, all implicated in immune suppression. Tumor cells evade host immune recognition by immune checkpoints utilizing the programmed death-1 (PD-1)/programmed death-ligand 1 (PD-L1) pathway to silence the immune system (1). PD-L1 is highly expressed on tumor-infiltrating lymphocytes as well as on the surface of many human solid tumors (2). The interaction of PD-1 and PD-L1 leads to reduction of PTEN activity and SHP2-mediated activation of the PI3K/AKT/mTOR pathway (3). mTOR inhibitors have been reported to increase antitumor activity in response to PD-1 blockade in nonsmall lung cancers (4).

The ability of MOL-211 to inhibit EGFR may also lead to intra-tumoral immune changes that contribute to its anticancer activity. EGFR inhibition has been reported to alter the immune environment in squamous cell carcinomas (5). Inhibition of EGFR has been reported to destabilize PD-L1, enhancing antitumor T-cell immunity and therapeutic efficacy of PD-1 blockade (6).

Collectively, there exists scientific rationale to anticipate that MOL-211 would exert immune-mediated single agent activity in a subset of human cancers. Furthermore, MOL-211 would be expected to augment the efficacy seen with monoclonal antibodies that target immune checkpoint ligands and receptors such as PD-L1 and PD-1.

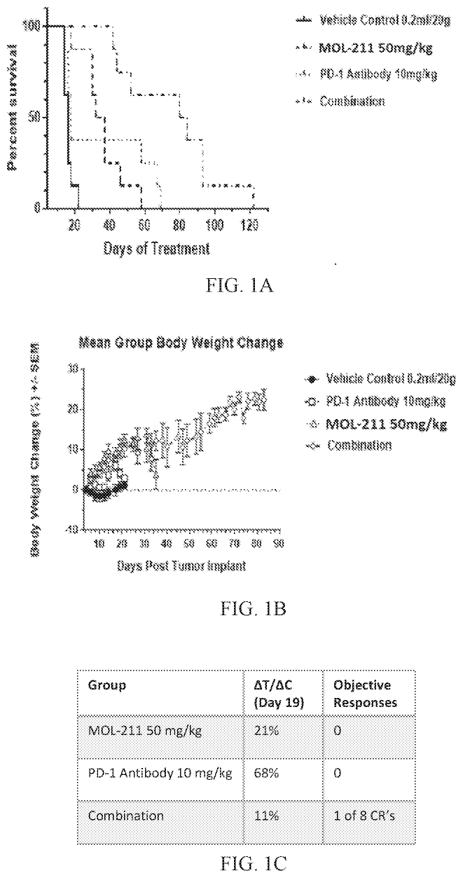

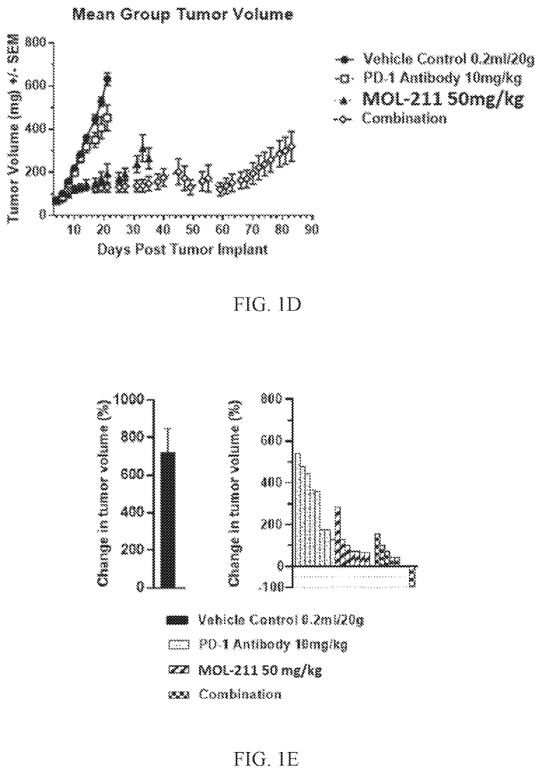

In vivo studies were conducted to evaluate the efficacy of MOL-211 alone and in combination with PD-1 antibody in mice bearing KPC pancreatic tumors (FIG. 1A-FIG. 1E). Mice were treated daily with MOL-211 (50 mg/kg) administered by oral gavage; anti-PD-1 antibody was administered twice a week via IP injection. Mice were analyzed subsequent to tumor implantation for over 90 days using a vehicle control; MOL-211 at 50 mg/kg; anti-PD-1 antibody at 10 mg/kg; and a combination of MOL-211 and anti-PD-1. A Kaplan-Meier survival plot demonstrated a synergistic and non-additive effect in the combination, in which mice receiving the combination treatment survived significantly longer than any of the other groups.

In mice receiving the combination, percent body weight continued to increase post-implantation, whereas mice receiving the control did not increase body weight. Moreover, the combination showed a synergistic effect in body weight gain when compared to either MOL-211 or anti-PD-1 alone (FIG. 1B).

Table 1 summarizes observed treatment effects in the indicated treatment groups. A partial responder (PR) is defined as a tumor that regressed to 50% of the baseline tumor volume. A complete responder (CR) is defined as a tumor below the limits of palpation (40 mg). The ΔT/ΔC ratio is the ratio of tumor volume change (treated/control) from first day of treatment to last day of treatment. Median ILS represents median increase in lifespan.

TABLE 1

| Group | ΔT/ΔC (Day 19) | PRs | CRs | Median ILS |
|---|---|---|---|---|
| MOL-211 50 mg/kg | 21% | 0 | 0 | 82% |
| PD-1 Antibody | 68% | 0 | 0 | 0% |
| 10 mg/kg | | | | |
| Combination | 11% | 0 | 13% (1/8) | 365% |

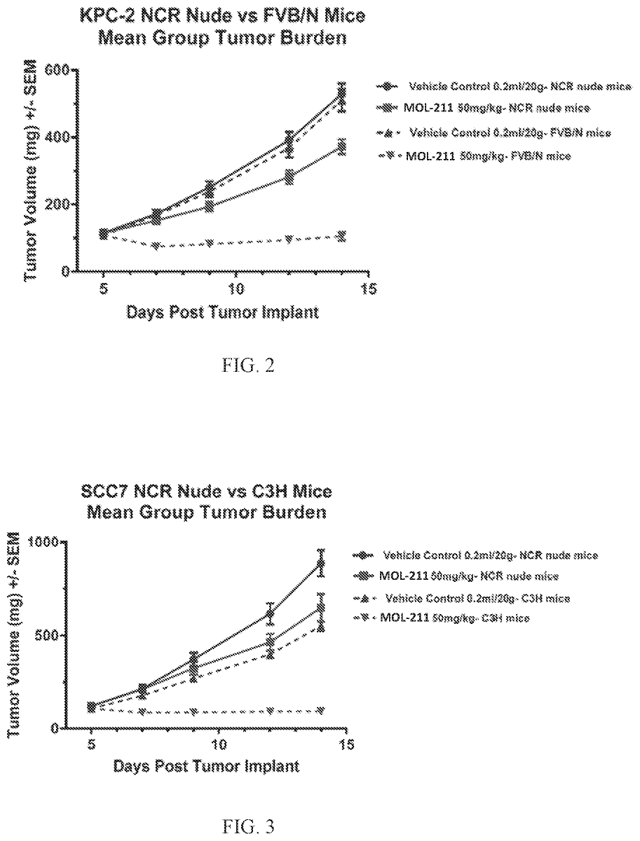

Further evidence supporting the utility of MOL-211 as an immunotherapeutic agent was generated by evaluating KPC pancreatic tumors (FIG. 2) as well as SCC7 head and neck tumors (FIG. 3), comparing efficacy when tumors were grown in immunocompromised (athymic) nude mice versus fully immunocompetent mice (using same strain of mouse in which the original tumor arose).

### Example 9: In Vivo Analyses of MOL-211 Activity as a Single or Combination Treatment for Tumors

This example shows effects of MOL-211 treatment alone or in combination with PD1-antibody in immunocompetent mice bearing xenograft tumors.

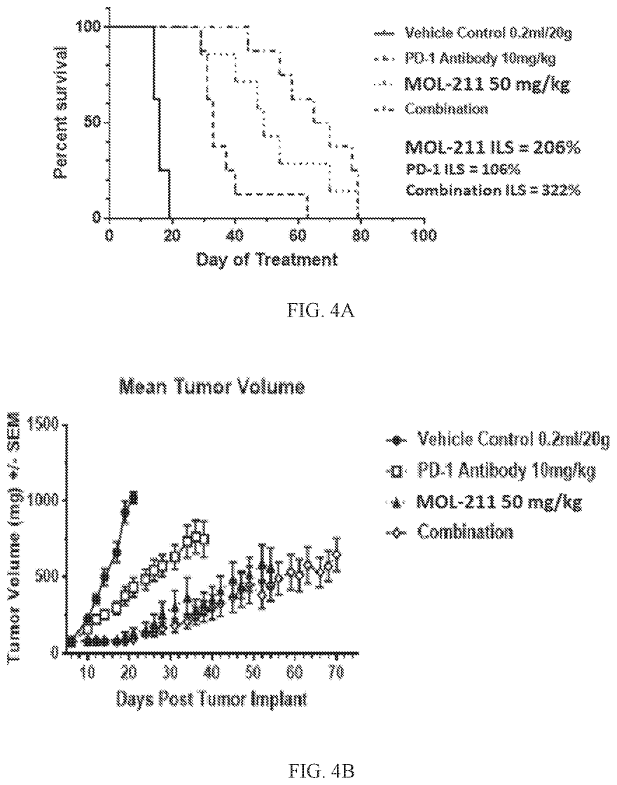

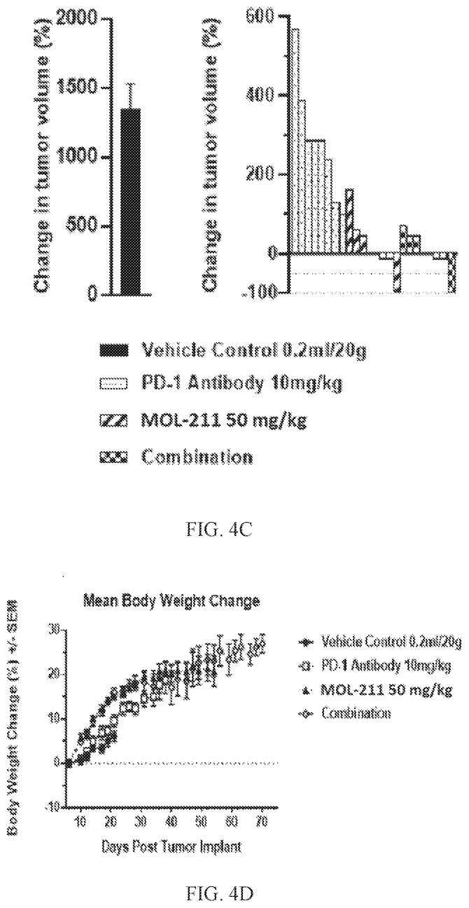

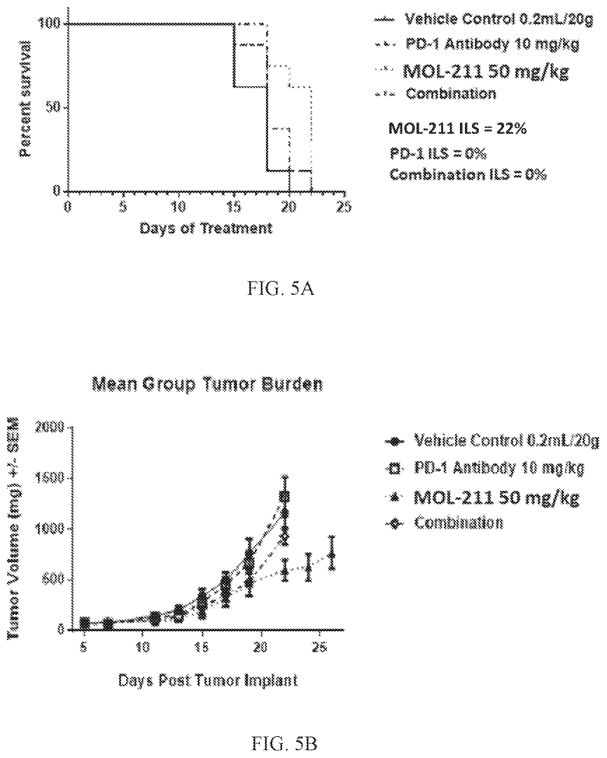

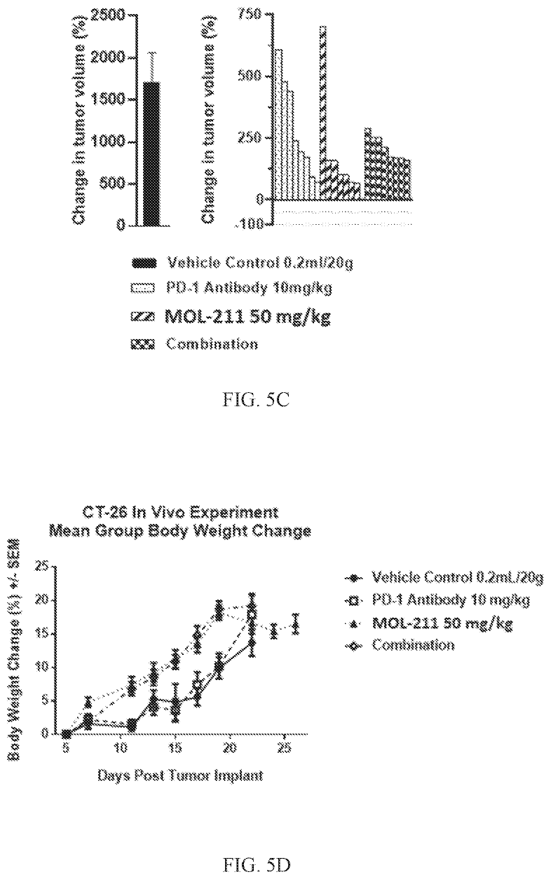

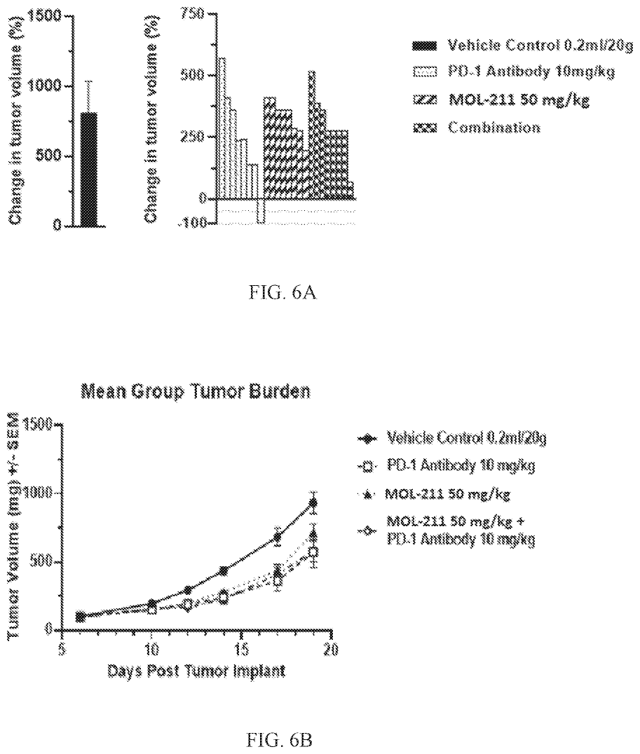

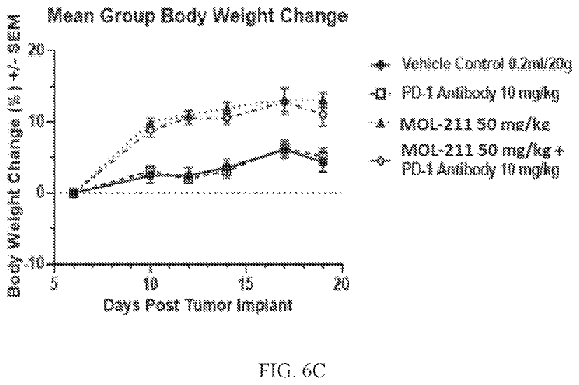

Female 6- to 7-week old C3H mice or BALB/c mice were implanted subcutaneously with 1×10⁶ cultured SCC7 cells or CT-26 (murine colorectal carcinoma) cells into the region of the right axilla. Female 6- to 7-week old BALB/c mice were implanted with 2×10⁶ cultured EMT-6 (murine mammary carcinoma) cells subcutaneously into the mammary fat pad. Mice were randomized into treatment groups and treatments initiated when tumors reached 60 to 100 mg. MOL-211 was administered daily by oral gavage at a dose of 50 mg/kg for the duration of the study and was prepared as a 5 mg/mL solution in 1:2 PG:1% Tween80/Na₃PO₄, based upon individual animal body weight (0.2 mL/20 g). PD-1 antibody was purchased from BioXcell (Lebanon, N.H.) and administered intraperitoneally (IP) at a dose of 10 mg/kg every third day. Subcutaneous tumor volume and body weights were measured three times a week. Tumor volumes were calculated by measuring two perpendicular diameters with calipers and using the formula: tumor volume=(length×width²)/2. Individual animals were dosed until progression, defined as the time at which tumor burden reached 1000 mg. Data are plotted as the effect of treatment on survival (FIG. 4A and FIG. 5A), mean tumor volume (FIG. 4B, FIG. 5B, FIG. 6B) and change in tumor volume from baseline at the start of treatment (FIG. 4C, FIG. 5C, FIG. 6A). Animal body weight was monitored three times weekly (FIG. 4D, FIG. 5D, FIG. 6C).

Kaplan-Meier survival analysis demonstrated additive activity of MOL-211 and PD-1 antibody in mice bearing SCC7 tumors (FIG. 4A). Consistent with survival analysis, MOL-211 monotherapy and combination treatment with MOL-211 and PD-1 antibody was superior to PD-1 antibody monotherapy as measured by a reduction in mean tumor volumes (FIG. 4B).

Neither single agent nor combination treatment with MOL-211 and PD-1 antibody resulted in a therapeutically meaningful increase in survival of mice bearing CT-26 tumors (FIG. 5A).

EMT-6 tumor-bearing mice treated with PD-1 antibody, MOL-211, or the combination did not result in a statistically significant reduction in tumor volume compared to vehicle control (FIG. 6B).

### Example 10: Analysis of PD-L1 Expression in Tumor Cells after Treatment with MOL-211

This example demonstrates reduced PD-L1 protein levels in tumors of mice treated with combined MOL-211 and PD-1 antibody. This example also shows reduced PD-L1 protein levels in tumor cells treated in vitro with MOL-211.

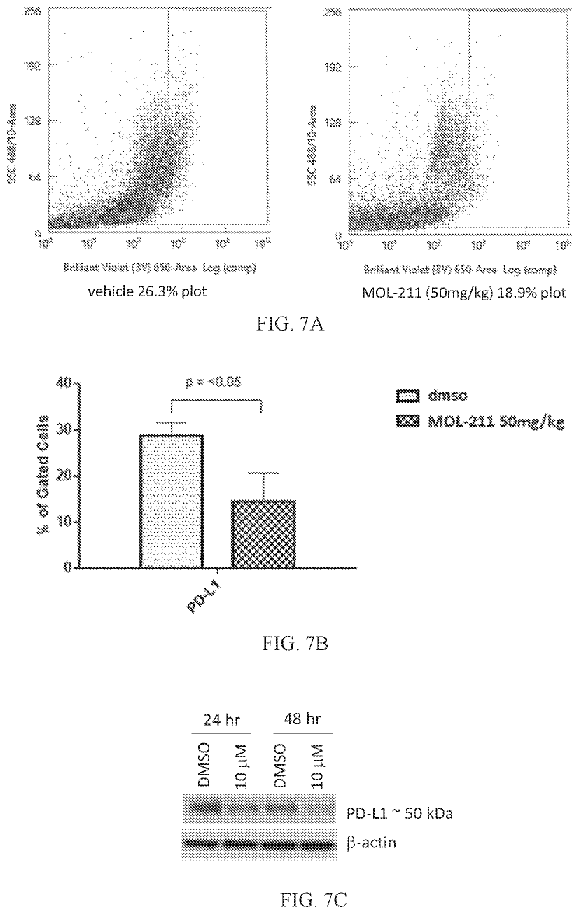

KRAS mutant p53 mutant transgenic (KPC-2) tumors were grown subcutaneously in FVB/N mice and treated as described above in Example 9 with MOL-211 and PD-1 antibody for 15 days. Tumors were excised 24 hours after the last treatment. Single-cell suspensions were prepared by mincing tumors on ice followed by digestion with 1 mg/mL collagenase V (Sigma-Aldrich, St. Louis, Mo.) in HBSS for 1 hour at 37° C. Cells were washed and passed through a 70 μm cell strainer. Red blood cells were removed using RBC Lysis Buffer (Invitrogen, Carlsbad, Calif.) and filtered through a cell strainer. Cells were stained with Zombie NIR live/dead fixable stain (BioLegend, San Diego, Calif.) to exclude dead cells and anti-PD-L1 BV 650 antibody (BioLegend, San Diego, Calif.) to identify PD-L1 positive cells by flow cytometry using a BioRad ZE5 Cell Analyzer. Flow cytometry analysis shows that combination treatment with MOL-211 and PD-1 antibody reduced PD-L1 positive tumor cells (FIG. 7A-FIG. 7B).

KPC-2 cells were also grown in culture and treated for either 24 or 48 hours with DMSO or MOL-211 at a final concentration of 10 micromolar. Cells were harvested and manually homogenized in lysis buffer [25 mmol/L Tris-HCl (pH7.6), 150 mmol/L NaCl, 1% Nonidet P-40, 10% glycerol, 1 mmol/L dithiothreitol, and protease and phosphatase inhibitors], rocked for 30 minutes at 4° C., and centrifuged at 14,000 rpm for 20 min at 4° C. Protein concentration was determined by BioRad Protein Assays and lysates were subsequently subjected to SDS gel electrophoresis. Proteins were transferred to polyvinylidene fluoride membranes and probed with a primary antibody recognizing PD-L1 (Abcam, Cambridge, UK) and beta-actin. After incubation with anti-rabbit HRP-linked secondary antibody (Jackson ImmunoResearch Laboratories, West Grove, Pa.), proteins were detected using chemiluminescence (GE Healthcare, Chicago, Ill.). Western blot analysis shows reduced PD-L1 protein levels in cells treated with MOL-211 compared to DMSO at both 24 and 48 hours (FIG. 7C).

### REFERENCES

Pedoeem, A. et al., Programmed death-1 pathway in cancer and autoimmunity. Clinical Immunology, 2014. 153 (1): p. 145-152.
Francisco, L. M. et al., The PD-1 pathway in tolerance and autoimmunity. Immunological Reviews, 2010. 236: p. 219-42.
Yokosuka, T. et al., Programmed cell death 1 forms negative costimulatory microclusters that directly inhibit T cell receptor signaling by recruiting phosphatase SHP2. The Journal of Experimental Medicine, 2012. 209 (6): p. 1201-1217.
Lastwika, K. J. et al., Control of PD-L1 Expression by Oncogenic Activation of the AKT-mTOR Pathway in Non-Small Cell Lung Cancer. Cancer Research, 2016. 76 (2): p. 227-238.
Mascia F. et al, Cell autonomous or systemic EGFR blockade alters the immune-environment in squamous cell carcinomas. International Journal of Cancer, 2016. 139(11):2593-7.
Li C. et al., Glycosylation and stabilization of programmed death ligand-1 suppresses T-cell activity. Nature Communications, 2016. 7:12632.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

We claim:

1. A combination comprising:
(a). a compound of Formula I, or a pharmaceutically acceptable salt thereof: 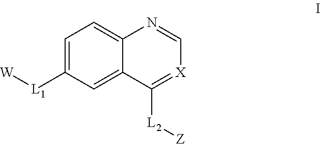
wherein X is N or C—R₄;
L₁ and L₂ are each independently a bond or a C₁-C₆ branched or straight alkylene group, wherein up to three carbon units of said alkylene group are optionally and independently replaced with a bivalent moiety selected from the group consisting of —CO—, —CS—, —CONR—, —CONRNR—, —CO₂—, —OCO—, —NRCO₂—, —O—, —CR═CR—, —C≡C—, —NRCONR—, —OCONR—, —NRNR—, —NRCO—, —S—, —S(O)—, —S(O)₂—, —NR—, —S(O)₂NR—, —NRS(O)₂—, and —NRS(O)₂NR—;
W is selected from the group consisting of halo, 5-10 membered heteroaryl, 5-10 membered heterocyclyl, C₃-C₁₀ carbocyclyl, naphthyl, and phenyl, wherein W is optionally substituted with up to three R₁ substituents;
Z is selected from the group consisting of 5-10 membered heteroaryl, 5-10 membered heterocyclyl, C₃-C₁₀ carbocyclyl, aryl, benzyl, and phenyl, wherein Z is optionally substituted with up to three R₃ substituents;
R₁ is selected from the group consisting of halo, CN, C₁-C₆ alkyl, phenyl, 5-6 membered heteroaryl, 5-6 membered heterocyclyl, C₃-C₆ carbocyclyl, —OR, —CONR₂, —CONRNR₂, —CO₂R, —S(O)₂R, —NR₂, —NRS(O)₂R, —S(O)₂NR₂, and —NRCONR₂, wherein R₁ is optionally substituted with up to two R₂ substituents. R₂ is selected from the group consisting of halo, C₁-C₆ alkyl, C₁-C₆ alkoxy, phenyl, 5-6 membered heteroaryl, 5-6 membered heterocyclyl, C₃-C₆ carbocyclyl, —OH, oxo, —NR₂, wherein each R₂ is optionally and independently substituted with 5-6 membered heterocyclyl;
R₃ is selected from the group consisting of R, halo, —OR, —O(CH₂)ₙR, and —(CH₂)ₙOR;
R₄ is selected from the group consisting of H, halo, C₁-C₄ alkyl, CN, OH, and —COOH;
R is selected from the group consisting of H, C₁-C₄ alkyl, C₂-C₄ alkenyl, C₂-C₄ alkynyl, phenyl, 5-6 membered heteroaryl, 5-6 membered heterocyclyl, C₃-C₆ carbocyclyl, alkylsulfonyl, and —CONH(C₁-C₄ alkyl); and
n is 1, 2, or 3; and
(b). an immune checkpoint modulator,
provided that the compound of Formula I is not

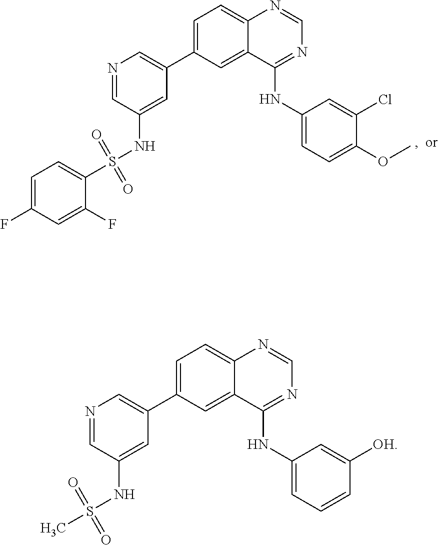

2. The combination of claim 1, wherein R₄ is CN.

3. The combination of claim 1, wherein W is selected from the group consisting of halo, 5-10 membered heteroaryl, and phenyl, wherein W is optionally substituted with up to three R₁ substituents.

4. The combination of claim 3, wherein W is halo, 5-10 membered heteroaryl, or phenyl, wherein W is optionally substituted with one or two R₁ substituents selected from the group consisting of halo, OH, CN, C₁-C₆ alkyl, —OC₁-C₆ alkyl, —NHS(O)₂(C₁-C₆ alkyl), —NHS(O)₂(C₂-C₆ alkenyl), —NHS(O)₂(C₃-C₆ carbocyclyl), —NHS(O)₂ (5-6 membered heterocyclyl), —N(S(O)₂(C₁-C₆ alkyl))₂, —NRS(O)₂-phenyl, —NH₂, —NHC(O)NH(C₁-C₆ alkyl), 5-6 membered heteroaryl, —CO₂(C₁-C₆ alkyl), —COOH, 5-6 membered heterocyclyl, 5-6 membered heteroaryl, and —CONHNHCONH(C₁-C₄ alkyl), wherein R₁ is optionally substituted with up to two R₂ substituents.

5. The combination of claim 4, wherein W is halo, pyridyl, pyrimidinyl, pyrrolo[2,3-b]pyridyl, pyrazolyl, pyrazolo[3,4-b]pyridyl, or phenyl, wherein W is optionally substituted with one or two R₁ substituents selected from the group consisting of halo, OH, CN, C₁-C₆ alkyl, —OC₁-C₆ alkyl, —NHS(O)₂(C₁-C₆ alkyl), —NHS(O)₂(C₂-C₆ alkenyl), —NHS(O)₂(C₃-C₆ carbocyclyl), —NHS(O)₂ (5-6 membered heterocyclyl), —N(S(O)₂(C₁-C₆ alkyl))₂, —NRS(O)₂-phenyl, —NH₂, —NHC(O)NH(C₁-C₆ alkyl), 5-6 membered heteroaryl, —CO₂(C₁-C₆ alkyl), —COOH, 5-6 membered heterocyclyl, 5-6 membered heteroaryl, and —CONHNHCONH(C₁-C₄ alkyl), wherein R₁ is optionally substituted with up to two R₂ substituents.

6. The combination of claim 4, wherein W is halo, pyridyl, pyrimidinyl, pyrrolo[2,3-b]pyridyl, pyrazolyl, pyrazolo[3,4-b]pyridyl, or phenyl, wherein W is optionally substituted with one or two R₁ substituents selected from the group consisting of halo, OH, CN, hydroxyl(C₁-C₆ alkyl), —OC₁-C₆ alkyl, —NHS(O)₂(C₁-C₆ alkyl), —NHS(O)₂(C₁-C₆ alkyl)-(5-6 membered heterocyclyl), —NHS(O)₂(C₂-C₆ alkenyl), —NHS(O)₂(C₃-C₆ carbocyclyl), —NHS(O)₂ (5-6 membered heterocyclyl), —NHS(O)₂ (5-6 membered heterocyclyl)-(C₁-C₆ alkyl), —N(S(O)₂(C₁-C₆ alkyl))₂, —NRS(O)₂-phenyl-halo, —NH₂, —NHC(O)NH(C₁-C₆ alkyl), 5-6 membered heteroaryl, 5-6 membered heteroaryl-NH(C₁-C₆ alkyl)-(5-6 membered heterocyclyl), —CO₂(C₁-C₆ alkyl), —COOH, 5-6 membered heterocyclyl, 5-6 membered heterocyclyl-oxo, —CONHNHCONH(C₁-C₄ alkyl)-(5-6 membered heterocyclyl), and —CONHNHCONH(C₁-C₄ alkyl).

7. The combination of claim 4, wherein W is halo, pyridyl, pyrimidinyl, pyrrolo[2,3-b]pyridyl, pyrazolyl, pyrazolo[3,4-b]pyridyl, or phenyl, wherein W is optionally substituted with one or two R₁ substituents selected from the group consisting of halo, OH, —NH₂, —COOH, CN, hydroxymethyl, methoxy, methylsulfonylamino, N-morpholinoethylsulfonylamino, ethenylsulfonylamino, cyclopropylsulfonylamino, N-methyl-N′-morpholinosulfonylamino, bis(methylsulfonyl)amino, fluorophenylsulfonylamino, methylaminocarbonylamino, tetrazolyl, N-morpholinoethylamino-oxadiazolyl, methoxycarbonyl, oxadiazole-2-oneyl, and N-morpholinoethylaminocarbonylhydrazylcarbonyl.

8. The combination of claim 4, wherein W is selected from Br,

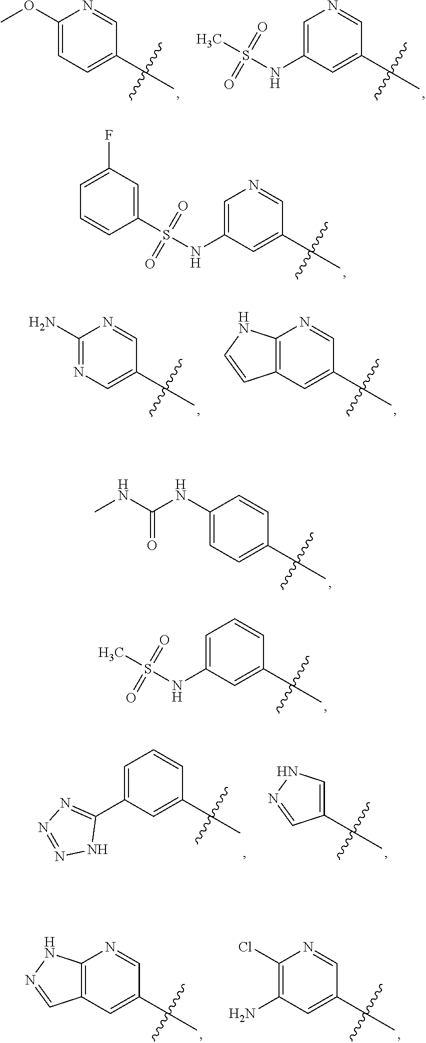

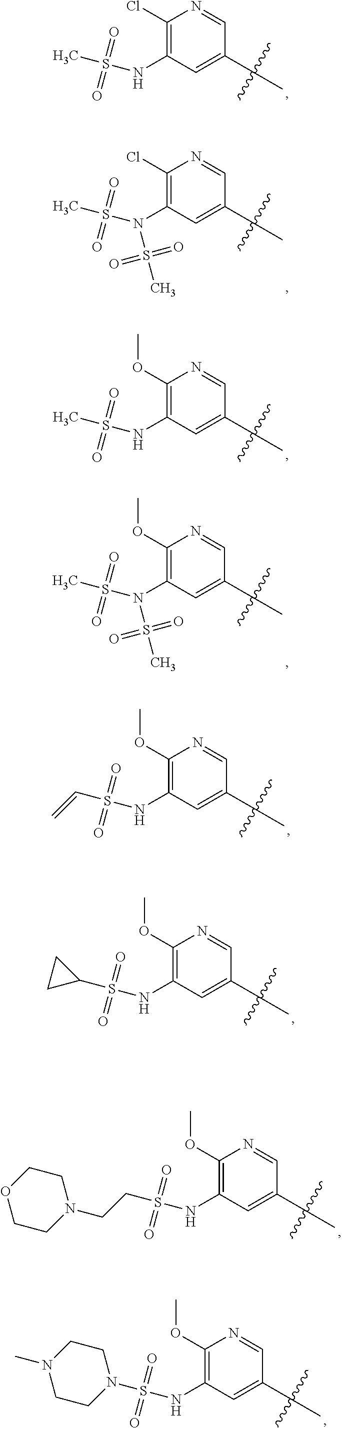

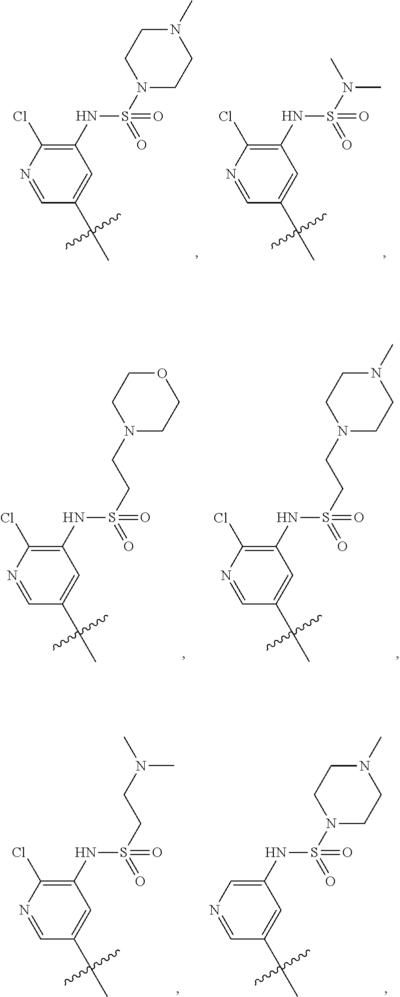 [SMILES: *c1cnc(Cl)c(NS(=O)(=O)N2CCN(C)CC2)c1 ; *c1cnc(Cl)c(NS(=O)(=O)N(C)C)c1 ; *c1cnc(Cl)c(NS(=O)(=O)CCN2CCOCC2)c1 ; *c1cnc(Cl)c(NS(=O)(=O)CCN2CCN(C)CC2)c1 ; *c1cnc(Cl)c(NS(=O)(=O)CCN(C)C)c1 ; *c1cncc(NS(=O)(=O)N2CCN(C)CC2)c1]

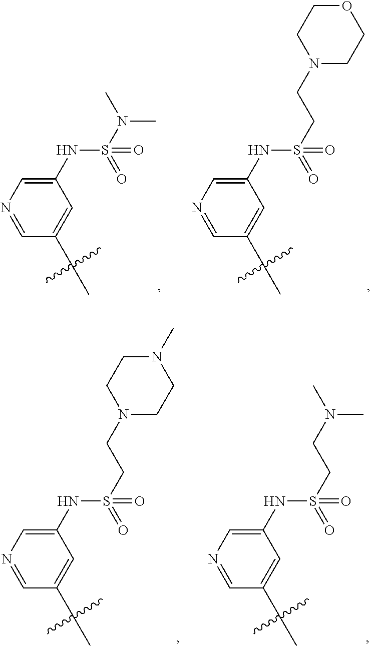 [SMILES: *c1cncc(NS(=O)(=O)N(C)C)c1 ; *c1cncc(NS(=O)(=O)CCN2CCOCC2)c1 ; *c1cncc(NS(=O)(=O)CCN2CCN(C)CC2)c1 ; *c1cncc(NS(=O)(=O)CCN(C)C)c1]

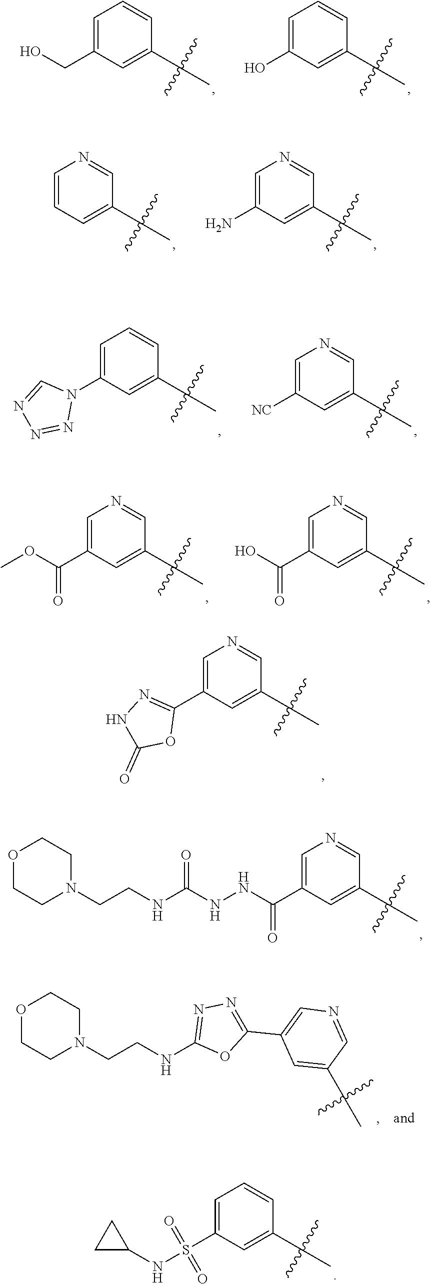

9. The combination of claim 1, wherein Z is selected from the group consisting of 5-6 membered heteroaryl, aryl, benzyl, and phenyl, wherein Z is optionally substituted with up to three R₃ substituents.

10. The combination of claim 9, wherein Z is selected from the group consisting of 5-6 membered heteroaryl and phenyl, wherein Z is optionally substituted with up to three substituents selected from halo, —O(C₁-C₄ alkyl), —O(5-6 membered heteroaryl), C₁-C₄ alkyl, C₂-C₄ alkynyl, —OCH₂ (5-6 membered heteroaryl), and —CH₂O (5-6 membered heteroaryl).

11. The combination of claim 10, wherein Z is selected from the group consisting of pyridyl and phenyl, wherein Z is optionally substituted with up to three substituents selected from halo, —O(C₁-C₄ alkyl), —O(5-6 membered heteroaryl), C₂-C₄ alkynyl, and —OCH₂ (5-6 membered heteroaryl).

12. The combination of claim 11, wherein Z is selected from the group consisting of fluorochlorophenyl, methoxychlorophenyl, ethynylphenyl, chloropyridyl, chlorophenyl, bromophenyl, pyridyloxyphenyl, phenyl, and pyridylmethyloxyphenyl.

13. The combination of claim 12, wherein Z is selected from the group consisting of

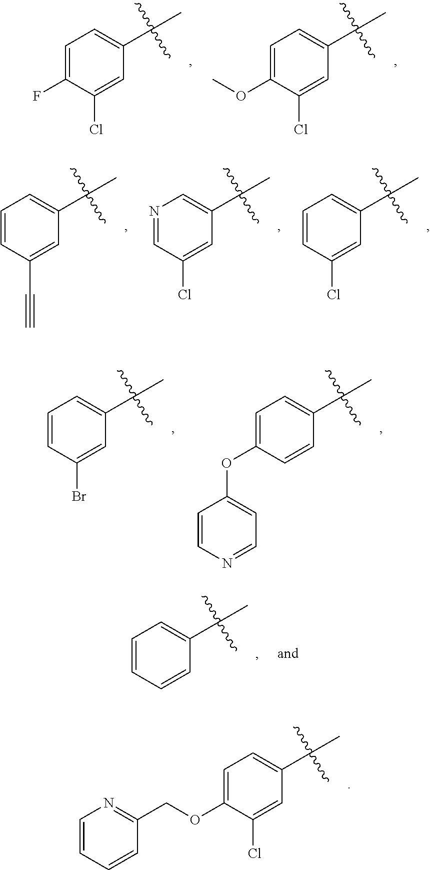

14. The combination of claim 1, wherein L₁ is selected from the group consisting of a bond or a C₁-C₆ branched or straight alkylene group, wherein up to three carbon units of said alkylene group are optionally and independently replaced with a bivalent moiety selected from the group consisting of —CO—, —CONH—, —CO₂—, —O—, —C≡C—, —NHCO—, —S(O)₂—, —NH—, —S(O)₂NH—, and —NHS(O)₂—.

15. The combination of claim 14, wherein L₁ is selected from the group consisting of a bond and —C≡C—.

16. The combination of claim 15, wherein L₁ is a bond.

17. The combination of claim 1, wherein L₂ is selected from the group consisting of a bond or a C₁-C₆ branched or straight alkylene group, wherein up to three carbon units of said alkylene group are optionally and independently replaced with a bivalent moiety selected from the group consisting of —CONR—, —CO₂—, —O—, —NRCO—, —NR—, —S(O)₂NR—, and —NRS(O)₂—.

18. The combination of claim 17, wherein L₂ is selected from the group consisting of —NH— and —NHCH₂—.

19. The combination of claim 18, wherein L₂ is —NH—.

20. The combination of claim 1, wherein X is N.

21. The combination of any one of claims 1-20, wherein the immune checkpoint modulator, is an antibody or an antigen binding fragment thereof, that binds to at least one of the following targets: targets: PD-1, PD-L1, PD-L2, CEACAM (e.g., CEACAM-1, -3 and/or -5), CTLA-4, TIM-3, LAG-3, VISTA, BTLA, TIGIT, LAIR1, CD160, 2B4, TGF beta, OX40, 41BB, LIGHT, CD40, GITR, TGF-beta, TIM-3, SIRP-alpha, VSIG8, BTLA, SIGLEC7, SIGLEC9, ICOS, B7H3, B7H4, FAS, or BTNL2.

22. The combination of claim 21, wherein the immune checkpoint modulator is an immune checkpoint inhibitor, wherein the immune checkpoint inhibitor binds to PD-1, PD-L1, PD-L2, CEACAM (e.g., CEACAM-1, -3 and/or -5), CTLA-4, TIM-3, LAG-3, VISTA, BTLA, TIGIT, LAIR1, CD160, 2B4, TGF beta, or a combination thereof.

23. The combination of claim 22, wherein the immune checkpoint inhibitor binds to PD-1, PD-L₁, PD-L2, CTLA-4, or combinations thereof.

24. The combination of claim 23, wherein the immune checkpoint inhibitor is any one or more of: Tremelimumab, Abatacept, AK104, REGN2810 (Cemiplimab), Nivolumab, Pembrolizumab, Sintilimab (IBI308), Tislelizumab (BGB-A317), SHR-1210 (Camrelizumab), Spartalizumab (PDR001), JS001, TSR-042, JNJ-63723283, BCD-100, TORIPALIMAB, Avelumab, Atezolizumab, TQB2450, KN035, CS1001, and Durvalumab (MEDI4736).

25. The combination of any one of claims 1-24, wherein the compound of Formula I is a compound selected from: 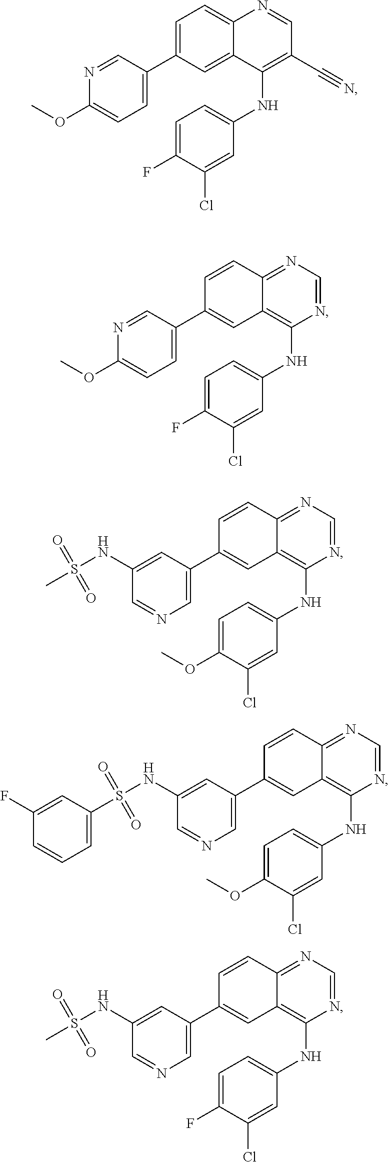 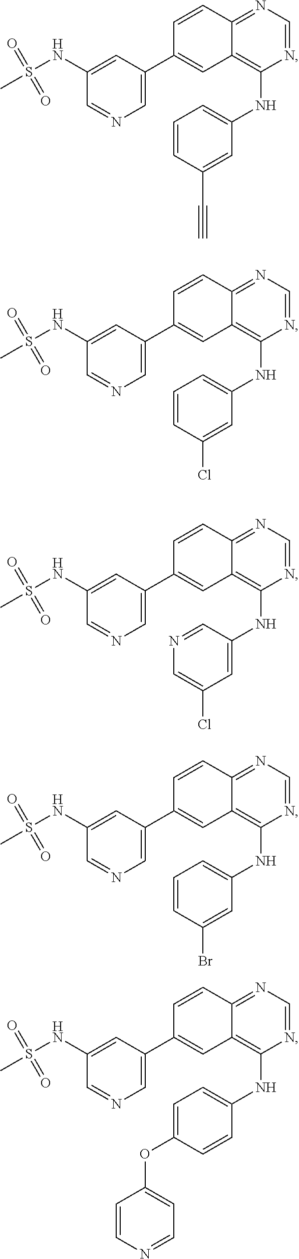 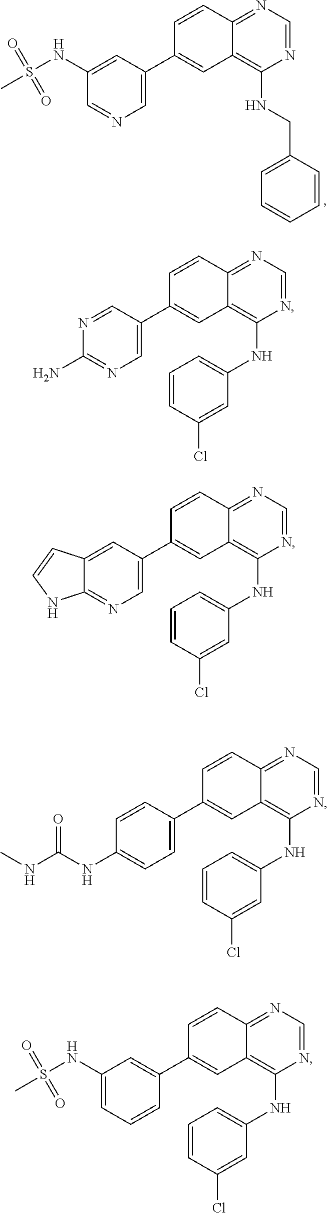 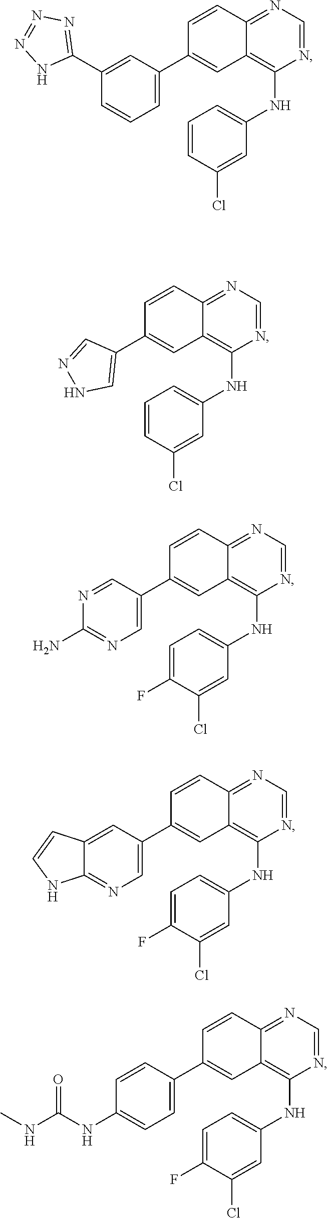 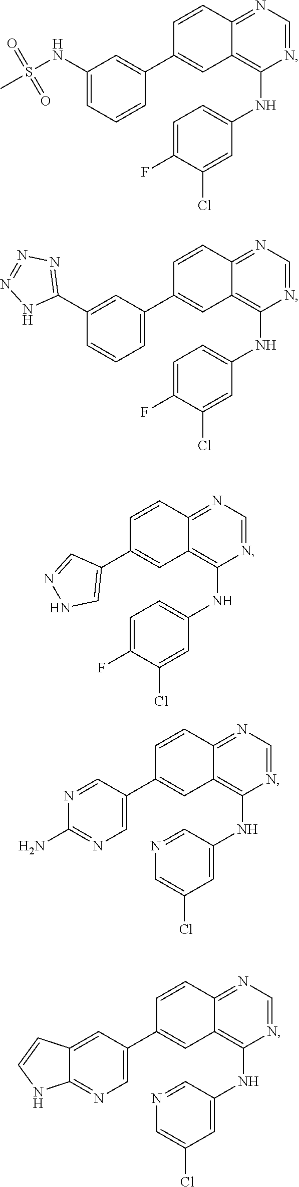 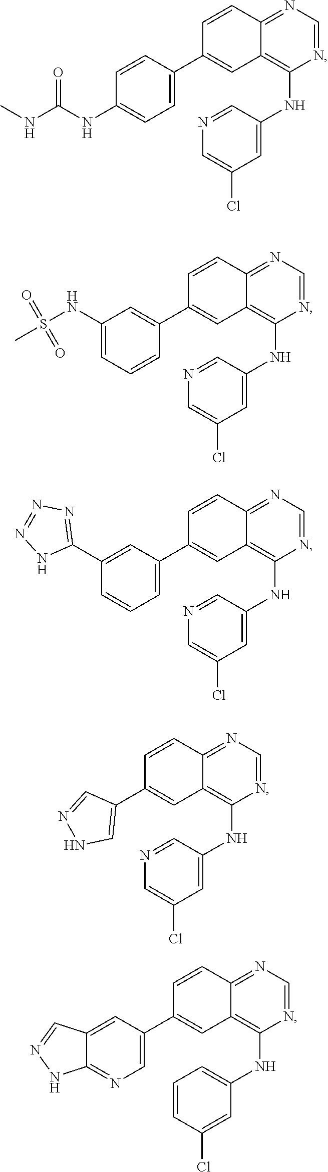 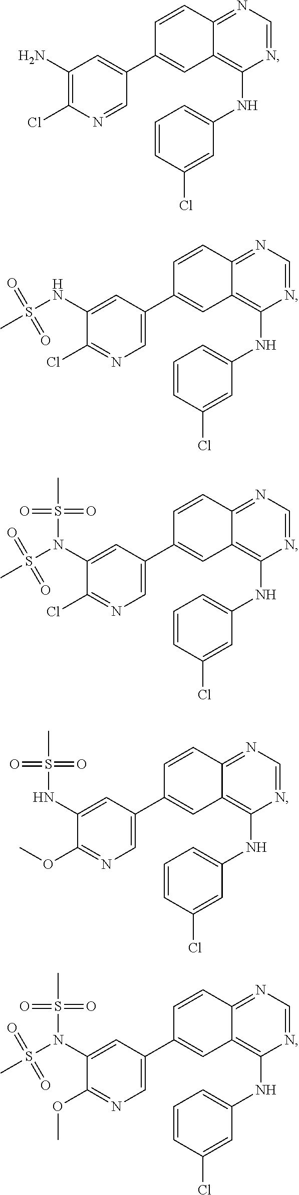 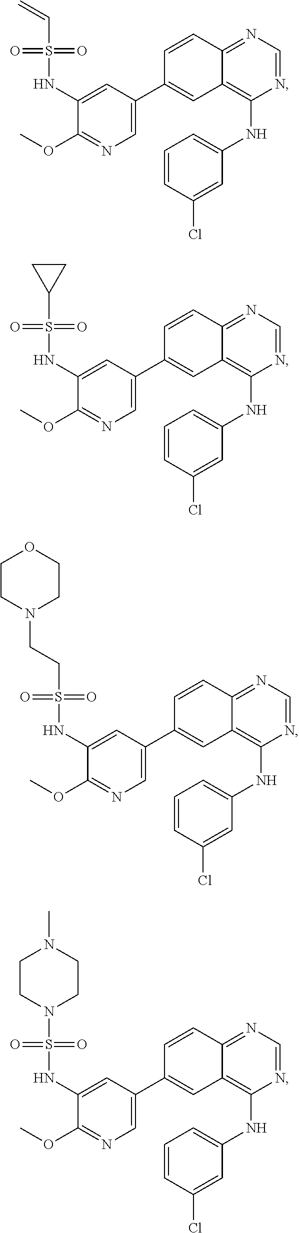 [SMILES: C=CS(=O)(=O)Nc1cc(-c2ccc3ncnc(Nc4cccc(Cl)c4)c3c2)cnc1OC ; COc1ncc(-c2ccc3ncnc(Nc4cccc(Cl)c4)c3c2)cc1NS(=O)(=O)C1CC1 ; COc1ncc(-c2ccc3ncnc(Nc4cccc(Cl)c4)c3c2)cc1NS(=O)(=O)CCN1CCOCC1 ; COc1ncc(-c2ccc3ncnc(Nc4cccc(Cl)c4)c3c2)cc1NS(=O)(=O)N1CCN(C)CC1] 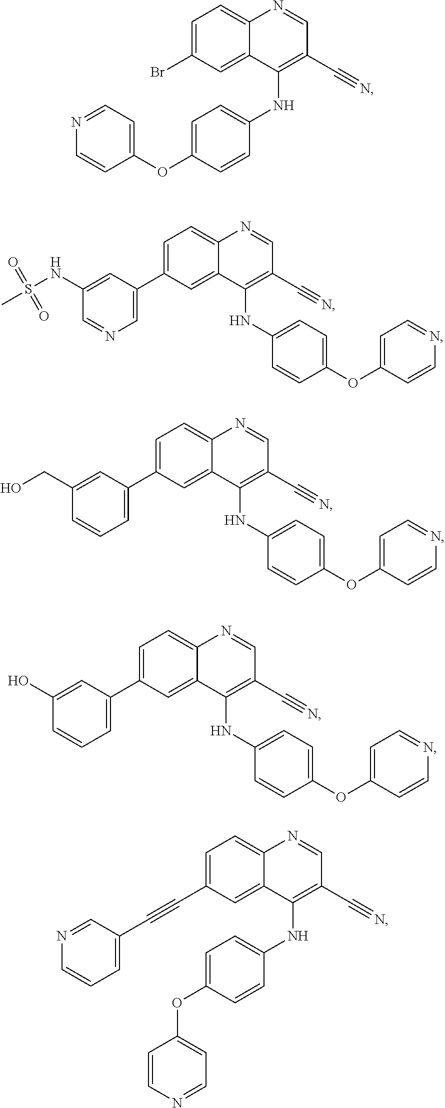 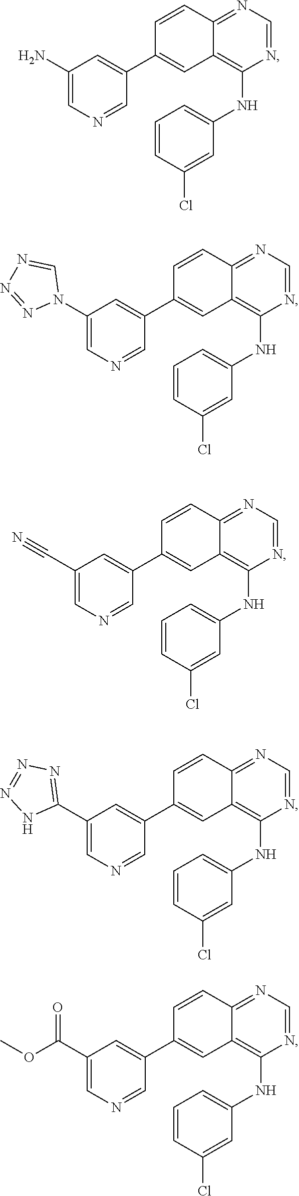 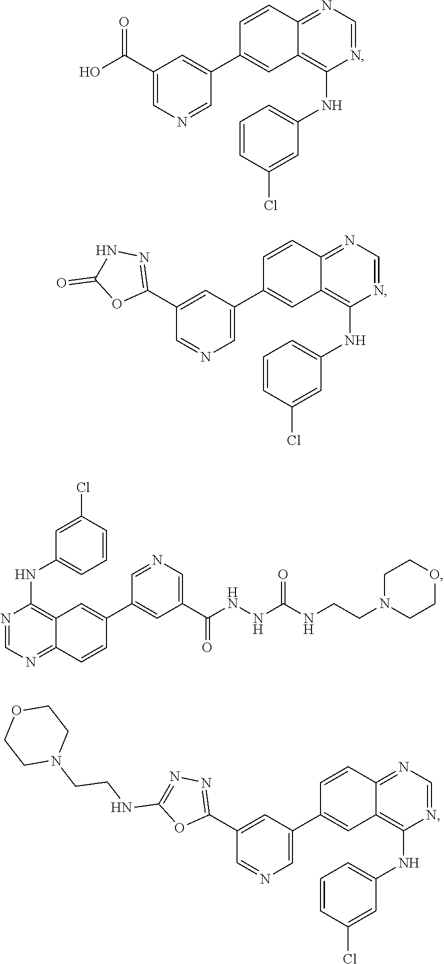 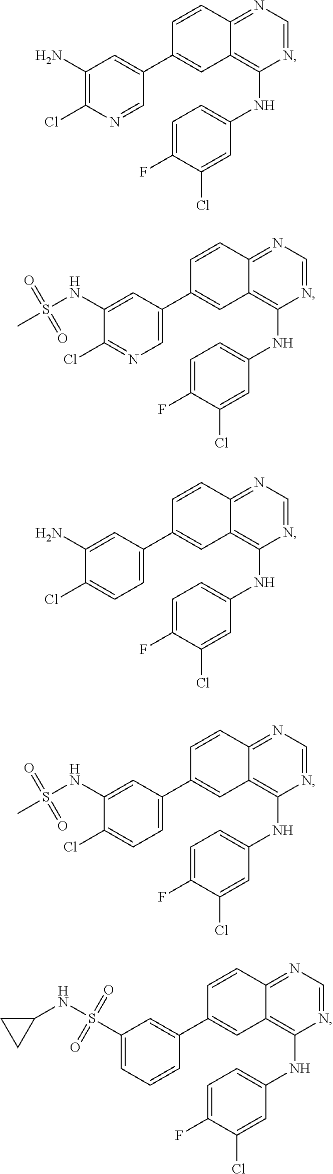 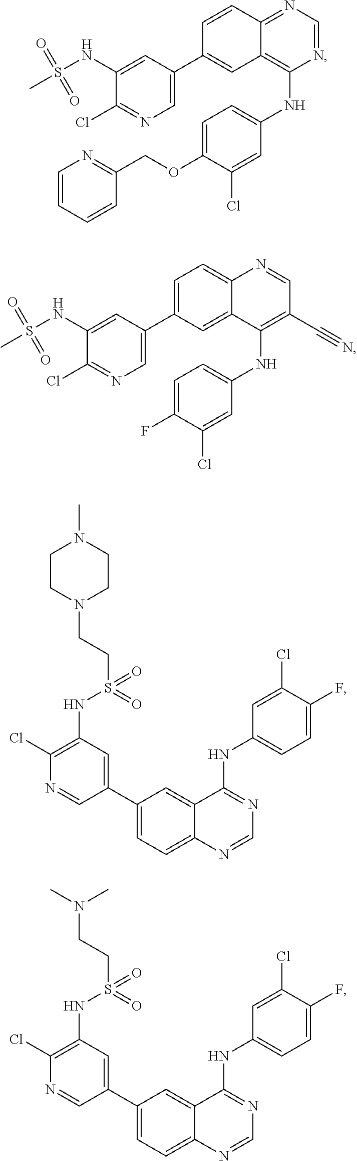 [SMILES: CS(=O)(=O)Nc1cc(-c2ccc3ncnc(Nc4ccc(OCc5ccccn5)c(Cl)c4)c3c2)cnc1Cl ; CS(=O)(=O)Nc1cc(-c2ccc3ncc(C#N)c(Nc4ccc(F)c(Cl)c4)c3c2)cnc1Cl ; CN1CCN(CCS(=O)(=O)Nc2cc(-c3ccc4ncnc(Nc5ccc(F)c(Cl)c5)c4c3)cnc2Cl)CC1 ; CN(C)CCS(=O)(=O)Nc1cc(-c2ccc3ncnc(Nc4ccc(F)c(Cl)c4)c3c2)cnc1Cl] 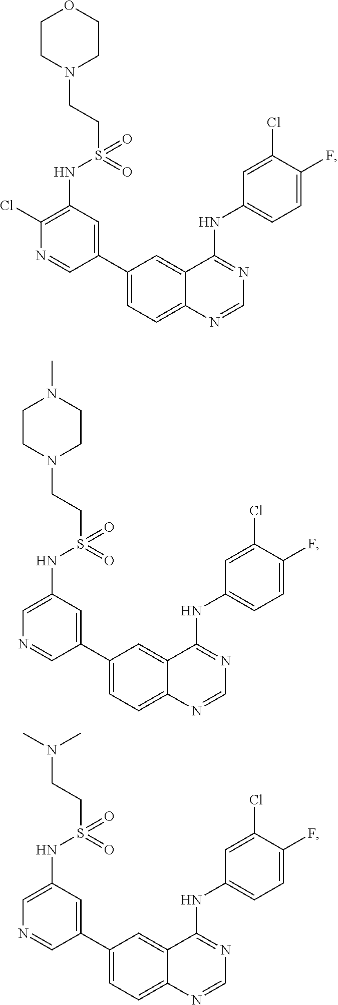 [SMILES: O=S(=O)(CCN1CCOCC1)Nc1cc(-c2ccc3ncnc(Nc4ccc(F)c(Cl)c4)c3c2)cnc1Cl ; CN1CCN(CCS(=O)(=O)Nc2cncc(-c3ccc4ncnc(Nc5ccc(F)c(Cl)c5)c4c3)c2)CC1 ; CN(C)CCS(=O)(=O)Nc1cncc(-c2ccc3ncnc(Nc4ccc(F)c(Cl)c4)c3c2)c1] 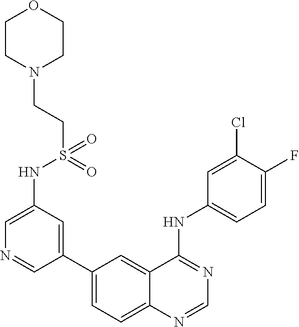 [SMILES: O=S(=O)(CCN1CCOCC1)Nc1cncc(-c2ccc3ncnc(Nc4ccc(F)c(Cl)c4)c3c2)c1]
or pharmaceutically acceptable salts thereof.

26. The combination of any one of claims 1-25, wherein the compound is 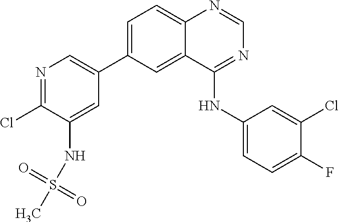 [SMILES: CS(=O)(=O)Nc1cc(-c2ccc3ncnc(Nc4ccc(F)c(Cl)c4)c3c2)cnc1Cl]
or a pharmaceutically acceptable salt thereof.

27. The combination of claim 26, comprising the compound: 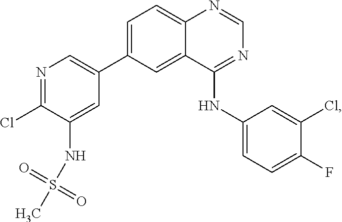 [SMILES: CS(=O)(=O)Nc1cc(-c2ccc3ncnc(Nc4ccc(F)c(Cl)c4)c3c2)cnc1Cl]
or a pharmaceutically acceptable salt thereof, and one or more immune checkpoint modulators selected from: Tremelimumab, Abatacept, AK104, REGN2810 (Cemiplimab), Nivolumab, Pembrolizumab, Sintilimab (IBI308), Tislelizumab (BGB-A317), SHR-1210 (Camrelizumab), Spartalizumab (PDR001), JS001, TSR-042, JNJ-63723283, BCD-100, TORIPALIMAB, Avelumab, Atezolizumab, TQB2450, KN035, CS1001, and Durvalumab (MEDI4736).

28. A combination comprising:
(a). a compound of Formula Ia, or a pharmaceutically acceptable salt thereof, 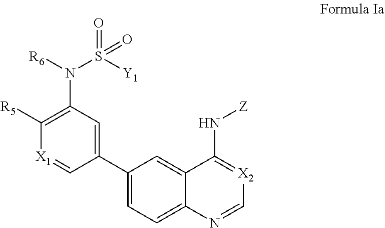
wherein
X₁ is N or CH;
X₂ is N or C—CN;
Z is selected from the group consisting of 5-6 membered heteroaryl and phenyl, wherein Z is optionally substituted with up to three R₃ substituents;
R₅ is H, OH, CN, NH₂, NO₂, O(C₁-C₄ alkyl), C₁-C₄ alkyl, C₂-C₄ alkenyl, C₂-C₄ alkynyl, 5-6 membered heteroaryl, 5-6 membered heterocyclyl, and C₃-C₆ carbocyclyl;
R₆ is H, C₁-C₄ alkyl, or —S(O)₂(C₁-C₄ alkyl); and
Y₁ is selected from the group consisting of H, OH, O(C₁-C₄ alkyl), C₁-C₄ alkyl, C₂-C₄ alkyl(R₇), C₂-C₄ alkenyl, C₂-C₄ alkynyl, 5-6 membered heteroaryl, 5-6 membered heterocyclyl, and C₃-C₆ carbocyclyl, wherein Y₁ is optionally substituted with up to two instances of 5-6 membered heterocyclyl, 5-6 membered carbocyclyl, O(C₁-C₄ alkyl), C₁-C₄ alkyl, OH, CN, halo, NO₂, and NH₂; and
R₇ is selected from NH₂, N(H)(C₁-C₄ alkyl), N(C₁-C₄ alkyl)₂, 3-7 membered heterocyclyl;
provided that the compound of Formula Ia is not 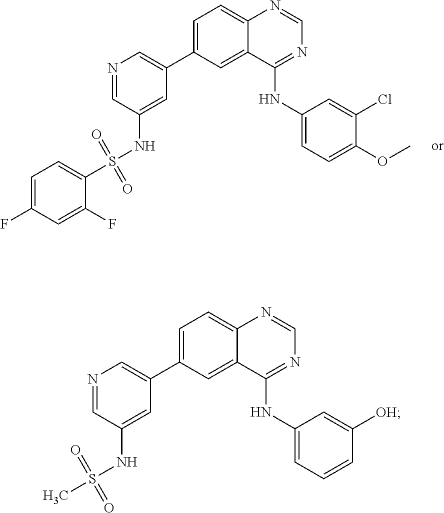
and,
(b). an immune checkpoint modulator, wherein the immune checkpoint inhibitor is an antibody or an antigen binding fragment thereof, that binds to at least one of the following targets: PD-1, PD-L1, PD-L2, CEACAM (e.g., CEACAM-1, -3 and/or -5), CTLA-4, TIM-3, LAG-3, VISTA, BTLA, TIGIT, LAIR1, CD160, 2B4, TGF beta, OX40, 41BB, LIGHT, CD40, GITR, TGF-beta, TIM-3, SIRP-alpha, VSIG8, BTLA, SIGLEC7, SIGLEC9, ICOS, B7H3, B7H4, FAS, or BTNL2.

29. The combination of claim 28, wherein Z is selected from the group consisting of 5-6 membered heteroaryl and phenyl, wherein Z is optionally substituted with up to three substituents selected from halo, —O(C₁-C₄ alkyl), —O(5-6 membered heteroaryl), C₁-C₄ alkyl, C₂-C₄ alkynyl, —OCH₂ (5-6 membered heteroaryl), and —CH₂O (5-6 membered heteroaryl).

30. The combination of claim 29, wherein Z is selected from the group consisting of pyridyl and phenyl, wherein Z is optionally substituted with up to three substituents selected from halo, —O(C₁-C₄ alkyl), —O(5-6 membered heteroaryl), C₂-C₄ alkynyl, and —OCH₂ (5-6 membered heteroaryl).

31. The combination of claim 30, wherein Z is selected from the group consisting of fluorochlorophenyl, methoxychlorophenyl, ethynylphenyl, chloropyridyl, chlorophenyl, bromophenyl, pyridyloxyphenyl, phenyl, and pyridylmethyloxyphenyl.

32. The combination of claim 31, wherein Z is selected from the group consisting of

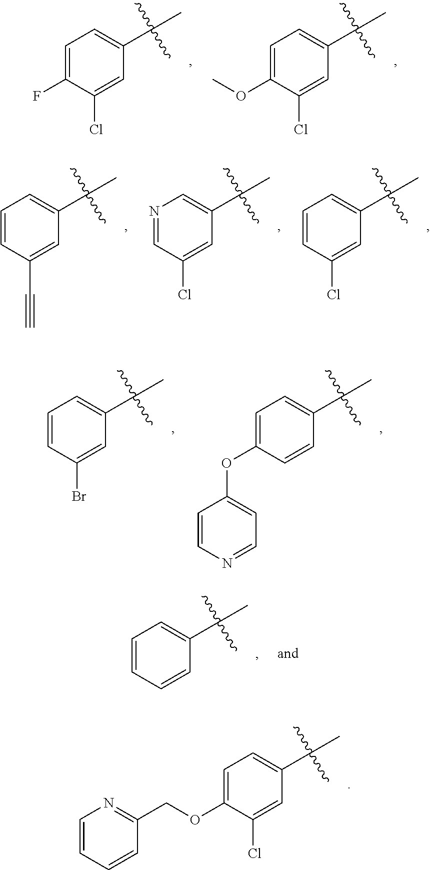

33. The combination of claim 28, wherein Y₁ is selected from the group consisting of O(C₁-C₄ alkyl), C₁-C₄ alkyl, C₂-C₄ alkenyl, 5-6 membered heterocyclyl, and C₃-C₆ carbocyclyl, wherein Y₁ is optionally substituted with up to two instances of 5-6 membered heterocyclyl, C₁-C₄ alkyl, OH, CN, and halo.

34. The combination of claim 33, wherein Y₁ is selected from the group consisting of C₁-C₄ alkyl, C₂-C₄ alkenyl, 5-6 membered heterocyclyl, and C₃-C₆ carbocyclyl, wherein Y₁ is optionally substituted with up to two instances of 5-6 membered heterocyclyl and C₁-C₄ alkyl.

35. The combination of claim 34, wherein Y₁ is selected from the group consisting of C₁-C₄ alkyl, C₂-C₄ alkenyl, alkyl-5-6 membered heterocyclyl, 5-6 membered heterocyclyl-C₁-C₄ alkyl, and C₃-C₆ carbocyclyl.

36. The combination of claim 35, wherein Y₁ is selected from the group consisting of methyl, ethenyl, cyclopropyl, N-methylpiperizinyl, and 2-morpholinoethyl.

37. The combination of claim 28, wherein R₅ is H, halo, NH₂, O(C₁-C₄ alkyl), C₁-C₄ alkyl;

38. The combination of claim 28, wherein R₆ is H or —S(O)₂(C₁-C₄ alkyl).

39. The combination of claim 37, wherein R₅ is H, halo, NH₂, or methoxy;

40. The combination of claim 38, wherein R₆ is H or —S(O)₂CH₃.

41. The combination of claim 28, wherein X₁ is N;

42. The combination of claim 28, wherein X₁ is CH;

43. The combination of claim 28, wherein X₂ is N;

44. The combination of claim 28, wherein X₂ is C—CN;

45. The combination of claim 28, wherein the immune checkpoint modulator is an immune checkpoint inhibitor, wherein the immune checkpoint inhibitor binds to PD-1, PD-L1, PD-L2, CEACAM (e.g., CEACAM-1, -3 and/or -5), CTLA-4, TIM-3, LAG-3, VISTA, BTLA, TIGIT, LAIR1, CD160, 2B4, TGF beta, or a combination thereof.

46. The combination of claim 45, wherein the immune checkpoint inhibitor binds to PD-1, PD-L1, PD-L2, CTLA-4, or combinations thereof.

47. The combination of any one of claims 28-46, wherein the compound of Formula Ia is selected from: 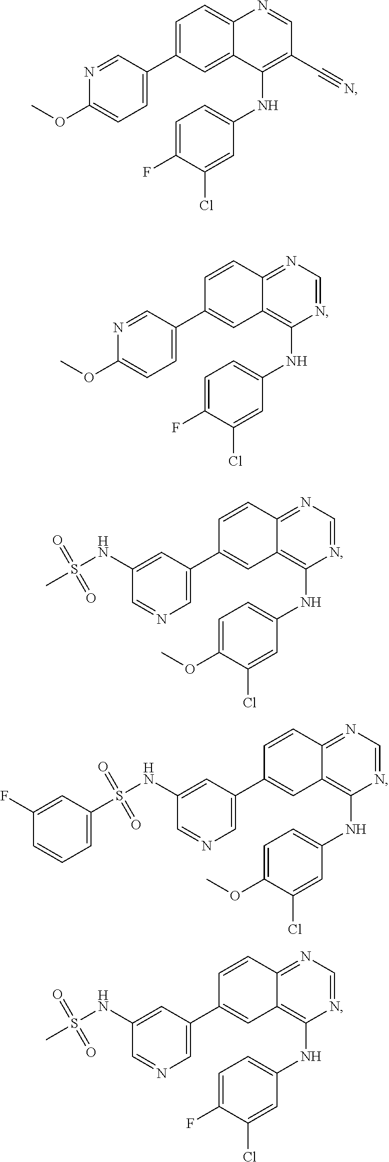 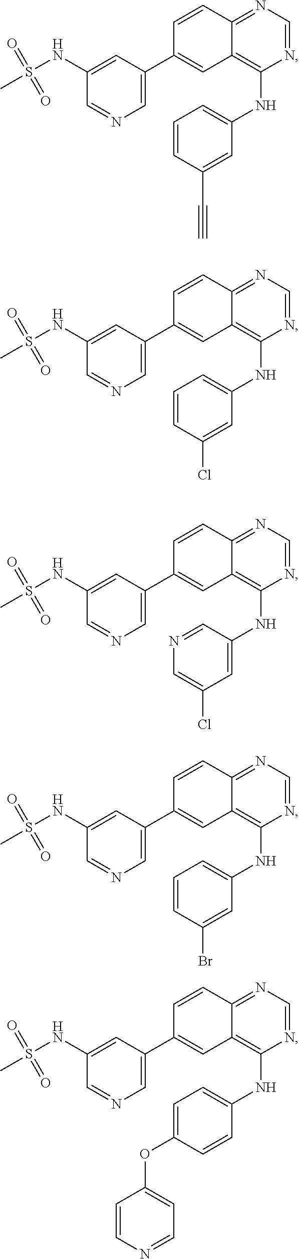 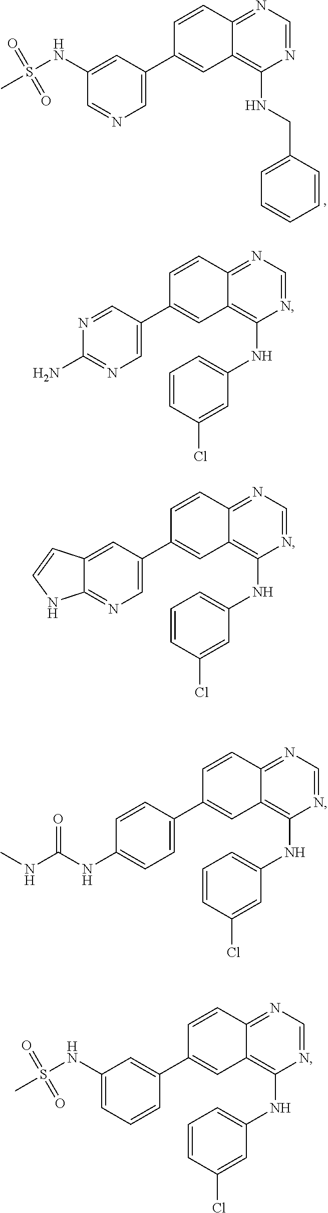 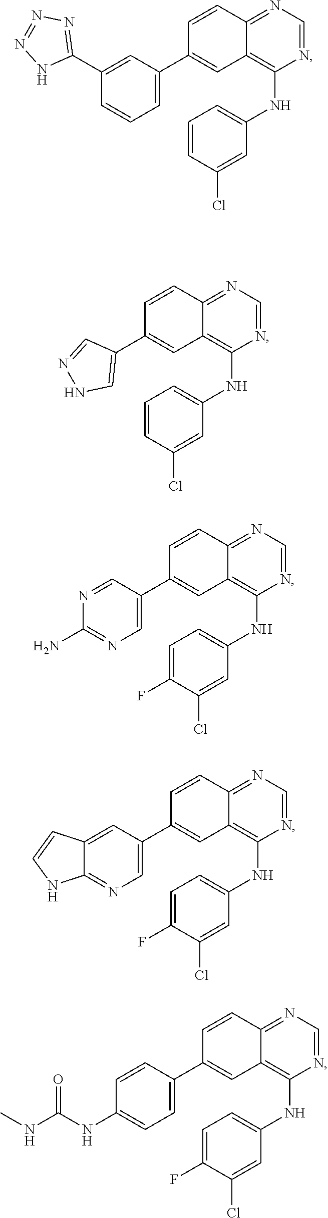 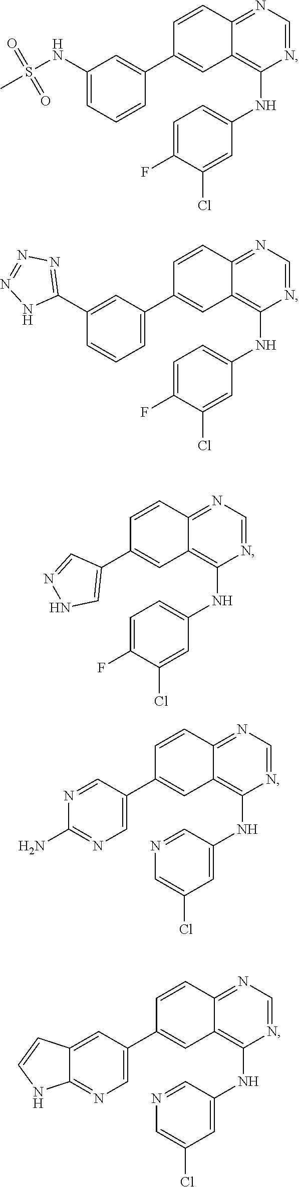 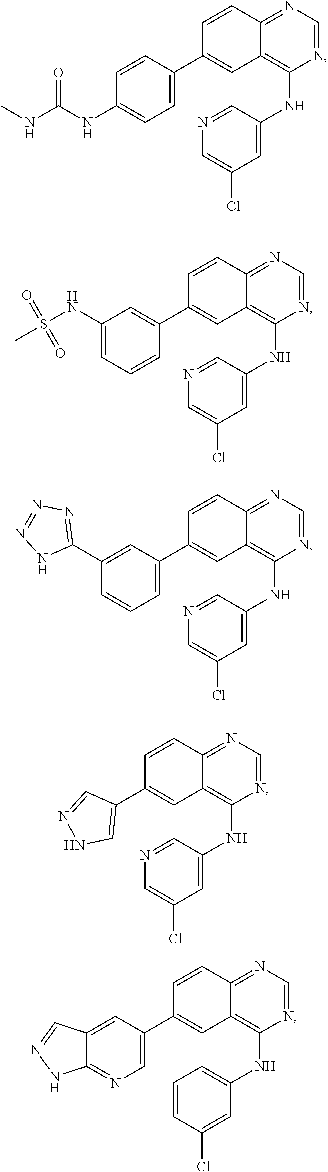 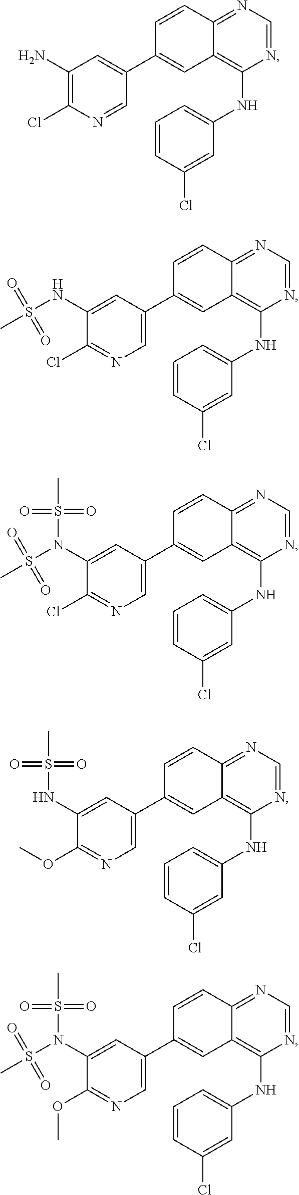 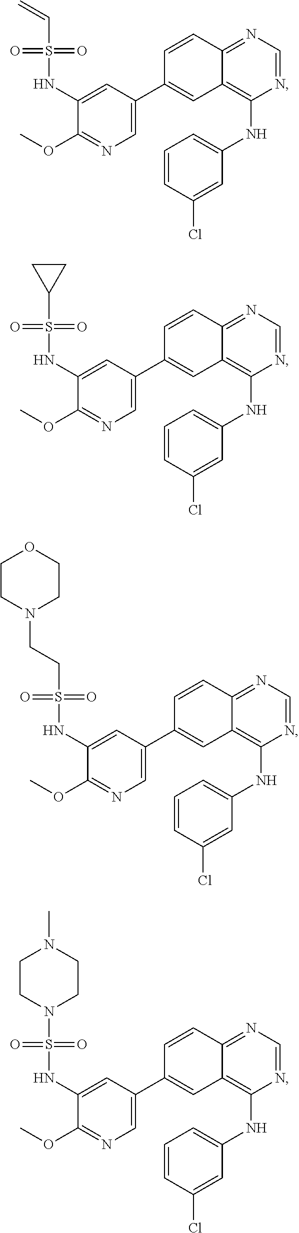 [SMILES: C=CS(=O)(=O)Nc1cc(-c2ccc3ncnc(Nc4cccc(Cl)c4)c3c2)cnc1OC ; COc1ncc(-c2ccc3ncnc(Nc4cccc(Cl)c4)c3c2)cc1NS(=O)(=O)C1CC1 ; COc1ncc(-c2ccc3ncnc(Nc4cccc(Cl)c4)c3c2)cc1NS(=O)(=O)CCN1CCOCC1 ; COc1ncc(-c2ccc3ncnc(Nc4cccc(Cl)c4)c3c2)cc1NS(=O)(=O)N1CCN(C)CC1] 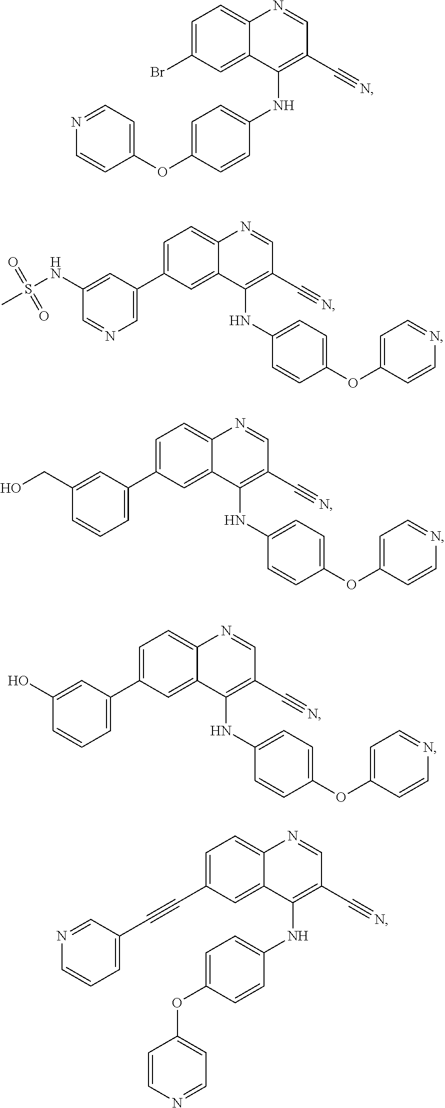 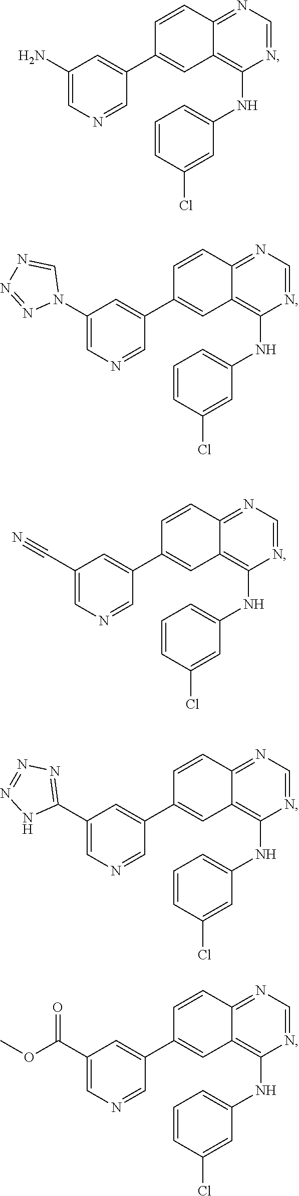 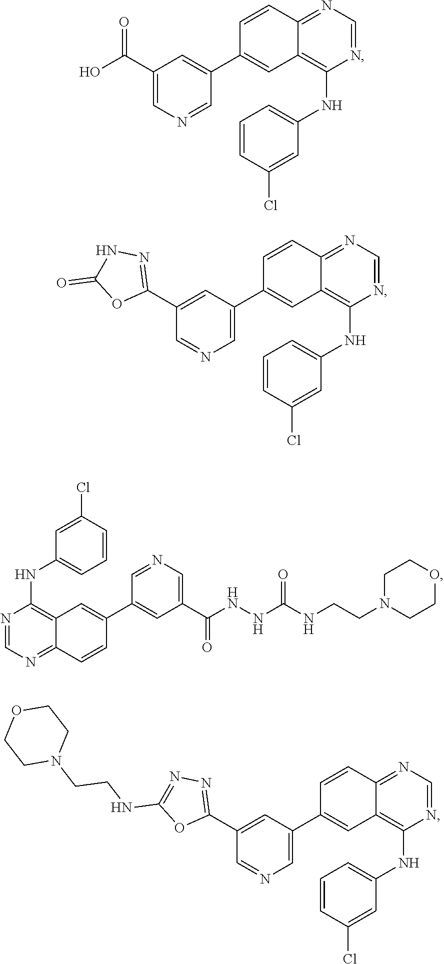 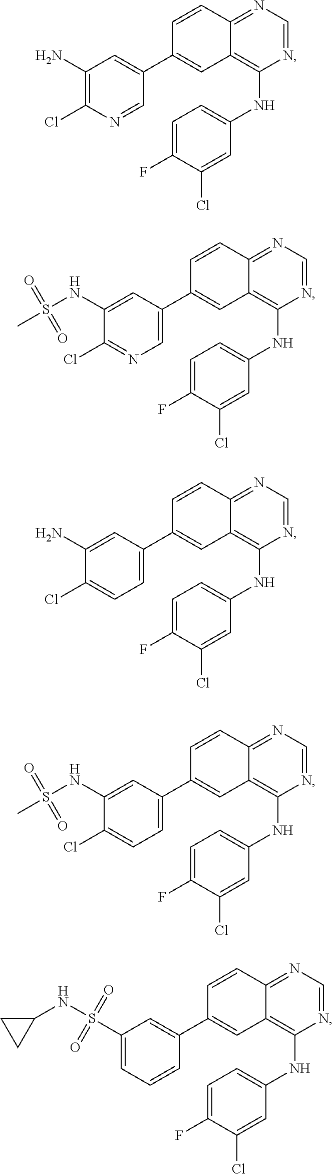 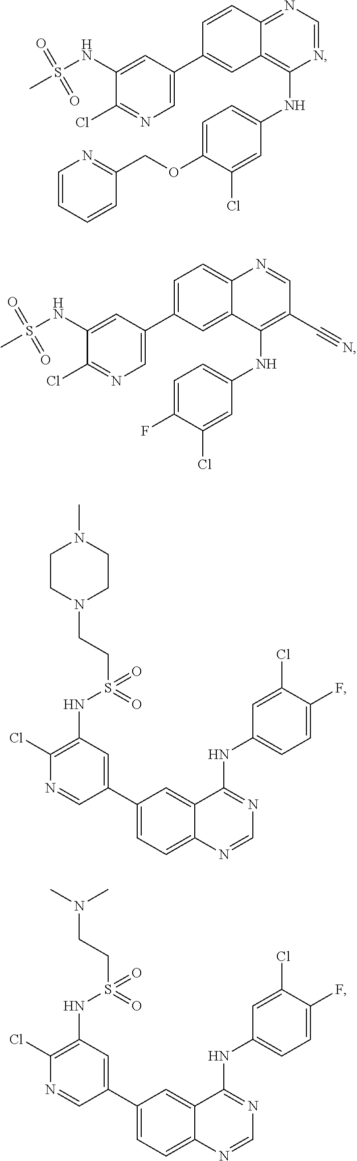 [SMILES: CS(=O)(=O)Nc1cc(-c2ccc3ncnc(Nc4ccc(OCc5ccccn5)c(Cl)c4)c3c2)cnc1Cl ; CS(=O)(=O)Nc1cc(-c2ccc3ncc(C#N)c(Nc4ccc(F)c(Cl)c4)c3c2)cnc1Cl ; CN1CCN(CCS(=O)(=O)Nc2cc(-c3ccc4ncnc(Nc5ccc(F)c(Cl)c5)c4c3)cnc2Cl)CC1 ; CN(C)CCS(=O)(=O)Nc1cc(-c2ccc3ncnc(Nc4ccc(F)c(Cl)c4)c3c2)cnc1Cl] 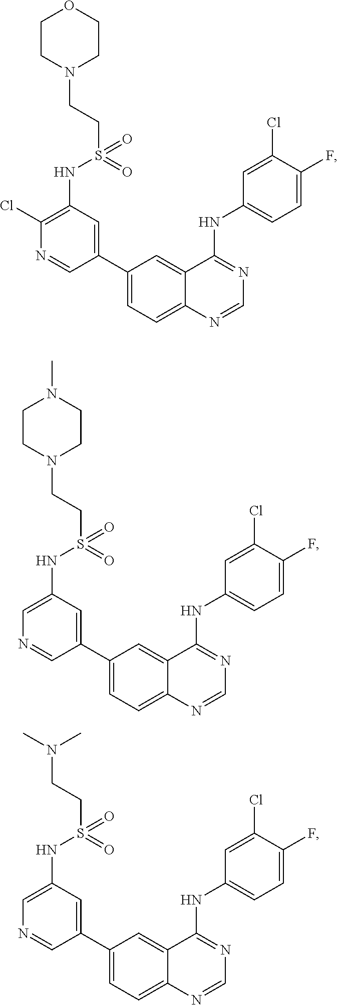 [SMILES: O=S(=O)(CCN1CCOCC1)Nc1cc(-c2ccc3ncnc(Nc4ccc(F)c(Cl)c4)c3c2)cnc1Cl ; CN1CCN(CCS(=O)(=O)Nc2cncc(-c3ccc4ncnc(Nc5ccc(F)c(Cl)c5)c4c3)c2)CC1 ; CN(C)CCS(=O)(=O)Nc1cncc(-c2ccc3ncnc(Nc4ccc(F)c(Cl)c4)c3c2)c1] 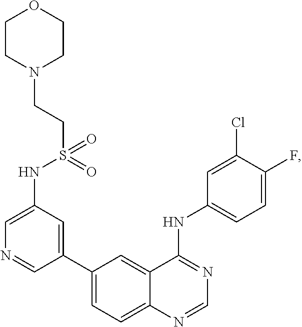 [SMILES: O=S(=O)(CCN1CCOCC1)Nc1cncc(-c2ccc3ncnc(Nc4ccc(F)c(Cl)c4)c3c2)c1]
or pharmaceutically acceptable salts thereof.

48. The combination of any one of claims 28-47, wherein the compound of Formula Ia is 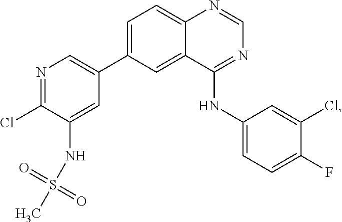 [SMILES: CS(=O)(=O)Nc1cc(-c2ccc3ncnc(Nc4ccc(F)c(Cl)c4)c3c2)cnc1Cl]
or a pharmaceutically acceptable salt thereof.

49. The combination of claim 48, comprising the compound: 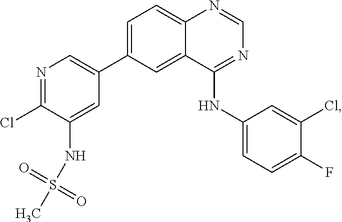 [SMILES: CS(=O)(=O)Nc1cc(-c2ccc3ncnc(Nc4ccc(F)c(Cl)c4)c3c2)cnc1Cl]
or a pharmaceutically acceptable salt thereof, and one or more immune checkpoint inhibitors selected from Tremelimumab, Abatacept, AK104, REGN2810 (Cemiplimab), Nivolumab, Pembrolizumab, Sintilimab (IBI308), Tislelizumab (BGB-A317), SHR-1210 (Camrelizumab), Spartalizumab (PDR001), JS001, TSR-042, JNJ-63723283, BCD-100, TORIPALIMAB, Avelumab, Atezolizumab, TQB2450, KN035, CS1001, and Durvalumab (MEDI4736).

50. A method of preventing or treating cancer, the method comprising administering a therapeutically effective amount of a combination comprising
(a). a compound of Formula I, or a pharmaceutically acceptable salt thereof: 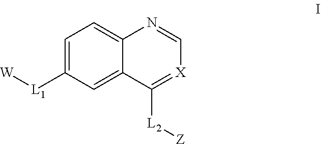
wherein X is N or C—R₄;
L₁ and L₂ are each independently a bond or a C₁-C₆ branched or straight alkylene group, wherein up to three carbon units of said alkylene group are optionally and independently replaced with a bivalent moiety selected from the group consisting of —CO—, —CS—, —CONR—, —CONRNR—, —CO₂—, —OCO—, —NRCO₂—, —O—, —CR═CR—, —C≡C—, —NRCONR—, —OCONR—, —NRNR—, —NRCO—, —S—, —S(O)—, —S(O)₂—, —NR—, —S(O)₂NR—, —NRS(O)₂—, and —NRS(O)₂NR—;
W is selected from the group consisting of halo, 5-10 membered heteroaryl, 5-10 membered heterocyclyl, C₃-C₁₀ carbocyclyl, naphthyl, and phenyl, wherein W is optionally substituted with up to three R₁ substituents;
Z is selected from the group consisting of 5-10 membered heteroaryl, 5-10 membered heterocyclyl, C₃-C₁₀ carbocyclyl, and phenyl, wherein Z is optionally substituted with up to three R₃ substituents;
R₁ is selected from the group consisting of halo, CN, C₁-C₆ alkyl, phenyl, 5-6 membered heteroaryl, 5-6 membered heterocyclyl, C₃-C₆ carbocyclyl, —OR, —CONR₂, —CONRNR₂, —CO₂R, —S(O)₂R, —NR₂, —NRS(O)₂R, —S(O)₂NR₂, and —NRCONR₂, wherein R₁ is optionally substituted with up to two R₂ substituents.
R₂ is selected from the group consisting of halo, C₁-C₆ alkyl, C₁-C₆ alkoxy, phenyl, 5-6 membered heteroaryl, 5-6 membered heterocyclyl, C₃-C₆ carbocyclyl, —OH, oxo, —NR₂, wherein each R₂ is optionally and independently substituted with 5-6 membered heterocyclyl;
R₃ is selected from the group consisting of R, halo, —OR, —O(CH₂)ₙR, and —(CH₂)ₙOR;
R₄ is selected from the group consisting of H, halo, C₁-C₄ alkyl, CN, OH, and —COOH;
R is selected from the group consisting of H, C₁-C₄ alkyl, C₂-C₄ alkenyl, C₂-C₄ alkynyl, phenyl, 5-6 membered heteroaryl, 5-6 membered heterocyclyl, C₃-C₆ carbocyclyl, alkylsulfonyl, and —CONH(C₁-C₄ alkyl); and n is 1, 2, or 3; and
(b). an immune checkpoint modulator,
provided that the compound of Formula I is not

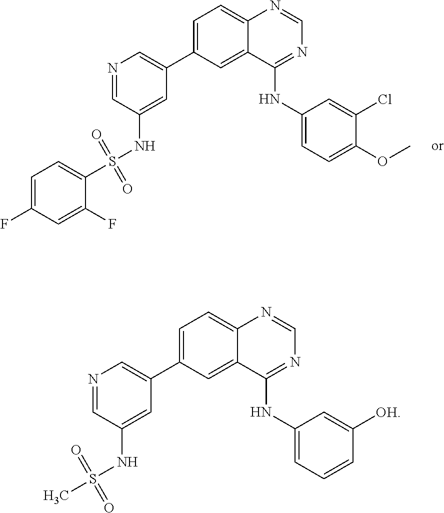

51. A method of preventing or treating cancer, the method comprising administering a therapeutically effective amount of a combination comprising
(a). a compound of Formula Ia, or a pharmaceutically acceptable salt thereof, 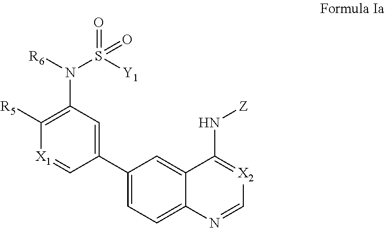
wherein
X₁ is N or CH;
X₂ is N or C—CN;
Z is selected from the group consisting of 5-6 membered heteroaryl and phenyl, wherein Z is optionally substituted with up to three R₃ substituents;
R₅ is H, OH, CN, NH₂, NO₂, O(C₁-C₄ alkyl), C₁-C₄ alkyl, C₂-C₄ alkenyl, C₂-C₄ alkynyl, 5-6 membered heteroaryl, 5-6 membered heterocyclyl, and C₃-C₆ carbocyclyl;
R₆ is H, C₁-C₄ alkyl, or —S(O)₂(C₁-C₄ alkyl); and
Y₁ is selected from the group consisting of H, OH, O(C₁-C₄ alkyl), C₁-C₄ alkyl, C₂-C₄ alkenyl, C₂-C₄ alkynyl, 5-6 membered heteroaryl, 5-6 membered heterocyclyl, and C₃-C₆ carbocyclyl, wherein Y₁ is optionally substituted with up to two instances of 5-6 membered heterocyclyl, 5-6 membered carbocyclyl, O(C₁-C₄ alkyl), C₁-C₄ alkyl, OH, CN, halo, NO₂, and NH₂;
provided that the compound of Formula Ia is not 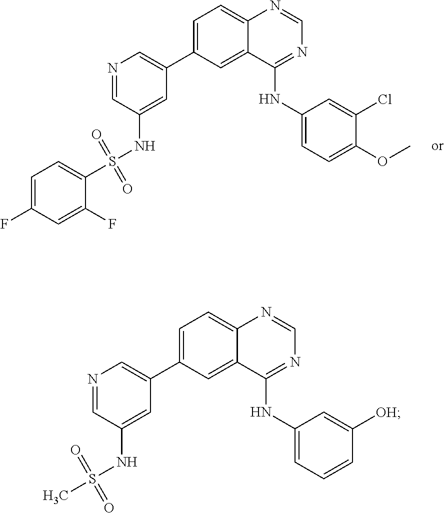
and,
(b). an immune checkpoint modulator, wherein the immune checkpoint inhibitor is an antibody or an antigen binding fragment thereof, that binds to at least one of the following targets: PD-1, PD-L1, PD-L2, CEACAM (e.g., CEACAM-1, -3 and/or -5), CTLA-4, TIM-3, LAG-3, VISTA, BTLA, TIGIT, LAIR1, CD160, 2B4, TGF beta, OX40, 41BB, LIGHT, CD40, GITR, TGF-beta, TIM-3, SIRP-alpha, VSIG8, BTLA, SIGLEC7, SIGLEC9, ICOS, B7H3, B7H4, FAS, or BTNL2.

52. The method of claim 51, wherein the immune checkpoint modulator is an immune checkpoint inhibitor, wherein the immune checkpoint inhibitor binds to PD-1, PD-L1, PD-L₂, CEACAM (e.g., CEACAM-1, -3 and/or -5), CTLA-4, TIM-3, LAG-3, VISTA, BTLA, TIGIT, LAIR1, CD160, 2B4, TGF beta, or a combination thereof.

53. The method of claim 52, wherein the immune checkpoint inhibitor binds to PD-1, PD-L1, PD-L2, CTLA-4, or combinations thereof.

54. The method of any one of claims 50-53, wherein the compound of Formula I or Formula Ia is selected from: 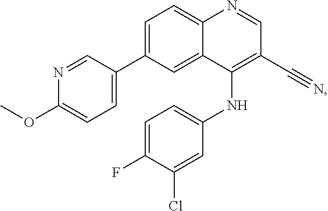 [SMILES: COc1ccc(-c2ccc3ncc(C#N)c(Nc4ccc(F)c(Cl)c4)c3c2)cn1] 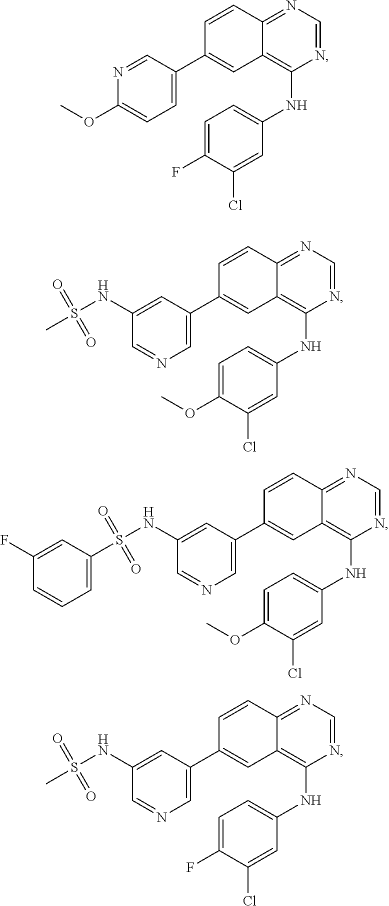 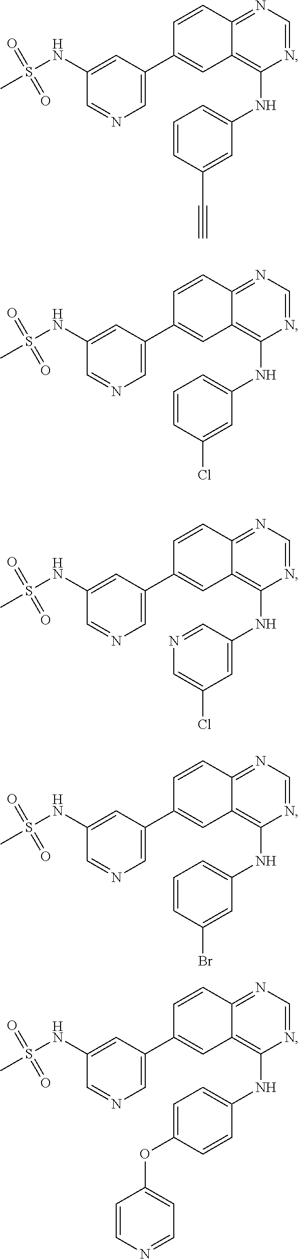 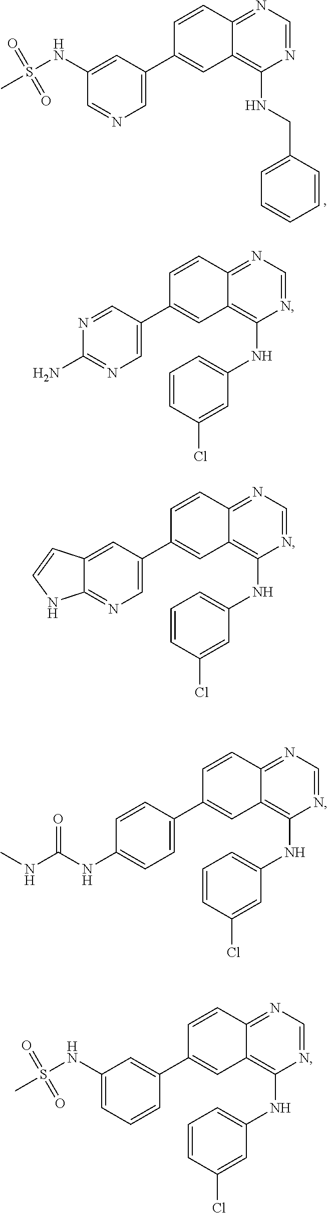 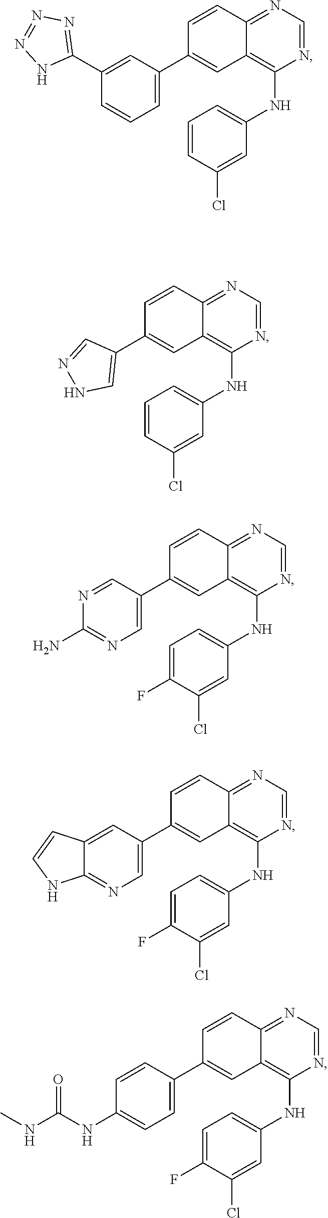 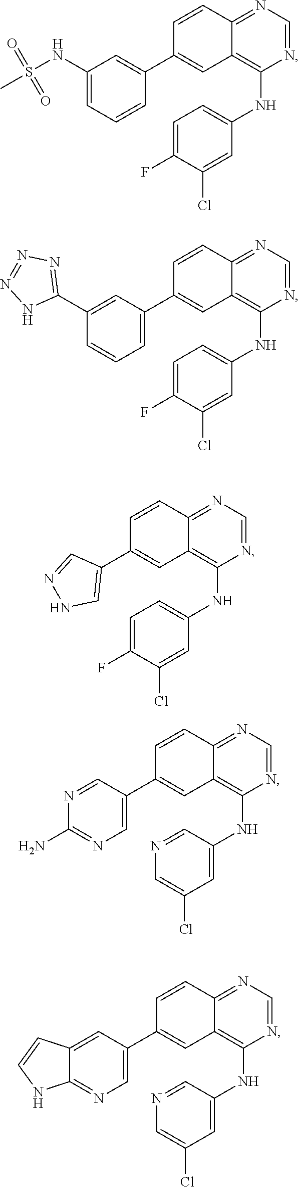 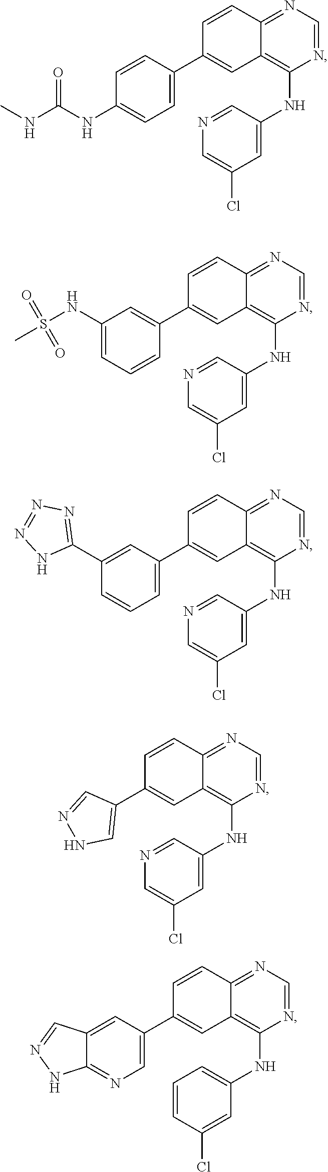 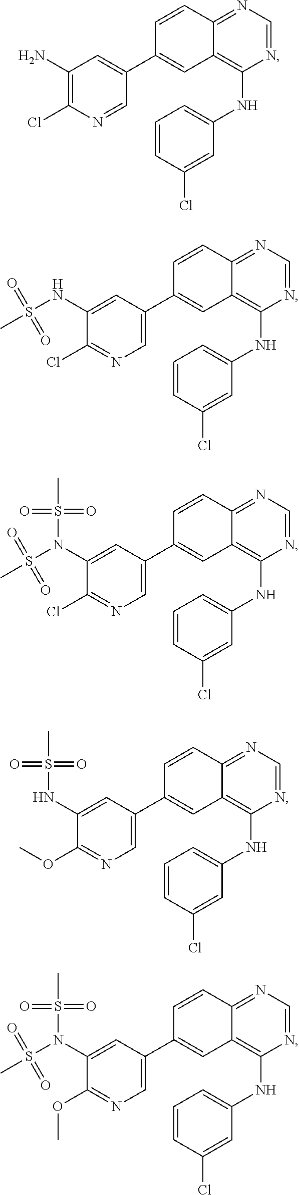 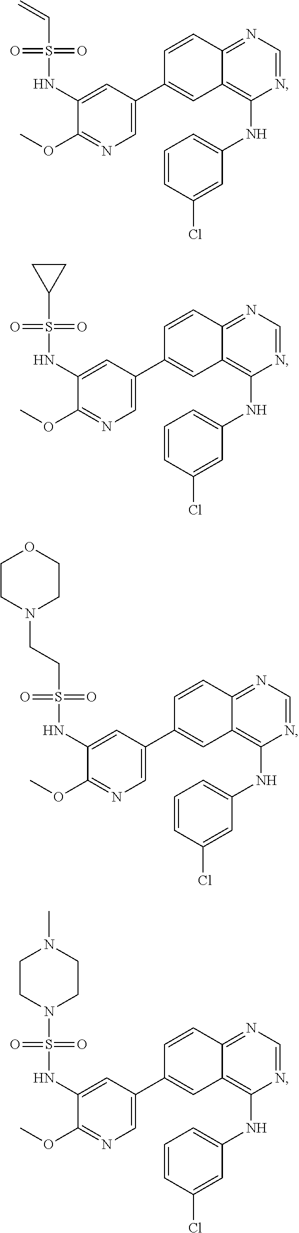 [SMILES: C=CS(=O)(=O)Nc1cc(-c2ccc3ncnc(Nc4cccc(Cl)c4)c3c2)cnc1OC ; COc1ncc(-c2ccc3ncnc(Nc4cccc(Cl)c4)c3c2)cc1NS(=O)(=O)C1CC1 ; COc1ncc(-c2ccc3ncnc(Nc4cccc(Cl)c4)c3c2)cc1NS(=O)(=O)CCN1CCOCC1 ; COc1ncc(-c2ccc3ncnc(Nc4cccc(Cl)c4)c3c2)cc1NS(=O)(=O)N1CCN(C)CC1] 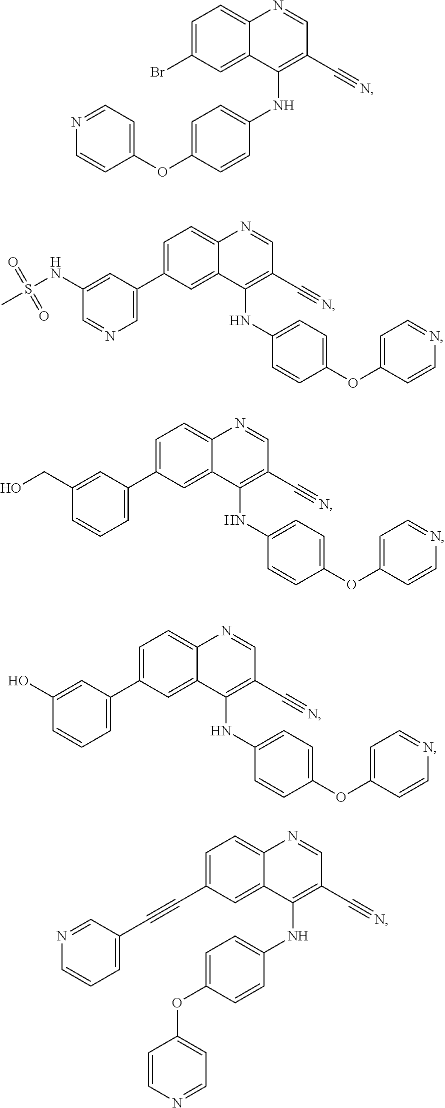 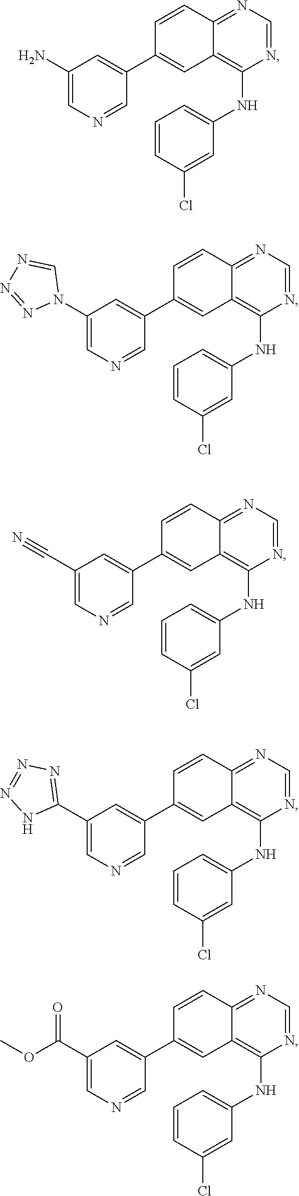 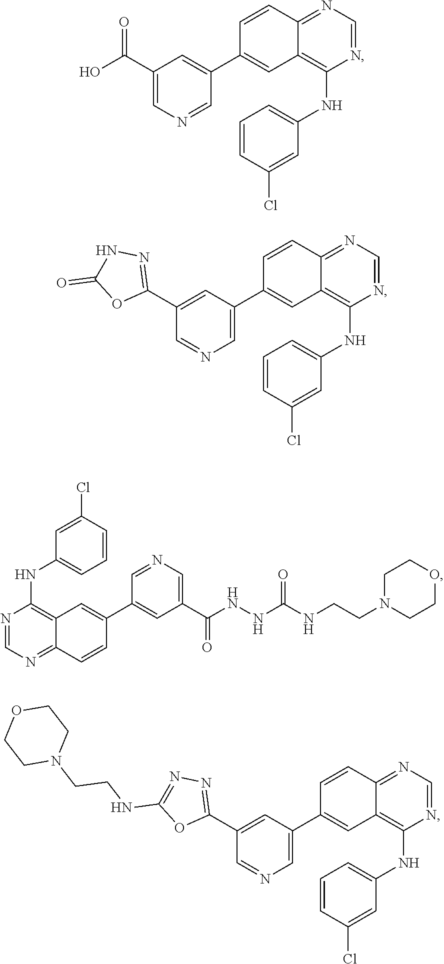 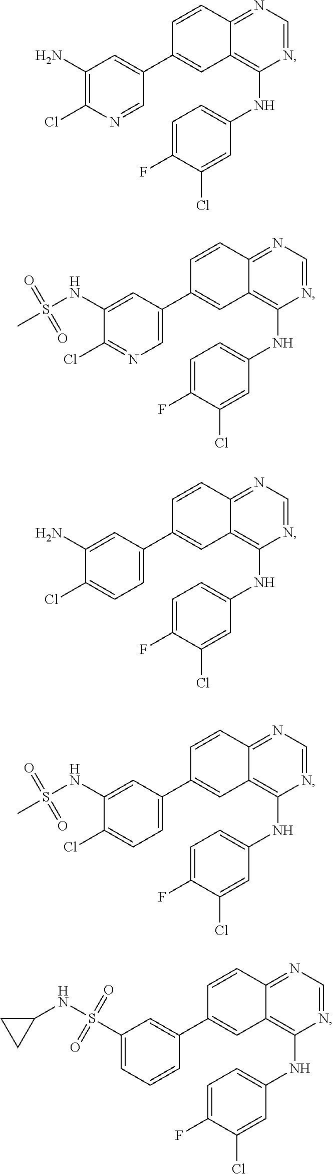 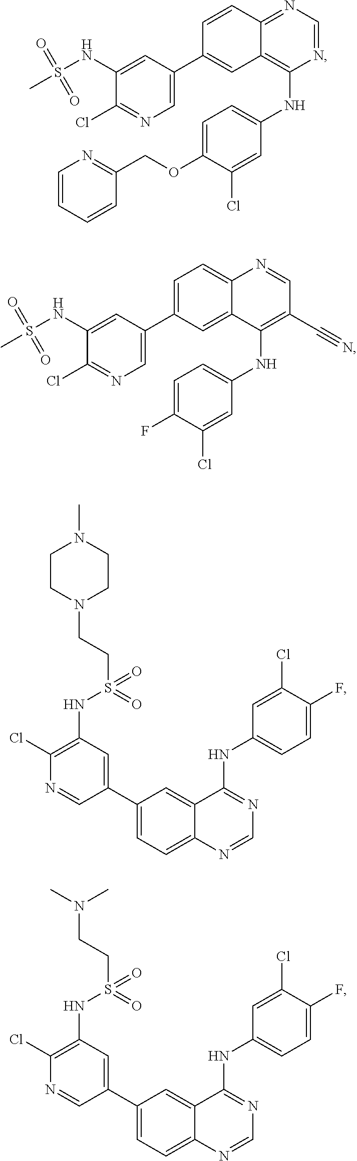 [SMILES: CS(=O)(=O)Nc1cc(-c2ccc3ncnc(Nc4ccc(OCc5ccccn5)c(Cl)c4)c3c2)cnc1Cl ; CS(=O)(=O)Nc1cc(-c2ccc3ncc(C#N)c(Nc4ccc(F)c(Cl)c4)c3c2)cnc1Cl ; CN1CCN(CCS(=O)(=O)Nc2cc(-c3ccc4ncnc(Nc5ccc(F)c(Cl)c5)c4c3)cnc2Cl)CC1 ; CN(C)CCS(=O)(=O)Nc1cc(-c2ccc3ncnc(Nc4ccc(F)c(Cl)c4)c3c2)cnc1Cl] 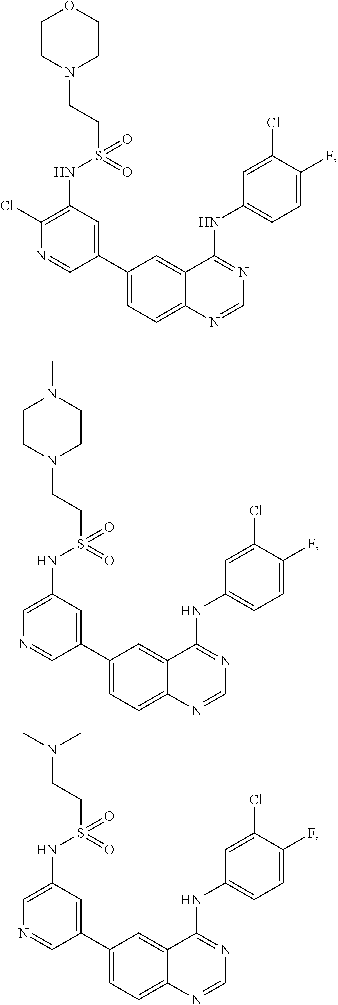 [SMILES: O=S(=O)(CCN1CCOCC1)Nc1cc(-c2ccc3ncnc(Nc4ccc(F)c(Cl)c4)c3c2)cnc1Cl ; CN1CCN(CCS(=O)(=O)Nc2cncc(-c3ccc4ncnc(Nc5ccc(F)c(Cl)c5)c4c3)c2)CC1 ; CN(C)CCS(=O)(=O)Nc1cncc(-c2ccc3ncnc(Nc4ccc(F)c(Cl)c4)c3c2)c1] 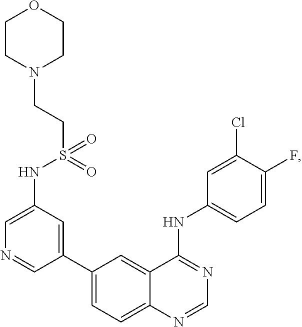 [SMILES: O=S(=O)(CCN1CCOCC1)Nc1cncc(-c2ccc3ncnc(Nc4ccc(F)c(Cl)c4)c3c2)c1]
or pharmaceutically acceptable salts thereof.

55. The method of claim 54, wherein the compound of Formula I or Formula Ia is 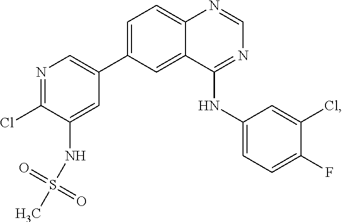 [SMILES: CS(=O)(=O)Nc1cc(-c2ccc3ncnc(Nc4ccc(F)c(Cl)c4)c3c2)cnc1Cl]
or a pharmaceutically acceptable salt thereof.

56. The method of claim 55, comprising the compound: 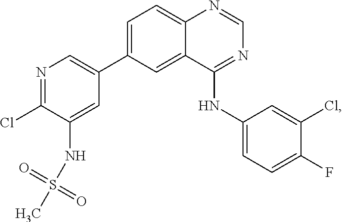 [SMILES: CS(=O)(=O)Nc1cc(-c2ccc3ncnc(Nc4ccc(F)c(Cl)c4)c3c2)cnc1Cl]
or a pharmaceutically acceptable salt thereof, and one or more immune checkpoint modulators selected from: Tremelimumab, Abatacept, AK104, REGN2810 (Cemiplimab), Nivolumab, Pembrolizumab, Sintilimab (IBI308), Tislelizumab (BGB-A317), SHR-1210 (Camrelizumab), Spartalizumab (PDR001), JS001, TSR-042, JNJ-63723283, BCD-100, TORIPALIMAB, Avelumab, Atezolizumab, TQB2450, KN035, CS1001, and Durvalumab (MEDI4736).
